(19) United States
(12) Patent Application Publication
(10) Pub. No.: US 2011/0230372 A1
(43) Pub. Date: Sep. 22, 2011

(54) GENE EXPRESSION CLASSIFIERS FOR RELAPSE FREE SURVIVAL AND MINIMAL RESIDUAL DISEASE IMPROVE RISK CLASSIFICATION AND OUTCOME PREDICTION IN PEDIATRIC B-PRECURSOR ACUTE LYMPHOBLASTIC LEUKEMIA

(75) Inventors: Cheryl L. Willman, Albuquerque, NM (US); Richard Harvey, Placitas, NM (US); Huining Kang, Albuquerque, NM (US); Edward Bedrick, Albuquerque, NM (US); Xuefei Wang, Creve Coeur, MO (US); Susan R. Atlas, Albuquerque, NM (US); I-Ming Chen, Albuquerque, NM (US)

(73) Assignee: STC UNM

(21) Appl. No.: 12/998,474

(22) PCT Filed: Nov. 16, 2009
(86) PCT No.: PCT/US2009/006117

§ 371 Date: Jun. 6, 2011

Related U.S. Application Data
(60) Provisional application No. 61/279,281, filed on Oct. 16, 2009.
(60) Provisional application No. 61/199,342, filed on Nov. 14, 2008.

(51) Int. Cl. C40B 40/06; C12Q 1/68; G01N 33/566; C12Q 1/44; C12Q 1/527; C12Q 1/48; G01N 33/573

(52) U.S. Cl. 506/16; 435/6.18; 435/6.17; 435/6.16; 435/6.1; 435/7.92; 435/19; 435/4; 435/15; 435/6.13; 435/7.1; 436/501

(57) ABSTRACT

The present invention relates to the identification of genetic markers patients with leukemia, especially including acute lymphoblastic leukemia (ALL) at high risk for relapse, especially high risk B-precursor acute lymphoblastic leukemia (B-ALL) and associated methods and their relationship to therapeutic outcome. The present invention also relates to diagnostic, prognostic and related methods using these genetic markers, as well as kits which provide microchips and/or immunoreagents for performing analysis on leukemia patients.

### RELATED APPLICATIONS

This application claims the benefit of priority of U.S. provisional applications US61/199,342, filed Nov. 14, 2008, entitled “Gene Expression Classifiers for Minimal Residual Disease and Relapse Free Survival Improve Outcome Prediction and Risk Classification and US61/279,281, filed Oct. 16, 2009, entitled “Gene Expression Classifiers for Relapse Free Survival and Minimal Residual Disease Improve Risk Classification and Outcome Prediction in Pediatric B-Precursor Acute Lymphoblastic Leukemia”, the entire contents of said applications being incorporated by reference in their entirety herein.

The present invention was made with support under one or more grants from the National Institutes of Health grant no. NIH NCI U01 CA114762, NCI U10 CA98543, NCI U10 CA98543, NCI P30 CA118100, U01 GM61393, U01GM61374 and U24 CA114766. Consequently, the government retains rights in the present invention.

### FIELD OF THE INVENTION

The present invention relates to the identification of genetic markers patients with leukemia, especially including acute lymphoblastic leukemia (ALL) at high risk for relapse, especially high risk B-precursor acute lymphoblastic leukemia (B-ALL) and associated methods and their relationship to therapeutic outcome. The present invention also relates to diagnostic, prognostic and related methods using these genetic markers, as well as kits which provide microchips and/or immunoreagents for performing analysis on leukemia patients.

### BACKGROUND OF THE INVENTION

Leukemia is the most common childhood malignancy in the United States. Approximately 3,500 cases of acute leukemia are diagnosed each year in the U.S. in children less than 20 years of age. The large majority (>70%) of these cases are acute lymphoblastic leukemias (ALL) and the remainder acute myeloid leukemias (AML). The outcome for children with ALL has improved dramatically over the past three decades, but despite significant progress in treatment, a large group of children with ALL develop recurrent disease. Conversely, another group of children who now receive dose intensification are likely “over-treated” and may well be cured using less intensive regimens resulting in fewer toxicities and long term side effects. Thus, a major challenge for the treatment of children with ALL in the next decade or so is to improve and refine ALL diagnosis and risk classification schemes in order to precisely tailor therapeutic approaches to the biology of the tumor and the genotype of the host.

Leukemia in the first 12 months of life (referred to as infant leukemia) is extremely rare in the United States, with about 150 infants diagnosed each year. There are several clinical and genetic factors that distinguish infant leukemia from acute leukemias that occur in older children. First, while the percentage of acute lymphoblastic leukemia (ALL) cases is far more frequent (approximately five times) than acute myeloid leukemia in children from ages 1-15 years, the frequency of ALL and AML in infants less than one year of age is approximately equivalent. Secondly, in contrast to the extensive heterogeneity in cytogenetic abnormalities and chromosomal rearrangements in older children with ALL and AML, nearly 60% of acute leukemias in infants have chromosomal rearrangements involving the MLL gene (for Mixed Lineage Leukemia) on chromosome 11q23. MLL translocations characterize a subset of human acute leukemias with a decidedly unfavorable prognosis. Current estimates suggest that about 60% of infants with AML and about 80% of infants with ALL have a chromosomal rearrangement involving MLL abnormality in their leukemia cells. Whether hematopoietic cells in infants are more likely to undergo chromosomal rearrangements involving 11q13 or whether this 11q13 rearrangement reflects a unique environmental exposure or genetic susceptibility remains to be determined.

The modern classification of acute leukemias in children and adults relies principally on morphologic and cytochemical features that may be useful in distinguishing AML from ALL, changes in the expression of cell surface antigens as a precursor cell differentiates, and the presence of specific recurrent cytogenetic or chromosomal rearrangements in leukemic cells. Using monoclonal antibodies, cell surface antigens (called clusters of differentiation (CD)) can be identified in cell populations; leukemias can be accurately classified by this means (immunophenotyping). By immunophenotyping, it is possible to classify ALL into the major categories of “common—CD10+ B-cell precursor” (around 50%), “pre-B” (around 25%), “T” (around 15%), “null” (around 9%) and “B” cell ALL (around 1%). All forms other than T-ALL are considered to be derived from some stage of B-precursor cell, and “null” ALL is sometimes referred to as “early B-precursor” ALL.

TABLE 1A
Recurrent Genetic Subtypes of B and T Cell ALL

| | Associated Genetic | Frequency in | Risk |
|---|---|---|---|
| Subtype | Abnormalities | Children | Category |
| B- | Hyperdiploid DNA | 25% of B | Low |
| Precursor | Content; Trisomies of | Precursor Cases | |
| ALL | Chromosomes 4, 10, 17 | | |
| | t(12; 21)(p13; q22): | 28% of B | Low |
| | TEL/AML1 | Precursor Cases | |
| | 11q23/MLL | 4% of B Precursor | High |
| | Rearrangements; | Cases; >80% of | |
| | particularly | Infant ALL | |
| | t(4; 11)(q21; q23) | | |
| | t(1; 19)9q23; p13) - | 6% of B Precursor | High |
| | E2A/PBX1 | Cases | |
| | t(9; 22)(q34; q11): | 2% of B Precursor | Very High |
| | BCR/ABL | Cases | |
| | Hypodiploidy | Relatively Rare | Very High |
| B-ALL | t(8; 14)(q24; q32) - | 5% of all B | High |
| | IgH/MYC | lineage ALL cases | |
| T-ALL | Numerous translocations | 7% of ALL cases | Not |
| | involving the TCR αβ | | Clearly |
| | (7q35) or TCR γδ (14q11) | | Defined |
| | loci | | |

Current risk classification schemes for ALL in children from 1-18 years of age use clinical and laboratory parameters such as patient age, initial white blood cell count, and the presence of specific ALL-associated cytogenetic abnormalities to stratify patients into “low,” “standard,” “high,” and “very high” risk categories. National Cancer Institute (NCI) risk criteria are first applied to all children with ALL, dividing them into “NCI standard risk” (age 1.00-9.99 years, WBC <50,000) and “NCI high risk” (age >10 years, WBC >50,000) based on age and initial white blood cell count (WBC) at disease presentation. In addition to these general NCI risk criteria, classic cytogenetic analysis and molecular genetic detection of frequently recurring cytogenetic abnormalities have been used to stratify ALL patients more precisely into “low,” “standard,” “high,” and “very high” risk categories. Table 1A shows the 4-year event free survival (EFS) projected for each of these groups.

Children with “low risk” disease (22% of all B precursor ALL cases) are defined as having standard NCI risk criteria, the presence of low risk cytogenetic abnormalities (t(12;21)/TEL; AML1 or trisomies of chromosomes 4 and 10), and a rapid early clearance of bone marrow blasts during induction chemotherapy. Children with “standard risk” disease (50% of ALL cases) are NCI standard risk without “low risk” or unfavorable cytogenetic features, or, are children with low risk cytogenetic features who have NCI high risk criteria or slow clearance of blasts during induction. Although therapeutic intensification has yielded significant improvements in outcome in the low and standard risk groups of ALL, it is likely that a significant number of these children are currently “over-treated” and could be cured with less intensive regimens resulting in fewer toxicities and long term side effects. Conversely, a significant number of children even in these good risk categories still relapse and a precise means to prospectively identify them has remained elusive. Nearly 30% of children with ALL have “high” or “very high” risk disease, defined by NCI high risk criteria and the presence of specific cytogenetic abnormalities (such as t(1;19), t(9;22) or hypodiploidy) (Table 1); again, precise measures to distinguish children more prone to relapse in this heterogeneous group have not been established.

Despite these efforts, current diagnosis and risk classification schemes remain imprecise. Children with ALL are more prone to relapse and require more intensive approaches than children with low risk disease who could be cured with less intensive therapies are not adequately predicted by current classification schemes and are distributed among all currently defined risk groups. Although pre-treatment clinical and tumor genetic stratification of patients has generally improved outcomes by optimizing therapy, variability in clinical course continues to exist among individuals within a single risk group and even among those with similar prognostic features. In fact, the most significant prognostic factors in childhood ALL explain no more than 4% of the variability in prognosis, suggesting that yet undiscovered molecular mechanisms dictate clinical behavior (Donadieu et al., Br J Haematol, 102:729-739, 1998). A precise means to prospectively identify such children has remained elusive.

With the advent of modem combination chemotherapy and transplantation, significant advances have been made in the treatment of the acute leukemias, particularly in children. Yet despite these advances, a large percentage of the thousands of children and adults diagnosed with leukemia each year will ultimately die of resistant or relapsed disease. The therapeutic advances that have been achieved in the acute leukemias, particularly in pediatric acute lymphoblastic leukemia (ALL), have come in part through the development of detailed risk classification schemes based on clinical features, the presence or absence of specific cytogenetic or molecular genetic abnormalities, and measures of early therapeutic response that may be used to tailor the choice of therapy and its intensity to a patient's relapse risk. Yet current risk classification schemes do not fully reflect the tremendous molecular heterogeneity of the acute leukemias and do not precisely identify those patients who are more prone to relapse, those who might be cured with less intensive regimens resulting in fewer toxicities and long term side effects, or those who will respond to newer targeted therapeutic agents. It has thus been the inventors' hypothesis that large scale genomic and proteomic technologies that measure global patterns of gene expression in leukemic cells will yield systematic profiles that can be used to improve outcome prediction, risk classification, and therapeutic targeting in the acute leukemias. The present inventors have worked with retrospective patient cohorts from which they derived rigorously cross-validated gene expression profiles. Over the years, the inventors have built highly collaborative multidisciplinary laboratory, statistical, and computational teams; developed reproducible and sensitive methods for performing gene expression arrays; designed data warehouses for storage of large gene expression datasets fully annotated with clinical, outcome, and experimental information; and developed and applied robust statistical and computational methods and novel visualization tools for array data analysis.

The major scientific challenge in pediatric ALL is to improve risk classification schemes and outcome prediction in order to: 1) identify those children who are most likely to relapse who require intensive or novel regimens for cure; and 2) identify those children who can be cured with less intensive regimens with fewer toxicities and long term side effects.

### BRIEF DESCRIPTION OF THE FIGURES

FIG. 1 shows the performance of the 42 Probe Set (38-Gene) Gene Expression Classifier for Prediction of Relapse-Free Survival (RFS). A and B. Kaplan-Meier survival estimates of RFS in the full cohort of 207 patients (Panel A) and in the low vs. high risk groups distinguished with the gene expression classifier for RFS (Panel B). HR is the hazard ratio estimated using Cox-regression. C. A gene expression heatmap is shown with the rows representing the 42 probe sets (containing 38 unique genes) composing the gene expression classifier for RFS. The columns represent patient samples sorted from left to right by time to relapse or last follow up. Red: high expression relative to the mean; green: low expression relative to the mean. The column labels R or C indicate whether the patients relapsed or were censored, respectively.

FIG. 2 shows the Kaplan-Meier Estimates of Relapse-free Survival (RFS) Based on the Gene Expression Classifier for RFS and End-Induction (Day 29) Minimal Residual Disease (MRD). A. Day 29 flow cytometric measures of MRD separated patients into two groups with significantly different RFS. B. and C. After dividing patients by their end-induction flow MRD status, an independent effect of the gene expression classifier for RFS is observed among both the flow MRD-negative (<0.01% blasts) (Panel B) and flow MRD-positive (>0.01% blasts) (Panel C) patients. D and E. Combining the risk scores determined from the gene expression classifier and flow MRD yields four distinct outcome groups; the two discordant groups show no significant difference in RFS (P=0.572) and are therefore collapsed into an intermediate risk group for RFS prediction (Panel E). The hazard ratios (HR) and corresponding Pvalues are based on the Cox regression (medium risk vs. low risk, HR=3.73, P=0.001; high risk vs. medium risk, HR=2.27, P=0.002). The P-value reported in the lower left hand corner corresponds to the test for differences among all groups.

FIG. 3 shows the Kaplan-Meier Estimates of Relapse-free Survival (RFS) Based on the Gene Expression Classifier for RFS Modeled on High-Risk ALL Cases Lacking Known Recurring Cytogenetic 29 Abnormalities and End-Induction (Day 29) Minimal Residual Disease (MRD). A. The second gene expression classifier modeled only on those high-risk ALL cases (n=163) (Supplement Table S8) from the COG 9906 ALL cohort lacking recurring cytogenetic abnormalities resolves two distinct risk groups of patients with significantly different RFS. B. Day 29 flow MRD status separated these 163 ALL cases into two groups with significantly different RFS. C and D. After dividing patients by their end-induction flow MRD status, an independent effect of the gene expression classifier for RFS is observed among both the flow MRD-negative (<0.01% blasts) (Panel C) and flow MRD-positive (>0.01% blasts) (Panel D) patients. E and F. Combining the risk scores determined from the gene expression classifier and flow MRD yields four distinct outcome groups (Panel E); the two discordant groups show no significant difference in RFS and are therefore collapsed into an intermediate risk group for RFS prediction (Panel F). The hazard ratios (HR) and corresponding P-values are based on the Cox regression regression (high risk vs. intermediate risk, HR=2.26, P=0.0066; intermediate risk vs. low risk, HR=2.77, P=0.008). The P-value reported in the lower left hand corner corresponds to the test for differences among all groups.

FIG. 4 shows the Gene Expression Classifier for Prediction of End-Induction (Day 29) Flow MRD in Pretreatment Samples Combined with the Gene Expression Classifier for RFS. A. A receiver operating curve (ROC) shows the high accuracy of the 23 probe set MRD classifier (LOOCV error rate of 24.61%; sensitivity 71.64%, specificity 77.42%) in predicting MRD. The area under the ROC curve (0.80) is significantly greater than an uninformative ROC curve (0.5) (P<0.0001). B. Heatmap of 23 probe set predictor of MRD presented in rows (false discovery rate <0.0001%, SAM). The columns represent patient samples with positive or negative end-induction flow MRD while the rows are the specific predictor genes. Red: high expression relative to the mean; green: low expression relative to the mean. C. Kaplan-Meier estimates of relapse free survival (RFS) for the risk groups determined by combining the gene expression classifiers for RFS and MRD, analogous to FIG. 2E, with the gene expression predictor for MRD replacing day 29 flow MRD. The three risk groups have significantly different RFS (log rank test, P<0.0001).

FIG. 5 shows the Kaplan-Meier Estimates of Relapse-free Survival (RFS) using the Combined Gene Expression Classifiers for RFS and Minimal Residual Disease in an Independent Cohort of 84 Children with High-Risk ALL. A. The gene expression classifier for RFS separates children into low and high risk groups in an independent cohort of 84 children with high-risk ALL treated on COG Trial 1961.14,16 B. Application of the combined gene expression classifiers for RFS and MRD shows significant separation of three risk groups: low (47/84, 56%), intermediate (22/84, 26%) and high (15/84, 18%), similar to our initial cohort (FIG. 3C).

FIG. 6 shows Kaplan-Meier Estimates of Relapse Free Survival using the Combined Gene Expression Classifier for RFS and Flow Cytometric Measures of MRD in the Presence of Kinase Signatures, JAK Mutations, and IKAROS/IKZF1 Deletions. A and B. Application of the original 42 probe set (38 gene; Supplement Table S4) gene expression classifier for RFS combined with end-induction flow cytometric measures of MRD distinguishes two distinct risk groups in COG 9906 ALL patients with a kinase signatures (Panel A) and three risk groups in those patients lacking kinase signatures (Panel B). C and D. Application of the combined classifier also resolves two distinct and statistically significant risk groups in ALL patients with JAK mutations (Panel C) and in three risk groups in those patients lacking JAK mutations (Panel D). E and F. Application of the combined classifier distinguishes three risk groups with statistically significant RFS and patients with (Panel E) and without IKAROS/IKZF1 deletions. The hazard ratios (HR) and corresponding P-values are based on the Cox regression. The P-value reported in the lower left hand corner corresponds to the log rank test for differences among all groups.

FIG. 7 (Figure S1) shows the difference in Relapse-Free Survival (RFS) between Study Cohort (n=207) and Remaining Patients Registered to COG P9906 (n=65). Comparison of relapse free survival between those studied (n=207) and remaining COG P9906 patients not included in this cohort (n=65).

FIG. 8 (Figure S2) shows the Number of Genes (Probe Sets) with the Number of ‘Present’ Calls Exceeding a Specified Cutoff. Number of probe sets with number of ‘Present’ calls exceeding a specified cutoff (here, n=104, corresponding to 50% of n=207 patient samples analyzed. This yields 23,775 final probe sets for further analysis.)

FIG. 9 (Figure S3) shows the Likelihood Ratio Test Statistic as a Function of SPCA Threshold.

FIG. 10 (Figure S4) shows the Box plots of Cross-validation Error Rates for DLDA Model Predicting Day 29 MRD Status.

FIG. 11 (Figure S5) shows the Cross-validation Procedure for Determining the Best Model for Predicting RFS.

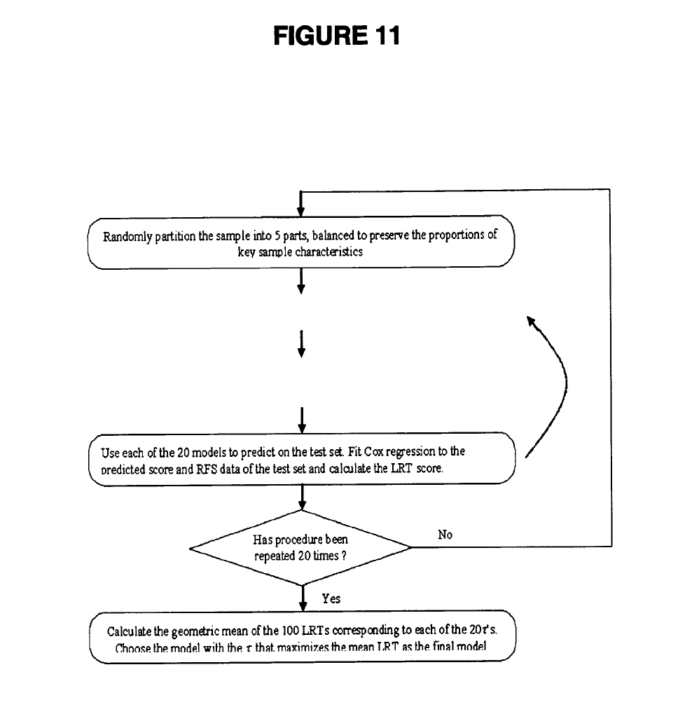

FIG. 12 (Figure S6) shows the Nested Cross-validation for Objective Prediction used in Significance Evaluation of the Gene Expression Risk Prediction Model.

FIG. 13 (Figure S7) shows the Cross-validation Procedure for Determining the Best Model for Predicting Day 29 MRD Status. Figure S7.

FIG. 14 (Figure S8) shows the Nested cross-validation for Objective Predictions used in Significance Evaluation of Gene Expression Risk Prediction Model for the 29 MRD Status.

FIG. 15 (Figure S9) shows the Likelihood Ratio Test Statistic as a Function of Gene Expression Classifier Threshold for RFS with t(1;19) Translocation and MLL Rearrangement Cases Removed.

FIG. 16 (Figure S10) shows Kaplan-Meier Estimates of Relapse-free Survival (RFS) Based on Gene Expression Classifier for RFS and Day 29 Minimal Residual Disease (MRD) Levels after Excluding t(1;19) Translocation and MLL Rearrangement Cases. These are presented in figures (A) through (F). A. The gene expression classifier separates patients into low and high risk groups with significantly different RFS. B. and C. After dividing patients by their end-induction flow MRD status, an independent effect of the gene expression classifier for RFS is observed among both the flow MRD-negative (<0.01% blasts) (Panel B) and flow MRD-positive (>0.01% blasts) (Panel C) patients. D. Combining the scores from the gene expression classifier for RFS and flow MRD yields three distinct outcome groups. The hazard ratio (HR) and corresponding p-value are based on the Cox regression. The p-value reported in the lower left hand corner corresponds to the test for differences among all groups.

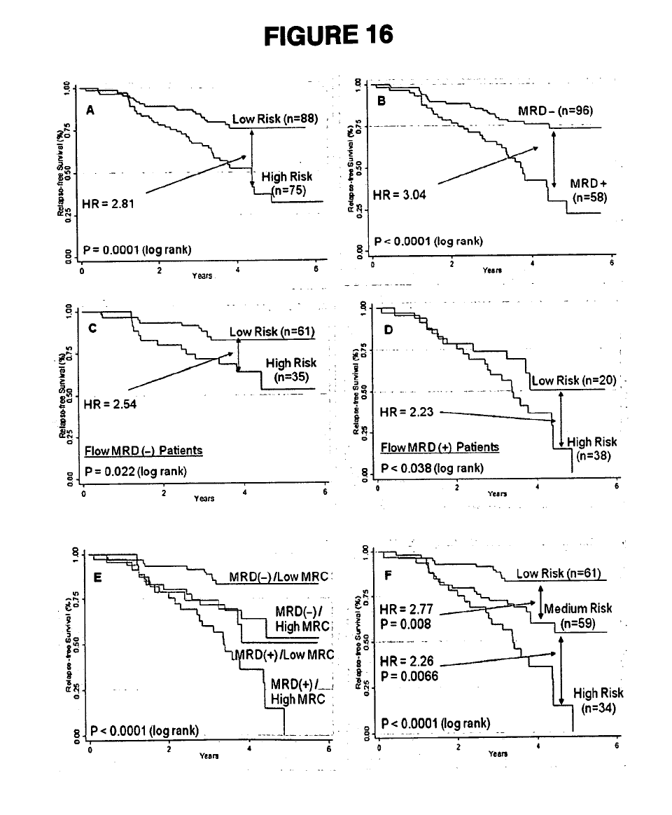

FIG. 17 shows Hierarchical Clustering Identifying 8 Cluster Groups in High Risk ALL. Hierarchical clustering using 254 genes (provided in Supplement, Table S7A) was used to identify clusters of patients with shared patterns of gene expression. (Rows: 207 P9906 patients; Columns: 254 Probe Sets). Shades of red depict expression levels higher than the median while green indicates levels lower than the median. The cluster groups are numbered and prefixed by their method of probe set selection: H=High CV, C=COPA and R=ROSE. Panel A. HC method for selection of probe sets. Panel B. COPA selection of probe sets. Panel C. ROSE selection of probe sets.

FIG. 18 shows Relapse-Free Survival in Gene Expression Cluster Groups. Relapse free-survival is shown for each of the High CV clusters (A), COPA clusters (B), and ROSE clusters (C). Only the H6, C6, and R6 clusters (curves shown in blue) have a significantly better outcome compared to the entire cohort (dense line), while the H8, C8, R8 clusters (curves shown in red) have a significantly poorer RFS. Hazard ratios and p-values are shown in the bottom left of each panel.

FIG. 19 shows Hierarchical Clustering Identifying Similar Clusters in a Second High Risk ALL Cohort. Hierarchical clustering using 167 probe sets (provided in Supplement, Table S7A) was used to identify clusters of patients with shared patterns of gene expression in CCG 1961. (Rows: 99 CCG 1961 patients; Columns: 167 Probe Sets). Shades of red depict expression levels higher than the median while green indicates levels lower than the median. The cluster groups are prefixed by their method of probe set selection: H=High CV, C=COPA and R=ROSE. Panel A. HC method for selection of probe sets. Panel B. COPA selection of probe sets. Panel C. ROSE selection of probe sets.

FIG. 20 shows Relapse-Free Survival in Second High Risk ALL Cohort. Relapse free-survival is shown for each of the High CV clusters (A), COPA clusters (B), and ROSE clusters (C). Only the C10 and R10 clusters (curves shown in blue) have a significantly better outcome compared to the entire cohort (dense line), while the H8, C8, R8 clusters (curves shown in red) have a significantly poorer RFS. Hazard ratios and p-values are shown in the bottom left of each panel.

FIG. 21 (Figure S1′) shows a comparison of relapse free survival between those studied (n=207) and remaining COG P9906 patients not included in this cohort (n=65).

FIG. 22 (Figure S2′) shows an example of probe set with outlier group at high end. Red line indicates signal intensities for all 207 patient samples for probe 212151_at. Vertical blue lines depict partitioning of samples into thirds. A least-squares curve fit is applied to the middle third of the samples and the resulting trend line is shown in yellow. Different sample groups are illustrated by the dashed lines at the top right. As shown by the double arrowed lines, the median value from each of these groups is compared to the trend line.

FIG. 23 (Figure S3′) shows a 3-D plot of cluster membership from different clustering methods. Each of the three clustering methods is shown on an axis: HC=hierarchical clusters, RC=ROSE/COPA clusters and Vx=VxInsight clusters. Cluster numbers are given across each axis with the exception of RC9, which represents cluster 2A.

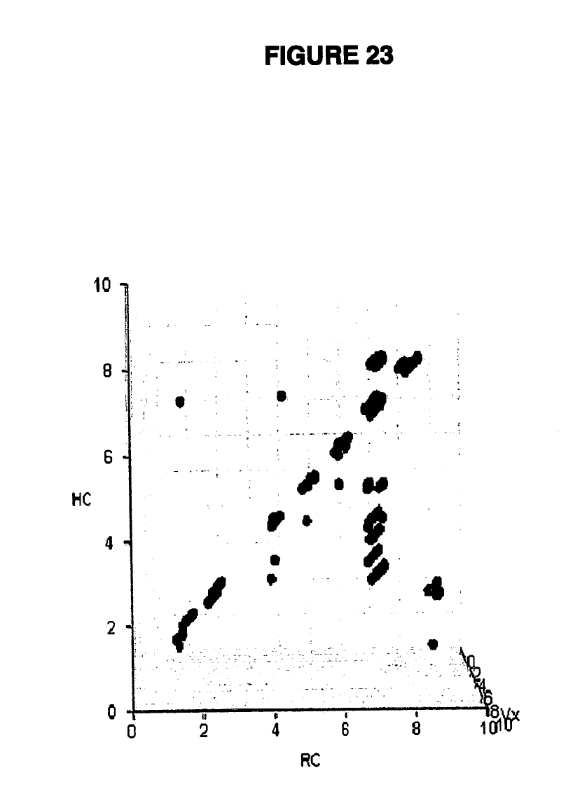

FIG. 24 shows the survival of IKZF1-positive patients in R8 compared to not-R8. IKZF1-positive patients were divided into those in cluster 8 (red line) and those in other clusters (black line). The p-value and hazard ratio for this comparison are given in the lower left panel.

### BRIEF DESCRIPTION OF THE INVENTION

Accurate risk stratification constitutes the fundamental paradigm of treatment in acute lymphoblastic leukemia (ALL), allowing the intensity of therapy to be tailored to the patient's risk of relapse. The present invention evaluates a gene expression profile and identifies prognostic genes of cancers, in particular leukemia, more particularly high risk B-precursor acute lymphoblastic leukemia (B-ALL), including high risk pediatric acute lymphoblastic leukemia. The present invention provides a method of determining the existence of high risk B-precursor ALL in a patient and predicting therapeutic outcome of that patient, especially a pediatric patient. The method comprises the steps of first establishing the threshold value of at least (2) or three (3) prognostic genes of high risk B-ALL, or four (4) prognostic genes, at least five (5) prognostic genes, at least 6, at least 7, at least 8, at least 9, at least 10, at least 11, at least 12, at least 13, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 or up to 30 or more prognostic genes which are described in the present specification, especially Table 1P and 1Q (see below, pages 14-17). Table 1P genes include the following 31 genes (gene products): BMPR1B (bone morphogenic receptor type 1B); BTG3 (B-cell translocation gene 3, also BTG family member 3); C14orf32 (chromosome 14 open reading frame 32); C8orf38 (Chromosome 8 open reading frame 38); CD2 (CD2 molecule); CDC42EP3 (CDC42 effector protein (Rho GTPase binding) 3); CHST2 (carbohydrate (N-acetylglucosamine-6-O) sulfotransferase 2); CTGF (connective tissue growth factor); DDX21 (DEAD (Asp-Glu-Ala-Asp) box polypeptide 21); DKFZP761M1511 (hypothetical protein DKFZP761M1511); ECM1 (extracellular matrix protein 1); FMNL2 (formin-like 2); GRAMD1C (GRAM domain containing 1C); IGJ (immunoglobulin J polypeptide); LDB3 (LIM domain binding 3); LOC400581 (GRB2-related adaptor protein-like); LRRC62 (leucine rich repeat containing 62); MDFIC (MyoD family inhibitor domain containing); MGC12916 (hypothetical protein MGC12916); NFKBIB (nuclear factor of kappa light polypeptide gene enhancer in B-cells inhibitor, beta); NR4A3 (nuclear receptor subfamily 4, group A, member 3); NT5E (5′-nucleotidase, ecto (CD73)); PON2 (paraoxonase 2); RGS1 (regulator of G-protein signalling 1); RGS2 (regulator of G-protein signalling 2, 24 kDa); SCHIP1 (schwannomin interacting protein 1); SEMA6A (sema domain, transmembrane domain (TM), and cytoplasmic domain, (semaphorin) 6A); TSPAN7 (tetraspanin 7); TTYH2 (tweety homolog 2 (Drosophila)); UBE2E3 (ubiquitin-conjugating enzyme E2E 3 (UBC4/5 homolog, yeast)) and VPREB1 (pre-B lymphocyte gene 1). Of the above genes/gene products (31) the following are high risk genes (gene products): BMPR1B; C8orf38; CDC42EP3; CTGF; DKFZP761M1511; ECM1; GRAMD1C; IGJ; LDB3; LOC400581; LRRC62; MDFIC; NT5E; PON2; SCHIP1; SEMA6A; TSPAN7; and TTYH2. Of these 31 genes, the following are low risk genes (gene products): BTG3; C14orf32; CD2; CHST2; DDX21; FMNL2; MGC12916; NFKBIB; NR4A3; RGS1; RGS2; UBE2E3 and VPREB1. It is noted that the gene product AGAP1 (Arf GAP with GTP-binding protein-like, ANK repeat and PH domains, also referred to as CENTG2) may also be added to this list for analysis in order to enhance diagnosis and evaluation of the patient and/or therapeutic agent.

Preferred table 1P genes to be measured include the following 8 genes products: BMPR1B; CTGF; IGJ; LDB3; PON2; RGS2; SCHIP1 and SEMA6A. Of these genes (gene products), BMPR1B; CTGF; IGJ; LDB3; PON2; SCHIP1 and SEMA6A are “high risk”, i.e., when overexpressed are predictive of an unfavorable therapeutic outcome (relapse, unsuccessful therapy) of the patient. One gene (gene product) within this group, RGS2, when overexpressed, is predictive of therapeutic success (remission, favorable therapeutic outcome). At least 2 or 3 genes, preferably at least 4 or 5 genes, at least 6 at least 7 or 8 of these genes within this smaller group are measured to provide a predictive outcome of therapy. It is noted that overexpression of a high risk gene (gene product) will be predictive of an unfavorable outcome; whereas the underexpression of a high risk gene will be (somewhat) predictive of a favorable outcome. It is also noted that the overexpression of a low risk gene (gene product) will be predictive of a favorable therapeutic outcome, whereas the underexpression of a low risk gene (gene product) will be predictive of an unfavorable therapeutic outcome.

Table 1Q genes include the following genes (gene products): BMPR1B (bone morphogenic receptor type 1B); BTBD11 (BTB (POZ) domain containing 11); C21orf87 (chromosome 21 open reading frame 87); CA6 (carbonic anhydrase VI); CDC42EP3 (CDC42 effector protein (Rho GTPase binding) 3); CKMT2 (creatine kinase, mitochondrial 2 (sarcomeric)); CRLF2 (cytokine receptor-like factor 2); CTGF (connective tissue growth factor); DIP2A (DIP2 disco-interacting protein 2 homolog A (Drosophila)); GIMAP6 (GTPase, IMAP family member 6); GPR110 (G protein-coupled receptor 110); IGFBP6 (insulin-like growth factor binding protein 6); IGJ (immunoglobulin J polypeptide); K1F1C (kinesin family member 1C); LDB3 (LIM domain binding 3); LOC391849 (Homo sapiens similar to neuralized 1); LOC650794 (Similar to FRAS1 related extracellular matrix protein 2 precursor (ECM3 homolog)); MUC4 (mucin 4, cell surface associated); NRXN3 (neurexin 3); PON2 (paraoxonase 2); RGS2 (regulator of G-protein signalling 2, 24 kDa); RGS3 (Regulator of G-protein signalling 3); SCHIP1 (schwannomin interacting protein 1); SCRN3 (secernin 3); SEMA6A (sema domain, transmembrane domain (TM), and cytoplasmic domain, (semaphorin) 6A) and ZBTB16 (Zinc finger and BTB domain containing 16). Of these 27 genes (gene products), the following are high risk: BMPR1B; BTBD11; C21orf87; CA6; CDC42EP3; CKMT2; CRLF2; CTGF; DIP2A; GIMAP6; GPR110; IGFBP6; IGJ; K1F1C; LDB3; LOC391849; LOC650794; MUC4; NRXN3; PON2; RGS3; SCHIP1; SCRN3; SEMA6A and ZBTB16. The following gene (gene product) is low risk: RGS2.

Preferred table 1Q (see below) genes to be measured include the following 11 genes products: BMPR1B; CA6; CRLF2; GPR110; IGJ; LDB3; MUC4; NRXN3; PON2; RGS2 and SEMA6A. At least 2 or 3 genes, preferably at least 4 or 5 genes, at least 6 at least 7, at least 8, at least 9, at least 10 or 11 of these genes are measured to provide a predictive outcome of therapy. A preferred list obtained from the above list of 11 genes includes BMPR1B; CA6; CRLF2; GPR110; IGJ; LDB3; MUE4; PON2 and RGS2. Preferred gene products within this list include CA6, IGJ, MUC4, GPR110, PON2, CRLF2 and optionally RGS2. CRLF2 is preferably included as a gene product in the most preferred list. It is noted that overexpression of a high risk gene (gene product) will be predictive of an unfavorable outcome; whereas the underexpression of a high risk gene will be (somewhat) predictive of a favorable outcome. It is also noted that the overexpression of a low risk gene (gene product) will be predictive of a favorable therapeutic outcome (remission), whereas the underexpression of a low risk gene (gene product) will be predictive of an unfavorable therapeutic outcome. Also noted is the fact that the gene products AGAP-1 (Arf GAP with GTP-binding protein-like, ANK repeat and PH domains, also CENTG2) and/or PCDH17 (Protocadherin-17) may also be used (analyzed) in the invention (in addition to Table 1P and/or Table 1Q gene products, including the preferred gene product lists from each of these Tables) to promote the accuracy of diagnosis and related methods.

TABLE 1P

| | | Overlap | | | |
|---|---|---|---|---|---|
| Rank | High => | with 54K | Probe set ID | Gene Symbol | Gene Description |
| 1 | High Risk | Yes | 242579_at | BMPR1B | Transcribed locus |
| 10 | High Risk | Yes | 232539_at | — | MRNA; cDNA DKFZp761H1023 (from clone |
| | | | | | DKFZp761H1023) |
| 18 | High Risk | | 236750_at | — | Transcribed locus |
| 19 | High Risk | | 215617_at | — | CDNA FLJ11754 fis, clone HEMBA1005588 |
| 25 | High Risk | | 244280_at | — | Homo sapiens, clone IMAGE: 5583725, mRNA |
| 26 | High Risk | | 215479_at | — | CDNA FLJ20780 fis, clone COL04256 |
| 31 | Low Risk | | 238623_at | — | CDNA FLJ37310 fis, clone BRAMY2016706 |
| 39 | Low Risk | | 244623_at | — | Transcribed locus |
| 24 | Low Risk | | 213134_x_at | BTG3 | BTG family, member 3 |
| 34 | Low Risk | | 212497_at | C14orf32 | chromosome 14 open reading frame 32 |
| 20 | High Risk | | 236766_at | C8orf38 | Chromosome 8 open reading frame 38 |
| 27 | Low Risk | | 205831_at | CD2 | CD2 molecule |
| 6 | High Risk | Yes | 209288_s_at | CDC42EP3 | CDC42 effector protein (Rho GTPase binding) |
| 41 | Low Risk | | 203921_at | CHST2 | carbohydrate (N-acetylglucosamine-6-O) |
| | | | | | sulfotransferase 2 |
| 12 | High Risk | Yes | 209101_at | CTGF | connective tissue growth factor |
| 30 | Low Risk | | 224654_at | DDX21 | DEAD (Asp-Glu-Ala-Asp) box polypeptide 21 |
| 36 | Low Risk | | 208152_s_at | DDX21 | DEAD (Asp-Glu-Ala-Asp) box polypeptide 21 |
| 14 | High Risk | | 225355_at | DKFZP761M1511 | hypothetical protein DKFZP761M1511 |
| 16 | High Risk | | 209365_s_at | ECM1 | extracellular matrix protein 1 |
| 33 | Low Risk | | 226184_at | FMNL2 | formin-like 2 |
| 13 | High Risk | | 219313_at | GRAMD1C | GRAM domain containing 1C |
| 11 | High Risk | Yes | 212592_at | IGJ | Immunoglobulin J polypeptide, linker protein |
| | | | | | for immunoglobulin alpha and mu polypeptide |
| 3 | High Risk | Yes | 213371_at | LDB3 | LIM domain binding 3 |
| 42 | High Risk | | 1560524_at | LOC400581 | GRB2-related adaptor protein-like |
| 38 | High Risk | | 1559072_a_at | LRRC62 | leucine rich repeat containing 62 |
| 28 | High Risk | | 211675_s_at | MDFIC | MyoD family inhibitor domain containing |
| 40 | Low Risk | | 224507_s_at | MGC12916 | hypothetical protein MGC12916 |
| 15 | Low Risk | | 228388_at | NFKBIB | nuclear factor of kappa light polypeptide gene |
| | | | | | enhancer in B-cells inhibitor, beta |
| 23 | Low Risk | | 209959_at | NR4A3 | nuclear receptor subfamily 4, group A, member 3 |
| 29 | Low Risk | | 207978_s_at | NR4A3 | nuclear receptor subfamily 4, group A, member 3 |
| 21 | High Risk | | 203939_at | NT5E | 5′-nucleotidase, ecto (CD73) |
| 4 | High Risk | Yes | 210830_s_at | PON2 | paraoxonase 2 |
| 5 | High Risk | Yes | 201876_at | PON2 | paraoxonase 2 |
| 22 | Low Risk | | 216834_at | RGS1 | regulator of G-protein signalling 1 |
| 2 | Low Risk | Yes | 202388 at | RGS2 | regulator of G-protein signalling 2, 24 kDa |
| 9 | High Risk | Yes | 204030_s_at | SCHIP1 | schwannomin interacting protein 1 |
| 7 | High Risk | Yes | 215028_at | SEMA6A | sema domain, transmembrane domain (TM), |
| | | | | | and cytoplasmic domain, (semaphorin) 6A |
| 8 | High Risk | Yes | 223449_at | SEMA6A | sema domain, transmembrane domain (TM), |
| | | | | | and cytoplasmic domain, (semaphorin) 6A |
| 32 | High Risk | | 202242_at | TSPAN7 | tetraspanin 7 |
| 17 | High Risk | | 223741_s_at | TTYH2 | tweety homolog 2 (Drosophila) |
| 37 | Low Risk | | 210024_s_at | UBE2E3 | ubiquitin-conjugating enzyme E2E 3 (UBC4/5 |
| | | | | | homolog, yeast) |
| 35 | Low Risk | | 221349_at | VPREB1 | pre-B lymphocyte gene 1 |

TABLE 1Q

| Rank | High => | Probe Set ID | Gene Symbol | Gene Description |
|---|---|---|---|---|
| 1 | High Risk | 236489_at | — | Transcribed locus |
| 8 | High Risk | 242579_at | BMPR1B | Transcribed locus |
| 19 | High Risk | 229975_at | — | Transcribed locus |
| 34 | High Risk | 232539_at | — | MRNA; cDNA DKFZp761H1023 (from clone |
| | | | | DKFZp761H1023) |
| 24 | High Risk | 241295_at | BTBD11 | BTB (POZ) domain containing 11 |
| 29 | High Risk | 1553069_at | C21orf87 | chromosome 21 open reading frame 87 |
| 38 | High Risk | 206873_at | CA6 | carbonic anhydrase VI |
| 35 | High Risk | 209288_s_at | CDC42EP3 | CDC42 effector protein (Rho GTPase binding) 3 |
| 33 | High Risk | 205295_at | CKMT2 | creatine kinase, mitochondrial 2 (sarcomeric) |
| 3 | High Risk | 208303_s_at | CRLF2 | cytokine receptor-like factor 2 |
| 32 | High Risk | 209101_at | CTGF | connective tissue growth factor |
| 18 | High Risk | 1554969_x_at | DIP2A | DIP2 disco-interacting protein 2 homolog A |
| | | | | (Drosophila) |
| 6 | High Risk | 219777_at | GIMAP6 | GTPase, IMAP family member 6 |
| 28 | High Risk | 229367_s_at | GIMAP6 | GTPase, IMAP family member 6 |
| 5 | High Risk | 235988_at | GPR110 | G protein-coupled receptor 110 |
| 23 | High Risk | 238689_at | GPR110 | G protein-coupled receptor 110 |
| 11 | High Risk | 203851_at | IGFBP6 | insulin-like growth factor binding protein 6 |
| 25 | High Risk | 212592_at | IGJ | Immunoglobulin J polypeptide, linker protein for |
| | | | | immunoglobulin alpha and mu polypeptides |
| 37 | High Risk | 209245_s_at | KIF1C | kinesin family member 1C |
| 9 | High Risk | 213371_at | LDB3 | LIM domain binding 3 |
| 12 | High Risk | 216887_s_at | LDB3 | LIM domain binding 3 |
| 22 | High Risk | 240457_at | LOC391849 | Similar to neuralized-like |
| 15 | High Risk | 237191_x_at | LOC650794 | Similar to FRAS1-related extracellular |
| | | | | matrix protein 2 precursor (ECM3 homolog) |
| 2 | High Risk | 217110_s_at | MUC4 | mucin 4, cell surface associated |
| 4 | High Risk | 217109_at | MUC4 | mucin 4, cell surface associated |
| 13 | High Risk | 204895_x_at | MUC4 | mucin 4, cell surface associated |
| 17 | High Risk | 205795_at | NRXN3 | neurexin 3 |
| 20 | High Risk | 215021_s_at | NRXN3 | neurexin 3 |
| 10 | High Risk | 210830_s_at | PON2 | paraoxonase 2 |
| 26 | High Risk | 201876_at | PON2 | paraoxonase 2 |
| 7 | Low Risk | 202388_at | RGS2 | regulator of G-protein signalling 2, 24 kDa |
| 14 | High Risk | 233390_at | RGS3 | Regulator of G-protein signalling 3 |
| 31 | High Risk | 204030_s_at | SCHIP1 | schwannomin interacting protein 1 |
| 36 | High Risk | 232108_at | SCHN3 | secemin 3 |
| 16 | High Risk | 225660_at | SEMA6A | sema domain, transmembrane domain (TM), and |
| | | | | cytoplasmic domain, (semaphorin) 6A |
| 21 | High Risk | 215028_at | SEMA6A | sema domain, transmembrane domain (TM), and |
| | | | | cytoplasmic domain, (semaphorin) 6A |
| 27 | High Risk | 223449_at | SEMA6a | sema domain, transmembrane domain (TM), and |
| | | | | cytoplasmic domain, (semaphorin) 6A |
| 30 | High Risk | 244697_at | ZBTB16 | Zinc finger and BTB domain containing 16 |

Then, the amount of the prognostic gene(s) from a patient inflicted with high risk B-ALL is determined. The amount of the prognostic gene present in that patient is compared with the established threshold value (a predetermined value) of the prognostic gene(s) which is indicative of therapeutic success (low risk) or failure (high risk), whereby the prognostic outcome of the patient is determined. The prognostic gene may be a gene which is indicative of a poor or unfavorable (bad) prognostic outcome (high risk) or a favorable (good) outcome (low risk). Analyzing expression levels of these genes provides accurate insight (diagnostic and prognostic) information into the likelihood of a therapeutic outcome in ALL, especially in a high risk B-ALL patient, including a pediatric patient.

In certain embodiments, the amount of the prognostic gene is determined by the quantitation of a transcript encoding the sequence of the prognostic gene; or a polypeptide encoded by the transcript. The quantitation of the transcript can be based on hybridization to the transcript. The quantitation of the polypeptide can be based on antibody detection or a related method. The method optionally comprises a step of amplifying nucleic acids from the tissue sample before the evaluating (PCR analysis). In a number of embodiments, the evaluating is of a plurality of prognostic genes, preferably at least two (2) prognostic genes, at least three (3) prognostic genes, at least four (4) prognostic genes, at least five (5) prognostic genes, at least six (6) prognostic genes, at least seven (7) prognostic genes, at least eight (8) prognostic genes, at least nine (9) prognostic genes, at least ten (10) prognostic genes, at least eleven (11) prognostic genes, at least twelve (12) prognostic genes, at least thirteen (13) prognostic genes, at least fourteen (14) prognostic genes, at least fifteen (15) prognostic genes, at least sixteen (16) prognostic genes, at least seventeen (17) prognostic genes, at least eighteen (18) prognostic genes, at least nineteen (19) prognostic genes, at least twenty (20) prognostic genes, at least twenty-one (21) prognostic genes, at least twenty-two (22) prognostic genes, at least twenty-three (23) prognostic genes, at least twenty-four (24), at least twenty-five (25), at least twenty-six (26), at least twenty-seven (27), at least twenty-eight (28), at least twenty-nine (29), at least thirty (30) or thirty-one (31) prognostic genes. The prognosis which is determined from measuring the prognostic genes contributes to selection of a therapeutic strategy, which may be a traditional therapy for ALL, including B-precursor ALL (where a favorable prognosis is determined from measurements), or a more aggressive therapy based upon a traditional therapy or a non-traditional therapy (where an unfavorable prognosis is determined from measurements).

The present invention is directed to methods for outcome prediction and risk classification in leukemia, especially a high risk classification in B precursor acute lymphoblastic leukemia (ALL), especially in children. In one embodiment, the invention provides a method for classifying leukemia in a patient that includes obtaining a biological sample from a patient; determining the expression level for a selected gene product, more preferably a group of selected gene products, to yield an observed gene expression level; and comparing the observed gene expression level for the selected gene product(s) to control gene expression levels (preferably including a predetermined level). The control gene expression level can be the expression level observed for the gene product(s) in a control sample, or a predetermined expression level for the gene product. An observed expression level (higher or lower) that differs from the control gene expression level is indicative of a disease classification and is predictive of a therapeutic outcome. In another aspect, the method can include determining a gene expression profile for selected gene products in the biological sample to yield an observed gene expression profile; and comparing the observed gene expression profile for the selected gene products to a control gene expression profile for the selected gene products that correlates with a disease classification, for example ALL, and in particular high risk B precursor ALL; wherein a similarity between the observed gene expression profile and the control gene expression profile is indicative of the disease classification (e.g., high risk B-all poor or favorable prognostic).

The disease classification can be, for example, a classification preferably based on predicted outcome (remission vs therapeutic failure); but may also include a classification based upon clinical characteristics of patients, a classification based on karyotype; a classification based on leukemia subtype; or a classification based on disease etiology. Measurement of all 31 genes (gene products) set forth in Table 1P and all 27 gene products set forth in Table 1Q, below, or a group of genes (gene products) falling within these larger lists as otherwise described herein may also be performed to provide an accurate assessment of therapeutic intervention.

The invention further provides for a method for predicting a patient falls within a particular group of high risk B-ALL patients and predicting therapeutic outcome in that B ALL leukemia patient, especially pediatric B-ALL that includes obtaining a biological sample from a patient; determining the expression level for selected gene products associated with outcome (high risk or low risk) to yield an observed gene expression level; and comparing the observed gene expression level for the selected gene product(s) to a control gene expression level for the selected gene product. The control gene expression level for the selected gene product can include the gene expression level for the selected gene product observed in a control sample, or a predetermined gene expression level for the selected gene product; wherein an observed expression level that is different from the control gene expression level for the selected gene product(s) is indicative of predicted remission or alternatively, an unfavorable outcome. The method preferably may determine gene expression levels of at least two gene products otherwise identified herein. The genes (gene product expression) otherwise described herein are measured, compared to predetermined values (e.g. from a control sample) and then assessed to determine the likelihood of a favorable or unfavorable therapeutic outcome and then providing a therapeutic approach consistent with the analysis of the express of the measured gene products. The present method may include measuring expression of at least two gene products up to 31 gene products according to Tables 1P and 1Q as otherwise described herein. In certain preferred aspects of the invention, the expression levels of all 31 gene products (Table 1P) or all 27 gene products Table 1Q) may be determined and compared to a predetermined gene expression level, wherein a measurement above or below a predetermined expression level is indicative of the likelihood of an unfavorable therapeutic response/therapeutic failure or a favorable therapeutic response (continuous complete remission or CCR). In the case where therapeutic failure is predicted, the use of more aggressive protocols of traditional anti-cancer therapies (higher doses and/or longer duration of drug administration) or experimental therapies may be advisable.

Optionally, the method further comprises determining the expression level for other gene products within the list of gene products otherwise disclosed herein and comparing in a similar fashion the observed gene expression levels for the selected gene products with a control gene expression level for those gene products, wherein an observed expression level for these gene products that is different from (above or below) the control gene expression level for that gene product (high risk or low risk) is further indicative of predicted remission (favorable prognosis) or relapse (unfavorable prognosis). It is noted that a higher expression (when compared to a control or predetermined value) of a high risk gene (gene product) is generally indicative of an unfavorable prognosis of therapeutic outcome; a higher expression (when compared to a control or predetermined value) of a low risk gene (gene product) is generally indicative of a favorable therapeutic outcome (remission, including continuous complete remission); a lower expression (when compared to a control or a predetermined value) of a high risk gene (gene product) is generally indicative of a favorable therapeutic outcome. Genes (gene products) are to be assessed in toto during an analysis to provide a predictive basis upon which to recommend therapeutic intervention in a patient.

The invention further includes a method for treating leukemia comprising administering to a leukemia patient a therapeutic agent that modulates the amount or activity of the gene product(s) associated with therapeutic outcome. Preferably, the method modulates (enhancement/upregulation of a gene product associated with a favorable or good therapeutic outcome (low risk) or inhibition/downregulation of a gene product associated with a poor or unfavorable therapeutic outcome (high risk) as measured by comparison with a control sample or predetermined value) at least two of the gene products as set forth above, three of the gene products, four of the gene products or all five of the gene products. In addition, the therapeutic method according to the present invention also modulates at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty or thirty one of a number of gene products as relevant in Tables 1P and 1Q as indicated or otherwise described herein. Preferred genes (gene products) useful in this aspect of the invention from Table 1P include BMPR1B; CTGF; IGJ; LDB3; PON2; RGS2; SCHIP1 and SEMA6A, all of which are high risk genes with the exception of RGS2.

Also provided by the invention is an in vitro method for screening a compound useful for treating leukemia, especially high risk B-ALL. The invention further provides an in vivo method for evaluating a compound for use in treating leukemia, especially high risk B-ALL. The candidate compounds are evaluated for their effect on the expression level(s) of one or more gene products associated with outcome in leukemia patients (for example, Table 1P and 1Q and as otherwise described herein), especially high risk B-ALL, preferably at least two of those gene products, at least three of those gene products, at least four of those gene products, at least five of those gene products, at least six of those gene products, at least seven of those gene products, at least eight of those gene products, at least nine of those gene products, at least ten of those gene products, at least eleven of those gene products, at least twelve of those gene products, at least thirteen of those gene products, at least fourteen of those gene products, at least fifteen of those gene products, at least sixteen of those gene products, at least seventeen of those gene products, at least eighteen of those gene products, at least twenty of those gene products, at least twenty-one of those gene products, at least twenty-two of those gene products, at least twenty-three of those gene products, at least twenty-four, at least twenty-five, at least twenty-six, at least twenty-seven, at least twenty-eight, at least twenty-nine, at least thirty or thirty-one of those gene products may be measured to determine a therapeutic outcome.

The preferred gene products may also include at least three of CA6, IGJ, MUC4, GPR110, LDB3, PON2, CRLF2 and RGS2 (preferably CRLF2 is included in the at least three gene products) and in certain instances may further include AGAP-1 (Arf GAP with GTP-binding protein-like, ANK repeat and PH domains, also CENTG2) and/or PCDH17 (Protocadherin-17). These genes/gene products and their expression above or below a predetermined expression level are more predictive of overall outcome. As shown below, at least two or more of the gene products which are presented in tables 1P or 1G may be used to predict therapeutic outcome. This predictive model is tested in an independent cohort of high risk pediatric B-ALL cases (20) and is found to predict outcome with extremely high statistical significance (p-value <1.0⁻⁸). It is noted that the expression of gene products of at least two of the five genes listed above, as well as additional genes from the list appearing in Tables 1P and 1Q and in certain preferred instances, the expression of all 24 gene products of Table 1P and 1Q may be measured and compared to predetermined expression levels to provide the greater degrees of certainty of a therapeutic outcome.

### DETAILED DESCRIPTION OF THE INVENTION

Gene expression profiling can provide insights into disease etiology and genetic progression, and can also provide tools for more comprehensive molecular diagnosis and therapeutic targeting. The biologic clusters and associated gene profiles identified herein may be useful for refined molecular classification of acute leukemias as well as improved risk assessment and classification, especially of high risk B precursor acute lymphoblastic leukemia (B-ALL), especially including pediatric B-ALL. In addition, the invention has identified numerous genes, including but not limited to the genes as presented in Tables 1P and 1Q hereof, that are, alone or in combination, strongly predictive of therapeutic outcome in high risk B-ALL, and in particular high risk pediatric B precursor ALL. The genes identified herein, and the gene products from said genes, including proteins they encode, can be used to refine risk classification and diagnostics, to make outcome predictions and improve prognostics, and to serve as therapeutic targets in infant leukemia and pediatric ALL, especially B-precursor ALL.

“Gene expression” as the term is used herein refers to the production of a biological product encoded by a nucleic acid sequence, such as a gene sequence. This biological product, referred to herein as a “gene product,” may be a nucleic acid or a polypeptide. The nucleic acid is typically an RNA molecule which is produced as a transcript from the gene sequence. The RNA molecule can be any type of RNA molecule, whether either before (e.g., precursor RNA) or after (e.g., mRNA) post-transcriptional processing. cDNA prepared from the mRNA of a sample is also considered a gene product. The polypeptide gene product is a peptide or protein that is encoded by the coding region of the gene, and is produced during the process of translation of the mRNA.

The term “gene expression level” refers to a measure of a gene product(s) of the gene and typically refers to the relative or absolute amount or activity of the gene product.

The term “gene expression profile” as used herein is defined as the expression level of two or more genes. The term gene includes all natural variants of the gene. Typically a gene expression profile includes expression levels for the products of multiple genes in given sample, up to about 13,000, preferably determined using an oligonucleotide microarray.

Unless otherwise specified, “a,” “an,” “the,” and “at least one” are used interchangeably and mean one or more than one.

The term “patient” shall mean within context an animal, preferably a mammal, more preferably a human patient, more preferably a human child who is undergoing or will undergo therapy or treatment for leukemia, especially high risk B-precursor acute lymphoblastic leukemia.

The term “high risk B precursor acute lymphocytic leukemia” or “high risk B-ALL” refers to a disease state of a patient with acute lymphoblastic leukemia who meets certain high risk disease criteria. These include: confirmation of B-precursor ALL in the patient by central reference laboratories (See Borowitz, et al., Rec Results Cancer Res 1993; 131: 257-267); and exhibiting a leukemic cell DNA index of ≦1.16 (DNA content in leukemic cells: DNA content of normal G₀/G₁ cells) (DI) by central reference laboratory (See, Trueworthy, et al., J Clin Oncol 1992; 10: 606-613; and Pullen, et al., “Immunologic phenotypes and correlation with treatment results”. In Murphy S B, Gilbert JR (eds). Leukemia Research: Advances in Cell Biology and Treatment. Elsevier: Amsterdam, 1994, pp 221-239) and at least one of the following: (1) WBC ≧10 000-99 000/μl, aged 1-2.99 years or ages 6-21 years; (2) WBC ≧100 000/μl, aged 1-21 years; (3) all patients with CNS or overt testicular disease at diagnosis; or (4) leukemic cell chromosome translocations t(1;19) or t(9;22) confirmed by central reference laboratory. (See, Crist, et al, Blood 1990; 76: 117-122; and Fletcher, et al., Blood 1991; 77: 435-439).

The term “traditional therapy” relates to therapy (protocol) which is typically used to treat leukemia, especially B-precursor ALL (including pediatric B-ALL) and can include Memorial Sloan-Kettering New York II therapy (NY II), UKALLR2, AL 841, AL851, ALHR88, MCP841 (India), as well as modified BFM (Berlin-Frankfurt-Munster) therapy, BMF-95 or other therapy, including ALinC 17 therapy as is well-known in the art. In the present invention the term “more aggressive therapy” or “alternative therapy” usually means a more aggressive version of conventional therapy typically used to treat leukemia, for example B-ALL, including pediatric B-precursor ALL, using for example, conventional or traditional chemotherapeutic agents at higher dosages and/or for longer periods of time in order to increase the likelihood of a favorable therapeutic outcome. It may also refer, in context, to experimental therapies for treating leukemia, rather than simply more aggressive versions of conventional (traditional) therapy.

### Diagnosis, Prognosis and Risk Classification

Current parameters used for diagnosis, prognosis and risk classification in pediatric ALL are related to clinical data, cytogenetics and response to treatment. They include age and white blood count, cytogenetics, the presence or absence of minimal residual disease (MRD), and a morphological assessment of early response (measured as slow or rapid early therapeutic response). As noted above however, these parameters are not always well correlated with outcome, nor are they precisely predictive at diagnosis.

Prognosis is typically recognized as a forecast of the probable course and outcome of a disease. As such, it involves inputs of both statistical probability, requiring numbers of samples, and outcome data. In the present invention, outcome data is utilized in the form of continuous complete remission (CCR) of ALL or therapeutic failure (non-CCR). A patient population of hundreds is included, providing statistical power.

The ability to determine which cases of leukemia, especially high risk B precursor acute lymphoblastic leukemia (B-ALL), including high risk pediatric B-ALL will respond to treatment, and to which type of treatment, would be useful in appropriate allocation of treatment resources. It would also provide guidance as to the aggressiveness of therapy in producing a favorable outcome (continuous complete remission or CCR). As indicated above, the various standard therapies have significantly different risks and potential side effects, especially therapies which are more aggressive or even experimental in nature. Accurate prognosis would also minimize application of treatment regimens which have low likelihood of success and would allow a more efficient aggressive or even an experimental protocol to be used without wasting effort on therapies unlikely to produce a favorable therapeutic outcome, preferably a continuous complete remission. Such also could avoid delay of the application of alternative treatments which may have higher likelihoods of success for a particular presented case. Thus, the ability to evaluate individual leukemia cases, especially B-precursor acute lymphoblastic leukemia, for markers which subset into responsive and non-responsive groups for particular treatments is very useful.

Current models of leukemia classification have become better at distinguishing between cancers that have similar histopathological features but vary in clinical course and outcome, except in certain areas, one of them being in high risk B-precursor acute lymphoblastic leukemia (B-ALL). Identification of novel prognostic molecular markers is a priority if radical treatment is to be offered on a more selective basis to those high risk leukemia patients with disease states which do not respond favorably to conventional therapy. A novel strategy is described to discover/assess/measure molecular markers for B-ALL leukemia, especially high risk B-ALL to determine a treatment protocol, by assessing gene expression in leukemia patients and modeling these data based on a predetermined gene product expression for numerous patients having a known clinical outcome. The invention herein is directed to defining different forms of leukemia, in particular, B-precursor acute lymphoblastic leukemia, especially high risk B-precursor acute lymphoblastic leukemia, including high risk pediatric B-ALL by measuring expression gene products which can translate directly into therapeutic prognosis. Such prognosis allows for application of a treatment regimen having a greater statistical likelihood of cost effective treatments and minimization of negative side effects from the different/various treatment options.

In preferred aspects, the present invention provides an improved method for identifying and/or classifying acute leukemias, especially B precursor ALL, even more especially high risk B precursor ALL and also high risk pediatric B precursor ALL and for providing an indication of the therapeutic outcome of the patient based upon an assessment of expression levels of particular genes. Expression levels are determined for two or more genes associated with therapeutic outcome, risk assessment or classification, karyotpe (e.g., MLL translocation) or subtype (e.g., B-ALL, especially high risk B-ALL). Genes that are particularly relevant for diagnosis, prognosis and risk classification, especially for high risk B precursor ALL, including high risk pediatric B precursor ALL, according to the invention include those described in the tables (especially Table 1P and 1Q) and figures herein. The gene expression levels for the gene(s) of interest in a biological sample from a patient diagnosed with or suspected of having an acute leukemia, especially B precursor ALL are compared to gene expression levels observed for a control sample, or with a predetermined gene expression level. Observed expression levels that are higher or lower than the expression levels observed for the gene(s) of interest in the control sample or that are higher or lower than the predetermined expression levels for the gene(s) of interest (as set forth in Table 1P and 1Q) provide information about the acute leukemia that facilitates diagnosis, prognosis, and/or risk classification and can aid in treatment decisions, especially whether to use a more of less aggressive therapeutic regimen or perhaps even an experimental therapy. When the expression levels of multiple genes are assessed for a single biological sample, a gene expression profile is produced.

Current models of leukemia classification have become better at distinguishing between cancers that have similar histopathological features but vary in clinical course and outcome, except in certain areas, one of them being in high risk B-precursor acute lymphoblastic leukemia (B-ALL). Identification of novel prognostic molecular markers is a priority if radical treatment is to be offered on a more selective basis to those high risk leukemia patients with disease states which do not respond favorably to conventional therapy. A novel strategy is described to discover/assess/measure molecular markers for B-ALL leukemia, especially high risk B-ALL to determine a treatment protocol, by assessing gene expression in leukemia patients and modeling these data based on a predetermined gene product expression for numerous patients having a known clinical outcome. The invention herein is directed to defining different forms of leukemia, in particular, B-precursor acute lymphoblastic leukemia, especially high risk B-precursor acute lymphoblastic leukemia, including high risk pediatric B-ALL by measuring expression gene products which can translate directly into therapeutic prognosis. Such prognosis allows for application of a treatment regimen having a greater statistical likelihood of cost effective treatments and minimization of negative side effects from the different/various treatment options.

In preferred aspects, the present invention provides an improved method for identifying and/or classifying acute leukemias, especially B precursor ALL, even more especially high risk B precursor ALL and also high risk pediatric B precursor ALL and for providing an indication of the therapeutic outcome of the patient based upon an assessment of expression levels of particular genes. Expression levels are determined for two or more genes associated with therapeutic outcome, risk assessment or classification, karyotpe (e.g., MLL translocation) or subtype (e.g., B-ALL, especially high risk B-ALL). Genes that are particularly relevant for diagnosis, prognosis and risk classification, especially for high risk B precursor ALL, including high risk pediatric B precursor ALL, according to the invention include those described in the tables (especially Table 1P and 1Q) and figures herein. The gene expression levels for the gene(s) of interest in a biological sample from a patient diagnosed with or suspected of having an acute leukemia, especially B precursor ALL are compared to gene expression levels observed for a control sample, or with a predetermined gene expression level. Observed expression levels that are higher or lower than the expression levels observed for the gene(s) of interest in the control sample or that are higher or lower than the predetermined expression levels for the gene(s) of interest (as set forth in Table 1P and 1Q) provide information about the acute leukemia that facilitates diagnosis, prognosis, and/or risk classification and can aid in treatment decisions, especially whether to use a more of less aggressive therapeutic regimen or perhaps even an experimental therapy. When the expression levels of multiple genes are assessed for a single biological sample, a gene expression profile is produced.

In one aspect, the invention provides genes and gene expression profiles that are correlated with outcome (i.e., complete continuous remission or good/favorable prognosis vs. therapeutic failure or poor/unfavorable prognosis) in high risk B-ALL. Assessment of at least two or more of these genes according to the invention, preferably at least three, at least four, at least five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six (Table 1Q shows 26 genes), twenty-seven, twenty-eight, twenty-nine, thirty or thirty-one as set forth in Tables 1Pin a given gene profile can be integrated into revised risk classification schemes, therapeutic targeting and clinical trial design. In one embodiment, the expression levels of a particular gene (gene products) are measured, and that measurement is used, either alone or with other parameters, to assign the patient to a particular risk category (e.g., high risk B-ALL good/favorable or high risk B-ALL poor/unfavorable). The invention identifies a preferred number of genes from Table P whose expression levels, either alone or in combination, are associated with outcome, including but not limited to at least two genes, preferably at least three genes, four genes, five genes, six genes, seven genes or eight genes selected from the group consisting of BMPR1B; CTGF; IGJ; LDB3; PON2; RGS2; SCHIP1 and SEMA6A. The invention identifies a preferred number of genes from Table Q whose expression levels, either alone or in combination, are associated with outcome, including but not limited to at least two genes, preferably at least three genes, four genes, five genes, six genes, seven genes, eight genes, nine genes, ten genes or eleven genes selected from the group consisting of BMPR1B; CA6; CRLF2; GPR110; IGJ; LDB3; MUC4; NRXN3; PON2; RGS2 and SEMA6A. Of this list of 11 genes the following 9 are more relevant and indicative of a predictive outcome: BMPR1B; CA6; CRLF2; GPR110; IGJ; LDB3; MUC4; PON2 and RGS2.

Some of these genes exhibit a positive association between expression level and outcome (low risk). For these genes, expression levels above a predetermined threshold level (or higher than that exhibited by a control sample) is predictive of a positive outcome (continuous complete remission). In particular, it is expected such measurements can be used to refine risk classification in children who are otherwise classified as having high risk B-ALL, but who can respond favorable (cured) with traditional, less intrusive therapies.

A number of genes, and in particular, CRLF2, MUC4 and LDB3 and to a lesser extent CA6, PON2 and BMPR1B, in particular, are strong predictors of an unfavorable outcome for a high risk B-ALL patient and therefore in preferred aspects, the expression of at least two genes, and preferably the expression of at least three or four of those three genes among those cited above are measured and compared with predetermined values for each of the gene products measured. This list may guide the choice of gene products to analyze to determine a therapeutic outcome or for evaluating a drug, compound or therapeutic regimen. The expression of RGS2 is a strong predictor of favorable outcome (low risk) and such can be used to further determine a predictive outcome.

In general, the expression of at least two genes in a single group is measured and compared to a predetermined value to provide a therapeutic outcome prediction and in addition to those two genes, the expression of any number of additional genes described in Tables 1P and 1Q can be measured and used for predicting therapeutic outcome. In certain aspects of the invention where very high reliability is desired/required, the expression levels of all 31 or 26 genes genes (as per Tables 1P and 1Q) may be measured and compared with a predetermined value for each of the genes measured such that a measurement above or below the predetermined value of expression for each of the group of genes is indicative of a favorable therapeutic outcome (continuous complete remission) or a therapeutic failure. In the event of a predictive favorable therapeutic outcome, conventional anti-cancer therapy may be used and in the event of a predictive unfavorable outcome (failure), more aggressive therapy may be recommended and implemented.

The expression levels of multiple (two or more, preferably three or more, more preferably at least five genes as described hereinabove and in addition to the five, up to twenty-four to thirty-one genes within the genes listed in Tables 1P and 1Q in one or more lists of genes associated with outcome can be measured, and those measurements are used, either alone or with other parameters, to assign the patient to a particular risk category as it relates to a predicted therapeutic outcome. For example, gene expression levels of multiple genes can be measured for a patient (as by evaluating gene expression using an Affymetrix microarray chip) and compared to a list of genes whose expression levels (high or low) are associated with a positive (or negative) outcome. If the gene expression profile of the patient is similar to that of the list of genes associated with outcome, then the patient can be assigned to a low risk (favorable outcome) or high risk (unfavorable outcome) category. The correlation between gene expression profiles and class distinction can be determined using a variety of methods. Methods of defining classes and classifying samples are described, for example, in Golub et al, U.S. Patent Application Publication No. 2003/0017481 published Jan. 23, 2003, and Golub et al., U.S. Patent Application Publication No. 2003/0134300, published Jul. 17, 2003. The information provided by the present invention, alone or in conjunction with other test results, aids in sample classification and diagnosis of disease.

Computational analysis using the gene lists and other data, such as measures of statistical significance, as described herein is readily performed on a computer. The invention should therefore be understood to encompass machine readable media comprising any of the data, including gene lists, described herein. The invention further includes an apparatus that includes a computer comprising such data and an output device such as a monitor or printer for evaluating the results of computational analysis performed using such data.

In another aspect, the invention provides genes and gene expression profiles that are correlated with cytogenetics. This allows discrimination among the various karyotypes, such as MLL translocations or numerical imbalances such as hyperdiploidy or hypodiploidy, which are useful in risk assessment and outcome prediction.

In yet another aspect, the invention provides genes and gene expression profiles that are correlated with intrinsic disease biology and/or etiology. In other words, gene expression profiles that are common or shared among individual leukemia cases in different patients can be used to define intrinsically related groups (often referred to as clusters) of acute leukemia that cannot be appreciated or diagnosed using standard means such as morphology, immunophenotype, or cytogenetics. Mathematical modeling of the very sharp peak in ALL incidence seen in children 2-3 years old (>80 cases per million) has suggested that ALL may arise from two primary events, the first of which occurs in utero and the second after birth (Linet et al., Descriptive epidemiology of the leukemias, in Leukemias, 5^{th} Edition. ES Henderson et al. (eds). WB Saunders, Philadelphia. 1990). Interestingly, the detection of certain ALL-associated genetic abnormalities in cord blood samples taken at birth from children who are ultimately affected by disease supports this hypothesis (Gale et al., Proc. Natl. Acad. Sci. U.S.A., 94:13950-13954, 1997; Ford et al., Proc. Natl. Acad. Sci. U.S.A., 95:4584-4588, 1998).

The results for pediatric B precursor ALL suggest that this disease is composed of novel intrinsic biologic clusters defined by shared gene expression profiles, and that these intrinsic subsets cannot reliably be defined or predicted by traditional labels currently used for risk classification or by the presence or absence of specific cytogenetic abnormalities. We have identified 31 genes (Table 1P) and 26 genes (Table 1Q) for determining outcome in high risk B-ALL, and in particular high risk pediatric B precursor ALL using the methods set forth hereinbelow, for identifying candidate genes associated with classification and outcome. We have identified 8 preferred genes (Table 1P) which are predictors of outcome in high risk B precursor ALL patients, especially high risk pediatric B precursor ALL patients. We have identified 11 genes (preferably 9 genes) which are predictors of outcome in high risk B precursor ALL patients, especially high risk pediatric B precursor ALL patients. Expression of two or more of these genes which is greater than a predetermined value or from a control may be indicative that traditional B-ALL therapy is appropriate (low risk) or inappropriate (high risk) for treating the patient's B precursor ALL. Where traditional therapy is viewed as being inappropriate (high risk), a measurement of the expression of these genes which is higher than predetermined values for each of these genes is predictive of a high likelihood of a therapeutic failure using traditional B precursor ALL therapies. High expression for these (high risk) genes would dictate an early aggressive therapy or experimental therapy in order to increase the likelihood of a favorable therapeutic outcome. Low expression for these (high risk) genes and/or expression of low risk genes would favor traditional therapy and a favorable result from that therapy.

Some genes in these clusters are metabolically related, suggesting that a metabolic pathway that is associated with cancer initiation or progression. Other genes in these metabolic pathways, like the genes described herein but upstream or downstream from them in the metabolic pathway, thus can also serve as therapeutic targets.

In yet another aspect, the invention provides genes and gene expression profiles which may be used to discriminate high risk B-ALL from acute myeloid leukemia (AML) in infant leukemias by measuring the expression levels of the gene product(s) correlated with B-ALL as otherwise described herein, especially B-precursor ALL.

It should be appreciated that while the present invention is described primarily in terms of human disease, it is useful for diagnostic and prognostic applications in other mammals as well, particularly in veterinary applications such as those related to the treatment of acute leukemia in cats, dogs, cows, pigs, horses and rabbits.

Further, the invention provides methods for computational and statistical methods for identifying genes, lists of genes and gene expression profiles associated with outcome, karyotype, disease subtype and the like as described herein.

In sum, the present invention has identified a group of genes which strongly correlate with favorable/unfavorable outcome in B precursor acute lymphoblastic leukemia and contribute unique information to allow the reliable prediction of a therapeutic outcome in high risk B precursor ALL, especially high risk pediatric B precursor ALL.

### Measurement of Gene Expression Levels

Gene expression levels are determined by measuring the amount or activity of a desired gene product (i.e., an RNA or a polypeptide encoded by the coding sequence of the gene) in a biological sample. Any biological sample can be analyzed. Preferably the biological sample is a bodily tissue or fluid, more preferably it is a bodily fluid such as blood, serum, plasma, urine, bone marrow, lymphatic fluid, and CNS or spinal fluid. Preferably, samples containing mononuclear bloods cells and/or bone marrow fluids and tissues are used. In embodiments of the method of the invention practiced in cell culture (such as methods for screening compounds to identify therapeutic agents), the biological sample can be whole or lysed cells from the cell culture or the cell supernatant.

Gene expression levels can be assayed qualitatively or quantitatively. The level of a gene product is measured or estimated in a sample either directly (e.g., by determining or estimating absolute level of the gene product) or relatively (e.g., by comparing the observed expression level to a gene expression level of another samples or set of samples). Measurements of gene expression levels may, but need not, include a normalization process.

Typically, mRNA levels (or cDNA prepared from such mRNA) are assayed to determine gene expression levels. Methods to detect gene expression levels include Northern blot analysis (e.g., Harada et al., Cell 63:303-312 (1990)), S1 nuclease mapping (e.g., Fujita et al., Cell 49:357-367 (1987)), polymerase chain reaction (PCR), reverse transcription in combination with the polymerase chain reaction (RT-PCR) (e.g., Example III; see also Makino et al., Technique 2:295-301 (1990)), and reverse transcription in combination with the ligase chain reaction (RT-LCR). Multiplexed methods that allow the measurement of expression levels for many genes simultaneously are preferred, particularly in embodiments involving methods based on gene expression profiles comprising multiple genes. In a preferred embodiment, gene expression is measured using an oligonucleotide microarray, such as a DNA microchip. DNA microchips contain oligonucleotide probes affixed to a solid substrate, and are useful for screening a large number of samples for gene expression. DNA microchips comprising DNA probes for binding polynucleotide gene products (mRNA) of the various genes from Table 1 are additional aspects of the present invention.

Alternatively or in addition, polypeptide levels can be assayed. Immunological techniques that involve antibody binding, such as enzyme linked immunosorbent assay (ELISA) and radioimmunoassay (RIA), are typically employed. Where activity assays are available, the activity of a polypeptide of interest can be assayed directly.

As discussed above, the expression levels of these markers in a biological sample may be evaluated by many methods. They may be evaluated for RNA expression levels. Hybridization methods are typically used, and may take the form of a PCR or related amplification method. Alternatively, a number of qualitative or quantitative hybridization methods may be used, typically with some standard of comparison, e.g., actin message. Alternatively, measurement of protein levels may performed by many means. Typically, antibody based methods are used, e.g., ELISA, radioimmunoassay, etc., which may not require isolation of the specific marker from other proteins. Other means for evaluation of expression levels may be applied. Antibody purification may be performed, though separation of protein from others, and evaluation of specific bands or peaks on protein separation may provide the same results. Thus, e.g., mass spectroscopy of a protein sample may indicate that quantitation of a particular peak will allow detection of the corresponding gene product. Multidimensional protein separations may provide for quantitation of specific purified entities.

The observed expression levels for the gene(s) of interest are evaluated to determine whether they provide diagnostic or prognostic information for the leukemia being analyzed. The evaluation typically involves a comparison between observed gene expression levels and either a predetermined gene expression level or threshold value, or a gene expression level that characterizes a control sample (“predetermined value”). The control sample can be a sample obtained from a normal (i.e., non-leukemic) patient(s) or it can be a sample obtained from a patient or patients with high risk B-ALL that has been cured. For example, if a cytogenic classification is desired, the biological sample can be interrogated for the expression level of a gene correlated with the cytogenic abnormality, then compared with the expression level of the same gene in a patient known to have the cytogenetic abnormality (or an average expression level for the gene that characterizes that population).

The present study provides specific identification of multiple genes whose expression levels in biological samples will serve as markers to evaluate leukemia cases, especially therapeutic outcome in high risk B-ALL cases, especially high risk pediatric B-ALL cases. These markers have been selected for statistical correlation to disease outcome data on a large number of leukemia (high risk B-ALL) patients as described herein.

### Treatment of Infant Leukemia and Pediatric B-Precursor ALL

The genes identified herein that are associated with outcome of a disease state may provide insight into a treatment regimen. That regimen may be that traditionally used for the treatment of leukemia (as discussed hereinabove) in the case where the analysis of gene products from samples taken from the patient predicts a favorable therapeutic outcome, or alternatively, the chosen regimen may be a more aggressive approach (e.g, higher dosages of traditional therapies for longer periods of time) or even experimental therapies in instances where the predictive outcome is that of failure of therapy.

In addition, the present invention may provide new treatment methods, agents and regimens for the treatment of leukemia, especially high risk B-precursor acute lymphoblastic leukemia, especially high risk pediatric B-precursor ALL. The genes identified herein that are associated with outcome and/or specific disease subtypes or karyotypes are likely to have a specific role in the disease condition, and hence represent novel therapeutic targets. Thus, another aspect of the invention involves treating high risk B-ALL patients, including high risk pediatric ALL patients by modulating the expression of one or more genes described herein in Table 1P or 1F to a desired expression level or below.

In the case of those gene products (Table 1P and 1Q) whose increased or decreased expression (whether above or below a predetermined value, for example obtained for a control sample) is associated with a favorable outcome or failure, the treatment method of the invention will involve enhancing the expression of one or more of those gene products in which a favorable therapeutic outcome is predicted (low risk) by such enhancement and inhibiting the expression of one or more of those gene products in which enhanced expression is associated with failed therapy (high risk).

The therapeutic agent can be a polypeptide having the biological activity of the polypeptide of interest (e.g., BTG3, CD2, RGS2 or other gene product, preferably a low risk gene/gene product) or a biologically active subunit or analog thereof. Alternatively, the therapeutic agent can be a ligand (e.g., a small non-peptide molecule, a peptide, a peptidomimetic compound, an antibody, or the like) that agonizes (i.e., increases) the activity of the polypeptide of interest. For example, in the case of BTG3, CD2, RGS2 or other gene product, these gene products may be administered to the patient to enhance the activity and treat the patient.

Gene therapies can also be used to increase the amount of a polypeptide of interest in a host cell of a patient. Polynucleotides operably encoding the polypeptide of interest can be delivered to a patient either as “naked DNA” or as part of an expression vector. The term vector includes, but is not limited to, plasmid vectors, cosmid vectors, artificial chromosome vectors, or, in some aspects of the invention, viral vectors. Examples of viral vectors include adenovirus, herpes simplex virus (HSV), alphavirus, simian virus 40, picornavirus, vaccinia virus, retrovirus, lentivirus, and adeno-associated virus. Preferably the vector is a plasmid. In some aspects of the invention, a vector is capable of replication in the cell to which it is introduced; in other aspects the vector is not capable of replication. In some preferred aspects of the present invention, the vector is unable to mediate the integration of the vector sequences into the genomic DNA of a cell. An example of a vector that can mediate the integration of the vector sequences into the genomic DNA of a cell is a retroviral vector, in which the integrase mediates integration of the retroviral vector sequences. A vector may also contain transposon sequences that facilitate integration of the coding region into the genomic DNA of a host cell.

Selection of a vector depends upon a variety of desired characteristics in the resulting construct, such as a selection marker, vector replication rate, and the like. An expression vector optionally includes expression control sequences operably linked to the coding sequence such that the coding region is expressed in the cell. The invention is not limited by the use of any particular promoter, and a wide variety is known. Promoters act as regulatory signals that bind RNA polymerase in a cell to initiate transcription of a downstream (3′ direction) operably linked coding sequence. The promoter used in the invention can be a constitutive or an inducible promoter. It can be, but need not be, heterologous with respect to the cell to which it is introduced.

Another option for increasing the expression of a gene is to reduce the amount of methylation of the gene. Demethylation agents, therefore, may be used to re-activate the expression of one or more of the gene products in cases where methylation of the gene is responsible for reduced gene expression in the patient.

For other genes identified herein as being correlated with therapeutic failure or without outcome in high risk B-ALL, such as high risk pediatric B-ALL, high expression of the gene is associated with a negative outcome rather than a positive outcome (high risk). In such instances, where the expression levels of these genes as described are high, the predicted therapeutic outcome in such patients is therapeutic failure for traditional therapies. In such case, more aggressive approaches to traditional therapies and/or experimental therapies may be attempted.

The genes described above (high risk, negative outcome) accordingly represent novel therapeutic targets, and the invention provides a therapeutic method for reducing (inhibiting) the amount and/or activity of these polypeptides of interest in a leukemia patient. Preferably the amount or activity of the selected gene product is reduced to less than about 90%, more preferably less than about 75%, most preferably less than about 25% of the gene expression level observed in the patient prior to treatment.

Genes (gene products) which are described as high risk from Table 1P include BMPR1B; C8orf38; CDC42EP3; CTGF; DKFZP761M1511; ECM1; GRAMD1C; IGJ; LDB3; LOC400581; LRRC62; MDFIC; NT5E; PON2; SCHIP1; SEMA6A; TSPAN7; and TTYH2. Of these, one or more of the following represent preferred therapeutic targets: BMPR1B; CTGF; IGJ; LDB3; PON2; RGS2; SCHIP1 and SEMA6A. Genes (gene products) which are described as high risk from Table 1Q include: BMPR1B; BTBD11; C21orf87; CA6; CDC42EP3; CKMT2; CRLF2; CTGF; DIP2A; GIMAP6; GPR110; IGFBP6; IGJ; K1F1C; LDB3; LOC391849; LOC650794; MUC4; NRXN3; PON2; RGS3; SCHIP1; SCRN3; EMA6A and ZBTB16. Of these, one or more of the following represent preferred therapeutic targets: BMPR1B; CA6; CRLF2; GPR110; IGJ; LDB3; MUC4; NRXN3; PON2; and SEMA6A

A cell manufactures proteins by first transcribing the DNA of a gene for that protein to produce RNA (transcription). In eukaryotes, this transcript is an unprocessed RNA called precursor RNA that is subsequently processed (e.g. by the removal of introns, splicing, and the like) into messenger RNA (mRNA) and finally translated by ribosomes into the desired protein. This process may be interfered with or inhibited at any point, for example, during transcription, during RNA processing, or during translation. Reduced expression of the gene(s) leads to a decrease or reduction in the activity of the gene product and, in cases where high expression leads to a theapeuric failure, an expected therapeutic success.

The therapeutic method for inhibiting the activity of a gene whose high expression (Table 1P/1Q) is correlated with negative outcome/therapeutic failure involves the administration of a therapeutic agent to the patient to inhibit the expression of the gene. The therapeutic agent can be a nucleic acid, such as an antisense RNA or DNA, or a catalytic nucleic acid such as a ribozyme, that reduces activity of the gene product of interest by directly binding to a portion of the gene encoding the enzyme (for example, at the coding region, at a regulatory element, or the like) or an RNA transcript of the gene (for example, a precursor RNA or mRNA, at the coding region or at 5′ or 3′ untranslated regions) (see, e.g., Golub et al., U.S. Patent Application Publication No. 2003/0134300, published Jul. 17, 2003). Alternatively, the nucleic acid therapeutic agent can encode a transcript that binds to an endogenous RNA or DNA; or encode an inhibitor of the activity of the polypeptide of interest. It is sufficient that the introduction of the nucleic acid into the cell of the patient is or can be accompanied by a reduction in the amount and/or the activity of the polypeptide of interest. An RNA captamer can also be used to inhibit gene expression. The therapeutic agent may also be protein inhibitor or antagonist, such as small non-peptide molecule such as a drug or a prodrug, a peptide, a peptidomimetic compound, an antibody, a protein or fusion protein, or the like that acts directly on the polypeptide of interest to reduce its activity.

The invention includes a pharmaceutical composition that includes an effective amount of a therapeutic agent as described herein as well as a pharmaceutically acceptable carrier. These therapeutic agents may be agents or inhibitors of selected genes (table 1P/1Q). Therapeutic agents can be administered in any convenient manner including parenteral, subcutaneous, intravenous, intramuscular, intraperitoneal, intranasal, inhalation, transdermal, oral or buccal routes. The dosage administered will be dependent upon the nature of the agent; the age, health, and weight of the recipient; the kind of concurrent treatment, if any; frequency of treatment; and the effect desired. A therapeutic agent(s) identified herein can be administered in combination with any other therapeutic agent(s) such as immunosuppressives, cytotoxic factors and/or cytokine to augment therapy, see Golub et al, Golub et al., U.S. Patent Application Publication No. 2003/0134300, published Jul. 17, 2003, for examples of suitable pharmaceutical formulations and methods, suitable dosages, treatment combinations and representative delivery vehicles.

The effect of a treatment regimen on an acute leukemia patient can be assessed by evaluating, before, during and/or after the treatment, the expression level of one or more genes as described herein. Preferably, the expression level of gene(s) associated with outcome, such as a gene as described above, may be monitored over the course of the treatment period. Optionally gene expression profiles showing the expression levels of multiple selected genes associated with outcome can be produced at different times during the course of treatment and compared to each other and/or to an expression profile correlated with outcome.

### Screening for Therapeutic Agents

The invention further provides methods for screening to identify agents that modulate expression levels of the genes identified herein that are correlated with outcome, risk assessment or classification, cytogenetics or the like. Candidate compounds can be identified by screening chemical libraries according to methods well known to the art of drug discovery and development (see Golub et al., U.S. Patent Application Publication No. 2003/0134300, published Jul. 17, 2003, for a detailed description of a wide variety of screening methods). The screening method of the invention is preferably carried out in cell culture, for example using leukemic cell lines (especially B-precursor ALL cell lines) that express known levels of the therapeutic target or other gene product as otherwise described herein (see Table 1G and 1P). The cells are contacted with the candidate compound and changes in gene expression of one or more genes relative to a control culture or predetermined values based upon a control culture are measured. Alternatively, gene expression levels before and after contact with the candidate compound can be measured. Changes in gene expression (above or below a predetermined value, depending upon the low risk or high risk character of the gene/gene product) indicate that the compound may have therapeutic utility. Structural libraries can be surveyed computationally after identification of a lead drug to achieve rational drug design of even more effective compounds.

The invention further relates to compounds thus identified according to the screening methods of the invention. Such compounds can be used to treat high risk B-ALL especially include high risk pediatric B-ALL as appropriate, and can be formulated for therapeutic use as described above.

Active analogs, as that term is used herein, include modified polypeptides. Modifications of polypeptides of the invention include chemical and/or enzymatic derivatizations at one or more constituent amino acids, including side chain modifications, backbone modifications, and N- and C-terminal modifications including acetylation, hydroxylation, methylation, amidation, and the attachment of carbohydrate or lipid moieties, cofactors, and the like.

In certain aspects of the present invention, a therapeutic method may rely on an antibody to one or more gene products predictive of outcome, preferably to one or more gene product which otherwise is predictive of a negative outcome, so that the antibody may function as an inhibitor of a gene product. Preferably the antibody is a human or humanized antibody, especially if it is to be used for therapeutic purposes. A human antibody is an antibody having the amino acid sequence of a human immunoglobulin and include antibodies produced by human B cells, or isolated from human sera, human immunoglobulin libraries or from animals transgenic for one or more human immunoglobulins and that do not express endogenous immunoglobulins, as described in U.S. Pat. No. 5,939,598 by Kucherlapati et al., for example. Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. U.S.A., 90:2551-2555 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Antibodies generated in non-human species can be “humanized” for administration in humans in order to reduce their antigenicity. Humanized forms of non-human (e.g., murine) antibodies are chimeric immunoglobulins, immunoglobulin chains or fragments thereof (such as Fv, Fab, Fab', F(ab′)2, or other antigen-binding subsequences of antibodies) which contain minimal sequence derived from non-human immunoglobulin. Residues from a complementary determining region (CDR) of a human recipient antibody are replaced by residues from a CDR of a non-human species (donor antibody) such as mouse, rat or rabbit having the desired specificity. Optionally, Fv framework residues of the human immunoglobulin are replaced by corresponding non-human residues. See Jones et al., Nature, 321:522-525 (1986); Riechmann et al., Nature, 332:323-327 (1988); and Presta, Curr. Op. Struct. Biol., 2:593-596 (1992). Methods for humanizing non-human antibodies are well known in the art. See Jones et al., Nature, 321:522-525 (1986); Riechmann et al., Nature, 332:323-327 (1988); Verhoeyen et al., Science, 239:1534-1536 (1988); and (U.S. Pat. No. 4,816,567).

### Laboratory Applications

The present invention further includes an exemplary microchip for use in clinical settings for detecting gene expression levels of one or more genes described herein as being associated with outcome, risk classification, cytogenics or subtype in high risk B-ALL, including high risk pediatric B-ALL. In a preferred embodiment, the microchip contains DNA probes specific for the target gene(s). Also provided by the invention is a kit that includes means for measuring expression levels for the polypeptide product(s) of one or more such genes, including any of the genes listed in Tables 1P and 1Q. In certain preferred embodiments, the microchip contains DNA probes for all 31 genes or 26 genes which are set forth in Tables 1P and 1Q. Various probes can be provided onto the microchip representing any number and any variation of gene products as otherwise described in Table 1P or 1Q. In a preferred embodiment, the kit is an immunoreagent kit and contains one or more antibodies specific for the polypeptide(s) of interest.

Relevant portions of the below cited references are referenced and incorporated herein. In addition, previously published WO 2004/053074 (Jun. 24, 2004) is incorporated by reference in its entirety herein.

In the present invention, sophisticated computational tools and statistical methods were used to reduce the comprehensive molecular profiles to a more limited set of 8 genes from Table 1P or 11 genes (preferably 9 genes) from Table 1Q (a gene expression “classifier”) that is highly predictive of overall outcome in high risk B-ALL, including high risk pediatric B-ALL.

As described in the following examples, the inventors examined pre-treatment specimens from 207 patients with high risk B-precursor acute lymphoblastic leukemia (ALL) who were uniformly treated on Children's Oncology Group Trial COG P9906. Gene expression profiles were correlated with clinical features, treatment responses, and relapse free survivals (RFS). The use of four different unsupervised clustering methods showed significant overlap in the classification of these patients. Two clusters contained all children with either t(1;19)(q23;p 13) translocations or MLL rearrangements. The other six clusters were novel and not associated with recurrent chromosomal abnormalities or distinctive clinical features. One of these clusters (R6; n=21) had significantly better 4-year RFS of 95% as compared to the 4-year RFS of 61% for the entire cohort (P=0.002). A cluster of children (R8; n=24) with dismal outcomes was found with a 4 year RFS of only 21% (P<.0.001). A significant proportion of these children (63%;15/24) were of Hispanic/Latino ethnicity. Specific gene alterations in this unique subset of ALL provide the basis for up-front identification of these extremely high risk individuals and allow for the possibility of targeted therapy.

### Examples

Through the optimization and progressive intensification of standard chemotherapeutic regimens, remarkable advances have been achieved in the treatment of pediatric acute lymphoblastic leukemia (ALL).1-3 (References-First Set) In parallel, laboratory investigations have provided remarkable insights into the biologic and genetic heterogeneity of this disease with the characterization of several recurring genetic abnormalities (hyperdiploidy, hypodiploidy, t(12;21)(ETV6-RUNX1), t(1;19)(TCF3-PBX1), t(9;22)(BCR-ABL1), and translocations involving 11q23(MLL)) that are associated with distinct therapeutic outcomes and clinical phenotypes.2 Detailed risk classification schemes, incorporating pre-treatment clinical characteristics (such as age, sex, and presenting white blood cell (WBC) count), the presence or absence of recurring cytogenetic abnormalities, and measures of minimal residual disease (MRD) at the end of induction therapy, are now used to tailor the intensity of therapy to a child's relative relapse risk (categorized as “low,” “standard/intermediate,” “high,” or “very high”). 4-6 Yet, despite refinements in risk classification and improvements in overall survival, the second most common cause of cancer-related mortality in children in the United States remains relapsed ALL.7 While relapses are more frequent in children with “very high risk” disease, associated with BCR-ABL1 or hypodiploidy, relapses occur within all currently defined risk groups.1,7 Indeed, the majority of relapses occur in children initially assigned to the “standard/intermediate” or “high” risk categories.7 Thus, a primary challenge in pediatric ALL is to prospectively identify those children with higher risk disease who do not benefit from therapeutic intensification and who require the development of new therapies for cure.⁷

In the present application, we determined if gene expression profiling could be used to improve risk classification and outcome prediction in “high-risk” pediatric ALL, a risk category largely defined by pretreatment clinical characteristics (age >10 years and presenting WBC >50,000/μL) and the absence of genetic abnormalities associated with “low” (hyperdiploidy, t(12;21)(ETV6-RUNX1)) or “very high” (hypodiploidy, t(9;22)(BCR-ABL1)) risk disease.4 Over 25% of children diagnosed with ALL are initially classified as “high-risk.” Outcomes in this form of ALL remain poor with high rates of relapse and relapse-free survivals of only 45-60%.7 Furthermore, the underlying genetic features associated with this form of ALL have not been well characterized. Thus, gene expression profiling and other comprehensive genomic technologies, such as assessment of genome copy number abnormalities or DNA sequencing, have the potential to resolve the underlying genetic heterogeneity of this form of ALL and to capture genetic differences that impact treatment response which can be exploited for improved risk classification and the identification of novel therapeutic targets.8-15

### Gene Expression Classifiers for Relapse Free Survival and Minimal Residual Disease

From the gene expression profiles obtained in the pre-treatment leukemic cells of 207 uniformly treated children with high-risk ALL, we used supervised learning algorithms and extensive cross-validation techniques to build a 42 probe-set (38 gene) expression classifier predictive of relapse-free survival (RFS). In multivariate analysis, the best predictive model for RFS was this gene expression classifier combined with either flow cytometric measures of minimal residual disease (MRD) determined at the end of induction therapy (day 29), or, a 23 probe-set (21 gene) molecular classifier derived from pre-treatment samples that could predict levels of end-induction flow MRD at initial diagnosis. The application of these classifiers separated children with “high-risk” ALL into three distinct risk groups with significantly different survivals in the initial patient cohort used for modeling and in a second independent cohort of high-risk ALL patients used for validation. The gene expression classifier for RFS alone and combined with flow MRD also retained independent prognostic significance in the presence of other genetic abnormalities (IKAROS/IKZF1 deletions,16 JAK mutations,17 and gene expression signatures reflective of activated tyrosine kinases16,18) that we and others have recently discovered and determined to be associated with a poor outcome in pediatric ALL. Thus, gene expression classifiers significantly enhance outcome prediction and risk classification in high-risk ALL and in particular, identify a group of children most likely to fail current therapeutic approaches and for whom novel therapies must be developed for cure.

### Materials and Methods

### Patient Selection

Patient samples and clinical and outcome data for this study were obtained from The Children's Oncology Group (COG) Clinical Trial P9906. COG P9906 enrolled 272 eligible “high-risk” B-precursor ALL patients between Mar. 15, 2000 and Apr. 25, 2003; all patients were uniformly treated with a modified augmented BFM regimen.6,19 This trial targeted a subset of newly diagnosed “high-risk” ALL patients that had experienced a poor outcome (44% RFS at 4 years) in prior studies.5,20 Patients with central nervous system disease (CNS3) or testicular leukemia were eligible for the trial regardless of age or WBC count at diagnosis. Patients with “very high” risk features (BCR-ABL1 or hypodiploidy) were excluded while those with “low-risk” features (trisomies of chromosomes 4 or 10; t(12;21)(ETV6-RUNX1)) were excluded unless they had CNS3 or testicular leukemia. The majority of patients had minimal residual disease (MRD) assessed by flow cytometry as previously described; cases were defined as MRD-positive or MRD-negative at the end of induction therapy (day 29) using a threshold of 0.01%.6 For this study, previously cryopreserved residual pre-treatment leukemia specimens were available on a representative cohort of 207 of the 272 (76%) registered patients. With the exception of differences in presenting WBC count, these 207 patients were highly similar in all other clinical and outcome parameters to all 272 patients accrued to this trial (see Supplement Table S1). For validation of the performance of the classifiers, an independent set of 84 children with “high-risk” ALL, previously treated on COG Trial 1961, was used as a validation cohort.14 (Supplement, Section 2 provides the detailed patient characteristics of the validation cohort). Treatment protocols were approved by the National Cancer Institute (NCI) and participating institutions through their Institutional Review Boards. Informed consent for clinical trial registration, sample submission, and participation in these research studies was obtained from all patients or their guardians.

### Microarray Analyses

RNA was purified from 207 pre-treatment diagnostic samples with >80% blasts (131 bone marrow, 76 peripheral blood) and hybridized to HG_U133A_Plus2.0 oligonucleotide microarrays (Affymetrix, Santa Clara, Calif., USA) after RNA quantification, cDNA preparation, and labeling (Supplement, Section 3, below). Signals were scanned (Affymetrix GeneChip Scanner) and analyzed with Affymetrix Microarray Suite (MAS 5.0). The expression signal matrix used for outcome analyses corresponded to a filtered list of 23,775 probe sets (Supplement, Section 4). This gene expression dataset may be accessed via the National Cancer Institute caArray site (see website array.nci.nih.gov/caarrayf) or at Gene Expression Omnibus (ncbi.nlm.nih.gov/geo/).

### Statistical Analyses

Relapse-free survival (RFS) was calculated from the date of trial enrollment to either the date of first event (relapse) or last follow-up. Patients in clinical remission, or with a second malignancy, or with a toxic death as a first event were censored at the date of last contact. As described in detail in the Supplement (Sections 4C, 5-9), a Cox score was used to rank genes based on their association with RFS and a Cox proportional hazards model-based supervised principal components analysis (SPCA)21 was used to build the gene expression classifier for RFS from the rank-ordered gene list. Similarly, for the development of the gene expression classifier predictive of end-induction minimal residual disease (MRD), a modified t-test was used to rank genes expressed in pre-treatment cells according to their association with day 29 flow MRD, defined as “positive” or “negative” at a threshold of 0.01%.6 Diagonal linear discriminant analysis (DLDA)22-23 was then used to build a prediction model and the classifier for MRD from the top-ranked genes. The likelihood-ratio-test (LRT) score and the prediction error rate were used in the model construction and evaluation. To avoid over-fitting, extensive crossvalidation was used to determine the numbers of top-ranked genes to be included.23 Nested crossvalidations provided predictions for individual cases as well as overall measures of the selected models' performance.22-23
For the first multivariate analysis testing the predictive power of the gene expression classifier for RFS relative to flow cytometric measures of MRD and to other clinical and genetic variables, a multivariate proportional Cox hazards regression analysis was performed with the risk score (determined by gene expression classifier for RFS), WBC (on a log scale) and flow cytometric measures of MRD as explanatory variables. The Likelihood Ratio Test (LRT) was performed to determine whether the risk score defined by the gene expression classifier for RFS was a significant predictor of time to relapse, adjusting for WBC and MRD. To determine if the gene expression classifier for RFS and the combined classifier (with flow cytometric measures of MRD) retained prognostic importance in the presence of new ALL-associated genetic abnormalities associated with a poor outcome that we and others have recently described, we accessed our recently published data reporting IKZFMKAROS deletionsl6 and JAK mutationsl7 in ALL as these studies were performed using DNA samples from the same cohort of patients with high-risk ALL (COG P9906) reported herein. The primary DNA copy number variation data reporting IKZF1 deletionsl6 may be accessed at the website: target.cancer.gov/data. The JAK mutation data17 may be accessed at pnas.org/content/suppl/2009/05/22/0811761106.DCSupplemental/0811761106SI.pdf (website). A multivariate Cox proportional hazards regression analysis was performed with each expression classifier and included IKZFMKAROS deletions, JAK mutations, and kinase gene expression signatures as additional explanatory variables. A likelihood ratio test was then performed to determine if the classifiers retained independent prognostic significance adjusting for the effects of all covariates. All statistical analyses utilized Stata Version 9 and R.

### Results

### Patients and Clinical Risk Factors

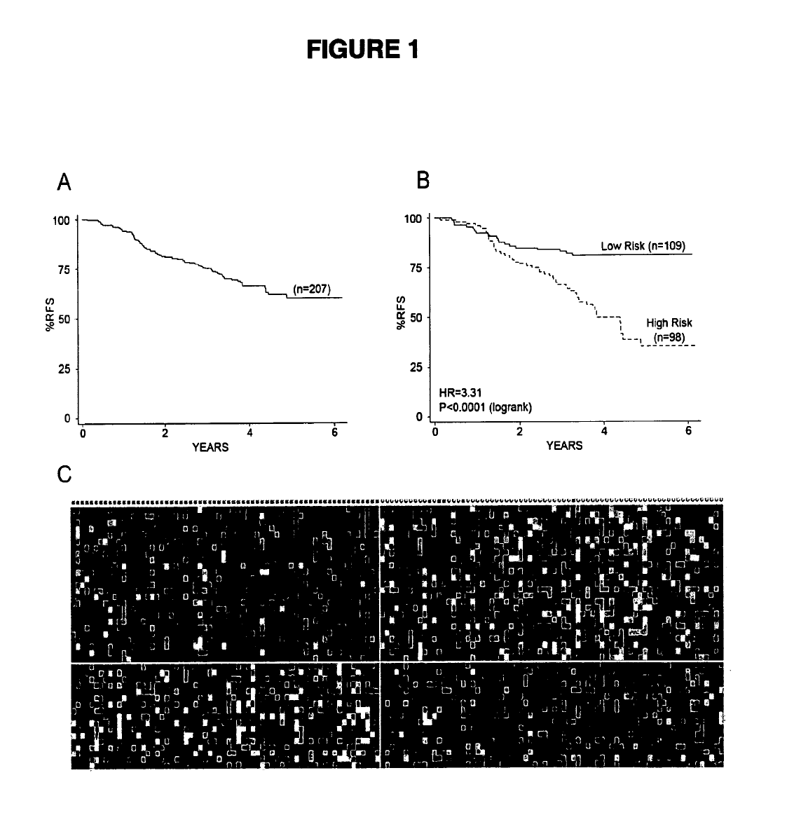

The median age of the 207 high-risk B-precursor ALL patients registered to COG Trial P9906 was 13 years (range: 1-20 years) (Table 1). While 23 of the 207 ALL patients had a t(1;19)(TCF3-PBX1) and 21 had various translocations involving MLL, the remaining 163 high-risk cases had no other known recurring cytogenetic abnormalities (Table 1). Relapse-free survival in these 207 patients was 66.3% at 4 years (95% CI: 59-73%) (FIG. 1A). Day 29 minimal residual disease, measured using flow cytometric techniques (end-induction flow MRD), was detected in 35% (67/191) (Table 1).6 Among pre-treatment clinical variables (age, sex, and CNS involvement), the presence of recurrent cytogenetic abnormalities (TCF3-PBX1 and MLL), and measures of minimal residual disease, only end-induction flow MRD and increasing WBC count were significantly associated with decreased RFS and both retained significance in multivariate analysis (LRT based on COX regression, P<0.001) (Table 1). A trend towards declining RFS was also observed among the 25% of children with Hispanic/Latino ethnicity (P=0.049) (Table 1).

TABLE 1
Association of Relapse Free Survival with Clinical
and Genetic Features in the High-Risk ALL Cohort

Association with Relapse
Free Survival²

| Characteristic | | Hazard Ratio | P-Value |
|---|---|---|---|
| Age | | | |
| ≧10 Yrs | 132 | 1 | |
| <10 Yrs | 75 | 1.152 | 0.561 |
| Age | | | |
| Median | 13 yrs | | |
| Range | 1-20 | .995 | 0.817 |
| Sex | | | |
| Male | 137 | 1 | |
| Female | 70 | 0.769 | 0.320 |
| WBC | | | |
| Median | 62.3K | | |
| Range | 1-959 | 1.003 | <0.001 |
| MRD at Day 29¹ | | | |
| Negative | 124 | 1 | |
| Positive | 67 | 2.805 | <0.001 |
| Race | | | |
| Hispanic | 51 | 1.644 | 0.049 |
| or Latino | | | |
| Others | 156 | 1 | |
| MLL | | | |
| Positive | 21 | 1.061 | 0.881 |
| Negative | 186 | 1 | |
| E2A/PBX1 | | | |
| Positive | 23 | .704 | 0.409 |
| Negative | 184 | 1 | |
| CNS | | | |
| No blasts | 160 | 1 | |
| <5 blasts | 26 | 1.078 | 0.826 |
| ≧5 blasts | 21 | 0.670 | 0.392 |

¹Only 191/207 patients in the high-risk ALL cohort had flow MRD results at end-induction.
²Hazard ratio and corresponding p value are based on Cox regression.

### A Gene Expression Classifier Predictive of Survival

Gene expression profiles were obtained from pre-treatment leukemic samples in each of the 207 high-risk ALL patients. To develop a gene expression-based classifier predictive of relapse free survival (RFS), each of the 23,775 informative probe-sets on the gene expression microarrays was ranked based on strength of association with RFS (Cox score).21 As detailed in the Supplement (Sections 4C, 5, 8), a Cox proportional hazards model-based supervised principal component analysis (SPCA) was used to build the expression classifier for RFS which was optimized by performing 20 iterations of 5-fold crossvalidation.21 The final model incorporated the top 42 Affymetrix microarray probe sets corresponding to 38 unique genes (see Supplement Table S4 for the gene list; false discovery rate=8.45%, SAM).24 The predicted gene expression classifier-based “risk score” for relapse for a given patient was computed via nested leave-one-out cross-validation (LOOCV) over the full model building procedure (Supplement, Section 5 and 8). With a threshold of zero, the gene expression classifier-derived risk scores significantly separated the 207 high-risk ALL patients into low (4 yr RFS: 81%, 95% CI: 72-87%; n=109) versus high (4 yr RFS: 50%, 95% CI: 39-60%; n=98) risk groups (FIGS. 1B and C). Increased expression of BMPR1B, CTGF (CCN2), TTYH2, IGJ, NT5E (CD73), CDC42EP3, TSPAN7, and decreased expression of NR4A3 (NOR-1), RGS1-2, and BTG3 were observed in the “high” gene expression risk group with the poorest outcome (FIG. 1C). In a multivariate Cox-regression analysis, the likelihood ratio test (LRT) revealed that the gene expression classifier for RFS provided significant independent information for outcome prediction, even after adjusting for flow MRD and WBC count (P=0.001).

### Improving Risk Classification and Outcome Prediction by Combining the Gene Expression Classifier and Flow Cytometric Measures of MRD

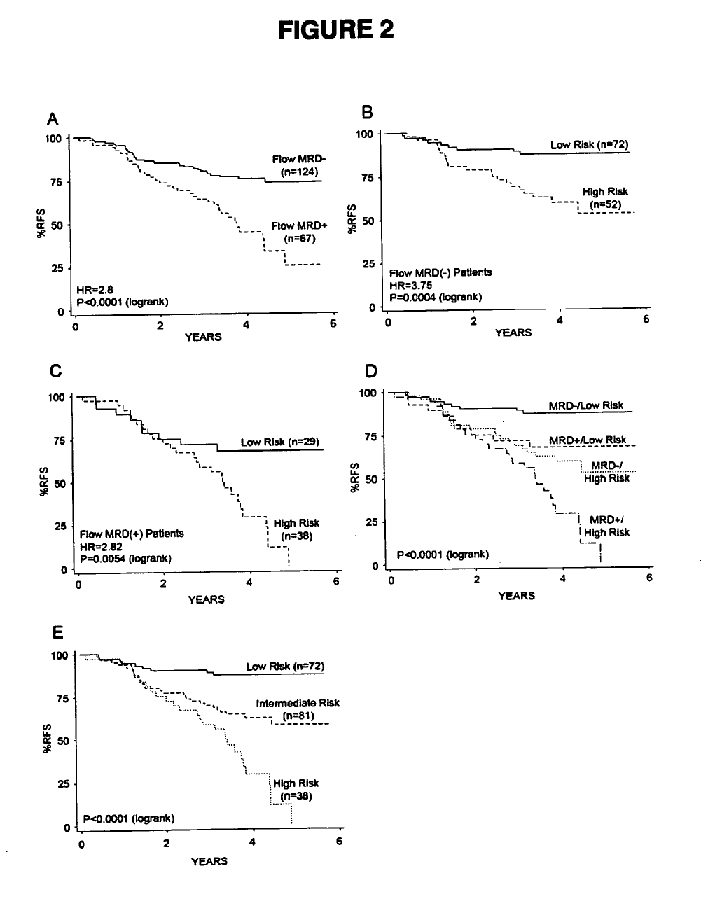

Flow cytometric measures of minimal residual disease (flow MRD), measured at the end of induction therapy (day 29), were also capable of distinguishing two groups of patients with significantly different outcomes within the high-risk ALL cohort (FIG. 2A).6 However, the independent prognostic impact of the gene expression-based classifier for RFS could further split both the flow MRD-negative patients (FIG. 2B) and flow MRD-positive patients (FIG. 2C) into two distinct patient groups with significantly different RFS (P=0.0004 and P=0.0054 respectively). It was particularly striking that the application of the gene expression classifier to the flow MRD-negative patients (FIG. 2B) distinguished a group of high-risk ALL patients who did extremely well in the COG P9906 clinical trial (87% RFS at 4 years; 95% CI: 77-93%). Similarly, applying the gene expression classifier to the flow MRD-positive patients distinguished a group of patients who did relatively well (68%% EFS at 4 years; 95% CI: 47-82%) from those who had an extremely poor outcome (FIG. 2C). As both the gene expression classifier for RFS and flow MRD provided independent prognostic information in a multivariate Cox-regression analysis (each P=0.001), we built a combined risk classifier using these two variables; this combined classifier was capable of distinguishing four distinct prognostic groups within this cohort of high-risk ALL patients (FIG. 2D). The 72 patients in the lowest risk group (38% of cases in the cohort; Table 2), who had low risk gene expression classifier scores and negative end-induction flow MRD, showed significantly better RFS than the other groups (P<0.0001). While all 20 cases with a t(1;19)(TCF3-PBX1) were contained within this lowest risk group (FIGS. 2D and E), it is of interest that another 52 patients lacking known recurring cytogenetic abnormalities were also assigned to this risk group (Table 2). Similarly, the 38 patients in the highest risk group (20% of cohort), who had high gene expression classifier risk scores and positive end-induction flow MRD, displayed significantly worse RFS (29% RFS at 4 years, 95% CI: 14-46%, which continued to decline at 5 yrs) (P<0.0001) (FIGS. 2C-E; Table 2). No significant survival differences (P=0.57) were observed among those with discordant predictors, either those patients with low gene expression classifier risk scores and positive end-induction flow MRD (28/191, 15% of cohort) or those with high gene expression classifier risk scores and negative endinduction flow MRD (52/191, 27% of cohort). These two groups were thus combined into an intermediate risk group (FIG. 2E). FIG. 2E provides the Kaplan-Meier survival estimates for the three risk groups defined by the combined classifier and highlight the significant differences in RFS. These three risk groups varied significantly in age and in the presence of the known recurring cytogenetic abnormalities (Table 2). While the 17 patients with MLL translocations were distributed within the low and intermediate risk groups, all 20 cases with t(1;19)(TCF3-PBX1) were in the lowest risk group, as discussed above (Table 2; FIG. 2E). Interestingly, of the 8 relapses that occurred in the lowest risk group, all 8 were ALL cases with t(1;19)(TCF3-PBX1). Children in each of the three risk groups had similar proportions of relapse within the bone marrow or isolated to the CNS (Table 2).

TABLE 2
Clinical and Genetic Features of The Three Risk
Groups Determined by the Combined Application of
the Gene Expression Classifier for RFS and Flow Cytometric
Measures of Minimal Residual Disease¹

| | Combined Risk Group | | | | P-value |
|---|---|---|---|---|---|
| | | Inter- | | Total | (Fisher |
| Characteristics | Low | mediate | High | Cohort | Exact) |
| RFS at 4 Years | 87% | 62% | 29% | 61% | <0.0001 |
| Number of | 72 | 81 | 38 | 191 | |
| cases | | | | | |
| Age | | | | | |
| ≧10 Yrs | 56 (78%) | 40 (49%) | 29 (76%) | 125 (65%) | <0.001 |
| <10 Yrs | 16 (22%) | 41 (51%) | 9 (24%) | 66 (35%) | |
| Age | | | | | |
| Median | 14.02 | 9.82 | 13.91 | 13.31 | |
| 5^{th}-95^{th} | 2.64-18.27 | 1.43-17.82 | 1.99-18.25 | 1.78-18.16 | |
| Percentiles | | | | | |
| Sex | | | | | |
| Female | 25 | 28 | 11 | 64 | 0.83 |
| Male | 47 | 53 | 27 | 127 | |
| WBC | | | | | |
| ≧50K | 30 | 50 | 19 | 99 | 99 |
| <50k | 42 | 31 | 19 | 92 | |
| WBC - count | | | | | |
| Median | 37.25 | 92.7 | 51.55 | 62.3 | |
| 5^{th}-95^{th} | 2.3-246.4 | 3-314.8 | 2.3-478 | 2.3-314.8 | |
| Percentiles | | | | | |
| Race | | | | | |
| Hispanic & | 17 | 16 | 13 | 46 | 0.242 |
| Latino | | | | | |
| Others | 54 | 64 | 25 | 143 | |
| MLL¹ | | | | | |
| Negative | 65 | 71 | 38 | 174 | 0.057 |
| Positive | 7 | 10 | 0 | 17 | |
| t(1; 19)(TCF3- | | | | | |
| PBX1)¹ | | | | | |
| Negative | 52 | 81 | 38 | 171 | <0.001 |
| Positive | 20 | 0 | 0 | 20 | |
| CNS | | | | | |
| No blasts | 57 | 57 | 32 | 146 | 0.457 |
| <5 blasts | 7 | 14 | 4 | 25 | |
| ≧5 blasts | 8 | 10 | 2 | 20 | |
| Relapse site | | | | | |
| Isolated | 3 | 15 | 5 | 23 | 0.095 |
| CNS² | | | | | |
| Marrow | 5 | 13 | 17 | 35 | |

¹Only 191 of the 207 patients in the high risk ALL cohort had flow MRD results at end-induction; hence this table reports on191 total patients. Flow MRD results were available on only 17/21 MLL and 20/23 t(1; 19)(TCF3-PBX1) patients.
²No association was seen between patients with isolated CNS relapse and those with CNS blasts at diagnosis (_{χ}2 test, P = 0.93).

To assure that the gene expression classifier could improve outcome prediction in high-risk ALL patients lacking known recurring cytogenetic abnormalities, we built a second gene expression classifier for RFS using a subset of 163 of the original 207 COG 9906 high-risk ALL patients excluding those cases with MLL (n=21) or E2A-PBX1 translocations (n=23), again using a Cox proportional hazards model-based supervised principal component analysis with extensive cross-validation (see Supplement Section 10). The resulting classifier for RFS contained 32 probe sets (29 unique genes; list provided in Supplement, Table S8) and had a high degree of overlap (84%) with the genes in the initial classifier (Supplement, Table S4).

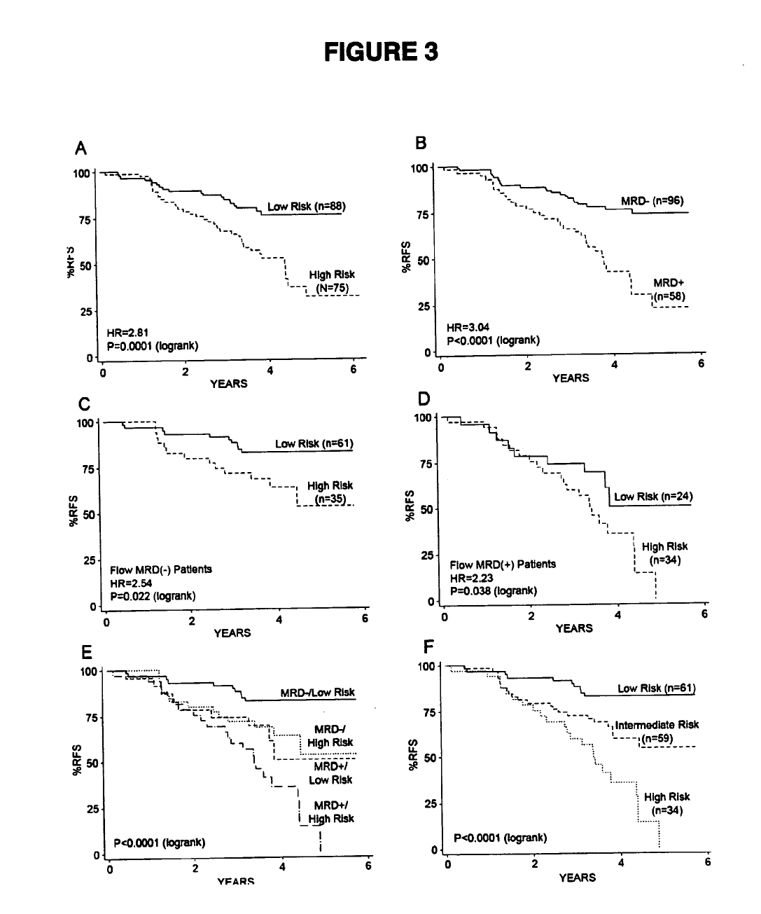

With a threshold of zero, the risk scores derived from this second classifier also significantly separated the 163 ALL cases into low (4 yr RFS: 76%, 95% CI: 64-84%; n=88) versus high (4 yr RFS: 52%, 95% CI: 40-64%; n=75) risk groups (P=0.0001) (FIG. 3A). Flow cytometric measures of end-induction MRD were also capable of distinguishing two risk groups within these 163 high-risk ALL cases (FIG. 3B) and application of the gene expression classifier further divided both the flow MRD-negative (FIG. 3C) and flow MRD-positive (FIG. 3D) patients into distinct risk groups with significantly different outcomes. Combining this second classifier for RFS with end induction flow MRD yielded four distinct risk groups with significantly different outcomes (P<0.0001; FIG. 3E). As no significant survival differences were observed among the two groups with discordant predictors, these groups were combined into an intermediate risk group (FIG. 3F). As shown in FIG. 3F, the Kaplan-Meier survival estimates for the three risk groups defined by this second combined classifier demonstrated highly significant differences in RFS (low (83% 4 year RFS, 95% CI: 70-90%), intermediate (60% 4 yr RFS, 95% CI:44-72%) and high (35% 4 yr RFS, 95% CI:19-44%) (P<0.0001). These results demonstrate that gene expression classifiers significantly refine risk classification in high-risk ALL cases lacking known cytogenetic abnormalities.

### A Gene Expression Classifier Predictive of End-Induction Flow MRD

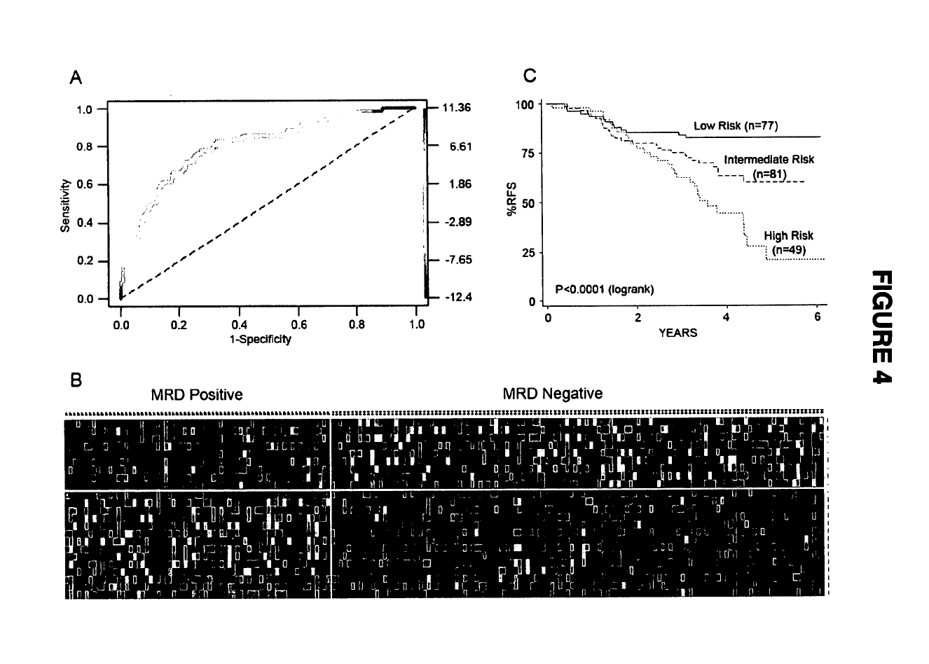

The clinical application of a combined classifier utilizing the gene expression classifier for RFS and day 29 flow MRD would require waiting until the end of induction therapy, precluding earlier intervention in patients who were destined to ultimately fail therapy. To develop a gene expression classifier predictive of end-induction MRD in diagnostic pre-treatment specimens, 23,775 informative probe sets from 191 patients (of the 207 patients who had day 29 MRD results available) were ranked on their association with MRD (Supplement, Sections 6 and 9). Using a threshold of 1% for the false discovery rate, SAM identified 352 probe sets significantly associated with positive end-induction flow MRD (Supplement, Table S6). A DLDA mode122,23 predicting MRD was built and optimized by performing 100 iterations of 10-fold cross-validation. The final model incorporated the top 23 probe sets (21 unique genes) (Supplement, Table S5), which separated the patients into two groups with significantly different outcomes (log rank test, P=0.014). FIG. 4A shows the receiver operating characteristic (ROC) curve for the nested LOOCV predictions of the classifier. The 23 probe sets in the gene expression classifier predictive of end-induction MRD (FIG. 4B) include the genes BAALC, P2RY5, TNFSF4, E2F8, IRF4 CDC42EP3, KLF4, and two probe sets each for EPB41L2 and PARP15. When the gene expression classifier predictive of MRD was substituted for the day 29 flow MRD data and then combined with the expression classifier for RFS, three distinct risk groups were resolved that had significantly different RFS at 4 years (low: 82%; intermediate: 63%; and high risk: 45%) (FIG. 4C). While still highly statistically significant (P<0.0001), the combined classifier using the gene expression classifier for RFS and the gene expression classifier predicting end-induction MRD (FIG. 4C) was slightly less discriminatory than the one combining the gene expression classifier for RFS and flow MRD (FIG. 2E).

### Validation of the Classifiers in an Independent Data Set

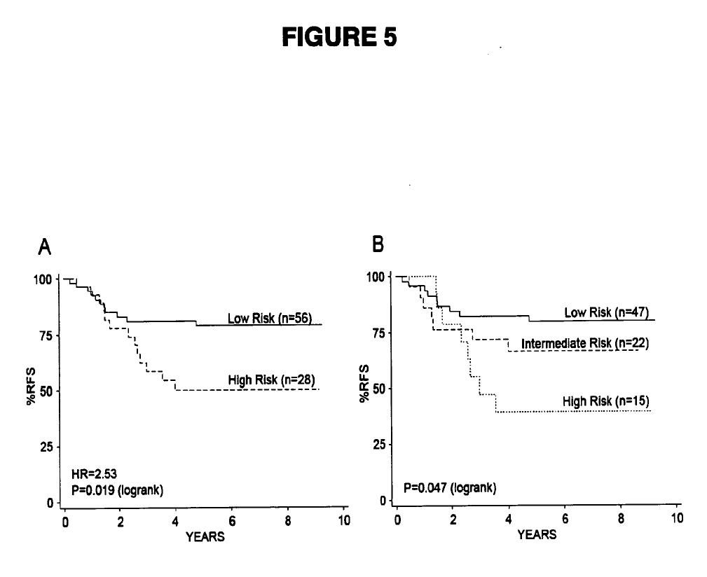

The inventors next determined whether the gene expression classifiers were predictive of outcome in a second independent cohort of 84 children with high-risk ALL treated on a different clinical trial (COG/CCG 1961).14,19 In contrast to the initial COG 9906 high-risk ALL cohort, a WBC count >50,000411 (LRT, P=0.014) and male sex (LRT, P=0.018) were associated with a worse RFS (Supplement, Section 2).14,19 Flow MRD was not evaluated in the CCG 1961 trial. The initial 38 gene expression classifier for RFS (Supplement Table S4) that we developed from COG P9906 predicted a risk score among these 84 patients that was significantly associated with RFS (Cox proportional hazard regression, P=0.006), even after adjusting for sex and WBC count (multivariate Cox regression, P=0.01). The gene expression classifier risk scores split the 84 children from CCG 1961 into high (n=28) and low (n=56) risk groups (FIG. 5A) Unlike our initial cohort, a significantly greater number of children with WBC counts >50,000/μl were in the high (82%, 23/28) compared to the lower risk groups defined by the expression classifier (55%, 31/56) (Fisher exact test, P=0.017). Similar to the COG 9906 cohort, all children with t(1;19)(TCF3-PBX1) were in the lowest risk group, although this cytogenetic abnormality by itself did not predict RFS. We next tested the effect of the combined gene expression classifiers for RFS and MRD and were able to resolve three distinct risk groups with significantly different outcomes (FIG. 5B), demonstrating that these classifiers were capable of resolving distinct risk groups in an independent cohort of children with high-risk ALL.

Gene Expression Classifiers Retain Independent Prognostic Significance in the Presence of New Genetic Factors Associated with a Poor Outcome in Pediatric ALL

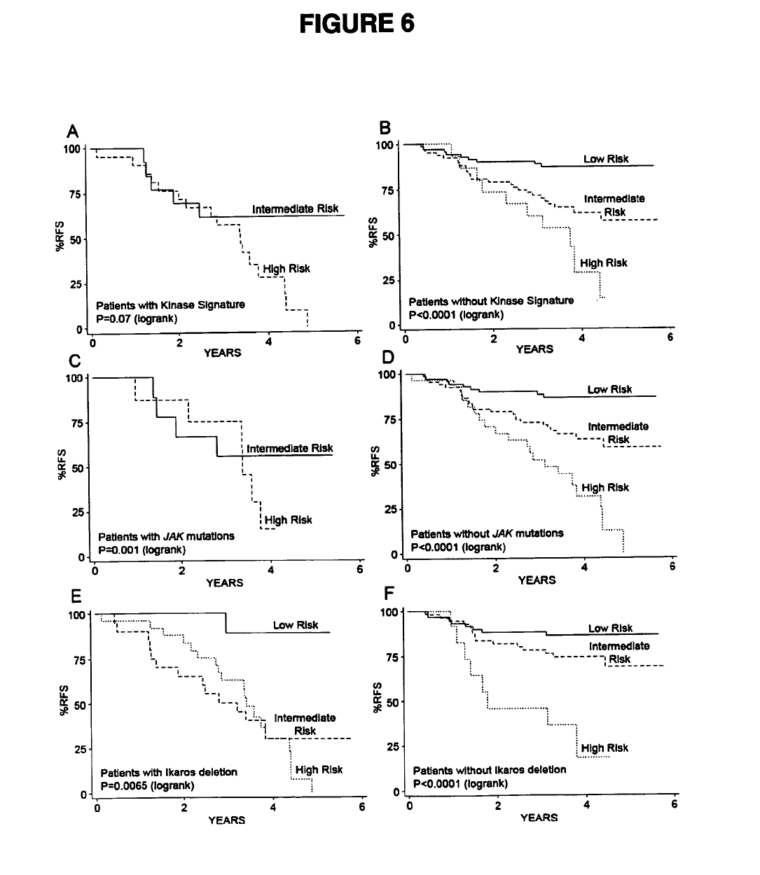

The inventors and others have recently identified new genetic features in pediatric ALL that are associated with a poor outcome, including IKAROS/IKZF1 deletions,16 JAK mutations,17 and gene expression signatures reflective of activated tyrosine kinase signaling pathways (termed “kinase signatures”).16,18 Two of these studies16,18 first reported the discovery of ALL cases that lacked a classic BCR-ABLJ translocation but which had gene expression profiles reflective of tyrosine kinase activation. Our more recent work17 has determined that the majority of these cases have activating mutations of the JAK family of tyrosine kinases. We thus wished to determine whether the gene expression classifier for RFS, or the combined classifier, retained independent prognostic significance in the presence of these genetic abnormalities. As detailed in the METHODS section, our studies reporting IKAROS/IKZF1 deletions,16 activated kinase signatures,16 and JAK mutations 17 used samples from the same COG 9906 high-risk ALL cohort; thus, we could readily perform this multivariate analysis. As shown in Table 3, below, activated kinase signatures, JAK family mutations, and IKAROS/IKZF1 deletions were each significantly associated with the highest risk group as defined by the gene expression classifier for RFS in the COG 9906 high-risk ALL cases. Not only did the gene expression classifier for RFS assign all 38 cases with a kinase signature to the highest risk group, it also assigned another 60 cases to this risk group (Table 3). Similarly, while all cases with JAK mutations were assigned to the highest risk group by the gene expression classifier for RFS, an additional 74 cases lacking these mutations were also assigned to this high risk group (Table 3, below). The gene expression classifier also refined risk classification in the presence of IKAROS/IKZF1 deletions (Table 3, below). In a multivariate Cox regression analysis, only the gene expression classifier for RFS (p=0.005) and IKAROS/IKZF1 deletions (p=0.003) retained prognostic significance (Table 4, below). A likelihood ratio test determined that the gene expression classifier for RFS retained independent prognostic significance (P=0.0143) when adjusting for all other covariates. We also examined the association between risk groups as defined by the combined gene expression classifier for RFS and end-induction flow MRD (the “combined” classifier) with kinase signatures, JAK family mutations, and IKAROS/IKZF1 deletions (Table 5, FIG. 6). Again, significant associations between each of these variables and the three risk groups (low, intermediate, and high) defined by the combined classifier were seen (Table 5, below). As shown in FIG. 6, the application of the combined classifier refined risk classification and distinguished different patient groups with statistically significant different RFS in the presence or absence of a kinase signature (FIGS. 6A and B), in the presence or absence of JAK mutations (FIGS. 6C and D), and in the presence or absence of IKAROS/IKZF1 deletions (FIGS. 6E and F). In a multivariate Cox regression analysis (Table 6, below), only the combined classifier retained independent prognostic significance for outcome prediction. The likelihood ratio test revealed that the combined classifier retained independent prognostic significance after adjusting for the effects of all other genetic abnormalities (P=0.0001).

TABLE 3
Association of Kinase Gene Expression Signatures, JAK Mutations,
and IKAROS/IKZF1 Deletions with the Low vs. High Risk Groups Defined
by the Gene Expression Classifier for RFS¹

| | | Risk Group Determined by Gene | | | | | p-value |
|---|---|---|---|---|---|---|---|
| | | Expression Classifier for RFS | | | | | (Fisher |
| Genetic Feature | | Low Risk | High Risk | | Total | | Exact) |
| Kinase Signature | Yes | 0 | 38 | (39%) | 38 | (18%) | <.001 |
| | No | 109 | 60 | (61%) | 169 | (82%) | |
| | Total | 109 | 98 | (100%) | 207 | (100%) | |
| JAK1/JAK2 | Yes | 0 | 19 | (20%) | 19 | (10%) | <.001 |
| Mutation | No | 105 | 74 | (100%) | 179 | (90%) | |
| | Total | 105 | 93 | (100%) | 198 | (100%) | |
| IKAROS/IKZF1 | Yes | 14 (13%) | 41 | (44%) | 55 | (28%) | <.001 |
| Deletion | No | 91 (87%) | 52 | (56%) | 143 | (72%) | |
| | Total | 105 (100%) | 93 | (100%) | 198 | (100%) | |

¹The gene expression classifier for RFS used in this analysis is the initial classifier developed with 42 probe sets (38 unique genes) provided in Supplement Table S4.

TABLE 4
Multivariate Cox-Regression Analysis of the Prognostic
Significance of the Risk Group Determined by the Gene Expression
Classifier for RFS¹ in the Presence of Genetic Factors
in ALL Associated with a Poor Outcome

| | Hazard Rato² | | |
|---|---|---|---|
| | | 95% Confidence | |
| Covariates | Estimate | Interval | P-Value |
| Gene Expression Classifier | | | |
| for RFS Risk Group | | | |
| High Risk vs. Low Risk | 2.380 | 2.3.6-4.338 | 0.005 |
| IKAROS/IKZF1 Deletions | | | |
| Positive vs. Negative | 2.237 | 1.316-3.803 | 0.003 |
| JAK Mutations | | | |
| Positive vs. Negative | 1.020 | .500-2.081 | 0.957 |
| Kinase Gene Expression | | | |
| Signature | | | |
| Positive vs. Negative | 1.094 | .590-2.030 | 0.774 |

¹The gene expression classifier for RFS used in this analysis is the initial classifier developed with 42 probe sets (38 unique genes) provided in Supplement Table S4.
²Hazard ratios and corresponding p value are based on Cox regression.

TABLE 5
Association of Kinase Gene Expression Signatures, JAK Mutations, and
IKAROS/IKZF1 Deletions with the Three Risk Groups Defined by the Combined Gene
Expression Classifier for RFS¹ and Flow Cytometric Measures of Minimal Residual
Disease

| | | | | | | p-value |
|---|---|---|---|---|---|---|
| | | Combined Risk Group | | | | (Fisher |
| Genetic Feature | | Low | Intermediate | High | Total | Exact) |
| Kinase | Yes | 0 | 13 (16%) | 22 (58%) | 35 (18%) | <0.001 |
| Signature | No | 72 (100%) | 68 (84%) | 16 (42%) | 156 (82%) | |
| | Total | 72 (100%) | 81 (100%) | 38 (100%) | 191 (100%) | |
| JAK1/JAK2 | Yes | 0 | 9 (12%) | 9 (24%) | 18 (10%) | <0.001 |
| Mutation | No | 69 (100%) | 67 (88%) | 28 (76%) | 164 (90%) | |
| | Total | 69 (100%) | 76 (100%) | 37 (100%) | 182 (100%) | |
| IKAROS/IKZF1 | Yes | 9 (13%) | 20 (26%) | 25 (68%) | 54 (30%) | <0.001 |
| Deletion | No | 60 (87%) | 56 (74%) | 12 (32%) | 128 (70%) | |
| | Total | 69 (100%) | 76 (100%) | 37 (100%) | 182 (100%) | |

¹The gene expression classifier for RFS used in this analysis is the initial classifier developed with 42 probe sets (38 unique genes) provided in Supplement Table S4.

TABLE 6
Multivariate Cox-Regression Analysis of the Prognostic
Significance of the Risk Group Determined by the Combined
Gene Expression Classifier for RFS¹ and Flow Cytometric
Measures of MRD in the Presence of Genetic Factors
in ALL Associated with a Poor Outcome

| | Hazard Ratio² | | |
|---|---|---|---|
| | | 95% Confidence | |
| Covariates | Estimate | Interval | P |
| Risk Group Determined | | | |
| by Gene Expression | | | |
| Classifier for RFS and Flow MRD | | | |
| Intermediate Risk vs. Low Risk | 3.366 | 1.569-7.222 | 0.002 |
| High Risk vs. Low Risk | 6.214 | 2.547-15.160 | 0.000 |
| IKAROS/IKZF1 Deletions | | | |
| Positive vs. Negative | 1.684 | .923-3.072 | 0.089 |
| JAK Mutations | | | |
| Positive vs. Negative | .987 | .469-2.076 | 0.973 |
| Kinase Gene Expression Signature | | | |
| Positive vs. Negative | .988 | .506-1.929 | 0.972 |

¹The gene expression classifier for RFS used in this analysis is the initial classifier developed with 42 probe sets (38 unique genes) provided in Supplement Table S4.
²Hazard ratios and corresponding p value are based on Cox regression.

### Discussion

While gene expression profiling studies in the acute leukemias have identified gene expression “signatures” associated with recurrent cytogenetic abnormalities8,25,26 and in vitro drug responsiveness,9-11,15 fewer studies have reported and validated gene expression classifiers predictive of survival.13,14 In this report, gene expression classifiers predictive of relapse free survival (RFS) and end-induction minimal residual disease were derived from the gene expression profiles obtained in the pre-treatment samples of 207 children with B-precursor high-risk ALL. A 42 probe-set (containing 38 unique genes) expression classifier predictive of relapse-free survival (RFS) was capable of resolving two distinct groups of patients with significantly different outcomes within the category of pediatric ALL patients traditionally defined as “high-risk.” In multivariate analyses, only the gene expression-based classifier for RFS and flow cytometric measures of end-induction MRD provided independent prognostic information for outcome prediction. By combining the risk scores derived from the gene expression classifier for RFS with end-induction flow MRD, three distinct groups of patients with strikingly different treatment outcomes could be identified. Similar results were obtained when modeling only those high-risk ALL cases that lacked any known recurring cytogenetic abnormalities. Perhaps most importantly, in terms of the future potential clinical utility of gene expression-based classifiers for risk classification, we further demonstrated that both the gene expression classifier for RFS and the combination of this classifier with end-induction flow MRD retained independent prognostic significance for outcome prediction in the presence of new genetic abnormalities that we and others have recently discovered and found to be associated with a poor outcome in pediatric ALL (IKAROS/IKZF1 deletions, JAK mutations, and kinase signatures). The combined classifier further refilled outcome prediction in the presence of each of these mutations or signatures, distinguishing which cases with JAK mutations, kinase signatures or IKAROS/IKZF1 deletions would have a good (“low risk”), intermediate, or poor (“high risk”) outcome (Table 5, FIG. 6). Thus, while IKZF1 deletions and JAK mutations are exciting new targets for the development of novel therapeutic approaches in pediatric ALL, ssessment of these genetic abnormalities alone may not be fully sufficient for risk classification or to predict overall outcome. As gene expression profiles reflect the full constellation and consequence of the multiple genetic abnormalities seen in each ALL patient and as measures of minimal residual disease are a functional biologic measure of residual or resistant leukemic cells, they may have an enhanced clinical utility for refinement of risk classification and outcome prediction.

The results reported herein, as well as those of other recent studies,16-18 reveal the striking molecular and biologic heterogeneity within children who have traditionally been classified as “high-risk” ALL. Unexpectedly, 72/207 (38%) of the “high-risk” ALL patients studied in the COG 9906 ALL cohort were found by the combined gene expression classifier for RFS and flow MRD classifier to have a significantly better survival (87% RFS at 4 years) when compared with the entire cohort (66% survival at 4 years). This group of patients, which included all 20 cases with t(1;19)(TCF3-PBX1) and an additional 52 cases whose underlying genetic abnormalities remain to be discovered, was characterized by high expression of the tumor suppressor genes and signaling proteins RGS2, NFKBIB, NR4A3, DDX21, and BTG3.27-30 Application of the combined classifier also identified 38/207 (20%) of patients in the COG 9906 cohort who had a dismal 4 year RFS of 29% (approaching 0% at 5 yrs). Highly expressed in this group of patients with the worst outcome were genes (BMPR1B, CTGF (CCN2), TTYH2, IGJ, PON2, CD73, CDC42EP3, TSPAN7, SEMA6A) involved in adaptive cell signaling responses to TGFP, stem cell function, B-cell development and differentiation, and the regulation of tumor growth.27-45 These highest risk cases lacked expression of the genes (NR4A3, BTG3, RGS1 and RGS2) whose relatively high expression characterized the ALL cases with the best outcome. Not surprisingly, given that all cases with an activated kinase signature were assigned to the highest risk group with the combined classifier, six of the genes associated with our kinase signature (BMPR1B, ECM1, PON2, SEMA6A, and TSPAN7) were contained within our gene expression classifier for RFS. The genes that characterize the risk groups defined by the combined classifier provide important clues to the multiple complex pathways and mechanisms of leukemic transformation in pediatric ALL.

The kinetics of early treatment response, best assessed by molecular or flow cytometric measures of minimal residual disease (MRD) after the first 1-3 months of therapy, are a potent predictor of outcome in leukemia. Yet, MRD data are not available at initial diagnosis and relapses occur in some pediatric ALL patients (such as those with t(1;19)TCF3-PBX1)), who have an excellent (negative) end-induction MRD response. Ideally, one would want to identify as early as possible those ALL patients who are most likely to fail therapy so that novel treatment interventions or alternative induction methods could be employed. Using the combined gene expression classifier for RFS and end-induction flow MRD, we identified 38 patients in the initial cohort of 207 patients who were destined to ultimately fail intensified traditional therapy for ALL. We therefore built a 23 probe-set (21 gene) gene expression classifier predictive of day 29 flow MRD in diagnostic, pre-treatment samples that could successfully replace end-induction flow MRD in our risk model. Among several interesting genes in the classifier predictive of end-induction MRD was BAALC, a novel marker of an early progenitor cells that has been reported to confer a worse outcome and primary resistance in acute leukemia, including ALL and AML in adults.46-47 Given the relatively old age (mean=13 years) of the children and adolescents in our ALL cohort and the presence of genes in our gene expression classifiers for RFS and MRD that have previously been associated with a poor outcome in adult ALL (such as CTGF43-44 and BAALC46-47), we hypothesize that the gene expression classifiers that we have developed for pediatric ALL may also be useful for risk classification and outcome prediction in adults with ALL. These studies are now in progress. The results of our studies provide evidence that improved outcome prediction and risk classification can be achieved in ALL through the development of gene expression classifiers. The application of gene expression classifiers allows for the prospective identification of a significant subgroup of ALL patients with little chance for cure on contemporary chemotherapeutic regimens. Further analysis of these expression profiles, coupled with other comprehensive genomic studies, will hopefully lead to the continued identification of novel targets and more effective therapies for these children.

### 1^{st} Supplement—Gene Expression Classifiers for Relapse Free Survival and Minimal Residual Disease

### Patients and Clinical Risk Factors

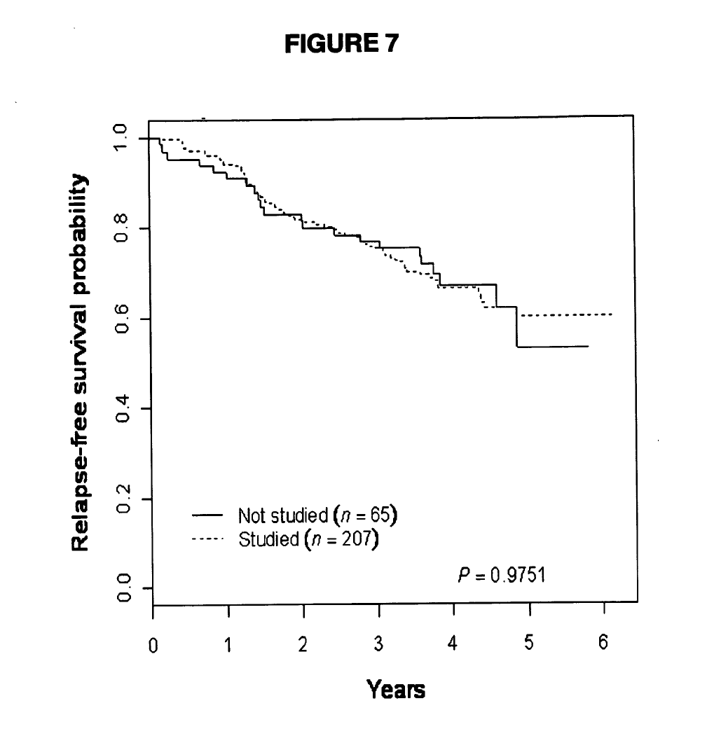

For this study, pre-treatment cryopreserved leukemia specimens were available on a representative cohort of 207 of the 272 (76%) patients registered to COG P9906.¹ With the exception of presenting white blood cell count (WBC), the clinical and outcome parameters of these 207 patients did not differ significantly from all 272 patients (see Table S1 and FIG. 7/S1). As shown in Table S1 and FIG. 7/S1, the differences in various characteristics between the entire group (n=272) and the present study cohort (n=207) were examined by the statistical comparisons between the present study cohort and remaining patients (n=65) not included in the present study. Each P-value in Table S1 and FIG. 7/S1 is that of the individual test which needs to be adjusted for multiple testing. A simple Bonferroni adjustment multiplies the P-values by the total number of tests.² After this adjustment, none of the characteristics are significantly different between the entire group and the cohort examined herein, except the test for WBC count when a cutoff value was considered. This trial targeted a subset (defined by age and WBC) of newly diagnosed NCI high risk ALL patients that had experienced a poor outcome (44% RFS) in prior studies.³ Patients with central nervous system disease (CNS3) or testicular leukemia were eligible regardless of age or white blood cell (WBC) count at diagnosis. Patients with “very high” risk features (BCR-ABL or hypodiploid) were excluded, while those with “low” risk features (trisomy 4+10; TEL-AML1) were excluded unless they had CNS3 or testicular leukemia. The majority of patients had minimal residual disease (MRD) assessed by flow cytometry as previously described; cases were defined as MRD-positive or MRD-negative at the end of induction therapy (day 29) using a threshold of 0.01%.¹ All treatment protocols were approved by the National Cancer Institute and all participating institutions through their Institutional Review Boards. Informed consent was obtained from all patients or their parents/guardians prior to enrollment.

TABLE S1
Comparison of High Risk ALL Patients Registered to COG P9906
(n = 272) and The Subset of Patients Examined and Modeled
for Gene Expression Signatures (n = 207)¹

| | | | | | | | Un- |
|---|---|---|---|---|---|---|---|
| | | | | | | | adjusted |
| | Not | | | | | | p-value |
| Char- | Studied | | Studied | | Total | | (Fisher's |
| acteristics | N | % | N | % | N | % | exact test) |
| Age - no. | | | | | | | |
| ≧10 Yrs | 51 | 78.46 | 132 | 63.77 | 183 | 67.28 | 0.0335 |
| <10 Yrs | 14 | 21.54 | 75 | 26.23 | 89 | 32.72 | |
| Sex - no. | | | | | | | |
| Male | 52 | 80 | 137 | 66.18 | 189 | 69.49 | 0.0442 |
| Female | 13 | 20 | 70 | 33.82 | 83 | 30.51 | |
| WBC - no. | | | | | | | |
| <50K | 52 | 80 | 99 | 47.83 | 151 | 55.51 | <0.0001² |
| ≧50k | 13 | 20 | 108 | 52.17 | 121 | 44.49 | |
| Race | | | | | | | |
| Hispanic | 15 | 23.08 | 51 | 24.64 | 66 | 24.26 | 0.9638 |
| or Latino | | | | | | | |
| Others | 47 | 72.31 | 154 | 74.39 | 201 | 73.90 | |
| Unknown | 3 | 4.61 | 2 | 0.97 | 5 | 1.84 | |
| MRD | | | | | | | |
| at day 29 | | | | | | | |
| Negative | 40 | 61.54 | 124 | 59.90 | 164 | 60.29 | 0.7550 |
| Positive | 19 | 29.23 | 67 | 32.37 | 86 | 31.62 | |
| Unknown | 6 | 9.23 | 16 | 7.73 | 22 | 8.09 | |
| MLL | | | | | | | |
| Negative | 61 | 93.85 | 186 | 89.86 | 247 | 90.81 | 0.4617 |
| Positive | 4 | 6.15 | 21 | 10.15 | 25 | 9.19 | |
| E2A/PBX1 | | | | | | | |
| Negative | 59 | 90.77 | 184 | 88.89 | 243 | 89.34 | 0.6384 |
| Positive | 5 | 7.69 | 23 | 11.11 | 28 | 10.29 | |
| Unknown | 1 | 1.54 | 0 | 0 | 1 | 0.37 | |
| CNS | | | | | | | |
| No blasts | 54 | 83.08 | 160 | 77.29 | 214 | 78.68 | 0.1009 |
| <5 blasts | 3 | 4.61 | 26 | 12.56 | 29 | 10.66 | |
| ≧5 blasts | 8 | 12.31 | 21 | 10.15 | 29 | 10.66 | |
| Total | 65 | 100 | 207 | 100 | 272 | 100 | |

¹All unknown data were removed before statistical tests were performed.
²After Bonferroni adjustment for multiple testing, only WBC remains significant at the significance level
α = 0.05.

### Validation Cohort

A subset of patients from COG 1961 “Treatment of Patients with Acute Lymphoblastic Leukemia with Unfavorable Features” was used as a validation cohort. As described in Bhojwani et al.,⁴ this trial enrolled a total of 2078 patients with NCI high risk features, i.e. WBC count ≧50,000/μl or age 10 years old, from September 1996 to May 2002. Gene expression microarray analyses were performed on pretreatment samples from 99 children treated on this study. This subset was selected to identify gene expression profiles related to early response and long term outcome and may not be representative of the entire high-risk population. These patients and their gene expression data were studied as a validation cohort for the gene expression classifier for RFS after removal of 8 children with the t(12;21), 6 with the t(9;22) translocations, and 1 who failed induction therapy. Data on the remaining 84 patients, that best reflect our patient population, are provided in the paper. Among the 6 children with the t(9;22) translocation, the two with lowest gene expression risk scores are in clinical remission, while 2 of 4 children with high gene expression risk scores have relapsed, and a third was censored. Validation of our molecular classifier for MRD was not feasible in this cohort due to the absence of flow MRD testing in the COG 1961 protocol.

### Microarray Experimental Procedures

RNA was prepared from thawed, cryopreserved samples with >80% blasts using TRIzol Reagent (Invitrogen, Carlsbad, Calif.) per the manufacturer's recommendations. Total RNA concentration was determined by spectrophotometer and quality assessed with an Agilent Bioanalyzer 2100 (Agilent Technologies). The isolated RNA was reverse transcribed into cDNA and re-transcribed into RNA.⁵ Biotinylated eRNA was fragmented and hybridized to HG_U133A Plus2 oligonucleotide microarrays (Affymetrix). Processing was performed in sets containing samples that had been statistically randomized with respect to known clinical covariates. Signal intensities and expression data were generated with the Affymetrix GCOS 1.4 software package using probe set masking as described below. All cases included in the cohort had good quality total RNA >2.5 μg and good quality scanned images. Experimental quality was assessed by GAPDH ≧1800, ≧20% expressed genes, GAPDH 3′/5′ ratios ≦4 and linear regression r-squared values of spiked poly(A) controls >0.90.

### Statistical Analysis

### Microarray Data Pre-Processing

The supervised analyses were performed using the expression signal matrix corresponding to a filtered list of 23,775 probe sets, reduced from the original 54,675. The experimental CEL files were first processed in conjunction with a tailored mask using the Affymetrix GeneChip® Operating Software 1.4.0 Statistical Algorithm package to generate a 207 patient×54,675 probe set signal data matrix and associated call matrix (Present/Absent/Marginal). The purpose of the masking was to remove those probe pairs found to be uninformative in a majority of the samples and to eliminate non-specific signals common to a particular sample type, thus improving the overall quality of the data. This was accomplished by evaluating the signals for all probes across all 207 samples and identifying those that gave mismatch (MM) signals greater than perfect match signals (PM) in more than 60% of the samples. This mask removed 94,767 probe pairs and had some impact on 38,588 probe sets (71%). As shown in Table S2, the net impact of masking was a significant increase in the number of present calls coupled with a dramatic decrease in the number of absent calls. The masked data also removed 7 probe sets entirely (none of which represented human genes). This resulted in the number of analyzable probe sets on the microarray being reduced from 54,675 to 54,668. Among the 54,668 probe sets, those with probe set ID starting with AFFX and those that did not receive present calls in at least 50% of the 207 samples were removed as described in the following section, leaving a total of 23,775 probe sets for analysis.

TABLE S2
Impact of masking on Affymetrix statistical calls (reported
as percentage of total probes: 54,675, raw; 54,668, masked).

| | Present | Marginal | Absent | No call |
|---|---|---|---|---|
| Raw | 34.9 | 1.7 | 63.3 | 0 |
| Masked | 48.0 | 3.1 | 48.9 | 0 (7) |

### Probe Set Filtering

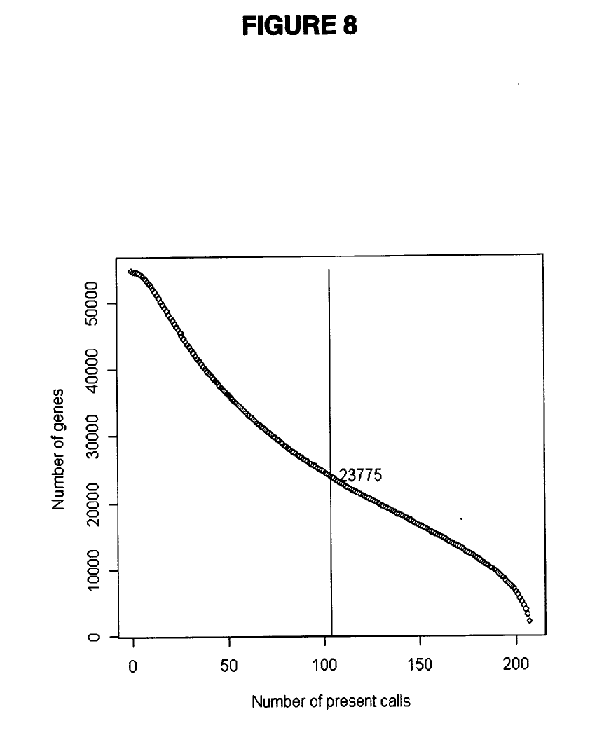

The filter required that a probe set be called ‘Present’ in at least 50% of the samples (n=104) in order for it to be retained in subsequent statistical analysis. This filter was fairly stringent, and it removed over 50% of the original probe sets, but was chosen to provide a reasonable tradeoff between signal reliability and the loss of some probe sets of potential biological relevance (FIG. 8/S2).
To assess whether the more reliable but reduced list of probe sets was indeed adequate for constructing our supervised models, we did our outcome (RFS) and 29-day MRD analyses using the full set of probe sets excluding those with probe set IDs starting with “AFFX”. Although there was only a very small overlap between the final sets of genes used in both models, the analyses that started from the filtered probe set list were found to be slightly superior statistically to those based on the unfiltered probe set list.

These results are consistent with similar observations made in the context of recent breast cancer studies. Two distinct expression profiling-derived gene panels for risk assessment are currently undergoing prospective evaluation by U.S. and European consortia.⁶ A meta-analysis⁷ found that notwithstanding minimal pairwise overlap between the respective sets of genes, a high concordance was observed between outcome predictions derived from the two predictors plus two others, in a large cohort of patients.⁸ In the present instance a similar biological redundancy is evidently operating with respect to the genes characterizing the newly-identified leukemic risk groups.

Based on these results, it appears that underlying patterns of gene expression corresponding to fundamental disease pathways and biological processes can manifest themselves as robust statistical associations with very different probe sets, depending on the precise analytic methodologies used to identify them.⁷ The choice of methodology depends in turn on the particular goals of a given study—for example, elucidating disease etiology, predicting outcome, or performing risk stratification at diagnosis.⁹ Here we have focused on the identification of gene sets as features for classifying acute leukemia patients into distinct risk categories. While non-unique, these probe sets provide important complementary clues for developing a unified understanding of the distinctive chromosomal lesions and disrupted regulatory pathways underlying the diverse prognostic subtypes of B-precursor ALL.

### Overview of Statistical Approach for Outcome Prediction

The primary indicator for outcome in this study is relapse-free survival (RFS), calculated as time from the date of trial enrollment to first event (relapse) or last follow-up. Patients in clinical remission or remission were censored at the date of last contact. RFS was estimated by the method of Kaplan and Meier and compared between groups using the logrank test. The supervised analyses for predicting outcome and MRD were performed using a cross-validation based scheme,¹⁰ in which an optimal gene expression model was determined through a number of iterations of cross-validations. The performance of the optimal model was evaluated through nested cross-validations of the entire model building process.
For outcome prediction, a Cox score² was used to examine the statistical significance of individual probe sets on the basis of how their expression values are associated with the RFS. Prediction analysis was carried out using the Cox proportional-hazards-model-based supervised principal components analysis (SPCA) method.^{11,12} The number of genes used in the SPCA model was determined by maximizing the average likelihood ratio test (LRT) scores obtained in a 20×5-fold cross-validation procedure, and a final model comprising that number of highest Cox score genes was built using the entire dataset. The model predicts a continuous risk score which is designed to be positively-associated with the risk to relapse. The gene expression risk classification was based on the predicted risk score. The gene expression high- (or low-) risk group was defined as having a positive (or negative) risk score. To avoid biasing the analysis results, an outer loop of leave-one-out cross-validation (LOOCV), independent from the internal loop (i.e., the 20 iterations of 5-fold cross-validation used to determine the final model) was performed to obtain cross-validated risk assignments used to assess the significance of the predictions. These cross-validated risk assignments were also used for outcome analyses and for presenting prediction statistics. The performance of the outcome predictor was evaluated by examining the association of patient outcome with predicted risk score and risk groups using a Kaplan-Meier estimator, Cox regression and the logrank test. For further technical details see Supplement, Section 8.

For prediction of MRD status at day 29, a modified t-test¹³ was used to examine the statistical significance of probe sets according to their association with positive/negative flow MRD at day 29, and a diagonal linear discriminant analysis (DLDA) model¹⁴ was used to make predictions. The number of genes used in the DLDA model was determined by minimizing the prediction error in a 100×10-fold cross-validation procedure, and a final model comprising that number of highest-scoring genes was computed using the entire dataset. A similar nested cross-validation procedure was performed to obtain the cross-validated predictions on MRD day 29 used to compute the misclassification error estimate. These predictions were also used for outcome analyses and for presenting prediction statistics. The performance of the MRD predictor was evaluated using the misclassification error rate and ROC accuracy. For further technical details see Supplement, Section 9.

### Gene Expression Classifier for Prediction of Relapse Free Survival (RFS)

A 20×5-fold cross validation as detailed in Section 8 was performed to determine the model for predicting the risk score of relapse. Twenty candidate thresholds were considered. The number of significant probe sets determined by each threshold and geometric mean of the likelihood ratio test statistic corresponding to each threshold are listed in Table S3, below.

TABLE S3
Candidate thresholds and corresponding numbers of significant genes
and geometric means of likelihood ratio test (LRT) statistic values.

| | | # Significant | LRT statistic |
|---|---|---|---|
| Threshold # | Threshold | Genes | (geometric mean) |
| 1 | 0.0000 | 23774 | 0.5289 |
| 2 | 0.1376 | 20262 | 0.7148 |
| 3 | 0.2752 | 16846 | 0.8135 |
| 4 | 0.4128 | 13619 | 0.8511 |
| 5 | 0.5505 | 10649 | 0.8174 |
| 6 | 0.6881 | 8007 | 0.8650 |
| 7 | 0.8257 | 5762 | 0.8248 |
| 8 | 0.9633 | 3940 | 0.7768 |
| 9 | 1.1009 | 2555 | 0.8843 |
| 10 | 1.2385 | 1571 | 0.8154 |
| 11 | 1.3761 | 915 | 0.9366 |
| 12 | 1.5137 | 509 | 1.0558 |
| 13 | 1.6513 | 273 | 1.3662 |
| 14 | 1.7889 | 144 | 1.6222 |
| 15 | 1.9265 | 75 | 1.8837 |
| 16 | 2.0641 | 42 | 1.9570 |
| 17 | 2.2017 | 24 | 1.7051 |
| 18 | 2.3393 | 14 | 1.6378 |
| 19 | 2.4770 | 8 | 0.8933 |
| 20 | 2.6146 | 4 | 0.5035 |

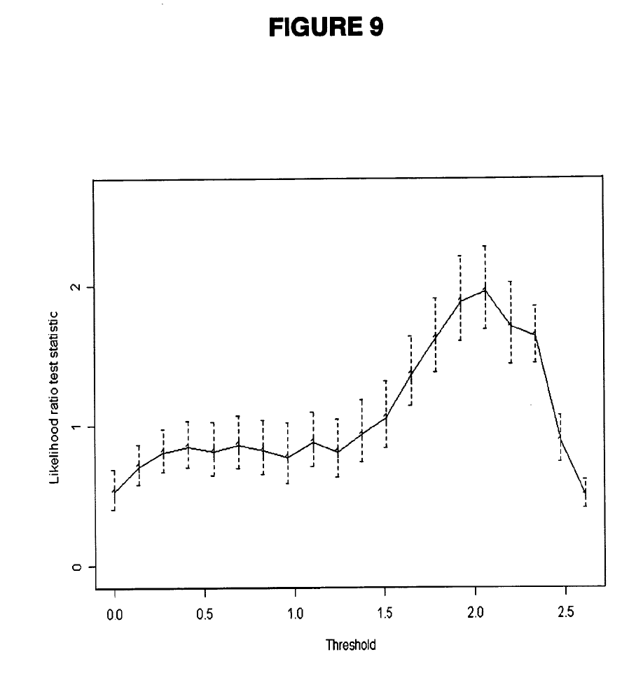

The mean of the LRT statistic is also plotted in FIG. 9/S3. We see that the geometric mean of the LRT reaches the maximum when the threshold is T=2.064. The “best” model determined by this threshold is a linear combination of expression values of 42 probe sets that are highly associated with RFS status (Table S4). SAM software was also used to calculate the false discovery rate (FDR) for each of those probe sets.

The final model for predicting RFS includes 42 probe sets (Table S4). Among the high-expressing genes in the high risk group are genes that play roles in the antioxidant defense system in the microvasculature (PON-2),¹⁵ adaptive cell signaling responses to TGF13 (CDC42EP3, CTGF),¹⁶ B-cell development and differentiation (IgJ), breast cancer growth, invasion and migration (CD73, CTGF), 17,18 colonic and/or renal cell carcinoma proliferation (TTYH2, BMPR1B),¹⁹⁻²¹ cell migration in acute myeloid leukemia (TSPAN7),²² and embryonic (SEMA6A) and mesenchymal (CD73) stem cell function.^{23,24} CTGF (CCN2) is also a growth factor secreted by pre-B ALL cells that is postulated to play a role in disease pathophysiology.²⁵ CD73 expressed on regulatory T cells mediates immune suppression²⁶ and plays a role in cellular multiresistance.²⁷ Two genes with tumor suppressor functions, NR4A3 and BTG3, are comparatively downregulated in the high risk group, as are the signaling proteins RGS1 and RGS2. RR4A3 (NOR-1) is a nuclear receptor of transcription factors involved in cellular susceptibility to tumorgenesis; downregulation is seen in acute myeloid leukemia.²⁸ BTG3 is a regulator of apoptosis and cell proliferation that controls cell cycle arrest following DNA damage and predicts relapse in T-ALL patients.²⁹ Decreased expression of RGS1 or RGS2 have a variety of consequences including effects on T-cell activation and migration³° and myeloid differentiation.31

TABLE S4
Probe sets (and associated genes) that are significantly associated with
relapse free survival

| Rank | High in | Cox Score | p-value | FDR | Probe set ID | Gene Symbol | Gene Description |
|---|---|---|---|---|---|---|---|
| 1 | High | 2.9873 | 0.000001 | <.0001 | 242579_at | BMPR1B | bone morphogenetic protein |
| | Risk | | | | | | receptor, type IB |
| 2 | Low Risk | −2.9540 | 0.000023 | <.0001 | 202388_at | RGS2 | regulator of G-protein signaling |
| | | | | | | | 2, 24 kDa |
| 3 | High | 2.9090 | 0.000012 | <.0001 | 213371_at | LDB3 | LIM domain binding 3 |
| | Risk | | | | | | |
| 4 | High | 2.8856 | 0.000020 | <.0001 | 210830_s_at | PON2 | paraoxonase 2 |
| | Risk | | | | | | |
| 5 | High | 2.6177 | 0.000230 | <.0001 | 201876_at | PON2 | paraoxonase 2 |
| | Risk | | | | | | |
| 6 | High | 2.6146 | 0.000009 | <.0001 | 209288_s_at | CDC42EP3 | CDC42 effector protein (Rho |
| | Risk | | | | | | GTPase binding) 3 |
| 7 | High | 2.6081 | 0.000570 | <.0001 | 215028_at | SEMA6A | sema domain, transmembrane |
| | Risk | | | | | | domain (TM), and cytoplasmic |
| | | | | | | | domain, (semaphorin) 6A |
| 8 | High | 2.5685 | 0.000620 | <.0001 | 223449_at | SEMA6A | sema domain, transmembrane |
| | Risk | | | | | | domain (TM), and cytoplasmic |
| | | | | | | | domain, (semaphorin) 6A |
| 9 | High | 2.5539 | 0.000310 | <.0001 | 204030_s_at | SCHIP1 | schwannomin interacting protein 1 |
| | Risk | | | | | | |
| 10 | High | 2.5511 | 0.000160 | <.0001 | 232539_at | — | MRNA; cDNA |
| | Risk | | | | | | DKFZp761H1023 (from clone |
| | | | | | | | DKFZp761H1023) |
| 11 | High | 2.5450 | 0.001300 | <.0001 | 212592_at | IGJ | Immunoglobulin J polypeptide, |
| | Risk | | | | | | linker protein for |
| | | | | | | | immunoglobulin alpha and mu |
| | | | | | | | polypeptides |
| 12 | High | 2.5287 | 0.000450 | <.0001 | 209101_at | CTGF | connective tissue growth factor |
| | Risk | | | | | | |
| 13 | High | 2.5223 | 0.000083 | <.0001 | 219313_at | GRAMD1C | GRAM domain containing 1C |
| | Risk | | | | | | |
| 14 | High | 2.4907 | 0.000110 | <.0001 | 225355_at | LOC54492 | hypothetical LOC54492 |
| | Risk | | | | | | |
| 15 | Low Risk | −2.4874 | 0.000045 | <.0001 | 228388_at | NFKBIB | nuclear factor of kappa light |
| | | | | | | | polypeptide gene enhancer in B- |
| | | | | | | | cells inhibitor, beta |
| 16 | High | 2.4545 | 0.000370 | <.0001 | 209365_s_at | ECM1 | extracellular matrix protein 1 |
| | Risk | | | | | | |
| 17 | High | 2.4211 | 0.000083 | <.0001 | 223741_s_at | TTYH2 | tweety homolog 2 (Drosophila) |
| | Risk | | | | | | |
| 18 | High | 2.3965 | 0.000062 | <.0001 | 236750_at | NRXN3 | Neurexin 3 |
| | Risk | | | | | | |
| 19 | High | 2.3725 | 0.000160 | <.0001 | 215617_at | LOC26010 | viral DNA polymerase- |
| | Risk | | | | | | transactivated protein 6 |
| 20 | High | 2.3715 | 0.000039 | <.0001 | 236766_at | — | Transcribed locus |
| | Risk | | | | | | |
| 21 | High | 2.3487 | 0.000280 | <.0001 | 203939_at | NT5E | 5′-nucleotidase, ecto (CD73) |
| | Risk | | | | | | |
| 22 | Low Risk | −2.3253 | 0.001700 | <.0001 | 216834_at | RGS1 | regulator of G-protein signaling 1 |
| 23 | Low Risk | −2.2848 | 0.002200 | <.0001 | 209959_at | NR4A3 | nuclear receptor subfamily 4, |
| | | | | | | | group A, member 3 |
| 24 | Low Risk | −2.2784 | 0.000490 | <.0001 | 213134_x_at | BTG3 | BTG family, member 3 |
| 25 | High | 2.2782 | 0.000850 | <.0001 | 244280_at | — | Homo sapiens, clone |
| | Risk | | | | | | IMAGE: 5583725, mRNA |
| 26 | High | 2.2729 | 0.000140 | <.0001 | 215479_at | — | CDNA FLJ20780 fis, clone |
| | Risk | | | | | | COL04256 |
| 27 | Low Risk | −2.2568 | 0.000053 | <.0001 | 205831_at | CD2 | CD2 molecule |
| 28 | High | 2.2532 | 0.000140 | <.0001 | 211675_s_at | MDFIC | MyoD family inhibitor domain |
| | Risk | | | | | | containing |
| 29 | Low Risk | −2.2474 | 0.001700 | <.0001 | 207978_s_at | NR4A3 | nuclear receptor subfamily 4, |
| | | | | | | | group A, member 3 |
| 30 | Low Risk | −2.2401 | 0.000009 | <.0001 | 224654_at | DDX21 | DEAD (Asp-Glu-Ala-Asp) box |
| | | | | | | | polypeptide 21 |
| 31 | Low Risk | −2.2316 | 0.000410 | <.0001 | 238623_at | — | CDNA FLJ37310 fis, clone |
| | | | | | | | BRAMY2016706 |
| 32 | High | 2.2094 | 0.002200 | <.0001 | 202242_at | TSPAN7 | tetraspanin 7 |
| | Risk | | | | | | |
| 33 | Low Risk | −2.2082 | 0.000880 | <.0001 | 226184_at | FMNL2 | formin-like 2 |
| 34 | Low Risk | −2.2010 | 0.000039 | <.0001 | 212497_at | MAPK1IP1L | mitogen-activated protein kinase |
| | | | | | | | 1 interacting protein 1-like |
| 35 | Low Risk | −2.1912 | 0.000960 | 8.4505 | 221349_at | VPREB1 | pre-B lymphocyte gene 1 |
| 36 | Low Risk | −2.1797 | 0.000005 | 8.4505 | 208152_s_at | DDX21 | DEAD (Asp-Glu-Ala-Asp) box |
| | | | | | | | polypeptide 21 |
| 37 | Low Risk | −2.1716 | 0.000820 | 8.4505 | 210024_s_at | UBE2E3 | ubiquitin-conjugating enzyme |
| | | | | | | | E2E 3 (UBC4/5 homolog, yeast) |
| 38 | High | 2.1635 | 0.001500 | <.0001 | 1559072_a_at | ELFN2 | extracellular leucine-rich repeat |
| | Risk | | | | | | and fibronectin type III domain |
| | | | | | | | containing 2 |
| 39 | Low Risk | −2.1634 | 0.002400 | 8.4505 | 244623_at | KCNQ5 | potassium voltage-gated channel, |
| | | | | | | | KQT-like subfamily, member 5 |
| 40 | Low Risk | −2.1378 | 0.001500 | 8.4505 | 224507_s_at | MGC12916 | hypothetical protein MGC12916 |
| 41 | Low Risk | −2.1275 | 0.001300 | 8.4505 | 203921_at | CHST2 | carbohydrate (N- |
| | | | | | | | acetylglucosamine-6-O) |
| | | | | | | | sulfotransferase 2 |
| 42 | High | 2.1196 | 0.000400 | 1.6184 | 1560524_at | LOC400581 | GRB2-related adaptor protein- |
| | Risk | | | | | | like |

Note
“High in” corresponds to “gene expression over-expressed in”
Cox Score is the modified score test statistic based on Cox regression.
P-value is for the Wald test based on univariate Cox regression.
FDR is the False Discovery Rate estimated using SAM

### Gene Expression Classifier for Prediction of Day 29 Minimal Residual Disease (MRD)

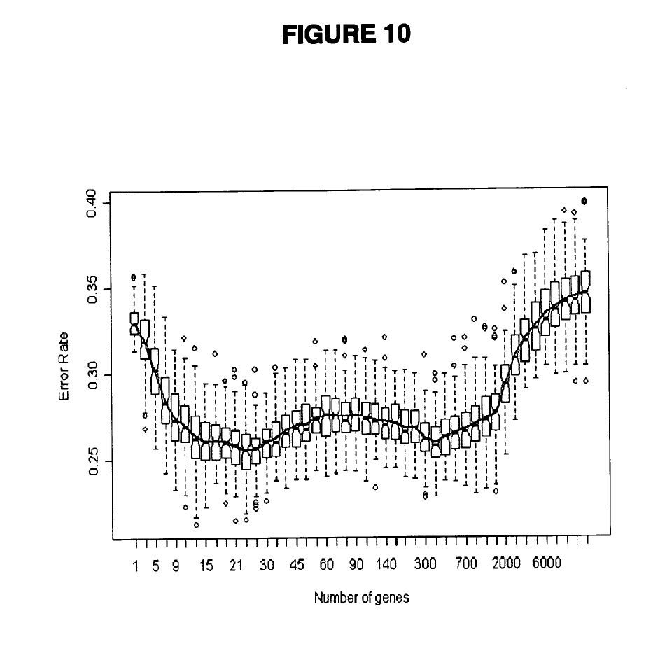

An optimal DLDA model for prediction of day 29 MRD was determined through a 100×10-fold cross-validation procedure as described in Section 9. FIG. 10/S4 shows the box plots of 100 average misclassification rates of each 10-fold cross-validation corresponding to each number of significant genes used in the models. The red line is the mean of 100 average error rates and the lower and upper bounds of the boxes represent the 25^{th} and 75^{th} quartiles, respectively.

The minimal mean error rate corresponds to the model using the 23 significant probe sets listed in Table S5. With a threshold of 1% for the False Discovery Rate (FDR), the SAM software identified 352 probe sets that are significantly associated with day 29 MRD status, which are listed in Table S6. Since DLDA as implemented here and SAM use the same method to assess the significance of the probe sets, the 23 probe sets included in the MRD prediction model (Table S5) also appear on the top of the list in Table S6. The 23 probe set includes the gene CDC42EP3 which is present among the top gene classifiers for both molecular MRD and RFS. A number of other probe sets overlap between the 352 probe sets predictive of MRD and gene expression predictors of RFS.

Genes with low expression among our high risk group include DTX-1, a regulator of Notch signaling,³² KLF4, a promoter of monocyte differentiation,³³ and TNSF4, a member of the tumor necrosis family. Other microarray studies of MRD have found cell-cycle progression and apoptosis-related genes to be involved in treatment resistance.³⁴⁻³⁷ Related genes present in our MRD classifier included P2RY5, E2F8, IRF4, but did not include CASP8AP2, described to be particularly significant in a few recent studies.^{35,36} Our two probe sets for CASP8AP2 (1570001, 222201) showed relatively weak signals with no discriminating function (P>0.1). High BAALC was a strong predictor for MRD. This gene has recently been shown to be associated with worse prognosis in acute myeloid leukemia.³⁸

TABLE S5
Probe sets (and associated genes) that are included in the MRD predictor

| Rank | High in | p-value | FDR (%) | Probe set ID | Gene Symbol | Gene Description |
|---|---|---|---|---|---|---|
| 1 | Neg | 0.00000005 | <.0001 | 242747_at | — | — |
| 2 | Neg | 0.00000147 | <.0001 | 205429_s_at | MPP6 | membrane protein, palmitoylated 6 (MAGUK p55 |
| | | | | | | subfamily member 6) |
| 3 | Neg | 0.00000036 | <.0001 | 221841_s_at | KLF4 | Kruppel-like factor 4 (gut) |
| 4 | Pos | 0.00000054 | <.0001 | 209286_at | CDC42EP3 | CDC42 effector protein (Rho GTPase binding) 3 |
| 5 | Neg | 0.00000000 | <.0001 | 1564310_a_at | PARP15 | poly (ADP-ribose) polymerase family, member 15 |
| 6 | Neg | 0.00000045 | <.0001 | 201719_s_at | EPB41L2 | erythrocyte membrane protein band 4.1-like 2 |
| 7 | Pos | 0.00000219 | <.0001 | 218899_s_at | BAALC | brain and acute leukemia, cytoplasmic |
| 8 | Neg | 0.00000101 | <.0001 | 213358_at | KIAA0802 | KIAA0802 |
| 9 | Neg | 0.00000100 | <.0001 | 1553380_at | PARP15 | poly (ADP-ribose) polymerase family, member 15 |
| 10 | Pos | 0.00000077 | <.0001 | 225685_at | — | CDNA FLJ31353 fis, clone MESAN2000264 |
| 11 | Neg | 0.00000042 | <.0001 | 227336_at | DTX1 | deltex homolog 1 (Drosophila) |
| 12 | Neg | 0.00000032 | <.0001 | 201718_s_at | EPB41L2 | erythrocyte membrane protein band 4.1-like 2 |
| 13 | Neg | 0.00000060 | <.0001 | 201710_at | MYBL2 | v-myb myeloblastosis viral oncogene homolog |
| | | | | | | (avian)-like 2 |
| 14 | Pos | 0.00000183 | <.0001 | 207426_s_at | TNFSF4 | tumor necrosis factor (ligand) superfamily, |
| | | | | | | member 4 (tax-transcriptionally activated |
| | | | | | | glycoprotein 1, 34 kDa) |
| 15 | Neg | 0.00000120 | <.0001 | 219990_at | E2F8 | E2F transcription factor 8 |
| 16 | Pos | 0.00000207 | <.0001 | 213817_at | — | CDNA FLJ13601 fis, clone PLACE1010069 |
| 17 | Pos | 0.00001106 | <.0001 | 220448_at | KCNK12 | potassium channel, subfamily K, member 12 |
| 18 | Pos | 0.00000110 | <.0001 | 232539_at | — | MRNA; cDNA DKFZp761H1023 (from clone |
| | | | | | | DKFZp761H1023) |
| 19 | Neg | 0.00000065 | <.0001 | 225688_s_at | PHLDB2 | pleckstrin homology-like domain, family B, |
| | | | | | | member 2 |
| 20 | Pos | 0.00000546 | <.0001 | 218589_at | P2RY5 | purinergic receptor P2Y, G-protein coupled, 5 |
| 21 | Neg | 0.00000073 | <.0001 | 204562_at | IRF4 | interferon regulatory factor 4 |
| 22 | Neg | 0.00000016 | <.0001 | 219032_x_at | OPN3 | opsin 3 |
| 23 | Pos | 0.00000598 | <.0001 | 242051_at | CD99 | CD99 molecule |

Note:
Neg = MRD negative;
Pos = MRD positive;
p-value via two sample t-test
FDR = False discovery rate as estimated by SAM

TABLE S6
Probe sets (and associated genes) that are significantly associated with distinction
between negative and positive MRD at day 29. Highlighted top-23 probe sets correspond to
those used in the final MRD predictor (Table S5).

| Rank | High in | p-value | FDR (%) | Probe set ID | Gene Symbol | Gene Description |
|---|---|---|---|---|---|---|
| 1 | Neg | 0.00000005 | <.0001 | [symbol] | — | — |
| 2 | Neg | 0.00000147 | <.0001 | [symbol] | MPP6 | membrane protein, palmitoylated 6 (MAGUK p55 |
| | | | | | | subfamily member 6) |
| 3 | Neg | 0.00000036 | <.0001 | [symbol] | KLF4 | Kruppel-like factor 4 (gut) |
| 4 | Pos | 0.00000054 | <.0001 | [symbol] | CDC42EP3 | CDC42 effector protein (Rho GTPase binding) 3 |
| 5 | Neg | 0.00000000 | <.0001 | [symbol] | PARP15 | poly (ADP-ribose) polymerase family, member 15 |
| 6 | Neg | 0.00000045 | <.0001 | [symbol] | EPB41L2 | erythrocyte membrane protein band 4.1-like 2 |
| 7 | Pos | 0.00000219 | <.0001 | [symbol] | BAALC | brain and acute leukemia, cytoplasmic |
| 8 | Neg | 0.00000101 | <.0001 | [symbol] | KIAA0802 | KIAA0802 |
| 9 | Neg | 0.00000100 | <.0001 | [symbol] | PARP15 | poly (ADP-ribose) polymerase family, member 15 |
| 10 | Pos | 0.00000077 | <.0001 | [symbol] | — | CDNA FLJ31353 fis, clone MESAN2000264 |
| 11 | Neg | 0.00000042 | <.0001 | [symbol] | DTX1 | deltex homolog 1 (Drosophila) |
| 12 | Neg | 0.00000032 | <.0001 | [symbol] | EPB41L2 | erythrocyte membrane protein band 4.1-like 2 |
| 13 | Neg | 0.00000060 | <.0001 | [symbol] | MYBL2 | v-myb myeloblastosis viral oncogene homolog |
| | | | | | | (avian)-like 2 |
| 14 | Pos | 0.00000183 | <.0001 | [symbol] | TNFSF4 | tumor necrosis factor (ligand) superfamily, member |
| | | | | | | 4 (tax-transcriptionally activated glycoprotein I, 34kDa) |
| 15 | Neg | 0.00000120 | <.0001 | [symbol] | E2F8 | E2F transcription factor 8 |
| 16 | Pos | 0.00000207 | <.0001 | [symbol] | — | CDNA FLJ13601 fis, clone PLACE1010069 |
| 17 | Pos | 0.00001106 | <.0001 | [symbol] | KCNK12 | potassium channel, subfamily K, member 12 |
| 18 | Pos | 0.00000110 | <.0001 | [symbol] | — | MRNA; cDNA DKFZp761H1023 (from clone |
| | | | | | | DKFZp761H1023) |
| 19 | Neg | 0.00000065 | <.0001 | [symbol] | PHLDB2 | pleckstrin homology-like domain, family B, member 2 |
| 20 | Pos | 0.00000546 | <.0001 | [symbol] | P2RY5 | purinergic receptor P2Y, G-protein coupled, 5 |
| 21 | Neg | 0.00000073 | <.0001 | [symbol] | IRF4 | interferon regulatory factor 4 |
| 22 | Neg | 0.00000016 | <.0001 | [symbol] | OPN3 | opsin 3 |
| 23 | Pos | 0.00000598 | <.0001 | [symbol] | CD99 | CD99 molecule |
| 24 | Neg | 0.00000092 | <.0001 | 220266_s_at | KLF4 | Kruppel-like factor 4 (gut) |
| 25 | Pos | 0.00002445 | <.0001 | 201028_s_at | CD99 | CD99 molecule |
| 26 | Pos | 0.00004247 | <.0001 | 204304_s_at | PROM1 | prominin 1 |
| 27 | Pos | 0.00007265 | <.0001 | 208886_at | H1F0 | H1 histone family, member 0 |
| 28 | Pos | 0.00012240 | <.0001 | 209101_at | CTGF | connective tissue growth factor |
| 29 | Neg | 0.00000003 | <.0001 | 236307_at | — | Transcribed locus |
| 30 | Neg | 0.00006038 | <.0001 | 206530_at | RAB30 | RAB30, member RAS oncogene family |
| 31 | Neg | 0.00004247 | <.0001 | 210094_s_at | PARD3 | par-3 partitioning defective 3 homolog (C. elegans) |
| 32 | Pos | 0.00000003 | <.0001 | 209288_s_at | CDC42EP3 | CDC42 effector protein (Rho GTPase binding) 3 |
| 33 | Neg | 0.00015116 | <.0001 | 221526_x_at | PARD3 | par-3 partitioning defective 3 homolog (C. elegans) |
| 34 | Neg | 0.00001630 | <.0001 | 210517_s_at | AKAP12 | A kinase (PRKA) anchor protein (gravin) 12 |
| 35 | Pos | 0.00010226 | <.0001 | 227998_at | S100A16 | S100 calcium binding protein A16 |
| 36 | Neg | 0.00000869 | <.0001 | 1559618_at | LOC100129447 | hypothetical protein LOC100129447 |
| 37 | Neg | 0.00000486 | <.0001 | 228390_at | — | CDNA clone IMAGE:5259272 |
| 38 | Pos | 0.00000726 | <.0001 | 207571_x_at | Clorf38 | chromosome 1 open reading frame 38 |
| 39 | Pos | 0.00003152 | <.0001 | 206674_at | FLT3 | fms-related tyrosine kinase 3 |
| 40 | Pos | 0.00006038 | <.0001 | 227923_at | SHANK3 | SH3 multiple ankyrin repeat domains 3 |
| 41 | Neg | 0.00001223 | <.0001 | 212022_s_at | MKI67 | antigen identified by monoclonal antibody Ki-67 |
| 42 | Pos | 0.00014623 | <.0001 | 203372_s_at | SOCS2 | suppressor of cytokine signaling 2 |
| 43 | Pos | 0.00006938 | <.0001 | 204646_at | DPYD | dihydropyrimidine dehydrogenase |
| 44 | Pos | 0.00001134 | <.0001 | 207610_s_at | EMR2 | egf-like module containing, mucin-like, hormone |
| | | | | | | receptor-like 2 |
| 45 | Pos | 0.00006858 | <.0001 | 204030_s_at | SCHIPI | schwannomin interacting protein 1 |
| 46 | Neg | 0.00002761 | <.0001 | 1552924_a_at | PITPNM2 | phosphatidylinositol transfer protein, membrane- |
| | | | | | | associated 2 |
| 47 | Pos | 0.00000765 | <.0001 | 217967_s_at | FAM129A | family with sequence similarity 129, member A |
| 48 | Neg | 0.00000443 | <.0001 | 227173_s_at | BACH2 | BTB and CNC homology 1, basic leucine zipper |
| | | | | | | transcription factor 2 |
| 49 | Pos | 0.00007520 | <.0001 | 203373_at | SOCS2 | suppressor of cytokine signaling 2 |
| 50 | Pos | 0.00023124 | <.0001 | 222154_s_at | LOC26010 | viral DNA polymerase-transactivated protein 6 |
| 51 | Pos | 0.00005697 | <.0001 | 201029_s_at | CD99 | CD99 molecule |
| 52 | Pos | 0.00012516 | <.0001 | 225524_at | ANTXR2 | anthrax toxin receptor 2 |
| 53 | Pos | 0.00000785 | <.0001 | 210785_s_at | Clorf38 | chromosome 1 open reading frame 38 |
| 54 | Neg | 0.00000020 | <.0001 | 1556451_at | — | MRNA; cDNA DKFZp667B1520 (from clone |
| | | | | | | DKFZp667B1520) |
| 55 | Pos | 0.00000038 | <.0001 | 1557626_at | — | CDNA FLJ39805 fis, clone SPLEN2007951 |
| 56 | Pos | 0.00011317 | <.0001 | 202242_at | TSPAN7 | tetraspanin 7 |
| 57 | Neg | 0.00000176 | <.0001 | 228361_at | E2F2 | E2F transcription factor 2 |
| 58 | Pos | 0.00006108 | <.0001 | 222780_s_at | BAALC | brain and acute leukemia, cytoplasmic |
| 59 | Pos | 0.00017824 | <.0001 | 201876_at | PON2 | paraoxonase 2 |
| 60 | Pos | 0.00001149 | <.0001 | 218847_at | IGF2BP2 | insulin-like growth factor 2 mRNA binding protein 2 |
| 61 | Pos | 0.00000598 | <.0001 | 228573_at | — | Transcribed locus |
| 62 | Neg | 0.00018824 | <.0001 | 225288_at | COL27A1 | collagen, type XXVII, alpha 1 |
| 63 | Neg | 0.00001336 | <.0001 | 227846_at | GPR176 | G protein-coupled receptor 176 |
| 64 | Pos | 0.00001735 | <.0001 | 213541_s_at | ERG | v-ets erythroblastosis virus E26 oncogene homolog |
| | | | | | | (avian) |
| 65 | Neg | 0.00008529 | <.0001 | 225246_at | STIM2 | stromal interaction molecule 2 |
| 66 | Pos | 0.00000082 | <.0001 | 224861_at | GNAQ | Guanine nucleotide binding protein (G protein), q |
| | | | | | | polypeptide |
| 67 | Pos | 0.00002061 | <.0001 | 211474_s_at | SERPINB6 | serpin peptidase inhibitor, clade B (ovalbumin), |
| | | | | | | member 6 |
| 68 | Neg | 0.00182593 | <.0001 | 219737_s_at | PCDH9 | protocadherin 9 |
| 69 | Neg | 0.00000225 | <.0001 | 226350_at | CHML | choroideremia-like (Rab escort protein 2) |
| 70 | Neg | 0.00000765 | <.0001 | 221234_s_at | BACH2 | BTB and CNC homology 1, basic leucine zipper |
| | | | | | | transcription factor 2 |
| 71 | Pos | 0.00006108 | <.0001 | 227013_at | LATS2 | LATS, large tumor suppressor, homolog 2 (Drosophila) |
| 72 | Pos | 0.00000033 | <.0001 | 235094_at | — | CDNA FLJ39413 fis, clone PLACE6015729 |
| 73 | Pos | 0.00007018 | <.0001 | 209543_s_at | CD34 | CD34 molecule |
| 74 | Neg | 0.00003041 | <.0001 | 205692_s_at | CD38 | CD38 molecule |
| 75 | Pos | 0.00008148 | <.0001 | 210993_s_at | SMAD1 | SMAD family member 1 |
| 76 | Neg | 0.00003115 | <.0001 | 203922_s_at | CYBB | cytochrome b-245, beta polypeptide (chronic |
| | | | <.0001 | | | granulomatous disease) |
| 77 | Pos | 0.00000240 | <.0001 | 202430_s_at | PLSCR1 | phospholipid scramblase 1 |
| 78 | Neg | 0.00010460 | <.0001 | 225293_at | COL27A1 | collagen, type XXVII, alpha 1 |
| 79 | Neg | 0.00056256 | <.0001 | 213273_at | ODZ4 | odz, odd Oz/ten-m homolog 4 (Drosophila) |
| 80 | Pos | 0.00033554 | <.0001 | 216565_x_at | — | — |
| 81 | Pos | 0.00000647 | <.0001 | 240432_x_at | — | Transcribed locus |
| 82 | Neg | 0.00000699 | <.0001 | 239946_at | — | Transcribed locus |
| 83 | Pos | 0.00002506 | <.0001 | 242565_x_at | C2lorf57 | Chromosome 21 open reading frame 57 |
| 84 | Pos | 0.00047774 | <.0001 | 201811_x_at | SH3BP5 | SH3-domain binding protein 5 (BTK-associated) |
| 85 | Pos | 0.00028636 | <.0001 | 200953_s_at | CCND2 | cyclin D2 |
| 86 | Pos | 0.00009998 | <.0001 | 220034_at | IRAK3 | interleukin-1 receptor-associated kinase 3 |
| 87 | Neg | 0.00000443 | <.0001 | 209760_at | KIAA0922 | KIAA0922 |
| 88 | Pos | 0.00000598 | <.0001 | 222762_x_at | LIMD1 | LIM domains containing 1 |
| 89 | Pos | 0.00004051 | <.0001 | 223741_s_at | TTYH2 | tweety homolog 2 (Drosophila) |
| 90 | Pos | 0.00081524 | <.0001 | 226018_at | C7orf41 | chromosome 7 open reading frame 41 |
| 91 | Neg | 0.00119278 | <.0001 | 210473_s_at | GPR125 | G protein-coupled receptor 125 |
| 92 | Pos | 0.00033203 | <.0001 | 239901_at | — | Transcribed locus |
| 93 | Pos | 0.00063516 | <.0001 | 1559315_s_at | LOC144481 | hypothetical protein LOC144481 |
| 94 | Neg | 0.00000234 | <.0001 | 236796_at | BACH2 | BTB and CNC homology 1, basic leucine zipper |
| | | | | | | transcription factor 2 |
| 95 | Pos | 0.00000213 | <.0001 | 240498_at | — | — |
| 96 | Pos | 0.00000186 | <.0001 | 219383_at | FLJ14213 | protor-2 |
| 97 | Pos | 0.00000134 | <.0001 | 221249_s_at | FAM117A | family with sequence similarity 117, member A |
| 98 | Neg | 0.00020983 | <.0001 | 1565951_s_at | CHML | choroideremia-like (Rab escort protein 2) |
| 99 | Neg | 0.00005128 | <.0001 | 205159_at | CSF2RB | colony stimulating factor 2 receptor, beta, low-affinity |
| | | | | | | (granulocyte-macrophage) |
| 100 | Pos | 0.00000512 | <.0001 | 228696_at | SLC45A3 | solute carrier family 45, member 3 |
| 101 | Pos | 0.00010343 | <.0001 | 213931_at | ID2 /// ID2B | inhibitor of DNA binding 2, dominant negative |
| | | | | | | helix-loop-helix protein /// inhibitor of DNA |
| | | | | | | binding 2B, dominant negative helix-loop-helix protein |
| 102 | Pos | 0.00032856 | <.0001 | 202481_at | DHRS3 | dehydrogenase/reductase (SDR family) member 3 |
| 103 | Neg | 0.00113666 | <.0001 | 226796_at | LOC116236 | hypothetical protein LOC116236 |
| 104 | Neg | 0.00001223 | <.0001 | 218032_at | SNN | stannin |
| 105 | Pos | 0.00007520 | <.0001 | 223380_s_at | LATS2 | LATS, large tumor suppressor, homolog 2 |
| | | | | | | (Drosophila) |
| 106 | Pos | 0.00014950 | <.0001 | 202023_at | EFNA1 | ephrin-A1 |
| 107 | Pos | 0.00001713 | <.0001 | 211275_s_at | GYG1 | glycogenin 1 |
| 108 | Neg | 0.00015453 | <.0001 | 204165_at | WASF1 | WAS protein family, member 1 |
| 109 | Pos | 0.00016874 | <.0001 | 219938_s_at | PSTPIP2 | proline-serine-threonine phosphatase interacting |
| | | | | | | protein 2 |
| 110 | Neg | 0.00090860 | <.0001 | 212985_at | — | MRNA; cDNA DKFZp434E033 (from clone |
| | | | | | | DKFZp434E033) |
| 111 | Neg | 0.00017248 | <.0001 | 231124_x_at | LY9 | lymphocyte antigen 9 |
| 112 | Neg | 0.00051853 | <.0001 | 206001_at | NPY | neuropeptide Y |
| 113 | Neg | 0.00047774 | <.0001 | 241679_at | — | — |
| 114 | Neg | 0.00015972 | <.0001 | 240718_at | LRMP | Lymphoid-restricted membrane protein |
| 115 | Pos | 0.00020534 | <.0001 | 214453_s_at | IFI44 | interferon-induced protein 44 |
| 116 | Neg | 0.00000017 | <.0001 | 203907_s_at | IQSEC1 | IQ motif and Sec7 domain 1 |
| 117 | Neg | 0.00006625 | <.0001 | 1556425_a_at | LOC284219 | hypothetical protein LOC284219 |
| 118 | Pos | 0.00028636 | <.0001 | 201810_s_at | SH3BP5 | SH3-domain binding protein 5 (BTK-associated) |
| 119 | Pos | 0.00006473 | <.0001 | 241824_at | — | Transcribed locus |
| 120 | Pos | 0.00000681 | <.0001 | 211675_s_at | MDFIC | MyoD family inhibitor domain containing |
| 121 | Pos | 0.00000858 | <.0001 | 232210_at | — | CDNA FLJ14056 fis, clone HEMBB1000335 |
| 122 | Pos | 0.00014623 | <.0001 | 204334_at | KLF7 | Kruppel-like factor 7 (ubiquitous) |
| 123 | Pos | 0.00002761 | <.0001 | 227002_at | FAM78A | family with sequence similarity 78, member A |
| 124 | Pos | 0.00051326 | <.0001 | 227798_at | SMAD1 | SMAD family member 1 |
| 125 | Pos | 0.00003470 | <.0001 | 209723_at | SERPINB9 | serpin peptidase inhibitor, clade B (ovalbumin), |
| | | | | | | member 9 |
| 126 | Neg | 0.00070928 | <.0001 | 202732_at | PKIG | protein kinase (cAMP-dependent, catalytic) inhibitor |
| | | | | | | gamma |
| 127 | Pos | 0.00032171 | <.0001 | 1563335_at | IRGM | immunity-related GTPase family, M |
| 128 | Pos | 0.00010226 | <.0001 | 243092_at | — | CDNA clone IMAGE:4817413 |
| 129 | Pos | 0.00006779 | <.0001 | 239809_at | — | Transcribed locus |
| 130 | Neg | 0.00001630 | <.0001 | 202806_at | DBN1 | drebrin 1 |
| 131 | Neg | 0.00011445 | <.0001 | 221520_s_at | CDCA8 | cell division cycle associated 8 |
| 132 | Neg | 0.00000512 | <.0001 | 204947_at | E2F1 | E2F transcription factor 1 |
| 133 | Pos | 0.00060391 | <.0001 | 244665_at | — | Transcribed locus |
| 134 | Neg | 0.00030841 | <.0001 | 236191_at | — | Transcribed locus |
| 135 | Pos | 0.00014623 | <.0001 | 218729_at | LXN | latexin |
| 136 | Neg | 0.00011704 | <.0001 | 230597_at | SLC7A3 | solute carrier family 7 (cationic amino acid |
| | | | | | | transporter, y+ system), member 3 |
| 137 | Neg | 0.00009131 | <.0001 | 243030_at | — | Transcribed locus |
| 138 | Pos | 0.00000035 | <.0001 | 209164_s_at | CYB561 | cytochrome b-561 |
| 139 | Pos | 0.00003909 | <.0001 | 219871_at | FLJ13197 /// | hypothetical FLJ13197 /// hypothetical protein |
| | | | | | LOC100132861 | LOC100132861 |
| 140 | Pos | 0.00000091 | <.0001 | 239740_at | ETV6 | ets variant gene 6 (TEL oncogene) |
| 141 | Neg | 0.00003956 | <.0001 | 208072_s_at | DGKD | diacylglycerol kinase, delta 130kDa |
| 142 | Pos | 0.00000174 | <.0001 | 237561_x_at | — | Transcribed locus |
| 143 | Neg | 0.00006180 | <.0001 | 235699_at | REM2 | RAS (RAD and GEM)-like GTP binding 2 |
| 144 | Pos | 0.00037651 | <.0001 | 218694_at | ARMCX1 | armadillo repeat containing, X-linked 1 |
| 145 | Pos | 0.00058585 | <.0001 | 238032_at | — | Transcribed locus |
| 146 | Neg | 0.00147143 | <.0001 | 244623_at | KCNQ5 | potassium voltage-gated channel, KQT-like subfamily, |
| | | | | | | member 5 |
| 147 | Neg | 0.00093573 | 0.2273 | 221527_s_at | PARD3 | par-3 partitioning defective 3 homolog (C. elegans) |
| 148 | Pos | 0.00023882 | 0.2273 | 208981_at | PECAM1 | platelet/endothelial cell adhesion molecule (CD31 |
| | | | | | | antigen) |
| 149 | Pos | 0.00025197 | 0.2273 | 204249_s_at | LMO2 | LIM domain only 2 (rhombotin-like 1) |
| 150 | Pos | 0.00090860 | 0.2273 | 243808_at | — | Transcribed locus |
| 151 | Pos | 0.00043543 | 0.2273 | 203139_at | DAPK1 | death-associated protein kinase 1 |
| 152 | Pos | 0.00025468 | 0.2273 | 209813_x_at | TARP | TCR gamma alternate reading frame protein |
| 153 | Neg | 0.00000336 | 0.2273 | 203185_at | RASSF2 | Ras association (RaIGDS/AF-6) domain family |
| | | | | | | member 2 |
| 154 | Pos | 0.00045848 | 0.2273 | 201656_at | ITGA6 | integrin, alpha 6 |
| 155 | Pos | 0.00036873 | 0.2273 | 208614_s_at | FLNB | filamin B, beta (actin binding protein 278) |
| 156 | Pos | 0.00000368 | 0.2273 | 232685_at | — | CDNA: FLJ21564 fis, clone COL06452 |
| 157 | Neg | 0.00004148 | 0.2273 | 218949_s_at | QRSL1 | glutaminyl-tRNA synthase (glutamine-hydrolyzing)- |
| | | | | | | like 1 |
| 158 | Pos | 0.00008055 | 0.2273 | 237591_at | FLJ42957 | FLJ42957 protein |
| 159 | Pos | 0.00001938 | 0.2273 | 231369_at | ZNF333 | Zinc finger protein 333 |
| 160 | Pos | 0.00077581 | 0.2273 | 236750_at | NRXN3 | Neurexin 3 |
| 161 | Pos | 0.00029877 | 0.2273 | 226545_at | CD109 | CD109 molecule |
| 162 | Pos | 0.00016328 | 0.2273 | 237009_at | — | — |
| 163 | Neg | 0.00141668 | 0.2273 | 229072_at | — | CDNA clone IMAGE:5259272 |
| 164 | Pos | 0.00038046 | 0.2273 | 1555638_a_at | SAMSN1 | SAM domain, SH3 domain and nuclear localization |
| | | | | | | signals 1 |
| 165 | Neg | 0.00002567 | 0.2273 | 221586_s_at | E2F5 | E2F transcription factor 5, p130-binding |
| 166 | Pos | 0.00002506 | 0.2273 | 205585_at | ETV6 | ets variant gene 6 (TEL oncogene) |
| 167 | Pos | 0.00007963 | 0.2273 | 221942_s_at | GUCY1A3 | guanylate cyclase 1, soluble, alpha 3 |
| 168 | Neg | 0.00023124 | 0.2273 | 238623_at | — | CDNA FLJ37310 fis, clone BRAMY2016706 |
| 169 | Pos | 0.00066791 | 0.2273 | 208982_at | PECAM1 | platelet/endothelial cell adhesion molecule |
| | | | | | | (CD31 antigen) |
| 170 | Pos | 0.00003152 | 0.2273 | 225913_at | SGK269 | NKF3 kinase family member |
| 171 | Pos | 0.00008825 | 0.2273 | 220560_at | C11orf21 | chromosome 11 open reading frame 21 |
| 172 | Pos | 0.00013087 | 0.2273 | 238893_at | LOC338758 | hypothetical protein LOC338758 |
| 173 | Pos | 0.00007607 | 0.2273 | 205423_at | AP1B1 | adaptor-related protein complex 1, beta 1 subunit |
| 174 | Neg | 0.00030516 | 0.2273 | 228461_at | SH3MD4 | SH3 multiple domains 4 |
| 175 | Pos | 0.00015116 | 0.2273 | 235171_at | — | Transcribed locus |
| 176 | Pos | 0.00000455 | 0.2273 | 239005_at | — | CDNA FLJ38785 fis, clone LIVER2001329 |
| 177 | Pos | 0.00102169 | 0.2273 | 242579_at | BMPR1B | bone morphogenetic protein receptor, type IB |
| 178 | Pos | 0.00013234 | 0.2273 | 227098_at | DUSP18 | dual specificity phosphatase 18 |
| 179 | Neg | 0.00036110 | 0.2273 | 206079_at | CHML | choroideremia-like (Rab escort protein 2) |
| 180 | Pos | 0.00000708 | 0.2273 | 202252_at | RAB13 | RAB13, member RAS oncogene family |
| 181 | Neg | 0.00191271 | 0.2273 | 214084_x_at | LOC648998 | similar to Neutrophil cytosol factor 1 (NCF-1) |
| | | | | | | (Neutrophil NADPH oxidase factor 1) (47 kDa |
| | | | | | | neutrophil oxidase factor) (p47-phox) (NCF-47K) |
| | | | | | | (47 kDa autosomal chronic granulomatous |
| | | | | | | disease protein) (NOXO2) |
| 182 | Neg | 0.00001178 | 0.2273 | 220768_s_at | CSNK1G3 | casein kinase 1, gamma 3 |
| 183 | Pos | 0.00002506 | 0.2273 | 209163_at | CYB561 | cytochrome b-561 |
| 184 | Pos | 0.00133807 | 0.2273 | 215177_s_at | ITGA6 | integrin, alpha 6 |
| 185 | Pos | 0.00024663 | 0.2273 | 238063_at | TMEM154 | transmembrane protein 154 |
| 186 | Neg | 0.00010226 | 0.2273 | 218662_s_at | NCAPG | non-SMC condensin I complex, subunit G |
| 187 | Neg | 0.00113666 | 0.2273 | 206255_at | BLK | B lymphoid tyrosine kinase |
| 188 | Neg | 0.00019449 | 0.2273 | 1557835_at | — | CDNA FLJ31592 fis, clone NT2RI2002447 |
| 189 | Pos | 0.00003956 | 0.2273 | 1552623_at | HSH2D | hematopoietic SH2 domain containing |
| 190 | Neg | 0.00029251 | 0.2273 | 204674_at | LRMP | lymphoid-restricted membrane protein |
| 191 | Pos | 0.00001891 | 0.2273 | 227235_at | — | CDNA clone IMAGE:5302158 |
| 192 | Pos | 0.00009664 | 0.2273 | 213280_at | GARNL4 | GTPase activating Rap/RanGAP domain-like 4 |
| 193 | Pos | 0.00011574 | 0.2273 | 242794_at | MAML3 | mastermind-like 3 (Drosophila) |
| 194 | Neg | 0.00030841 | 0.3445 | 35974_at | LRMP | lymphoid-restricted membrane protein |
| 195 | Pos | 0.00000171 | 0.3445 | 243121_x_at | — | — |
| 196 | Pos | 0.00000455 | 0.3445 | 222079_at | ERG | v-ets erythroblastosis virus E26 oncogene |
| | | | | | | homolog (avian) |
| 197 | Neg | 0.00101179 | 0.3445 | 222760_at | ZNF703 | zinc finger protein 703 |
| 198 | Pos | 0.00030516 | 0.3445 | 229307_at | ANKRD28 | ankyrin repeat domain 28. |
| 199 | Pos | 0.00011445 | 0.3445 | 1563392_at | — | Chromosome 21, Down syndrome critical region |
| | | | | | | transcript, T7 end of clone a-1-g12 |
| 200 | Neg | 0.00032171 | 0.3445 | 211404_s_at | APLP2 | amyloid beta (A4) precursor-like protein 2 |
| 201 | Neg | 0.00003387 | 0.3445 | 40148_at | APBB2 | amyloid beta (A4) precursor protein-binding, |
| | | | | | | family B, member 2 (Fe65-like) |
| 202 | Neg | 0.00084811 | 0.3445 | 202478_at | TRIB2 | tribbles homolog 2 (Drosophila) |
| 203 | Neg | 0.00001735 | 0.3445 | 230671_at | — | Full length insert cDNA clone ZD43G04 |
| 204 | Neg | 0.00177561 | 0.3445 | 243780_at | — | CDNA FLJ46553 fis, clone THYMU3038879 |
| 205 | Pos | 0.00000664 | 0.3445 | 213233_s_at | KLHL9 | kelch-like 9 (Drosophila) |
| 206 | Pos | 0.00290806 | 0.3445 | 203543_s_at | KLF9 | Kruppel-like factor 9 |
| 207 | Pos | 0.00001735 | 0.3445 | 1561167_at | — | Full length insert cDNA clone YA75A09 |
| 208 | Pos | 0.00140329 | 0.3445 | 210830_s_at | PON2 | paraoxonase 2 |
| 209 | Pos | 0.00038046 | 0.3445 | 206631_at | PTGER2 | prostaglandin E receptor 2 (subtype EP2), 53kDa |
| 210 | Neg | 0.00007349 | 0.3445 | 220999_s_at | CYFIP2 | cytoplasmic FMR1 interacting protein 2 |
| 211 | Neg | 0.00000532 | 0.3445 | 229551_x_at | ZNF367 | zinc finger protein 367 |
| 212 | Neg | 0.00023882 | 0.3445 | 225606_at | BCL2L11 | BCL2-like 11 (apoptosis facilitator) |
| 213 | Neg | 0.00207853 | 0.3445 | 204730_at | RIMS3 | regulating synaptic membrane exocytosis 3 |
| 214 | Pos | 0.00202185 | 0.3445 | 228434_at | BTNL9 | butyrophilin-like 9 |
| 215 | Neg | 0.00008432 | 0.3445 | 219493_at | SHCBP1 | SHC SH2-domain binding protein 1 |
| 216 | Pos | 0.00332312 | 0.3445 | 229902_at | FLT4 | fms-related tyrosine kinase 4 |
| 217 | Neg | 0.00043543 | 0.3445 | 214185_at | KHDRBS1 | KH domain containing, RNA binding, signal |
| | | | | | | transduction associated 1 |
| 218 | Neg | 0.00169458 | 0.3445 | 240593_x_at | — | Transcribed locus |
| 219 | Pos | 0.00009448 | 0.3445 | 209344_at | TPM4 | tropomyosin 4 |
| 220 | Neg | 0.00000938 | 0.3445 | 218350_s_at | GMNN | geminin, DNA replication inhibitor |
| 221 | Neg | 0.00021911 | 0.3445 | 213607_x_at | NADK | NAD kinase |
| 222 | Neg | 0.00530278 | 0.3445 | 205603_s_at | DIAPH2 | diaphanous homolog 2 (Drosophila) |
| 223 | Pos | 0.00016149 | 0.3445 | 213572_s_at | SERPINB1 | serpin peptidase inhibitor, clade B (ovalbumin), |
| | | | | | | member 1 |
| 224 | Pos | 0.00119278 | 0.3445 | 201601_x_at | IFITM1 | interferon induced transmembrane protein 1 (9-27) |
| 225 | Pos | 0.00023124 | 0.3445 | 224565_at | TncRNA | trophoblast-derived noncoding RNA |
| 226 | Pos | 0.00004401 | 0.3445 | 211521_s_at | PSCD4 | pleckstrin homology, Sec7 and coiled-coil domains 4 |
| 227 | Pos | 0.00288215 | 0.3445 | 214349_at | — | Transcribed locus |
| 228 | Pos | 0.00054013 | 0.3445 | 227297_at | ITGA9 | integrin, alpha 9 |
| 229 | Neg | 0.00596604 | 0.3445 | 228737_at | TOX2 | TOX high mobility group box family member 2 |
| 230 | Neg | 0.00000903 | 0.3445 | 215785_s_at | CYFIP2 | cytoplasmic FMR1 interacting protein 2 |
| 231 | Pos | 0.00018218 | 0.3445 | 228726_at | — | Transcribed locus |
| 232 | Neg | 0.00036110 | 0.3445 | 228003_at | RAB30 | RAB30, member RAS oncogene family |
| 233 | Neg | 0.00001255 | 0.3445 | 235170_at | ZNF92 | zinc finger protein 92 |
| 234 | Neg | 0.00002301 | 0.3445 | 203377_s_at | CDC40 | cell division cycle 40 homolog (S. cerevisiae) |
| 235 | Pos | 0.00008725 | 0.3445 | 236114_at | — | Transcribed locus |
| 236 | Pos | 0.00080721 | 0.3445 | 230389_at | FNBP1 | Formin binding protein 1 |
| 237 | Pos | 0.00000063 | 0.3445 | 244871_s_at | USP32 | ubiquitin specific peptidase 32 |
| 238 | Neg | 0.00119278 | 0.3445 | 227530_at | AKAP12 | A kinase (PRKA) anchor protein (gravin) 12 |
| 239 | Pos | 0.00044913 | 0.3445 | 201565_s_at | ID2 | inhibitor of DNA binding 2, dominant negative |
| | | | | | | helix-loop-helix protein |
| 240 | Pos | 0.00079925 | 0.3445 | 219753_at | STAG3 | stromal antigen 3 |
| 241 | Neg | 0.00005009 | 0.3445 | 218782_s_at | ATAD2 | ATPase family, AAA domain containing 2 |
| 242 | Pos | 0.00018418 | 0.3445 | 201554_x_at | GYG1 | glycogenin 1 |
| 243 | Pos | 0.00103168 | 0.3445 | 227062_at | TncRNA | trophoblast-derived noncoding RNA |
| 244 | Pos | 0.00007963 | 0.5864 | 207180_s_at | HTATIP2 | HIV-1 Tat interactive protein 2, 30kDa |
| 245 | Pos | 0.00004453 | 0.5864 | 212203_x_at | IFITM3 | interferon induced transmembrane protein 3 (1-8U) |
| 246 | Pos | 0.00022389 | 0.5864 | 210644_s_at | LAIR1 | leukocyte-associated immunoglobulin-like receptor 1 |
| 247 | Pos | 0.00102169 | 0.5864 | 213620_s_at | ICAM2 | intercellular adhesion molecule 2 |
| 248 | Neg | 0.01241763 | 0.5864 | 218373_at | AKTIP | AKT interacting protein |
| 249 | Pos | 0.00107255 | 0.5864 | 209365_s_at | ECM1 | extracellular matrix protein 1 |
| 250 | Neg | 0.00002165 | 0.5864 | 204822_at | TTK | TTK protein kinase |
| 251 | Pos | 0.00015116 | 0.5864 | 213035_at | ANKRD28 | ankyrin repeat domain 28 |
| 252 | Neg | 0.00048765 | 0.5864 | 221969_at | — | Transcribed locus |
| 253 | Neg | 0.00024929 | 0.5864 | 234140_s_at | STIM2 | stromal interaction molecule 2 |
| 254 | Neg | 0.00006625 | 0.5864 | 222680_s_at | DTL | denticleless homolog (Drosophila) |
| 255 | Neg | 0.00187756 | 0.5864 | 208650_s_at | CD24 | CD24 molecule |
| 256 | Pos | 0.00018824 | 0.5864 | 242121_at | RNF12 | Ring finger protein 12 |
| 257 | Pos | 0.00164760 | 0.5864 | 204759_at | RCBTB2 | regulator of chromosome condensation (RCC1) and |
| | | | | | | BTB (POZ) domain containing protein 2 |
| 258 | Neg | 0.00026865 | 0.5864 | 1565693_at | DTYMK | Deoxythymidylate kinase (thymidylate kinase) |
| 259 | Neg | 0.00002933 | 0.5864 | 224162_s_at | FBXO31 | F-box protein 31 |
| 260 | Pos | 0.00006702 | 0.5864 | 235142_at | RP1-27O5.1 /// | zinc finger and BTB domain containing 8 /// zinc |
| | | | | | ZBTB8 | finger and BTB domain containing 8-like |
| 261 | Pos | 0.00643099 | 0.5864 | 226905_at | FAM101B | family with sequence similarity 101, member B |
| 262 | Neg | 0.00031499 | 0.5864 | 212611_at | DTX4 | deltex 4 homolog (Drosophila) |
| 263 | Pos | 0.00066791 | 0.5864 | 228617_at | XAF1 | XIAP associated factor 1 |
| 264 | Pos | 0.00002358 | 0.5864 | 202615_at | GNAQ | Guanine nucleotide binding protein (G protein), q |
| | | | | | | polypeptide |
| 265 | Pos | 0.00132537 | 0.5864 | 243366_s_at | — | Transcribed locus |
| 266 | Pos | 0.00041347 | 0.5864 | 224566_at | TncRNA | trophoblast-derived noncoding RNA |
| 267 | Neg | 0.00001476 | 0.5864 | 223471_at | RAB3IP | RAB3A interacting protein (rabin3) |
| 268 | Pos | 0.00061623 | 0.5864 | 60471_at | RIN3 | Ras and Rab interactor 3 |
| 269 | Neg | 0.02530326 | 0.5864 | 217968_at | TSSC1 | tumor suppressing subtransferable candidate 1 |
| 270 | Pos | 0.00085651 | 0.5864 | 219806_s_at | C11orf75 | chromosome 11 open reading frame 75 |
| 271 | Pos | 0.00059783 | 0.5864 | 202771_at | FAM38A | family with sequence similarity 38, member A |
| 272 | Pos | 0.00622046 | 0.5864 | 1555705_a_at | CMTM3 | CKLF-like MARVEL transmembrane domain |
| | | | | | | containing 3 |
| 273 | Neg | 0.00043543 | 0.5864 | 237104_at | — | Transcribed locus |
| 274 | Neg | 0.00171051 | 0.5864 | 225019_at | CAMK2D | calcium/calmodulin-dependent protein kinase |
| | | | | | | (CaM kinase) II delta |
| 275 | Pos | 0.00167878 | 0.5864 | 203542_s_at | KLF9 | Kruppel-like factor 9 |
| 276 | Neg | 0.00205947 | 0.5864 | 201189_s_at | ITPR3 | inositol 1,4,5-triphosphate receptor, type 3 |
| 277 | Neg | 0.00382473 | 0.5864 | 231067_s_at | — | Transcribed locus |
| 278 | Pos | 0.00265825 | 0.5864 | 228113_at | RAB37 | RAB37, member RAS oncogene family |
| 279 | Neg | 0.00070928 | 0.5864 | 219135_s_at | LMF1 | lipase maturation factor 1 |
| 280 | Pos | 0.00009998 | 0.5864 | 37384_at | PPM1F | protein phosphatase 1F (PP2C domain containing) |
| 281 | Pos | 0.00503951 | 0.5864 | 209555_s_at | CD36 | CD36 molecule (thrombospondin receptor) |
| 282 | Neg | 0.00000083 | 0.5864 | 225649_s_at | STK35 | serine/threonine kinase 35 |
| 283 | Pos | 0.00010819 | 0.5864 | 1555486_a_at | FLJ14213 | protor-2 |
| 284 | Neg | 0.00018620 | 0.5864 | 218009_s_at | PRC1 | protein regulator of cytokinesis 1 |
| 285 | Pos | 0.05823921 | 0.5864 | 212592_at | IGJ | Immunoglobulin J polypeptide, linker protein for |
| | | | | | | immunoglobulin alpha and mu polypeptides |
| 286 | Pos | 0.00004247 | 0.5864 | 208109_s_at | C15orf5 | chromosome 15 open reading frame 5 |
| 287 | Neg | 0.00071640 | 0.5864 | 201792_at | AEBP1 | AE binding protein 1 |
| 288 | Pos | 0.00101179 | 0.5864 | 231431_s_at | — | CDNA clone IMAGE:4798730 |
| 289 | Pos | 0.00053465 | 0.5864 | 209287_s_at | CDC42EP3 | CDC42 effector protein (Rho GTPase binding) 3 |
| 290 | Pos | 0.00010578 | 0.5864 | 218749_s_at | SLC24A6 | solute carrier family 24 |
| | | | | | | (sodium/potassium/calcium exchanger), member 6 |
| 291 | Pos | 0.00001915 | 0.5864 | 240960_at | — | Transcribed locus |
| 292 | Pos | 0.00062248 | 0.5864 | 227567_at | AMZ2 | Archaelysin family metallopeptidase 2 |
| 293 | Neg | 0.00046323 | 0.5864 | 214875_x_at | APLP2 | amyloid beta (A4) precursor-like protein 2 |
| 294 | Neg | 0.00007963 | 0.5864 | 201397_at | PHGDH | phosphoglycerate dehydrogenase |
| 295 | Pos | 0.00028034 | 0.5864 | 220558_x_at | TSPAN32 | tetraspanin 32 |
| 296 | Pos | 0.00155722 | 0.9484 | 229530_at | — | CDNA clone IMAGE:5302158 |
| 297 | Neg | 0.00098262 | 0.9484 | 200790_at | ODC1 | ornithine decarboxylase 1 |
| 298 | Neg | 0.00270658 | 0.9484 | 219396_s_at | NEIL1 | nei endonuclease VIII-like 1 (E. coli) |
| 299 | Neg | 0.00102169 | 0.9484 | 242468_at | — | — |
| 300 | Pos | 0.00080721 | 0.9484 | 229015_at | LOC286367 | FP944 |
| 301 | Neg | 0.00396044 | 0.9484 | 214835_s_at | SUCLG2 | succinate-CoA ligase, GDP-forming, beta subunit |
| 302 | Pos | 0.00001286 | 0.9484 | 209321_s_at | ADCY3 | adenylate cyclase 3 |
| 303 | Neg | 0.00073084 | 0.9484 | 1555372_at | BCL2L11 | BCL2-like 11 (apoptosis facilitator) |
| 304 | Neg | 0.00007434 | 0.9484 | 205005_s_at | NMT2 | N-myristoyltransferase 2 |
| 305 | Neg | 0.00013234 | 0.9484 | 235258_at | DCP2 | DCP2 decapping enzyme homolog (S. cerevisiae) |
| 306 | Pos | 0.00016508 | 0.9484 | 51146_at | PIGV | phosphatidylinositol glycan anchor biosynthesis, |
| | | | | | | class V |
| 307 | Pos | 0.00140329 | 0.9484 | 220330_s_at | SAMSN1 | SAM domain, SH3 domain and nuclear |
| | | | | | | localization signals 1 |
| 308 | Pos | 0.00032171 | 0.9484 | 1557501_a_at | — | Full length insert cDNA clone YB22B02 |
| 309 | Pos | 0.00013087 | 0.9484 | 235922_at | — | CDNA FLJ39413 fis, clone PLACE6015729 |
| 310 | Pos | 0.00030841 | 0.9484 | 1554250_s_at | TRIM73 | tripartite motif-containing 73 |
| 311 | Pos | 0.00126350 | 0.9484 | 209604_s_at | GATA3 | GATA binding protein 3 |
| 312 | Pos | 0.00064807 | 0.9484 | 225883_at | ATG16L2 | ATG16 autophagy related 16-like 2 (S. cerevisiae) |
| 313 | Pos | 0.00006548 | 0.9484 | 209627_s_at | OSBPL3 | oxysterol binding protein-like 3 |
| 314 | Pos | 0.00213666 | 0.9484 | 201170_s_at | BHLHB2 | basic helix-loop-helix domain containing, class B, 2 |
| 315 | Pos | 0.00022148 | 0.9484 | 226267_at | JDP2 | jun dimerization protein 2 |
| 316 | Pos | 0.00005968 | 0.9484 | 232614_at | — | CDNA FLJ12049 fis, clone HEMBB1001996 |
| 317 | Pos | 0.00041778 | 0.9484 | 204689_at | HHEX | hematopoietically expressed homeobox |
| 318 | Pos | 0.00010226 | 0.9484 | 205462_s_at | HPCAL1 | hippocalcin-like 1 |
| 319 | Neg | 0.00020534 | 0.9484 | 210279_at | GPR18 | G protein-coupled receptor 18 |
| 320 | Neg | 0.00643099 | 0.9484 | 208703_s_at | APLP2 | amyloid beta (A4) precursor-like protein 2 |
| 321 | Pos | 0.00011574 | 0.9484 | 207986_x_at | CYB561 | cytochrome b-561 |
| 322 | Neg | 0.00001756 | 0.9484 | 218344_s_at | RCOR3 | REST corepressor 3 |
| 323 | Neg | 0.00082334 | 0.9484 | 225147_at | PSCD3 | pleckstrin homology, Sec7 and coiled-coil domains 3 |
| 324 | Pos | 0.00102169 | 0.9484 | 202371_at | TCEAL4 | transcription elongation factor A (SII)-like 4 |
| 325 | Pos | 0.00410051 | 0.9484 | 205407_at | RECK | reversion-inducing-cysteine-rich protein with |
| | | | | | | kazal motifs |
| 326 | Pos | 0.00005631 | 0.9484 | 227502_at | KIAA1147 | KIAA1147 |
| 327 | Pos | 0.00127566 | 0.9484 | 224697_at | WDR22 | WD repeat domain 22 |
| 328 | Pos | 0.00100198 | 0.9484 | 228412_at | LOC643072 | hypothetical LOC643072 |
| 329 | Pos | 0.00229906 | 0.9484 | 236395_at | — | Transcribed locus |
| 330 | Pos | 0.00064807 | 0.9484 | 207761_s_at | METTL7A | methyltransferase like 7A |
| 331 | Neg | 0.00097307 | 0.9484 | 209383_at | DDIT3 | DNA-damage-inducible transcript 3 |
| 332 | Pos | 0.00104176 | 0.9484 | 227001_at | NPAL2 | NIPA-like domain containing 2 |
| 333 | Pos | 0.00011574 | 0.9484 | 241916_at | — | Transcribed locus |
| 334 | Pos | 0.00060391 | 0.9484 | 201328_at | ETS2 | v-ets erythroblastosis virus E26 oncogene |
| | | | | | | homolog 2 (avian) |
| 335 | Pos | 0.00089972 | 0.9484 | 228623_at | — | Transcribed locus |
| 336 | Neg | 0.00001012 | 0.9484 | 226233_at | B3GALNT2 | beta-1,3-N-acetylgalactosaminyltransferase 2 |
| 337 | Neg | 0.00042213 | 0.9484 | 204998_s_at | ATF5 | activating transcription factor 5 |
| 338 | Pos | 0.00215637 | 0.9484 | 218400_at | OAS3 | 2′-5′-oligoadenylate synthetase 3, 100kDa |
| 339 | Pos | 0.00019238 | 0.9484 | 243279_at | — | Transcribed locus |
| 340 | Pos | 0.00251794 | 0.9484 | 230161_at | — | Transcribed locus |
| 341 | Neg | 0.00019449 | 0.9484 | 228049_x_at | — | Transcribed locus, strongly similar to XP_001172939.1 |
| | | | | | | PREDICTED: hypothetical protein [Pan troglodytes] |
| 342 | Neg | 0.00023374 | 0.9484 | 226118_at | CENPO | centromere protein O |
| 343 | Pos | 0.00003596 | 0.9484 | 209195_s_at | ADCY6 | adenylate cyclase 6 |
| 344 | Pos | 0.00000409 | 0.9484 | 227132_at | ZNF706 | zinc finger protein 706 |
| 345 | Neg | 0.00611754 | 0.9484 | 215772_x_at | SUCLG2 | succinate-CoA ligase, GDP-forming, beta subunit |
| 346 | Pos | 0.00039664 | 0.9484 | 212326_at | VPS13D | vacuolar protein sorting 13 homolog D (S. cerevisiae) |
| 347 | Pos | 0.00049267 | 0.9484 | 209933_s_at | CD300A | CD300a molecule |
| 348 | Neg | 0.00028636 | 0.9484 | 220719_at | FLJ13769 | hypothetical protein FLJ13769 |
| 349 | Pos | 0.00009998 | 0.9484 | 243356_at | — | Transcribed locus |
| 350 | Neg | 0.00144382 | 0.9484 | 204735_at | PDE4A | phosphodiesterase 4A, cAMP-specific |
| | | | | | | (phosphodiesterase E2 dunce homolog, Drosophila) |
| 351 | Neg | 0.00196658 | 0.9484 | 203505_at | ABCA1 | ATP-binding cassette, sub-family A (ABC1), member 1 |
| 352 | Pos | 0.00003863 | 0.9484 | 1555420_a_at | KLF7 | Kruppel-like factor 7 (ubiquitous) |

Note:
Neg = MRD negative; Pos = MRD positive; p-value via two sample t-test
FDR = False discovery rate as estimated by SAM
Probe sets (top 23) used for final model building are shaded

### Consideration of Diagnostic White Blood Cell (WBC) Count as a Predictive Variable

The WBC count at diagnosis had an independent effect on predicting RFS in our population but was deemed untenable for use in modeling building due to the requirement of a binary WBC cutoff value instead of a continuous variable. We believed that a cutoff value would be over-influenced by the cohort composition and patient age, particularly given that trial eligibility and enrollment may itself be based on an age-adjusted WBC count. A WBC cutoff of 50 K/uL was shown to have significance in the validation cohort but not in our cohort, yet the gene expression classifier for RFS derived in the present work proved informative despite differences in clinical parameters and therapies between the external validation group and our cohort.

### Technical Details on the Construction and Evaluation of the Gene Expression Classifier for RFS

This section describes the detailed analysis techniques that were used to construct and evaluate the gene expression classifier. Throughout this section and the next, the gene expression data will be denoted by xᵢⱼ, i=1, 2, . . . , p, j=1, 2, . . . , n, where p and n are the numbers of genes and samples, respectively. Here a gene refers to a probe set. The prediction model was constructed in two stages—gene selection and model building.
Gene selection based on association with outcome, here RFS, is a necessary step for removing irrelevant genes and thus improving the accuracy of the final prediction model. It also reduces the dimensionality of the feature space so that a small subset of genes can be used to build a stable predictor. In this paper we based our gene selection on the Cox score² calculated for each gene i:

$${h}_{i} = \frac{{r}_{i}}{{s}_{i} + {s}_{0}} ; i = 1 , 2 , … , p .$$

Given a threshold τ>0, a gene will be excluded if the absolute value of its Cox score is less than τ. The Cox score for gene i is calculated as follows. We denote the censored RFS data for sample jas yⱼ=(tⱼ,Δⱼ), where tⱼ is time and Δᵢ=1 if the observation is relapse, 0 if censored. Let D be the indices of the K unique death times z₁, z₂, . . . z_{K}. Let R₁, R₂, . . . , R_{K} denote the sets of indices of the observations at risk at these unique relapse times, that is Rₖ={i:tᵢ≧zₖ}. Let mₖ=the number of indices in Rₖ. Let dₖ be the number of deaths at time zₖ and xᵢₖ*=Σₜ_{ⱼ}_{=z}_{ₖ}xᵢⱼ and x̅ᵢₖ=Σ_{jεR}_{ₖ}xᵢⱼ/mₖ. Then

$${r}_{i} = {\sum }_{k = 1}^{K} ( {x}_{ij}^{*} - {d}_{k} {\overline{x}}_{ik} )$$

$$and$$

$${s}_{i} = {[ {\sum }_{k = 1}^{K} ( {d}_{k} / {m}_{k} ) {\sum }_{j ∈ R} {( {x}_{ij} - {\overline{x}}_{ik} )}^{2} ]}^{\frac{1}{2}} .$$

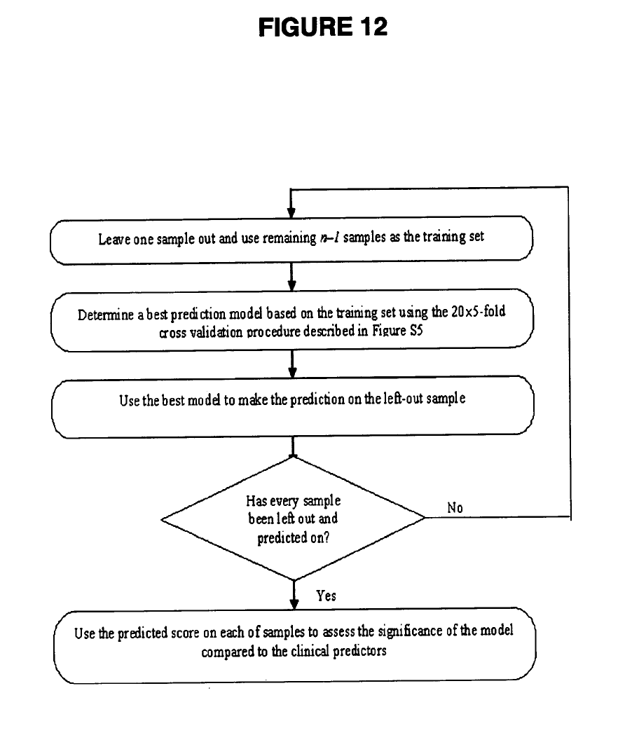

s₀ is the median of all sᵢ.
After excluding the irrelevant genes, principal component analysis is performed on the standardized expression values of the remaining genes. Cox proportional hazard regression is then performed on the scores of the first principal component. The linear part of the fitted regression model, which is also a linear combination of the probe sets, is used as the prediction model. This model predicts a continuous score, either positive or negative, on a new sample, which is associated with the risk to relapse: the higher the score, the higher the risk. The performance of the predictions on a set of new samples can be evaluated by examining the association between the predicted score and RFS status of the samples. This was done in our analysis by performing a Cox proportional hazard regression and calculating the likelihood ratio test (LRT) statistic. Larger LRT implies better performance.
The number of genes included in the prediction model and the performance of the model both depend on the threshold τ. In this study 20 candidate thresholds were considered and the one corresponding to the best model was determined through a 20×5-fold cross-validation
Once we have obtained a prediction model we would like to assess the significance of the model compared with known clinical predictors. One approach to doing this would be to use the model to make predictions back on the samples and then compare the predicted risk scores with the clinical predictors. It is known that such an approach is biased which would overestimate the significance of the final model because the same data were used both to develop the model and to evaluate its significance.⁹ Another alternative approach that can avoid this bias is to separate the data into a training set for developing the model through the above procedure and a test set used for evaluating the performance of the model. The disadvantage of such an approach is that it does not make efficient use of the data, since the training set may be too small to develop an accurate model, and the test set may be too small to evaluate its significance.⁹ To obtain an objective and unbiased prediction on each of the all samples and make best use of the data we therefore employed a nested cross-validation procedure as suggested by Simon⁹ and used by Asgharzadeh et. al.¹⁰ This procedure, detailed in FIG. 12/S6, consists of Leave-One-Out Cross-Validation (LOOCV) with each fold including a 20×5-fold cross-validation.

### Technical Details on the Construction and Evaluation of the Gene Expression Classifier for Predicting Day 29 MRD

The methodology for constructing and evaluating the gene expression predictor for MRD is essentially the same as that described in the previous section. Because the response variable is binary (either MRD positive or negative), constructing the model is significantly less computationally-intensive, which allows more folds of cross-validation.

Gene selection is performed using the filter method with the modified t-test statistic calculated for each gene i:^{10,39}

$${h}_{i} = \frac{{\hat{μ}}_{P , i} - {\hat{μ}}_{N , i}}{{\hat{σ}}_{i} + {\hat{σ}}_{0}} ; i = 1 , 2 , … , p .$$

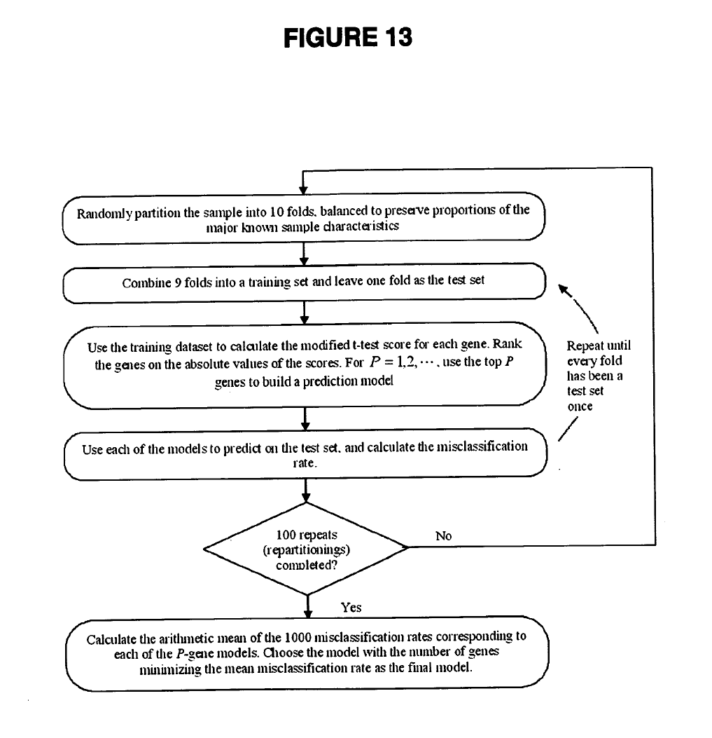

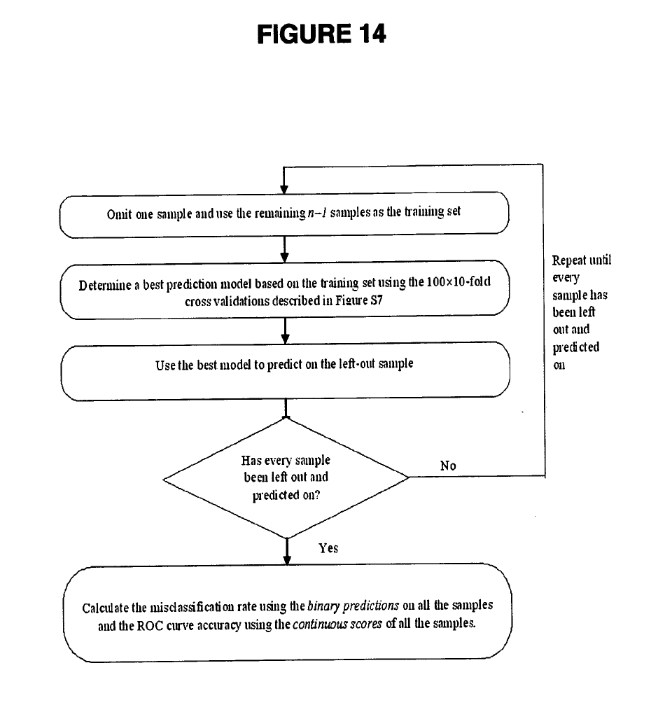

Here the numerator corresponds to the difference of the sample means of the two classes (MRD positive and negative), and the denominator is an estimate \hat{σ}ᵢ of the standard deviation plus a positive number \hat{σ}₀, where \hat{σ}₀ is the median of all \hat{σ}₁.
The prediction analysis is based on the diagonal linear discriminant analysis (DLDA) method.¹⁴ After calculating the modified t-test statistic hᵢ for all genes, we ranked the genes in descending order by the absolute value |hᵢ|. The top P genes were used to build the discriminant function:

$$g ( x ) = log ( \frac{{\hat{p}}_{p}}{{\hat{p}}_{n}} ) + {\sum }_{i}^{P} {h}_{i} \frac{{x}_{i} - {\hat{μ}}_{i}}{{\hat{σ}}_{i} + {\hat{σ}}_{0}} ,$$

where \hat{p}ₚ and \hat{p}ₙ are the proportions of the MRD positive and negative samples, and \hat{μ}ᵢ is the mean expression value of the ith gene. This model predicts a continuous score, either positive or negative, on a new sample, where a higher value is more indicative of MRD positive. The model uses zero as a binary prediction threshold and predicts MRD positive if the predicted score is positive and MRD negative otherwise. The prediction performance depends on the number P of top significant genes included in the model. The value of P corresponding to the best model was determined through a 100×10-fold cross-validation procedure, as illustrated schematically in FIG. 13/S7.
As with the performance evaluation for the RFS predictor, we employed a nested cross-validation procedure as suggested by Simon⁹ and used by Asgharzadeh et. al.¹⁰ to obtain an objective and unbiased performance evaluation for the DLDA model, which also makes best use of the data. This procedure, detailed in FIG. 14/S8, consists of Leave-One-Out Cross-Validation (LOOCV), with each fold including a 100×10-fold cross-validation as illustrated in FIG. 13/S7.

Development pf a Gene Expression Classifier for RFS in High-Risk ALL Excluding Cases with Known Recurring Cytogenetic Abnormalities (t(1;19) and MLL)

In this analysis we rebuilt the gene expression classifier for RFS from the beginning through the extensive nested cross validation. Please note that we removed the probe sets using the rule of 50% present call. After removing t(1;19) translocation and MLL rearrangement cases we were left with 163 patients. A 20×5-fold cross validation as detailed in original manuscript was performed to determine the model for predicting the risk score of relapse. Twenty candidate thresholds were considered. The number of significant probe sets determined by each threshold and geometric mean of the likelihood ratio test statistic corresponding to each threshold are listed in Table S7.

TABLE S7
Candidate thresholds and corresponding numbers of significant genes
and geometric means of likelihood ratio test (LRT) statistic values.

| | | # significant | LRT Statistic |
|---|---|---|---|
| Threshold # | Threshold | Genes | (Geometric mean) |
| 1 | 0.00007 | 23773.15 | 0.668258 |
| 2 | 0.14674 | 20191.85 | 0.688759 |
| 3 | 0.29341 | 16699.37 | 0.779984 |
| 4 | 0.44007 | 13379.21 | 0.849028 |
| 5 | 0.58674 | 10351.13 | 0.883603 |
| 6 | 0.73341 | 7689.64 | 0.857314 |
| 7 | 0.88007 | 5434.52 | 0.842705 |
| 8 | 1.02674 | 3647.99 | 0.917711 |
| 9 | 1.17341 | 2313.88 | 0.938914 |
| 10 | 1.32008 | 1383.15 | 1.01001 |
| 11 | 1.46674 | 780.68 | 1.212886 |
| 12 | 1.61341 | 420.9 | 1.474257 |
| 13 | 1.76008 | 219.08 | 1.932876 |
| 14 | 1.90674 | 111.1 | 2.328886 |
| 15 | 2.05341 | 58.25 | 2.193993 |
| 16 | 2.20008 | 31.5 | 2.564132 |
| 17 | 2.34674 | 17.56 | 2.443301 |
| 18 | 2.49341 | 10.13 | 1.978379 |
| 19 | 2.64008 | 5.99 | 1.531674 |
| 20 | 2.78674 | 3.53 | 0.948933 |

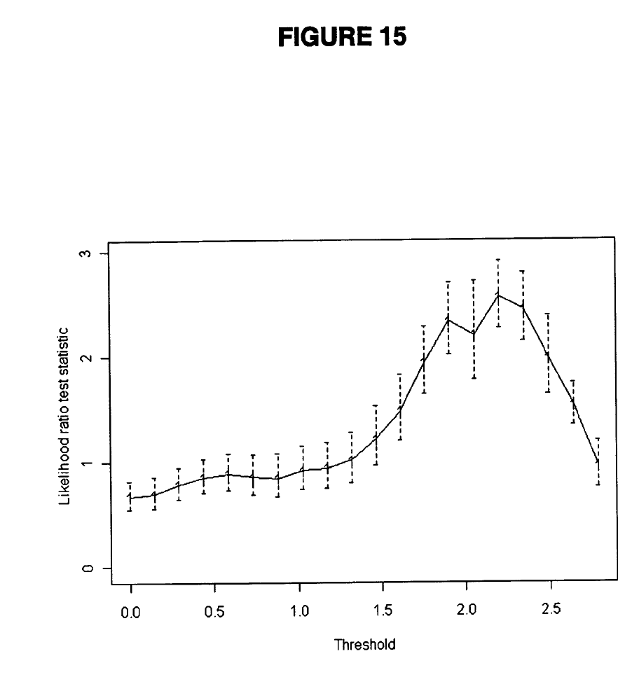

The mean of the LRT statistic is also plotted in FIG. 15/S9. We see that the geometric mean of the LRT reaches the maximum when the threshold is The “best” model determined by this threshold is a linear combination of expression values of 32 probe sets that are highly associated with RFS status. The information about the 32 probe sets are presented in Table S8, below.

TABLE S8
Probe sets (and associated genes) that are significantly associated with RFS

| Rank | score | Probe Set ID | Gene Symbol | Gene Title |
|---|---|---|---|---|
| 1 | 3.25 | 210830_s_at | PON2 | paraoxonase 2 |
| 2 | 3.24 | 242579_at | BMPR1B | bone morphogenetic protein receptor, type IB |
| 3 | 3.07 | 201876_at | PON2 | paraoxonase 2 |
| 4 | 2.97 | 236750_at | — | — |
| 5 | 2.94 | 212592_at | IGJ | immunoglobulin J polypeptide, linker protein for |
| | | | | immunoglobulin alpha and mu polypeptides |
| 6 | −2.79 | 216834_at | RGS1 | regulator of G-protein signaling 1 |
| 7 | 2.72 | 232539_at | — | — |
| 8 | 2.71 | 209288_s_at | CDC42EP3 | CDC42 effector protein (Rho GTPase binding) 3 |
| 9 | −2.69 | 202388_at | RGS2 | regulator of G-protein signaling 2, 24 kDa |
| 10 | 2.68 | 213371_at | LDB3 | LIM domain binding 3 |
| 11 | 2.64 | 215028_at | SEMA6A | sema domain, transmembrane domain (TM), and |
| | | | | cytoplasmic domain, (semaphorin) 6A |
| 12 | 2.63 | 215617_at | LOC26010 | viral DNA polymerase-transactivated protein 6 |
| 13 | 2.61 | 209101_at | CTGF | connective tissue growth factor |
| 14 | 2.59 | 204030_s_at | SCHIP1 | schwannomin interacting protein 1 |
| 15 | −2.55 | 209959_at | NR4A3 | nuclear receptor subfamily 4, group A, member 3 |
| 16 | 2.53 | 222780_s_at | BAALC | brain and acute leukemia, cytoplasmic |
| 17 | 2.53 | 203939_at | NT5E | 5′-nucleotidase, ecto (CD73) |
| 18 | 2.51 | 236766_at | — | — |
| 19 | 2.47 | 202242_at | TSPAN7 | tetraspanin 7 |
| 20 | 2.44 | 225355_at | LOC54492 | neuralized-2 |
| 21 | 2.41 | 211675_s_at | MDFIC | MyoD family inhibitor domain containing |
| 22 | 2.40 | 219313_at | GRAMD1C | GRAM domain containing 1C |
| 23 | −2.40 | 203921_at | CHST2 | carbohydrate (N-acetylglucosamine-6-O) |
| | | | | sulfotransferase 2 |
| 24 | 2.39 | 219871_at | FLJ13197 | hypothetical FLJ13197 |
| 25 | −2.39 | 207978_s_at | NR4A3 | nuclear receptor subfamily 4, group A, member 3 |
| 26 | −2.38 | 221349_at | VPREB1 | pre-B lymphocyte 1 |
| 27 | 2.36 | 244280_at | — | — |
| 28 | 2.34 | 209365_s_at | ECM1 | extracellular matrix protein 1 |
| 29 | 2.33 | 239673_at | — | — |
| 30 | 2.33 | 223449_at | SEMA6A | sema domain, transmembrane domain (TM), and |
| | | | | cytoplasmic domain, (semaphorin) 6A |
| 31 | −2.32 | 202506_at | SSFA2 | sperm specific antigen 2 |
| 32 | −2.32 | 205241_at | SCO2 | SCO cytochrome oxidase deficient homolog 2 |
| | | | | (yeast) |

Through the nested cross validation procedure as described in the manuscript the gene expression-based risk classifier predicted a risk score on each of the 163 patients. With a threshold of zero the risk score separated the 163 patients into low (n=66) vs. high (n=97) risk groups. Table S9 shows the association between the risk groups with day 29 MRD.

TABLE S9
Two-Way Classification Table of
Risk Groups and Day 29 MRD Status

| | |
|---|---|
| MRD day 28 | Risk Group |

| (binary) | Low Risk | High Risk | Total |
|---|---|---|---|
| Negative | 61 | 35 | 96 |
| | 63.54 | 36.46 | 100.00 |
| Positive | 24 | 34 | 58 |
| | 41.38 | 58.62 | 100.00 |
| Missing | 3 | 6 | 9 |
| | 33.33 | 66.67 | 100.00 |
| Total | 88 | 75 | 163 |
| | 53.99 | 46.01 | 100.00 |

Fisher Exact Test (after removing missing data): 0.006

The Kaplan-Meier estimates of relapse-free survival (RFS) for the various groups based on gene expression classifer-based risk group for RFS and end-induction flow cytometric MRD status were plotted in Figures S10 (A) through (F) as follows

### Identification of Novel Cluster Groups in Pediatric Higher Risk B-Precursor Acute Lymphoblastic Leukemia by Unsupervised Gene Expression Profiling

The cure rate of pediatric B-precursor acute lymphoblastic leukemia (ALL) now exceeds 80% with contemporary treatment regimens. These therapeutic advances have come through the progressive refinement of chemotherapy and the development of risk classification schemes that target children to more intensive therapies based on their relapse risk.¹ Current risk classification schemes incorporate pre-treatment clinical characteristics (white blood cell count (WBC), age, and the presence of extramedullary disease), the presence or absence of sentinel cytogenetic lesions (such as t(12;21)(ETV6-RUNX1) and t(9;22)(BCR-ABL1), translocations involving MLL, and chromosomal trisomies or hypodiploidy), and measures of minimal residual disease (MRD) at the end of induction therapy, to classify children with ALL into “low,” “standard/intermediate,” “high,” or “very high” risk categories.² Despite improvements in treatment and in risk classification over the past three decades, up to 20% of children with ALL still relapse. The majority of relapses occur in those children who are initially classified as “standard/intermediate” or “high” risk. Thus, while overall outcomes have significantly improved, children classified with “high” or “very high” risk disease, those who have relapsed, or those of Hispanic or American Indian descent continue to have relatively poor survivals.³ These latter groups require the development of novel therapies for cure.

Shuster previously showed that the group of children with high-risk B-precursor ALL based on the “NCl/Rome” criteria (age ≧10 years and/or presenting WBC ≧50,000/μL) could be refined using age, sex and WBC to identify a subgroup of ˜12% of B-precursor ALL patients, referred to herein as “higher” risk, that had a very poor outcome with <50% expected survival.⁴ In contrast to children with favorable, “low” risk ALL (associated with the presence of t(12;21)(ETV6-RUNX1) or trisomies of chromosomes 4, 10, and 17) or those with unfavorable, “very high” risk disease (associated with t(9;22)(BCR-ABL1) or hypodiploidy), the biologic and genetic features of these higher risk ALL patients are only now becoming well characterized.⁵ To identify novel, biologically defined subgroups within higher risk ALL and to identify genes defining these subgroups that might serve as new diagnostic or therapeutic targets for this form of disease, we performed GEP analysis in a cohort of 207 uniformly treated higher risk ALL patients who were enrolled in the Children's Oncology Group (COG) P9906 clinical trial (http://www.acor.org/pedonc/diseases/ALLtrials/9906.html). Under the auspices of a National Cancer Institute TARGET Project (Therapeutically Applicable Research to Generate Effective Treatments; www.target.cancer.gov), we have also assessed genome-wide DNA copy number abnormalities in leukemic DNA in this same cohort⁵ and have performed selective gene resequencing to identify genes consistently mutated in the leukemias cells of the cohort.⁶ Herein we report the discovery of 8 gene expression-based cluster groups of patients within higher risk pediatric ALL, identified through shared patterns of gene expression. While two of these clusters were found to be associated with known recurrent cytogenetic abnormalities (either t(1;19)(TCF3-PBX1) or MLL translocations), the remaining 6 cluster groups had no detectable conserved cytogenetic aberrations, but 2 of the groups were associated with strikingly different therapeutic outcomes and clinical characteristics. The gene expression-based cluster groups were also associated with distinct patterns of genome-wide DNA copy number abnormalities and with the aberrant expression of “outlier” genes. These genes provide new targets for improved diagnosis, risk classification, and therapy for this poor risk form of ALL.

### Materials and Methods

### Patient Selection and Characteristics

The COG Trial P9906 enrolled 272 eligible children and adolescents with higher-risk ALL between Mar. 15, 2000 and Apr. 25, 2003. This trial targeted a subset of patients with higher risk features (older age and higher WBC) that had experienced relatively poor outcomes (<50% 4-year relapse-free survival (RFS)) in prior COG clinical trials.⁴ Patients were first enrolled on the COG P9000 classification study and received a four-drug induction regimen.⁷ Those with 5-25% blasts in the bone marrow (BM) at day 29 of therapy received 2 additional weeks of extended induction therapy using the same agents. Patients in complete remission (CR) with less than 5% BM blasts following either 4 or 6 weeks of induction were then eligible to participate in COG P9906 if they met the age and WBC criteria described previously⁴ or had overt central nervous system (CNS3) or testicular involvement at diagnosis. Patients that met the higher risk age/sex/WBC criteria but had favorable genetic features [t(12;21)(ETV6-RUNX1) or trisomy of chromosomes 4 and 10] or those with unfavorable, “very high” risk features [t(9;22)(BCR-ABL1) or hypodiploidy] were excluded.⁸ Patients enrolled in COG P9906 were uniformly treated with a modified augmented BFM regimen that included two delayed intensification phases.^{9,10} The majority of patients had MRD assessed by flow cytometric analysis of bone marrow samples at day 29 of induction therapy as previously described¹¹; cases were defined as MRD-positive or MRD-negative at day 29 using a threshold of 0.01%.

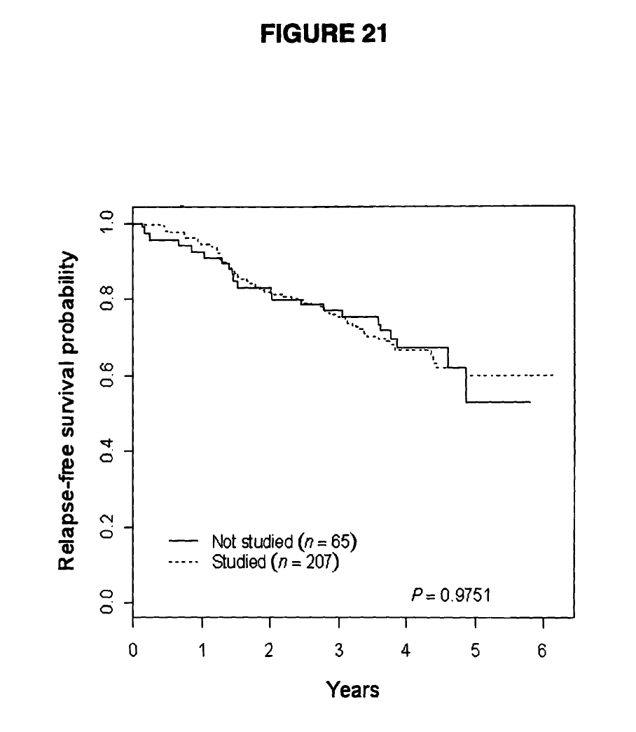

For this study, cryopreserved pre-treatment leukemia specimens were available on a representative cohort of 207 of the 272 (76%) patients registered to this trial. The 65 unstudied patients included a greater proportion of older boys with lower WBC counts, but otherwise were similar and showed no significant outcome differences (Supplement Table S1′; FIG. 21). Treatment protocols were approved by the National Cancer Institute (NCI) and participating institutions through their Institutional Review Boards. Informed consent for participation in these research studies was obtained from all patients or their guardians. Outcome data for all patients were frozen as of October 2006; the median time to event or censoring was 3.7 years. A validation cohort consisted of an independent studyl² of 99 cases of NCl/Rome high risk ALL that were derived from COG Trial CCG 1961 and used the same Affymetrix microarray platform.

### Gene Expression Profiling

RNA was isolated from pre-treatment, diagnostic samples in the 207 ALL cases (131 bone marrow, 76 peripheral blood) using TRIzol (Invitrogen, Carlsbad, Calif.); all samples had >80% leukemic blasts. cDNA labeling, hybridization and scanning were performed as previously described (detailed in Supplement).¹³ A mask to remove uninformative probe pairs was applied to all the arrays (detailed in Supplement, Section 3). The default MAS 5.0 normalization was used. Array experimental quality was assessed using the following parameters and all arrays met these criteria for inclusion: GAPDH ≧5,000; ≧20% expressed genes; GAPDH 3′/5′ ratios ≦4; and linear regression r-squared values of spiked poly(A) controls >0.90. This gene expression dataset may be accessed via the National Cancer Institute caArray site (https://array.nci.nih.gov/caarray/) or at Gene Expression Omnibus (http://www.ncbi.nlm.nih.gov/geo/).

### Unsupervised Clustering Methods and Selection of Outlier Genes

Microarray gene expression data were available from an initial 54,504 probe sets after masking and filtering (see Supplement, Section 30. Three distinctly different methods were used to select genes for hierarchical clustering: High Coefficient of variation (HC), Cancer Outlier Profile Analysis (COPA) and Recognition of Outliers by Sampling Ends (ROSE). In HC, the 54,504 probe sets were ordered by their coefficients of variation (CV) and the highest 254 probe sets were used for clustering. This method identifies probe set having an overall high variance relative to mean intensity. COPA (previously described by Tomlins et al)¹⁴ selects outlier probe sets on the basis of their absolute deviation from median at a fixed point (typically 95^{th} percentile). ROSE was developed in our laboratory as an alternative to COPA, and selects probe sets both on the basis of the size of the outlier group they identify as well as the magnitude of the deviation from expected intensity (see Supplement, Sections 4B and C for detailed methods of ROSE and COPA).

For all three probe selection methods, the top 254 probe sets were clustered using EPCLUST (http://www.bioinf.ebc.ee/EP/EP/EPCLUST/, v0.9.23 beta, Euclidean distance, average linkage UPGMA). A threshold branch distance was applied and the largest distinct branches above this threshold containing more than 8 patients were retained and labeled. The HC method was used as the basis of cluster nomenclature, with each new cluster being assigned a number. All clusters are prefixed by the method of their probe set selection (H=High CV, C=COPA and R=ROSE), with COPA and ROSE numbers being assigned by the similarity of their group's membership to H-clusters. The top 100 median rank order probe sets for each ROSE cluster are listed in the Supplement, Section 6.

In the validation cohort (CCG 1961) the same initial filtering criteria were applied to the raw data. Each method began with 54,504 probe sets. Applying the ROSE method, with the same cutoffs used in P9906, 167 probe sets were retained and used for clustering. COPA and HC also used the same selection criteria as in P9906, and the top 167 probe sets were used in clustering (Supplement, Table S7A′).

### Assessment of Genome-Wide DNA Copy Number Abnormalities (CNA)

Copy number alterations were detected as described in Mullighan et al, and the initial CNA data for this cohort are also presented there.⁵ Briefly, DNA from the diagnostic leukemic cells and from a sample obtained after remission induction therapy (germline) was extracted and genotyped using either the 250K Sty and Nsp single-nucleotide-polymorphism (SNP) arrays (Affymetrix, Santa Clara, Calif.). SNP array data preprocessing and inference of DNA copy number abnormalities (CNA) and loss-of-heterozygosity (LOH) was performed as previously described.^{15,16}

### Statistical Analyses

Log rank analysis was used to evaluate relapse-free survival (RFS).¹⁷ Kaplan-Meier survival analyses and hazard ratios were also calculated for comparisons of group RFS.^{18,19} Kruskal-Wallis rank sum tests were used to analyze age and WBC counts; Fisher's exact test was used to evaluate the binary variables.¹⁸ All statistical analyses were performed using R²⁰ (http://www.R-project.org, version 2.9.1, with stats and survival packages).

### Results

Reflective of their classification as higher risk, the 207 children and adolescents had a median age of 13 years (range: 1-20 years), a median WBC at disease presentation of 62,300/μL, a male predominance (66%), and 35% were MRD positive at day 29 of induction therapy⁷ (Supplement, Table S2′). Nearly 25% (51/205) of these children were of Hispanic/Latino ethnicity, while 10% (21/207) had translocations involving the MLL gene on chromosome 11q23 and 11% (23/207) had t(1;19)(TCF3-PBX1) translocations (Supplement, Table S1′). The remaining cases (79%) did not have known recurring chromosomal translocations. Relapse-free survival (RFS) and overall survival (OS) in the 207 patients were 66.3±3.5% and 83% at 4 years, respectively (FIG. 21).

### Unsupervised Hierarchical Clustering Defines Eight Gene Expression Cluster Groups

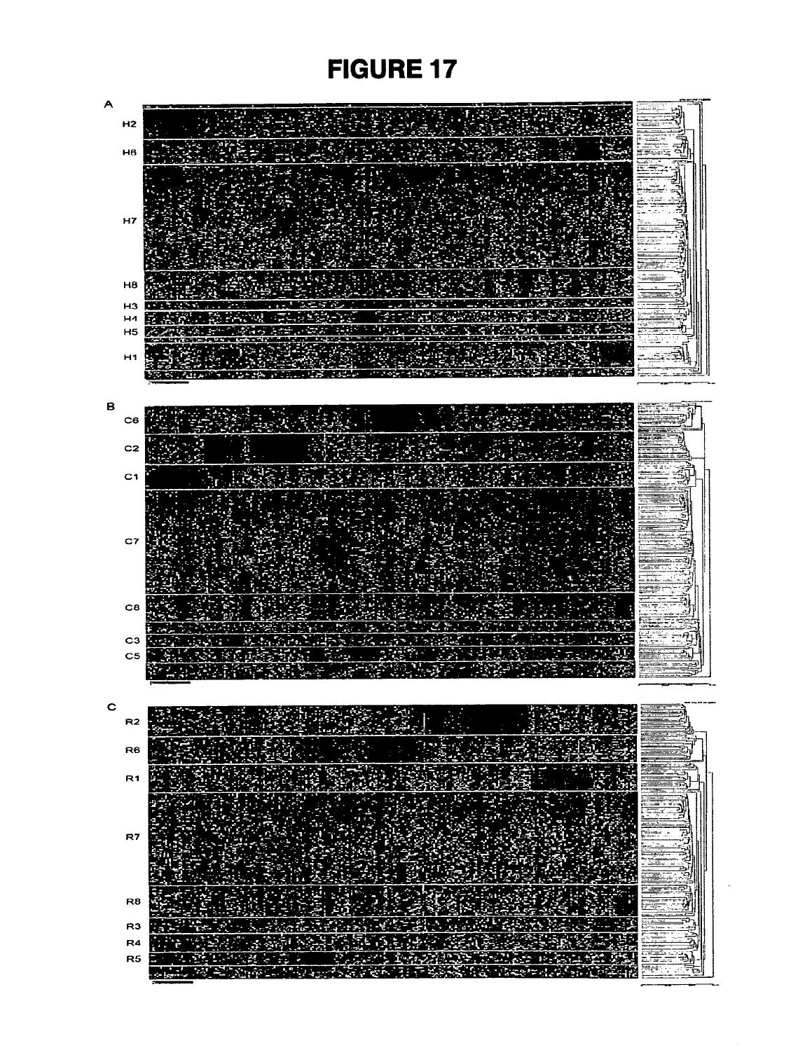

Based upon the assumption that the most robust clusters would be repeatedly and consistently identified by more than one clustering approach, several methods of selecting probe sets for unsupervised clustering were applied to the gene expression data. First, using the top 254 genes selected by CV (the full gene list is provided in Supplement, Table S7A′), we identified 8 distinct gene expression-based cluster groups which were labeled H1 through H8 (FIG. 17A). Interestingly, while 20 of 21 cases with an MLL translocation were in cluster H1 (Table 1′) and all 23 cases with a t(1;19)(TCF3-PBX1) were in cluster H2 (FIG. 17A), the remaining 6 clusters (labeled H3-H8) lacked a clear association with any previously described cytogenetic abnormality.

TABLE 1′
Association of Clinical and Outcome Features with High CV Expression Cluster Groups¹

| | | | | | | | | | | P- |
|---|---|---|---|---|---|---|---|---|---|---|
| | H1 | H2 | H3 | H4 | H5 | H6 | H7 | H8 | Total | Value² |
| # Cases/Cluster | 20 | 23 | 8 | 11 | 9 | 19 | 95 | 22 | 207 | — |
| Median Age (Yrs) | 6.9 | 13.1 | 13.8 | 14.2 | 14.7 | 14.5 | 11.4 | 13.8 | 13.1 | 0.002 |
| Sex (Male) | 11/20 | 11/23 | 4/8 | 10/11 | 7/9 | 15/19 | 64/95 | 15/22 | 137/207 | 0.165 |
| Ethnicity (Hispanic) | 3/20 | 6/23 | 2/8 | 2/11 | 0/8 | 3/18 | 22/95 | 13/22 | 51/205 | 0.018 |
| MLL | 20/20 | 0/23 | 0/8 | 0/11 | 0/9 | 0/19 | 1/94 | 0/22 | 21/207 | <0.001 |
| TCF3-PBX1 | 0/20 | 23/23 | 0/8 | 0/11 | 0/9 | 0/19 | 0/95 | 0/22 | 23/207 | <0.001 |
| D29 MRD | 8/16 | 0/20 | 0/7 | 2/11 | 7/9 | 6/19 | 27/88 | 17/21 | 67/191 | <0.001 |
| Median WBC | 129.4 | 67.2 | 139.0 | 13.3 | 32.6 | 31.4 | 59.9 | 197.5 | 62.3 | <0.001 |
| RFS - 1 Yr ± SE | 75.0 ± 9.7 | 91.3 ± 5.9 | 87.5 ± 11.7 | 100 ± NA | 100 ± NA | 100 ± NA | 97.9 ± 1.5 | 90.7 ± 6.3 | 94.1 ± 1.7 | — |
| RFS - 2 Yrs ± SE | 65.0 ± 10.7 | 73.9 ± 9.2 | 87.5 ± 11.7 | 81.8 ± 11.6 | 100 ± NA | 100 ± NA | 83.0 ± 3.8 | 71.6 ± 9.8 | 81.7 ± 2.7 | — |
| RFS - 3 Yrs ± SE | 65.0 ± 10.7 | 73.9 ± 9.2 | 87.5 ± 11.7 | 72.7 ± 13.4 | 88.9 ± 10.5 | 94.1 ± 5.7 | 77.2 ± 4.4 | 52.5 ± 10.9 | 75.1 ± 3.0 | — |
| RFS - 4 Yrs ± SE | 65.0 ± 10.7 | 73.9 ± 9.2 | 75.0 ± 15.3 | 58.2 ± 16.9 | 88.9 ± 10.5 | 94.1 ± 5.7 | 67.4 ± 5.1 | 23.0 ± 10.3 | 66.3 ± 3.5 | — |
| RFS - 5 Yrs ± SE | 65.0 ± 10.7 | 73.9 ± 9.2 | 75.0 ± 15.3 | 58.2 ± 16.9 | 88.9 ± 10.5 | 94.1 ± 5.7 | 57.0 ± 6.5 | 0 ± NA | 61.9 ± 3.9 | — |
| Logrank p-value³ | 0.722 | 0.409 | 0.582 | 0.930 | 0.185 | 0.0184 | 0.993 | <0.001 | — | — |
| Hazard Ratio³ | 1.152 | 0.704 | 0.675 | 1.046 | 0.286 | 0.133 | 0.998 | 3.491 | | |

¹Abbreviations and Notations: MRD: Minimal Residual Disease; RFS: Relapse-Free Survival; MLL: the presence of MLL translocations; TCF3-PBX1: the presence of a t (1; 19)/TCF3-PBX1. Median WBC reported in 10³/μL.
²All P-values are calculated for Fisher's Exact Test (all variables except age and WBC) or Kruskal-Wallis Rank Sum Test (age and WBC) using R (version 2.9.1, survival and stats packages).
³Logrank p-values and hazard ratios calculated separately for each cluster using R (version 2.9.1, stats package)

Using probe sets selected by methods designed to find outliers (COPA and ROSE), nearly all of these same clusters were detected (FIGS. 17B and C; Tables 2′ and 3′). The sole exception to this is cluster 4, which was not evident using the COPA probe sets. The degree of the overlap across these three methods was also quite extensive (Table 4′ shows the cluster identity). HC and ROSE were the most similar (93.2% identical), however a pair-wise comparison revealed all to have nearly 90% common members. Even in the absence of cluster 4 in COPA clusters, the consensus overlap of all three methods was 86.5%. This is particularly noteworthy since only 37% of the clustering probe sets were shared by all three methods (Supplement, Table S7B′).

TABLE 2′
Association of Clinical and Outcome Features with COPA Gene Expression Cluster Groups¹

| | C1 | C2 | C3 | C5 | C6 | C7 | C8 | Total | P-Value² |
|---|---|---|---|---|---|---|---|---|---|
| # Cases/Cluster | 20 | 23 | 10 | 11 | 21 | 102 | 20 | 207 | — |
| Median Age (Yrs) | 6.9 | 13.1 | 15.2 | 14.7 | 14.5 | 11.7 | 14.3 | 13.1 | <0.001 |
| Sex (Male) | 11/20 | 11/23 | 5/10 | 8/11 | 17/21 | 71/102 | 14/20 | 137/207 | 0.196 |
| Ethnicity (Hispanic) | 3/20 | 6/23 | 2/10 | 0/10 | 3/20 | 25/102 | 12/20 | 51/205 | 0.008 |
| MLL | 20/20 | 0/23 | 0/10 | 0/11 | 0/21 | 1/102 | 0/20 | 21/207 | <0.001 |
| TCF3-PBX1 | 0/20 | 23/23 | 0/10 | 0/11 | 0/21 | 0/102 | 0/20 | 23/207 | <0.001 |
| D29 MRD | 9/17 | 0/20 | 1/9 | 8/11 | 6/21 | 26/94 | 17/19 | 67/191 | <0.001 |
| Median WBC | 129.4 | 67.2 | 33.5 | 32.6 | 26.0 | 52.5 | 158.3 | 623 | 0.028 |
| RFS - 1 Yr ± SE | 80.0 ± 8.9 | 91.3 ± 5.9 | 90.0 ± 9.5 | 100 ± NA | 100 ± NA | 97.1 ± 1.7 | 89.7 ± 6.9 | 94.1 ± 1.7 | — |
| RFS - 2 Yrs ± SE | 70.0 ± 10.3 | 73.9 ± 9.2 | 80.0 ± 12.7 | 100 ± NA | 100 ± NA | 84.1 ± 3.7 | 63.3 ± 11.0 | 81.7 ± 2.7 | — |
| RFS - 3 Yrs ± SE | 70.0 ± 10.3 | 73.9 ± 9.2 | 80.0 ± 12.7 | 90.0 ± 9.5 | 94.7 ± 5.1 | 77.0 ± 4.2 | 42.2 ± 11.3 | 75.1 ± 3.0 | — |
| RFS - 4 Yrs ± SE | 70.0 ± 10.3 | 73.9 ± 9.2 | 70.0 ± 14.5 | 78.7 ± 13.4 | 94.7 ± 5.1 | 66.4 ± 5.0 | 15.1 ± 9.3 | 66.3 ± 3.5 | — |
| RFS - 5 Yrs ± SE | 70.0 ± 10.3 | 73.9 ± 9.2 | 70.0 ± 14.5 | 78.7 ± 13.4 | 94.7 ± 5.1 | 56.1 ± 6.4 | 0.0 ± NA | 61.9 ± 3.9 | — |
| Logrank p-value³ | 0.808 | 0.409 | 0.788 | 0.364 | 0.010 | 0.944 | <0.001 | — | — |
| Hazard Ratio³ | 0.901 | 0.704 | 0.853 | 0.527 | 0.117 | 1.017 | 4.382 | | |

¹Abbreviations and Notations: MRD: Minimal Residual Disease; RFS: Relapse-Free Survival; MLL: the presence of MLL translocations; TCF3-PBX1: the presence of a t (1; 19)/TCF3-PBX1. Median WBC reported in 10³/μL.
²All P-values are calculated for Fisher's Exact Test (all variables except age and WBC) or Kruskal-Wallis Rank Sum Test (age and WBC) using R (version 2.9.0, survival and stats packages.
³Logrank p-values and hazard ratios calculated separately for each cluster using R (version 2.9.1, stats package)

TABLE 3′
Association of Clinical and Outcome Features with ROSE Gene Expression Cluster Groups

| | R1 | R2 | R3 | R4 | R5 | R6 | R7 | R8 | Total | P-Value² |
|---|---|---|---|---|---|---|---|---|---|---|
| # Cases/Cluster | 21 | 23 | 12 | 14 | 10 | 21 | 82 | 24 | 207 | — |
| Median Age (Yrs) | 4.7 | 13.1 | 15.2 | 14.3 | 14.5 | 14.5 | 7.8 | 14.1 | 13.1 | <0.001 |
| Sex (Male) | 11/21 | 11/23 | 6/12 | 13/14 | 8/10 | 17/21 | 54/82 | 17/24 | 137/207 | 0.043 |
| Ethnicity | 4/21 | 6/23 | 2/12 | 3/14 | 0/9 | 3/20 | 18/82 | 15/24 | 51/205 | 0.004 |
| (Hispanic) | | | | | | | | | | |
| MLL | 21/21 | 0/23 | 0/12 | 0/14 | 0/10 | 0/21 | 0/82 | 0/24 | 21/207 | <0.001 |
| TCF3-PBX1 | 0/21 | 23/23 | 0/12 | 0/14 | 0/10 | 0/21 | 0/82 | 0/24 | 23/207 | <0.001 |
| D29 MRD | 9/17 | 0/20 | 1/11 | 3/14 | 8/10 | 6/21 | 21/75 | 19/23 | 67/191 | <0.001 |
| Median WBC | 125.8 | 67.2 | 49.6 | 9.2 | 31.5 | 26.0 | 68.8 | 153.8 | 62.3 | <0.001 |
| RFS - 1 Yr ± SE | 76.2 ± 9.3 | 91.3 ± 5.9 | 90.9 ± 8.7 | 100 ± NA | 100 ± NA | 100 ± NA | 97.6 ± 1.7 | 91.5 ± 5.8 | 94.1 ± 1.7 | — |
| RFS - 2 Yrs ± SE | 66.7 ± 10.3 | 73.9 ± 9.2 | 81.8 ± 11.6 | 92.9 ± 6.9 | 100 ± NA | 100 ± NA | 82.6 ± 4.2 | 69.7 ± 9.6 | 81.7 ± 2.7 | — |
| RFS - 3 Yrs ± SE | 66.7 ± 10.3 | 73.9 ± 9.2 | 81.8 ± 11.6 | 85.7 ± 9.4 | 90.0 ± 9.5 | 94.7 ± 5.1 | 76.3 ± 4.8 | 47.9 ± 10.4 | 75.1 ± 3.0 | — |
| RFS - 4 Yrs ± SE | 66.7 ± 10.3 | 73.9 ± 9.2 | 72.7 ± 13.4 | 75.0 ± 12.9 | 78.7 ± 13.4 | 94.7 ± 5.1 | 66.2 ± 5.5 | 21.0 ± 9.5 | 66.3 ± 3.5 | — |
| RFS - 5 Yrs ± SE | 66.7 ± 10.3 | 73.9 ± 9.2 | 72.7 ± 13.4 | 75.0 ± 12.9 | 78.7 ± 13.4 | 94.7 ± 5.1 | 53.4 ± 7.4 | 0 ± NA | 61.9 ± 3.9 | — |
| Logrank p-value³ | 0.881 | 0.409 | 0.615 | 0.259 | 0.366 | 0.010 | 0.680 | <0.001 | — | — |
| Hazard Ratio³ | 1.060 | 0.704 | 0.744 | 0.520 | 0.528 | 0.117 | 1.110 | 3.878 | | |

¹Abbreviations and Notations: MRD: Minimal Residual Disease; RFS: Relapse-Free Survival; MLL: the presence of MLL translocations; TCF3-PBX1: the presence of a t (1; 19)/TCF3-PBX1. Median WBC reported in 10³/μL
²All P-values are calculated for Fisher's Exact Test (all variables except age and WBC) or Kruskal-Wallis Rank Sum Test (age and WBC) using R (version 2.9.1)
³Logrank p-values and hazard ratios calculated separately for each cluster using R (version 2.9.1, stats package

TABLE 4′
Comparison of Membership of P9906 Clusters

| | Cluster | | | | | | | | Overall |
|---|---|---|---|---|---|---|---|---|---|
| | 1 | 2 | 3 | 4 | 5 | 6 | 7 | 8 | Identity |
| HC v COPA | 19 | 23 | 8 | 0 | 9 | 19 | 88 | 19 | 89.4% |
| HC v ROSE | 20 | 23 | 8 | 10 | 9 | 19 | 82 | 22 | 93.2% |
| COPA v ROSE | 20 | 23 | 10 | 0 | 10 | 21 | 82 | 20 | 89.9% |
| HC v COPA v ROSE | 19 | 23 | 8 | 0 | 9 | 19 | 82 | 19 | 86.5% |

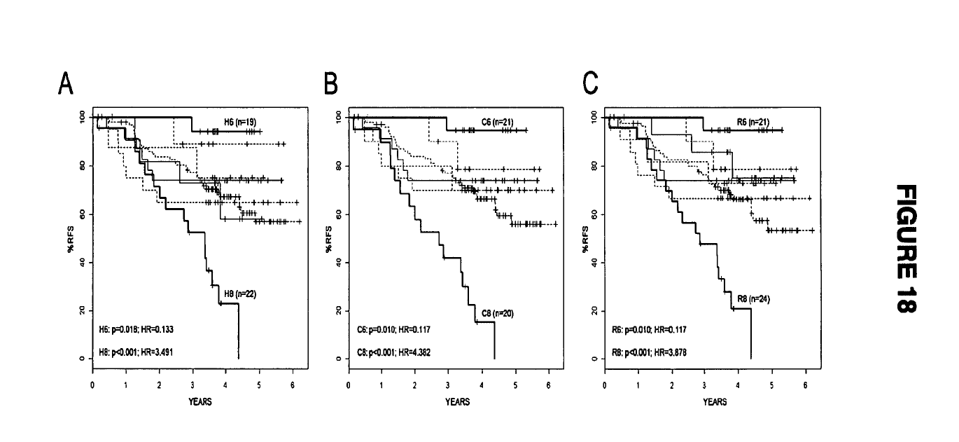

In addition to the significant association (p<0.001) between recurrent cytogenetic abnormalities and clusters 1 and 2, we observed significant associations between the clusters and several clinical features, including age (p<0.001-0.002), race (p=0.004-0.018), the presence of MRD at the end of induction therapy (p<0.001), and relapse free survival (RFS) (Tables 1′-3′, FIG. 18). Of particular note was the significant variation in RFS among the cluster groups (FIG. 18). Two of these (clusters 6 and 8) reached levels of statistical significance by independent logrank analysis in all three methods (cluster 6: p=0.010-0.018, HR=0.117-0.133; cluster 8: p<0.001, HR=3.491-4.382). While the overall 4-year RFS was 66.3±3.5%, cluster 6 ranged from 94.1±5.7 to 94.7±5.1%, with COPA and ROSE identifying the largest cluster (21 members) with the highest RFS. In contrast, the 4-year RFS for cluster 8 ranged from 15.1±9.3% for COPA to 23.0±10.3% for HC. Again, the ROSE cluster (R8) was the largest, with 24 members, and was intermediate in its RFS (21.0±9.5%). All 18 members of C8 were all contained within the R8 cluster.

The timing of relapse also differed between the cluster groups. While all relapses in clusters 1, 2 and 6 occurred within the first three years, patients in the remaining clusters, particularly in cluster 8, continued to experience relapses in years 3-5. Cluster 8 was also distinguished by a high frequency of MRD positivity at the end of induction therapy (81.0-89.5% of cases) and a preponderance of Hispanic/Latino ethnicity (59.1-62.5%) (Tables 1′-3′). Due to the extensive overlap of cluster membership, the larger size of the clusters, and the fact that R1 and R2 identified all MLL and TCF3-PBX1 samples, ROSE was selected as the reference clustering method.

Table 5′ lists the 113 probe sets that overlap between the ROSE clustering probe sets and those that were among the top 100 rank order for each cluster (Supplement, Sections 5 and 6). The majority of those associated with R1 (the cluster containing all the MLL translocated samples), including MEIS1, PROM1, RUNX2 and members of the HOX gene family, are consistent with previous reports describing the elevated expression of these genes in samples with underlying MLL translocations.^{21,22} We also found a number of other interesting outlier genes associated with MLL translocations, such as CTGF, which has previously been reported to be associated with a poor outcome in adult ALL²³; the correlation of CTGF expression and MLL translocations in that study was not reported. The outlier genes that distinguished cluster R2, containing all 23 cases with t(1;19)/TCF3-PBX1, included PBX1, which is directly involved in the underlying translocation. Surprisingly, while many of the probe sets associated with the other clusters formed very clear blocks of elevated expression (FIG. 17), they were neither comprised of any obvious pathways nor located within a particular chromosomal vicinity. These blocks of probe sets with very elevated expression, however, strongly suggest that a small subset might be used to distinguish the sample clusters.

Since several of the genes exhibiting outlier expression in clusters R1 and R2 are involved in or activated by their underlying cytogenetic abnormalities, this suggests that outlier genes associated with the other ROSE clusters might also be involved in, or perturbed by, a comparable genetic abnormality. Consistent with this hypothesis is the presence of notable outlier genes defining cluster R8 (including GAB1, MUC4, PON2, GPR110, SEMA6, SERPINB9; Supplement, Tables S15 S17′ and S18′) whose expression has been associated with t(9;22)/BCR-ABL1 and with overall outcome in ALL.^{5,21,24} Although patients in R8 were, by definition, all BCR-ABL1 negative, the strong similarity in expression patterns suggests a shared root pathway. Two recent reports of CRLF2 translocations and deletions in pediatric ALL also implicate this as a potential candidate for perturbation within cluster 8.^{25,26} While the elevated expression of CRLF2 is a feature of many R8 samples, however, it is not highly expressed in all. None of the other highly expressed genes associated with the other clusters has yet been shown to be directly involved in a translocation or activated by such an event.

TABLE 5′
ROSE Outlier Probe Sets/Genes Present in Top Rank Order of Clusters

| R1 | | R2 | | R3 | | R4 | |
|---|---|---|---|---|---|---|---|
| 220416_at | ATP8B4 | 227441_s_at | ANKS1B | 213808_at | ADAM23* | 203949_at | MPO |
| 219463_at | C20orf103 | 227440_at | ANKS1B | 203865_s_at | ADARB1 | 203948_s_at | MPO |
| 205899_at | CCNA1 | 227439_at | ANKS1B | 230128_at | IGL@ | 202273_at | PDGFRB |
| 209101_at | CTGF | 243533_x_at | ANKS1B* | 231513_at | KCNJ2* | 203476_at | TPBG |
| 218468_s_at | GREM1 | 234261_at | ANKS1B* | 203726_s_at | LAMA3 | | |
| 213150_at | HOXA10 | 202207_at | ARL4C | 232914_s_at | SYTL2 | | |
| 235521_at | HOXA3 | 202206_at | ARL4C | 225496_s_at | SYTL2 | | |
| 213844_at | HOXA5 | 212077_at | CALD1 | | | | |
| 214651_s_at | HOXA9 | 223786_at | CHST6 | | | | |
| 209905_at | HOXA9 | 205489_at | CRYM | | | | |
| 218847_at | IGF2BP2 | 206070_s_at | EPHAJ | | | | |
| 201105_at | LGALS1 | 201579_at | FAT1 | | | | |
| 1557534_at | LOC339862 | 231455_at | FLJ42418 | | | | |
| 202890_at | MAP7 | 239657_x_at | FOXO6 | | | | |
| 242172_at | MEIS1 | 235666_at | ITGA8? | | | | |
| 204069_at | MEIS1 | 235911_at | K03200* | | | | |
| 1559477_s_at | MEIS1 | 213005_s_at | KANK1 | | | | |
| 204304_s_at | PROM1 | 208567_s_at | KCNJ12 | | | | |
| 202976_s_at | RHOBTB3 | 210150_s_at | LAMA5 | | | | |
| 232231_at | RUNX2 | 228262_at | MAP7D2 | | | | |
| 226415_at | VATIL | 206028_s_at | MERTK | | | | |
| 231899_at | ZC3H12C | 204114_at | NID2 | | | | |
| | | 212151_at | PBX1 | | | | |
| | | 212148_at | PBX1 | | | | |
| | | 205253_at | PBX1 | | | | |
| | | 227949_at | PHACTR3 | | | | |
| | | 202178_at | PRKCZ | | | | |
| | | 242385_at | RORB | | | | |
| | | 231040_at | RORB? | | | | |
| | | 46665_at | SEMA4C | | | | |
| | | 206181_at | SLAMF1 | | | | |
| | | 225483_at | VPS26B | | | | |

| R5 | | R6 | | R7 | | R8 | |
|---|---|---|---|---|---|---|---|
| 212062_at | ATP9A | 242457_at | — | 219837_s_at | CYTL1 | 229975_at | BMPR1B |
| 228297_at | CNN3* | 241535_at | — | 212192_at | KCTD12 | 208303_s_at | CRLF2 |
| 209604_s_at | GATA3 | 204066_s_at | AGAP1 | | | 238689_at | GPR110 |
| 213362_at | PTPRD | 240758_at | AGAP1* | | | 235988_at | GPR110 |
| 229661_at | SALL4 | 233225_at | AGAP1* | | | 236489_at | GPR110? |
| 213258_at | TFPI | 219470_x_at | CCNJ | | | 207651_at | GPR171 |
| 210665_at | TFPI | 203921_at | CHST2 | | | 212592_at | IGJ |
| 210664_s_at | TFPI | 206756_at | CHST7 | | | 213371_at | LDB3 |
| | | 1552398_a_at | CLEC12A/B | | | 217110_s_at | MUC4 |
| | | 231166_at | GPR155 | | | 217109_at | MUC4 |
| | | 202409_at | IGF2 | | | 204895_x_at | MUC4 |
| | | 215177_s_at | ITGA6 | | | | |
| | | 201656_at | ITGA6 | | | | |
| | | 211340_s_at | MCAM | | | | |
| | | 210869_s_at | MCAM | | | | |
| | | 215692_s_at | MPPED2 | | | | |
| | | 205413_at | MPPED2 | | | | |
| | | 202336_s_at | PAM | | | | |
| | | 228863_at | PCDH17 | | | | |
| | | 227289_at | PCDH17 | | | | |
| | | 205656_at | PCDH17 | | | | |
| | | 230537_at | PCDH17? | | | | |
| | | 203335_at | PHYH | | | | |
| | | 203329_at | PTPRM | | | | |
| | | 1555579_s_at | PTPRM | | | | |
| | | 220059_at | STAP1 | | | | |
| | | 1554343_a_at | STAP1 | | | | |

Correlation of Genome-Wide Copy DNA Number Changes with ROSE Clusters

To gain insights into the genetic heterogeneity within higher risk B-precursor ALL and to identify underlying genetic lesions, particularly in the novel ROSE-defined cluster groups, we further correlated the gene expression profiles we had obtained with genome-wide DNA copy number abnormalities measured using SNP arrays, as previously described.⁶ The genome-wide copy number abnormalities in this higher-risk ALL cohort were recently reported,⁶ but herein we correlate these copy number abnormalities with the novel gene expression-based cluster groups that we have defined through ROSE outlier gene analysis (Table 6′; Supplement, Table S16′). As shown in Table 6′, while certain copy number abnormalities (such as those in seen in CDKN2A/B and PAX5) were found in several ROSE clusters, other abnormalities were more uniquely associated with each cluster group. As expected, 1 q gain and TCF3 loss were highly associated with the R2 cluster that contains TCF3-PBX1 cases, reflecting the unbalanced t(1;19) translocations that lead to duplication of chromosome 1 telomeric to PBX1 and deletion of chromosome 19 telomeric to TCF3. ERG deletions, as previously described by Mullighan, et al.²⁸, were seen almost exclusively (8 of 9) in R6. EBF1 deletions were seen only in R8, and a number of other DNA deletions were significantly associated with the R8 cluster, including IKZF1 (which was also deleted in 6 of 21 cases in the R6 cluster), RAG1-2, NUP160-PTPRJ, IL3RA-CSF2RA, C20orf94, and ADD3.

Correlation of Acquired Mutations with ROSE Clusters

A recent report on the significance of JAK1 and JAK2 mutations in higher-risk childhood precursor-B ALL included 198 of 207 patients studied here.⁷ We have correlated the JAK mutation status with ROSE clusters (Table 6′). Of the 198 patients for which sequencing was possible, 19 had mutations of either JAK1 (3) or JAK2 (16). There was a highly significant association of JAK1 and JAK2 mutations with R8, with all 19 of the mutations being either in R8 (n=12) or in the non-clustered group (n=7).

TABLE 6′
Correlation of Genome-Wide DNA Copy Number Abnormalities and
Acquired Mutations With ROSE Gene-Expression Cluster Groups¹

| | Rose Cluster Group | | | | | | | | |
|---|---|---|---|---|---|---|---|---|---|
| | R1 | R2 | R3 | R5 | R6 | R8 | R7 | P-Value | Comments |
| # Cases/ | 20 | 22 | 11 | 11 | 21 | 24 | 89 | | |
| Cluster | | | | | | | | | |
| DNA Copy | | | | | | | | | |
| Number | | | | | | | | | |
| Abnormality² | | | | | | | | | |
| 1q (gain) | 0 | 14 | 0 | 1 | 0 | 0 | 2 | <0.0001 | R2 has |
| | | | | | | | | | TCF3- |
| | | | | | | | | | PBX1 |
| EBF1 | 0 | 0 | 0 | 0 | 0 | 9 | 4 | <0.0001 | |
| IKZF1 | 1 | 0 | 0 | 2 | 6 | 20 | 26 | <0.0001 | |
| CDKN2A-B | 4 | 9 | 10 | 2 | 5 | 15 | 51 | <0.0001 | |
| TCF3 | 0 | 14 | 0 | 2 | 2 | 0 | 2 | <0.0001 | R2 has |
| | | | | | | | | | TCF3- |
| | | | | | | | | | PBX1 |
| ERG | 0 | 0 | 0 | 0 | 8 | 0 | 1 | <0.0001 | |
| VPREB1 | 0 | 0 | 0 | 1 | 8 | 14 | 28 | <0.0001 | |
| B cell | 5 | 17 | 5 | 4 | 12 | 23 | 66 | <0.0001 | |
| pathway** | | | | | | | | | |
| B cell | 5 | 17 | 5 | 5 | 14 | 24 | 68 | <0.0001 | |
| pathway | | | | | | | | | |
| including | | | | | | | | | |
| VPREB1** | | | | | | | | | |
| TBL1XR1 | 0 | 0 | 3 | 1 | 1 | 0 | 0 | 0.0002 | |
| PAX5 CNA | 1 | 9 | 4 | 0 | 3 | 7 | 39 | 0.0005 | |
| RAG1-2 | 1 | 0 | 1 | 0 | 0 | 5 | 0 | 0.0005 | |
| NUP160- | 0 | 0 | 0 | 0 | 0 | 4 | 0 | 0.0014 | |
| PTPRJ | | | | | | | | | |
| ETV6 | 1 | 0 | 3 | 4 | 1 | 0 | 15 | 0.0031 | |
| DMD | 0 | 5 | 1 | 2 | 3 | 0 | 3 | 0.0059 | |
| IL3RA- | 0 | 0 | 1 | 1 | 0 | 7 | 6 | 0.0061 | High |
| CSF2RA | | | | | | | | | CRLF2 |
| | | | | | | | | | expression |
| C20orf94 | 0 | 0 | 0 | 1 | 0 | 7 | 8 | 0.0073 | |
| ADD3 | 0 | 1 | 0 | 0 | 0 | 7 | 9 | 0.0144 | |
| NF1 | 1 | 1 | 0 | 2 | 0 | 1 | 0 | 0.0188 | |
| ARMC2- | 0 | 2 | 0 | 2 | 0 | 5 | 4 | 0.0291 | |
| SESN1 | | | | | | | | | |
| JAK1/2 | 0 | 0 | 0 | 0 | 0 | 1/11 | 2/5 | <0.0001 | |
| (mutation) | | | | | | | | | |

¹All p-values are derived from Fisher's Exact Test.
²All abnormalities are losses unless otherwise indicated

### Assessment of the Significance of ROSE Cluster Groups in a Second High Risk ALL Cohort

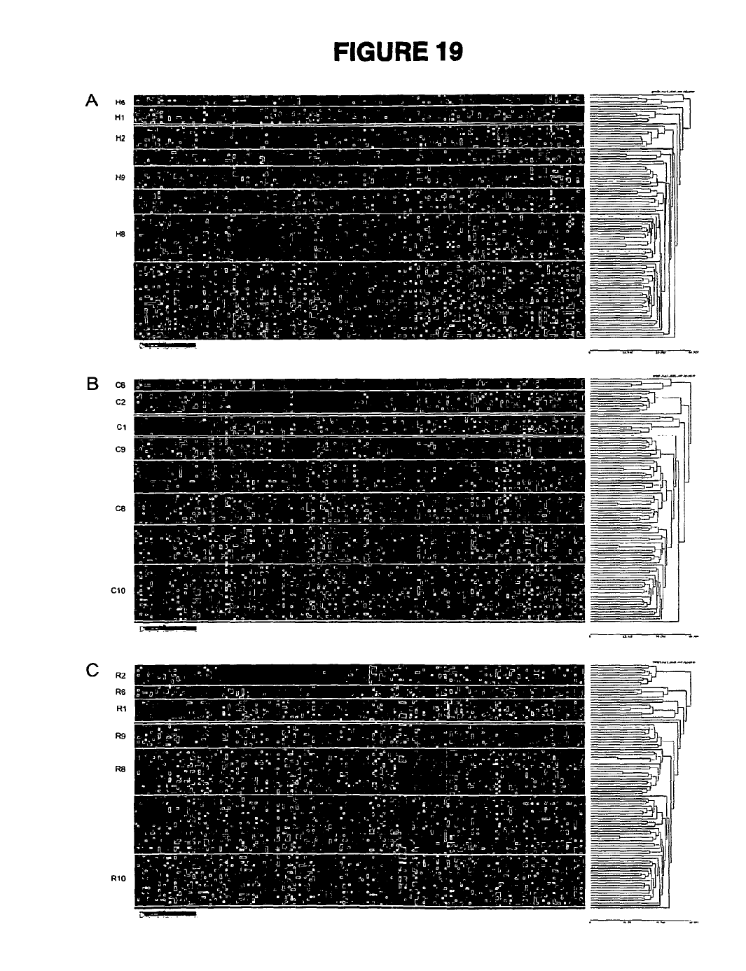

Given the striking genetic and clinical heterogeneity that we had found in the COG P9906 higher-risk ALL patients, we were interested in determining whether such distinct patient cluster groups could be found in other high risk ALL cohorts. We thus applied ROSE outlier methods to microarray data from an independent cohort of 99 children and adolescents with NCl/Rome who were treated on CCG Trial 1961.^{10,12} These 99 patients had been selected as a case:control cohort of high-risk ALL balanced for good vs. poor early marrow responses and for continuous complete remission vs. relapse; their gene expression profiles were also derived from the same platform used in this report. Although a smaller cohort than COG P9906, these 99 leukemias had a more diverse set of sentinel cytogenetic lesions, including patients with a t(12;21)/ETV6-AML1, BCR-ABL1, and favorable trisomies.¹² As shown in FIG. 19, all three methods identified the largest four clusters seen in P9906 (clusters 1, 2, 6 and 8). Due to the smaller size of the CCG 1961 study it is likely that the other three clusters seen in P9906 (clusters 3, 4 and 5) were not detected because of their low numbers. Two new clusters were also evident in the CCG 1961 analysis (clusters 9 and 10). Based upon the similarity of gene expression patterns, and limited clinical data, cluster 9 was determined to represent samples with t(12;21) ETV6-AML1 translocations. Cluster 10, however, did not share noticeable expression similarities to any previously identified cluster.

As was the case in P9906, clusters 1 and 2 contained all of the known MLL and TCF3-PBX1 translocated samples, respectively. The methods for selecting probe sets yielded more divergent lists (only 25.1% in common to all three methods; Supplement, Table S7B) than seen in P9906. This was primarily due to the difference between those identified by HC and those found by the two outlier methods. ROSE and COPA shared 130 (77.8%) of the probe sets used for clustering in CCG 1961, while HC had only 32.9% in common with COPA and 27.5% in common with ROSE. There were also relatively few probe sets in common with the P9906 clustering (Supplement, Table S7C′). In large part this is likely due to the different composition of the CCG 1961 cohort (e.g., inclusion of BCR-ABL1 and ETV6-AML1 translocations).

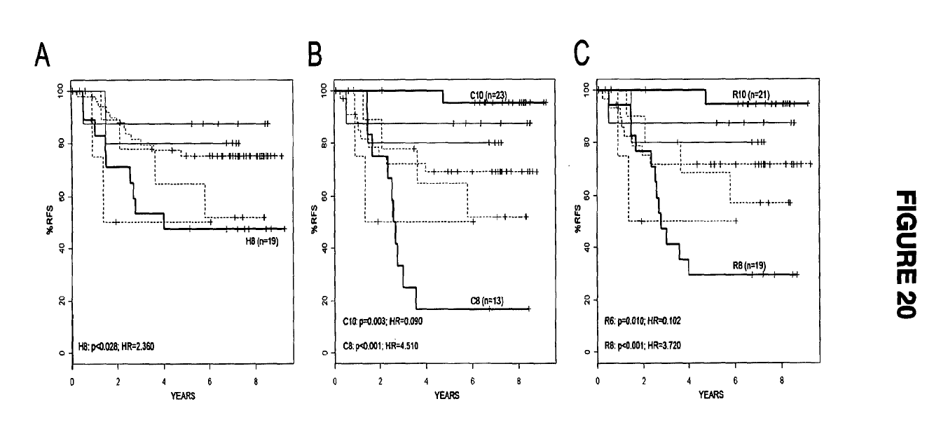

FIG. 20 depicts the survival curves for the CCG 1961 clusters. Too few samples were present in cluster 6 (only 5 patients, one of whom relapsed) to make any statistical inferences about RFS. Cluster 8, however, reached levels of significance in all three methods (p<0.001-0.028) and had very poor RFS (HR=2.36-4.51). All 13 C8 members were contained within the 19 R8. Interestingly, of the 6 BCR-ABL1 positive samples in CCG 1961, only one was in C8 and four in R8. Although H8 contained 5 of the 6 BCR-ABL1 positive samples, its RFS was the most favorable of the three cluster 8 groups. Overall, these results confirm the robust nature of the outlier clustering methods, the genetic and clinical heterogeneity within high risk ALL, and the very poor outcome consistently associated with cluster 8 gene expression profiles.

### Discussion

Using unsupervised methods to analyze gene expression profiles, we have identified multiple gene expression-based cluster groups among children and adolescents with ALL who are classified using today's risk classification schemes as higher risk. These novel cluster groups were distinguished by high levels of expression of unique sets of “outlier” genes, distinct DNA copy number abnormalities, variable clinical features, and significantly different rates of relapse-free survival. These studies reveal the striking biologic, genetic, and clinical heterogeneity within ALL currently categorized as higher risk and point to novel genes that may serve as new targets for improved diagnosis, risk classification, and therapy.

Particularly striking among the gene expression-based clusters were two groups of patients found by all methods (clusters 6 and 8) that had strikingly different rates of RFS, despite being classified as higher risk at initial diagnosis. In contrast to the overall cohort with an RFS of 66.3±% 3.5% at 4 years, patients in cluster 6 had significantly superior 4-year relapse-free survivals of (94.1±5.7−94.7±5.1%; p=0.010-0.018); HR=0.117-0.133). The representative ROSE cluster (R6) was characterized by high expression of several unique “outlier” genes (AGAP1, CCNJ, CHST2/7, CLEC12A/B, and PTPRM) and by relatively frequent ERG deletions. This cluster group appears highly similar in its gene expression pattern and intragenic ERG deletions to a “novel” cluster of ALL patients originally identified by Yeoh et al.²⁸ and Ross et al.²¹ and further characterized by Mullighan et al.²⁷ Unlike these earlier studies, however, in P9906 we find a strong correlation of this cluster with a very favorable outcome.

In contrast to the superior relapse-free survival seen in some of the novel gene expression cluster groups, the ALL patients initially categorized as higher risk who were in cluster 8 had an extremely poor survival (15.1±9.3−23.0±10.3%; p<0.001; HR=3.491−4.382). A particularly interesting finding in our study was the statistically significant association between cluster 8 and self-reported Hispanic/Latino ethnicity; within H8, C8 and R8 this association was highly significant (p<0.001). Unfortunately, ethnic data were not available for CCG 1961 so this finding could not be validated in our validation cohort. Hispanic and American Indian children with ALL have previously been reported to have poorer outcomes than non-Hispanic white children when treated with conventional ALL therapy.^{29,30} Interestingly, our most recent studies correlating ALL outcomes with racial ancestry determined by genome-wide single nucleotide polymorphism markers, rather than self-reported race, in large cohorts of children treated at St. Jude Children's Research Hospital and the Children's Oncology Group have found that Hispanic and American Indian ancestry are associated with a significantly increased risk of relapse independent of other known prognostic factors (J. Yang, M. Relling, et al., submitted). Whether these outcome differences result from differences in disease biology, pharmacogenetic differences in host response to therapy, or social and cultural factors remains to be determined. Whether children of different ethnic groups are uniquely susceptible to the acquisition of different genetic abnormalities that predispose to the development of ALL is also an important area for future investigation.

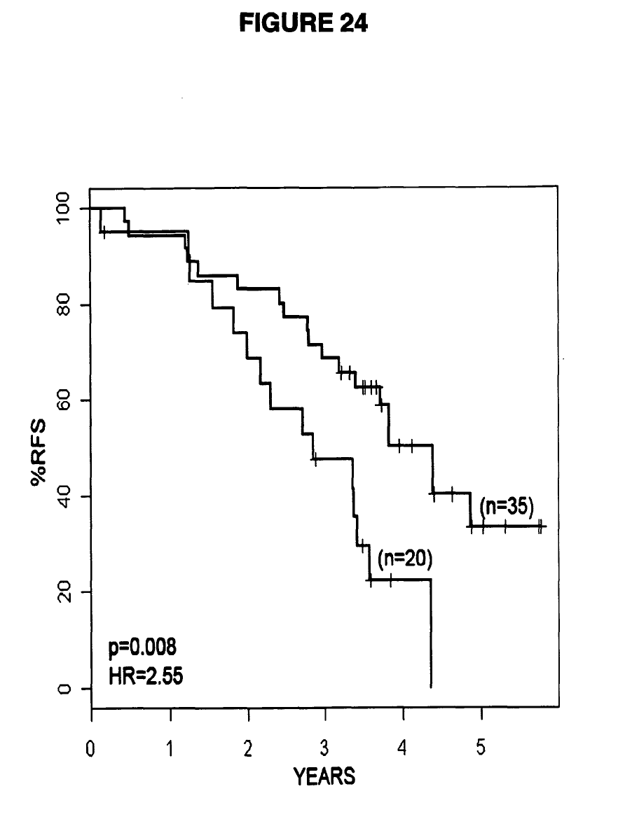

Cluster 8 patients were also distinguished by the expression of a highly unique and interesting set of “outlier” genes, including BMPR1B, CRLF2, GPR110, GPR171, IGJ, LDB3, and MUCO (Table 5′). Our studies of whole-genome DNA copy number abnormalities have also found deletions in several genes and chromosomal regions that are highly associated with this cluster group: EBF1, NUP160-PTPRJ, IL3RA-CSF2RA, C20orf94, and ADD3 (Table 6′). Deletions of IKZFland VPREB1 were also very frequent in the R8 cluster, occurring in 20/24 and 14/24 R8 cases respectively, and have been associated with a poorer outcome in ALL.^{5,31} The IKZF1 status of most of these current cases (197/207) have been previously reported (10/207 did not have DNA available for testing).⁵ Deletions in these genes were also prevalent in the R6 cluster (IKZF1 6/21 cases, VPREB1 8/21 cases) which was associated with a superior outcome (Table 6′). Although IKZF1 alterations are generally associated with poor outcome, only one of the six R6 cases with an IZKF1 lesion relapsed. The survival of IKZF1 patients in R8 was also significantly worse than IKZF1 patients overall (FIG. 24; p=0.008; HR=2.55). Thus, overall outcome is likely to reflect a constellation of genetic abnormalities within a specific patient cluster group rather than on a single genetic lesion. In this regard, assays that measure the expression of R8 cluster-specific genes or gene expression-based classifiers that are predictive of outcome (Kang et al, Blood 2009) may be useful in the clinical setting for the prospective identification of patients at very high risk of treatment failure. It is likely that the elevated expression of some of the cluster 8 genes, while not necessarily sufficient to result in their clustering together, will be useful in predicting RFS. Clustering, as performed here, is more of a discovery tool to identify related prognostic factors instead of a diagnostic tool on its own. While 24/207 (11.6%) of P9906 clusters in R8, the expression of some of these cluster 8 genes is shared among other members and will likely be useful in stratifying their risk.

The presence of CRLF2 as an outlier gene³² combined with the DNA deletions that we have found in the pseudo-autosomal region of Xp and Yp adjacent to the CRLF2 locus (IL3RA-CSF2RA) in cluster R8 are particularly intriguing in light of a report correlating CRLF2 overexpression with either IGH@-CRLF2 translocations or with interstitial deletions adjacent to CRLF2 and involving CSF2RA and IL3RA.^{33,34} We are currently examining CRLF2 alterations in our cases with elevated expression and IL3RA-CSF2RA deletions to determine if similar events exist in P9906. Another distinguishing feature of cluster 8, which lacked t(9;22)/BCR-ABL1 translocations, was elevated expression of several genes such as GAB1 that have been shown to be predictive of outcome and imatinib response in BCR-ABL1 ALL.³⁵ We have also found that ALL cases containing IKZF1 deletions, such as those in the cluster 8, frequently have an “activated tyrosine kinase” gene expression signature despite the lack of BCR-ABL1 translocations.⁵ Den Boer and colleagues have also recently reported the existence of a subset of ALL cases with a “BCR-ABL-like” gene expression signature and a relatively poor outcome.³¹ Despite these related signatures, as was shown with CCG 1961 cases, when BCR-ABL1 samples are clustered together with other high-risk samples using outlier genes, they do not necessarily segregate to cluster 8.

As part of a comprehensive approach to the genetic analysis of high-risk B-precursor ALL, we have undertaken a focused targeted gene sequencing effort of the COG P9906 cohort under the auspices of a National Cancer Institute TARGET Initiative (www.target.cancer.gov). Through this effort, we discovered mutations in two members of the JAK family of tyrosine kinases (JAK1 and JAK2) in 12/24 R8 cluster members and 7 patients that did not cluster (R7).⁶ Of these 12 JAK mutant R8 cases, 9 also had IKZF1 deletions (while 11/12 without JAK mutations had IKZF1 lesions). It is likely that other unidentified mutations are responsible for the “activated kinase” gene expression signature in the R8 cases without JAK mutations, and we are currently performing a range of complementary genomic analysis, including sequencing of the tyrosine kinome, in search of them.

The identification of cluster 8 illustrates the power of applying complementary molecular biology tools to clinically annotated leukemia specimens such as those from the COG P9906 cohort. Analysis for DNA copy number alterations and DNA sequencing defines the genomic basis for these cases, while GEP with unsupervised analysis provides an integrated picture of the overall effect of the complex genomic, and as yet undefined epigenomic, alterations that these leukemia cells possess. Future studies will address how the complex constellation of characteristics in cluster 8, including outlier gene expression signature, DNA deletions, and mutations in genes such as JAK, interact to produce such poor outcome relative to the other cluster groups. These future studies will provide the understanding needed to determine which of these molecular characteristics are best suited for clinical application in terms of prospectively identifying this patient cohort that is at high risk for treatment failure and in terms of developing new treatments that effectively address the aggressive leukemia phenotype of the cluster 8 patients.

### 2″ Supplement-Identification of Novel Cluster Groups in Pediatric Higher Risk B-Precursor Acute Lymphoblastic Leukemia by Unsupervised Gene Expression Profiling

### Patients and Clinical Risk Factors

For this study, pre-treatment cryopreserved leukemia specimens were available on a representative cohort of 207 of the 272 (76%) patients registered to COG P9906; the clinical and outcome parameters of these 207 patients did not differ significantly from all 272 patients (see Table S1′ and FIG. 21/S1′). As shown in Table S1′ and FIG. 21/S1′, the differences in various characteristics between the entire group (n=272) and the present study cohort (n=207) were examined by the statistical comparisons between the present study cohort and remaining patients (n=65) not included in the present study. Each P-value in Table S1 and Figure S1′ is that of the individual test which needs to be adjusted for multiple testing. A simple Bonferroni adjustment multiplies the P-values by the total number of tests (10). After this adjustment, none of the characteristics are significantly different between the entire group and the cohort examined herein, except the test for WBC count when a cutoff value was considered.

TABLE S1′
Comparison of HR-ALL Patients Registered to COG P9906
(n = 272) and The Subset of Patients Examined and
Modeled for Gene Expression Signatures (n = 207)¹

| | Not | | | | | | p-value |
|---|---|---|---|---|---|---|---|
| Char- | Studied | | Studied | | Total | | (Fisher's |
| acteristics | N | % | N | % | N | % | exact test) |
| Age - no. | | | | | | | |
| ≧10 Yrs | 51 | 78.46 | 132 | 63.77 | 183 | 67.28 | 0.0335 |
| <10 Yrs | 14 | 21.54 | 75 | 26.23 | 89 | 32.72 | |
| Sex - no. | | | | | | | |
| Male | 52 | 80 | 137 | 66.18 | 189 | 69.49 | 0.0442 |
| Female | 13 | 20 | 70 | 33.82 | 83 | 30.51 | |
| WBC - no. | | | | | | | |
| <50K/μL | 52 | 80 | 99 | 47.83 | 151 | 55.51 | <0.0001 |
| ≧50K/μL | 13 | 20 | 108 | 52.17 | 121 | 44.49 | |
| Race | | | | | | | |
| Hispanic | 15 | 23.08 | 51 | 24.64 | 66 | 24.26 | 0.9638 |
| or Latino | | | | | | | |
| Others | 47 | 72.31 | 154 | 74.39 | 201 | 73.90 | |
| Unknown | 3 | 4.61 | 2 | 0.97 | 5 | 1.84 | |
| MRD | | | | | | | |
| at day 29 | | | | | | | |
| Negative | 40 | 61.54 | 124 | 59.90 | 164 | 60.29 | 0.7550 |
| Positive | 19 | 29.23 | 67 | 32.37 | 86 | 31.62 | |
| Unknown | 6 | 9.23 | 16 | 7.73 | 22 | 8.09 | |
| MLL | | | | | | | |
| Negative | 61 | 93.85 | 186 | 89.86 | 247 | 90.81 | 0.4617 |
| Positive | 4 | 6.15 | 21 | 10.15 | 25 | 9.19 | |
| TCF3/PBX1 | | | | | | | |
| Negative | 59 | 90.77 | 184 | 88.89 | 243 | 89.34 | 0.6384 |
| Positive | 5 | 7.69 | 23 | 11.11 | 28 | 10.29 | |
| Unknown | 1 | 1.54 | 0 | 0 | 1 | 0.37 | |
| CNS | | | | | | | |
| No blasts | 54 | 83.08 | 160 | 77.29 | 214 | 78.68 | 0.1009 |
| <5 blasts | 3 | 4.61 | 26 | 12.56 | 29 | 10.66 | |
| ≧5 blasts | 8 | 12.31 | 21 | 10.15 | 29 | 10.66 | |
| Total | 65 | 100 | 207 | 100 | 272 | 100 | |

¹All unknown data were removed before statistical tests were performed.

The 207 patient cohort had slight male predominance (66%) and included a subset (23%, 47/201) with blasts in the CNS at diagnosis (CNS2+CNS3). Approximately 35% of the 191 specimens evaluated by flow cytometry on day 29 of induction therapy had subclinical MRD (>0.01% blasts).¹ As shown in Table S2, only MRD at the end of induction therapy and increasing WBC count were significantly associated with decreased relapse free survival (RFS). The significant effect of WBC count as a continuous variable on decreased RFS was no longer seen when the cutoff of 50 K/μL was applied (see Section 7). A trend towards declining RFS was also observed among the 25% of children with Hispanic/Latino ethnicity contained within this cohort. In multivariate analysis, both MRD and WBC count retained significance when adjusted for one another (likelihood ratio test based on COX regression, P-value <0.001).

TABLE S2′
Association of Relapse Free Survival with Clinical
and Genetic Features in the High Risk ALL Cohort

Association with Relapse
Free Survival

| | | Hazard | |
|---|---|---|---|
| Characteristic | | Ratio | p-value |
| Age | | | |
| ≧10 Yrs | 132 | 1 | |
| <10 Yrs | 75 | 1.152 | 0.561 |
| Age | | | |
| Median | 13.5 yrs | | |
| Range | 1-20 | .995 | 0.817 |
| Sex | | | |
| Male | 137 | 1 | |
| Female | 70 | 0.769 | 0.320 |
| WBC | | | |
| Median | 62.3 K/μL | | |
| Range | 1-959 | 1.003 | <0.001 |
| MRD at Day 29 | | | |
| Negative | 124 | 1 | |
| Positive | 67 | 2.805 | <0.001 |
| Race | | | |
| Hispanic | 51 | 1.644 | 0.049 |
| or Latino | | | |
| Others | 154 | 1 | |
| MLL | | | |
| Positive | 21 | 1.061 | 0.881 |
| Negative | 186 | 1 | |
| TCF3/PBX1 | | | |
| Positive | 23 | .704 | 0.409 |
| Negative | 184 | 1 | |
| CNS | | | |
| No blasts | 160 | 1 | |
| <5 blasts | 26 | 0.897 | 0.708 |
| ≧5 blasts | 21 | | |

### Validation Cohort

A subset of patients from COG CCG 1961 “Treatment of Patients with Acute Lymphoblastic Leukemia with Unfavorable Features” was used as a validation cohort to determine whether similar clusters were present in a different set of high-risk patients. As described in Bhojwani et al.,² COG CCG 1961 enrolled a total of 2078 patients with NCI high risk features, i.e. WBC count ≧50,000/μL or age ≧10 years old, from September 1996 to May 2002. Microarray data from these 99 patients were analyzed using the methods described in this paper.

### 3. Data Processing

### A. Microarray Preparation and Scanning

After RNA quantification, cDNA preparation, and labeling, biotinylated cRNA was fragmented and hybridized to HG_U133_Plus2.0 oligonucleotide microarrays (Affymetrix, Santa Clara, Calif.) containing 54,675 probe sets. Signals were scanned (Affymetrix GeneChip Scanner) and analyzed with the Affymetrix Microarray Suite (MAS 5.0). Signal intensities and expression data were generated with the Affymetrix GCOS1.4 software package.

### B. Microarray Data Masking

Prior to any intensity analysis, the microarray data were first masked to remove those probes found to be uninformative in a majority of the samples. Removal of these probe pairs improves the overall quality of the data and eliminates many non-specific signals that are shared by a particular sample type (i.e., cross-hybridizing messages present in blood and marrow samples). Each probe pair (across all 207 samples) was evaluated and masked if the mismatch (MM) was greater than the perfect match (PM) in more than 60% of the samples. This mask removed 94,767 probe pairs (15.7% of the 604,258) and had some impact on 38,588 probe sets (71%). As shown in Table S3, the net impact of masking was a significant increase in the number of present calls coupled with a dramatic decrease in the number of absent calls. The mask removed only seven probe sets (0.01% of the 54,675), all of which represented non-human control genes.

TABLE S3′
Impact of Masking on Affymetrix Statistical Calls (Reported
as Percentage of Total Probes: 54,675 raw; 54,668 masked).

| | Present | Marginal | Absent | No call |
|---|---|---|---|---|
| Raw | 34.9 | 1.7 | 63.3 | 0 |
| Masked | 48.0 | 3.1 | 48.9 | 0 (7) |

### C. Microarray Data Filtering

Prior to any clustering, the data were filtered to remove probe sets deemed to be unrelated to disease: genes from sex-determining regions of X and Y (which simply correlate with sex), spiked control genes and globin genes (presumed to arise from contaminating normal blood cells). All filtered probe sets were selected based upon their gene symbols or chromosomal location. Table S4 lists the 89 probe sets mapped within sex-determining regions. These include the XIST gene from chromosome X and probe sets from Yp11-Yq11. All probe sets from PAR1 and PAR2 regions of both sex chromosomes are retained. Table S5 lists the 62 Affymetrix spiked control genes. Table S6 lists the twenty excluded globin probe sets with a gene symbol beginning with “HB” and the word “globin” contained within the gene title. After the filtering of these probe sets 54,504 were available for clustering.

TABLE S4′
X- and Y- Specific Transcripts Excluded from the Analysis (89)

| Probe Set ID | Gene Symbol | Cytoband |
|---|---|---|
| 214218_s_at | XIST | Xq13.2 |
| 221728_x_at | XIST | Xq13.2 |
| 224588_at | XIST | Xq13.2 |
| 224589_at | XIST | Xq13.2 |
| 224590_at | XIST | Xq13.2 |
| 227671_at | XIST | Xq13.2 |
| 243712_at | XIST | Xq13.2 |
| 201909_at | LOC100133662 /// RPS4Y1 | Yp11.3 |
| 204409_s_at | EIF1AY | Yq11.222 |
| 204410_at | EIF1AY | Yq11.222 |
| 205000_at | DDX3Y | Yq11 |
| 205001_s_at | DDX3Y /// LOC100130220 | Yq11 |
| 206279_at | PRKY | Yp11.2 |
| 206624_at | LOC100130216 /// USP9Y | Yq11.2 |
| 206700_s_at | JARID1D | Yq11\|Yq11 |
| 206769_at | LOC100130227 /// TMSB4Y | Yq11.221 |
| 207063_at | CYorf14 | Yq11.222 |
| 207246_at | LOC100130829 /// ZFY | Yp11.3 |
| 207646_s_at | CDY1 /// CDY1B /// CDY2A /// | Yq11.221 /// |
| | CDY2B | Yq11.223 /// |
| | | Yq11.23 |
| 207647_at | CDY1 | Yq11.23 |
| 207703_at | NLGN4Y | Yq11.221 |
| 207893_at | LOC100130809 /// SRY | Yp11.3 |
| 207909_x_at | DAZ1 /// DAZ2 /// DAZ3 /// | Yq11.223 |
| | DAZ4 /// LOC732447 | |
| 207912_s_at | DAZ1 /// DAZ2 /// DAZ3 /// | Yq11.223 |
| | DAZ4 /// LOC732447 | |
| 207916_at | RBMY1E | Yq11.223 |
| 207918_s_at | LOC728137 /// LOC728395 /// | Yp11.2 |
| | LOC728412 /// TSPY1 | |
| 208067_x_at | LOC100130224 /// UTY | Yq11 |
| 208220_x_at | AMELY | Yp11.2 |
| 208281_x_at | DAZ1 /// DAZ2 /// DAZ3 /// | Yq11.223 |
| | DAZ4 /// LOC732447 | |
| 208282_x_at | DAZ1 /// DAZ2 /// DAZ3 /// | Yq11.223 |
| | DAZ4 /// LOC732447 | |
| 208307_at | RBMY1A1 /// RBMY1B /// | Yp11.2 /// |
| | RBMY1D /// RBMY1E /// | Yq11.223 |
| | RBMY1F /// RBMY1J /// | |
| | RBMY3AP | |
| 208331_at | BPY2 | Yq11 |
| 208332_at | PRY /// PRY2 | Yq11.223 |
| 208339_at | XKRY /// XKRY2 | Yq11.221 |
| 210322_x_at | UTY | Yq11 |
| 211149_at | LOC100130224 /// UTY | Yq11 |
| 211227_s_at | PCDH11Y | Yp11.2 |
| 211460_at | TTTY9A /// TTTY9B | Yq11.221 /// |
| | | Yq11.222 |
| 211461_at | CSPG4LYP1 /// CSPG4LYP2 | Yq11.223 /// |
| | | Yq11.23 |
| 211462_s_at | TBL1Y | Yp11.2 |
| 214131_at | CYorf15B | Yq11.222 |
| 214983_at | TTTY15 | Yq11.1 |
| 216351_x_at | DAZ1 /// DAZ2 /// DAZ3 /// | Yq11.223 |
| | DAZ4 /// LOC732447 | |
| 216374_at | LOC728137 /// LOC728395 /// | Yp11.2 |
| | LOC728412 /// TSPY1 | |
| 216544_at | RBMY2FP | Yq11.223 |
| 216665_s_at | TTTY2 | Yp11.2 |
| 216673_at | LOC100101116 /// TTTY1 | Yp11.2 |
| 216786_at | LOC159110 | Yq11.221 |
| 216842_x_at | RBM /// RBMY1A1 /// RBMY1B /// | Yp11.2 /// |
| | RBMY1D /// RBMY1E /// RBMY1F /// | Yq11.223 /// |
| | RBMY1H /// RBMY1J /// RBMY3AP | Yq11.23 |
| 216922_x_at | DAZ1 /// DAZ2 /// DAZ3 /// | Yq11.223 |
| | DAZ4 /// LOC732447 | |
| 217049_x_at | PCDH11Y | Yp11.2 |
| 217160_at | TSPY1 | Yp11.2 |
| 217261_at | LOC100101117 /// TTTY2 | Yp11.2 |
| 222229_x_at | LOC441533 | Yp11.2 |
| 223645_s_at | CYorf15B | Yq11.222 |
| 223646_s_at | CYorf15B | Yq11.222 |
| 224003_at | TTTY14 | Yq11.222 |
| 224007_at | HSFY1 /// HSFY2 | Yq11.222 |
| 224040_at | TTTY5 | Yq11.223 |
| 224041_at | TTTY6 | Yq11.223 |
| 224052_at | HSFY1 /// HSFY2 | Yq11.222 |
| 224142_s_at | LOC100101118 /// TTTY8 | Yp11.2 |
| 224143_at | LOC100101118 /// TTTY8 | Yp11.2 |
| 224174_at | TTTY11 | Yp11.2 |
| 224195_at | TTTY12 | Yp11.2 |
| 224292_at | TTTY13 | Yq11.223 |
| 224293_at | TTTY10 | Yq11.221 |
| 228492_at | LOC100130216 /// USP9Y | Yq11.2 |
| 230760_at | LOC100130829 /// ZFY | Yp11.3 |
| 232618_at | CYorf15A | Yq11.222 |
| 233151_s_at | TTTY7 | Yp11.2 |
| 233178_at | TGIF2LY | Yp11.2 |
| 234309_at | TTTY7 | Yp11.2 |
| 234715_at | GOLGA2LY1 /// GOLGA2LY2 | Yq11.223 |
| 234913_at | TTTY4 /// TTTY4B /// TTTY4C | Yq11.2 /// |
| | | Yq11.223 |
| 234931_at | AYP1p1 | Yp11.31 |
| 235941_s_at | LOC159110 /// LOC401629 /// | Yq11.221 |
| | LOC401630 | |
| 235942_at | LOC401629 /// LOC401630 | Yq11.221 |
| 236694_at | CYorf15A | Yq11.222 |
| 1552952_at | RBMY2FP | Yq11.223 |
| 1554125_a_at | NLGN4Y | Yq11.221 |
| 1561185_at | TTTY7 | Yp11.2 |
| 1561390_at | FAM41AY | Yq11.221 |
| 1562313_at | BCORL2 | Yq11.222 |
| 1563420_at | XGPY2 | Yp11.31 |
| 1565132_at | RBMY3AP | Yp11.2 |
| 1565320_at | RBMY3AP | Yp11.2 |
| 1570359_at | DDX3Y | Yq11 |
| 1570360_s_at | DDX3Y /// LOC100130220 | Yq11 |

TABLE S5′
AFFX Probe Sets Excluded from the Analysis (62)
Probe Set ID

AFFX-BioB-5_at
AFFX-BioB-M_at
AFFX-BioB-3_at
AFFX-BioC-5_at
AFFX-BioC-3_at
AFFX-BioDn-5_at
AFFX-BioDn-3_at
AFFX-CreX-5_at
AFFX-CreX-3_at
AFFX-DapX-5_at
AFFX-DapX-M_at
AFFX-DapX-3_at
AFFX-LysX-5_at
AFFX-LysX-M_at
AFFX-LysX-3_at
AFFX-PheX-5_at
AFFX-PheX-M_at
AFFX-PheX-3_at
AFFX-ThrX-5_at
AFFX-ThrX-M_at
AFFX-ThrX-3_at
AFFX-TrpnX-5_at
AFFX-TrpnX-M_at
AFFX-TrpnX-3_at
AFFX-r2-Ec-bioB-5_at
AFFX-r2-Ec-bioB-M_at
AFFX-r2-Ec-bioB-3_at
AFFX-r2-Ec-bioC-5_at
AFFX-r2-Ec-bioC-3_at
AFFX-r2-Ec-bioD-5_at
AFFX-r2-Ec-bioD-3_at
AFFX-r2-P1-cre-5_at
AFFX-r2-P1-cre-3_at
AFFX-r2-Bs-dap-5_at
AFFX-r2-Bs-dap-M_at
AFFX-r2-Bs-dap-3_at
AFFX-r2-Bs-lys-5_at
AFFX-r2-Bs-lys-M_at
AFFX-r2-Bs-lys-3_at
AFFX-r2-Bs-phe-5_at
AFFX-r2-Bs-phe-M_at
AFFX-r2-Bs-phe-3_at
AFFX-r2-Bs-thr-3_s_at
AFFX-r2-Bs-thr-M_s_at
AFFX-r2-Bs-thr-5_s_at
AFFX-HUMISGF3A/M97935_5_at
AFFX-HUMISGF3A/M97935_MA_at
AFFX-HUMISGF3A/M97935_MB_at
AFFX-HUMISGF3A/M97935_3_at
AFFX-HUMRGE/M10098_5_at
AFFX-HUMRGE/M10098_M_at
AFFX-HUMRGE/M10098_3_at
AFFX-HUMGAPDH/M33197_5_at
AFFX-HUMGAPDH/M33197_M_at
AFFX-HUMGAPDH/M33197_3_at
AFFX-HSAC07/X00351_5_at
AFFX-HSAC07/X00351_M_at
AFFX-HSAC07/X00351_3_at
AFFX-M27830_5_at
AFFX-M27830_M_at
AFFX-M27830_3_at
AFFX-hum_alu_at

TABLE S6′
Globin Probe Sets Excluded from the Analysis (20)

| Probe Set ID | Gene Symbol | Cytoband |
|---|---|---|
| 1562981_at | HBB | 11p15.5 |
| 204018_x_at | HBA1 /// HBA2 | 16p13.3 |
| 204419_x_at | HBG1 /// HBG2 | 11p15.5 |
| 204848_x_at | HBG1 /// HBG2 | 11p15.5 |
| 205919_at | HBE1 | 11p15.5 |
| 206647_at | HBZ | 16p13.3 |
| 206834_at | HBD | 11p15.5 |
| 209116_x_at | HBB | 11p15.5 |
| 209458_x_at | HBA1 /// HBA2 | 16p13.3 |
| 211696_x_at | HBB | 11p15.5 |
| 211699_x_at | HBA1 /// HBA2 | 16p13.3 |
| 211745_x_at | HBA1 /// HBA2 | 16p13.3 |
| 213515_x_at | HBG1 /// HBG2 | 11p15.5 |
| 214414_x_at | HBA1 /// HBA2 | 16p13.3 |
| 216036_at | HBBP1 | 11p15.5 |
| 217232_x_at | HBB | 11p15.5 |
| 217414_x_at | HBA1 /// HBA2 | 16p13.3 |
| 217683_at | HBE1 | 11p15.5 |
| 220807_at | HBQ1 | 16p13.3 |
| 240336_at | HBM | 16p13.3 |

### 4. Selection of Clustering Probe Sets: High CV, ROSE and COPA

### A. Selection of High CV Probe Sets

Each of the remaining 54,504 filtered probe sets was ordered by its coefficient of variation (CV=standard devation/mean). The 254 probe sets with the highest CVs were used for the H clustering.

### B. Selection of COPA Probe Sets

The COPA method was applied essentially as described by Tomlins et a1.5 First, the median expression for each probe set was adjusted to zero. Secondly, the median absolute deviation from median (MAD) was calculated and the intensities for each probe set were divided by its MAD. Finally, these MAD-normalized intensities at the 95th percentile were sorted. In order to make the comparison of all clustering methods more comparable, an equal number of probe sets (254) was selected from the top of the sorted list and was used for clustering.

### C. Selection of ROSE Probe Sets

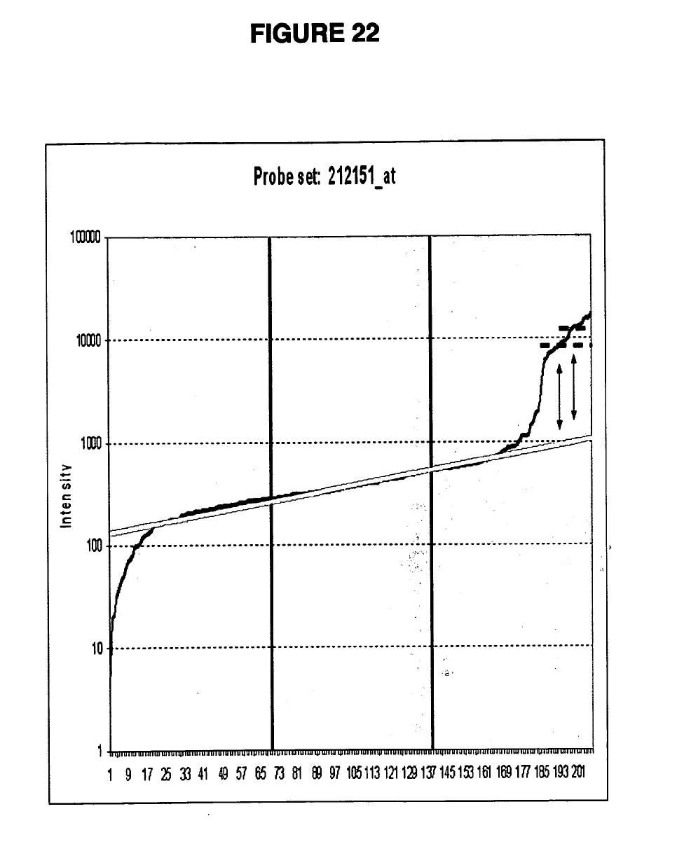

ROSE (Recognition of Outlier by Sampling Ends) was developed as an alternative method for outlier detection. In COPA, units of MAD at a fixed point (typically either the 90th or 95th percentile) rank the outliers. This fixed-point threshold confers a size bias for the clusters (higher percentile levels favor smaller groups of outlier signals). More importantly, the ranking of probe sets is by the magnitude of their deviation. Those with the greatest deviations will dominate the top of the list. The potential drawback to this is that larger groups of related samples with outlier signals may be missed if the magnitude of their variance is not extremely high.
In contrast, ROSE applies a single threshold for the magnitude of the deviation and then orders the probe sets by the size of the largest sampled group that satisfies this cutoff. Regardless of the magnitude of the difference from median, all probe sets that satisfy the threshold cutoff and are within the designated size range are considered equal. Details of the ROSE method, as it was applied in this study, follow. The intensity values for each of the 54,504 probe sets were plotted individually in ascending order. The plots were divided into thirds and the intensities from the middle third were used to generate trend lines by least squares fitting. Groups of 2*k (where k is an integer from 2 to one third of the sample size) were sampled from each end of the intensity plots and the median intensities of these groups were compared to the trend lines. The choice of a trend line as the metric, rather than simply median, is meant to reduce the number of probe sets than simply have a high variance, but do not necessarily contain distinct clusters of outlier samples.
FIG. 22 (S2′) illustrates how this is accomplished. Increasing sized groups are sampled from each end until the median intensity of a group fails to exceed the desired threshold. The largest value of k at which each probe set surpasses the threshold is recorded. The probe sets are then ordered by their maximum k values. In this study a probe set was selected for clustering if k≧6 and the median intensity of the sampled group was at least 7-fold its corresponding point on the trend line. This threshold for k was selected in order to enrich for groups in the range of 10 or more members (greater than 5% of the population size). Smaller groups, although still possibly quite interesting, are much less likely to yield statistically significant results. The 7-fold threshold was chosen to minimize the impact of signal noise on probe set selection and also to limit the total number of probe sets to be used for clustering. Only 254 probe sets out of 54,504 (0.5%) satisfied these criteria of 7× threshold and k values ≧6.

### D. Outlier Probe Set Selection for CCG 1961 (Validation Cohort)

Masking and filtering was applied to the CCG 1961 data set exactly the same way as in P9906. ROSE used the same 7-fold threshold for intensity and k≧6. 167 probe sets (0.3% of the 54,504) satisfied these criteria. COPA clustering used the top 167 probe sets at the 95^{th} percentile level. HC used the top 167 probe sets ranked by their CV.

### E. Probe Sets Used for Clustering

TABLE S7A′
Probe Sets Used in P9906 and CCG1961
The probe sets common to HC and either COPA or ROSE are
shown in bold; those shared between COPA and either
HC or ROSE are italicized.

| | | |
|---|---|---|
| HC | COPA | ROSE |

P9906 Probe Sets (254)

| | | |
|---|---|---|
| 117_at | 38487_at | 38487_{—}at |
| [symbol] | 46665_at | 46665_{—}at |
| 1553328_a_at | [symbol] | 200799_{—}at |
| 1553613_s_at | [symbol] | [symbol] |
| 1554633_{—}a_{—}at | 201566_x_at | 201012_at |
| 1554892_a_at | 201579_at | [symbol] |
| [symbol] | 201656_at | 201215_{—}at |
| [symbol] | 201669_s_at | 201579_{—}at |
| [symbol] | [symbol] | 201656_{—}at |
| 1559696_at | [symbol] | [symbol] |
| 1559697_a_at | 202206_at | [symbol] |
| 1566772_at | 202410_x_at | 202206_{—}at |
| 200799_{—}at | [symbol] | 202207_at |
| [symbol] | [symbol] | 202273_at |
| [symbol] | [symbol] | 202289_s_at |
| 201215_{—}at | 202976_s_at | 202336_s_at |
| 201839_s_at | 202988_s_at | 202409_at |
| [symbol] | [symbol] | [symbol] |
| 202018_s_at | [symbol] | [symbol] |
| [symbol] | [symbol] | 202890_at |
| [symbol] | [symbol] | [symbol] |
| [symbol] | [symbol] | 202976_{—}s_{—}at |
| [symbol] | [symbol] | 202988_{—}s_{—}at |
| 203131_at | 203865_s_at | [symbol] |
| 203153_at | 203910_at | [symbol] |
| [symbol] | 203921_at | 203335_{—}at |
| [symbol] | [symbol] | 203394_{—}s_{—}at |
| 203335_{—}at | [symbol] | [symbol] |
| 203394_{—}s_{—}at | [symbol] | [symbol] |
| [symbol] | [symbol] | [symbol] |
| [symbol] | [symbol] | 203726_{—}s_{—}at |
| [symbol] | [symbol] | [symbol] |
| 203726_{—}s_{—}at | [symbol] | 203865_{—}s_{—}at |
| [symbol] | 204439_at | 203910_{—}at |
| [symbol] | 204456_s_at | 203921_{—}at |
| [symbol] | [symbol] | [symbol] |
| 203973_s_at | [symbol] | [symbol] |
| 204014_{—}at | [symbol] | 204014_{—}at |
| 204015_s_at | [symbol] | [symbol] |
| [symbol] | [symbol] | [symbol] |
| [symbol] | [symbol] | [symbol] |
| [symbol] | 205347_s_at | [symbol] |
| 204134_at | 205413_at | [symbol] |
| [symbol] | [symbol] | 204439_{—}at |
| 204273_at | [symbol] | 204614_{—}at |
| [symbol] | [symbol] | [symbol] |
| 204326_x_at | [symbol] | [symbol] |
| 204351_at | 205914_s_at | [symbol] |
| 204363_at | 205980_s_at | [symbol] |
| 204469_at | 206028_s_at | 204999_s_at |
| 204482_at | 206040_s_at | 205237_at |
| 204614_{—}at | 206067_s_at | [symbol] |
| 204684_at | [symbol] | [symbol] |
| 204745_x_at | 206150_at | 205286_at |
| [symbol] | 206181_at | 205347_{—}s_{—}at |
| [symbol] | [symbol] | 205402_{—}x_{—}at |
| [symbol] | 206298_at | 205413_{—}at |
| [symbol] | [symbol] | 205445_{—}at |
| 204971_at | [symbol] | 205488_at |
| [symbol] | 206637_at | [symbol] |
| [symbol] | [symbol] | 205493_{—}s_{—}at |
| 205402_{—}x_{—}at | 207173_x_at | [symbol] |
| 205405_at | 207261_at | [symbol] |
| 205445_{—}at | 207453_s_at | [symbol] |
| [symbol] | 207696_at | 205950_{—}s_{—}at |
| 205493_{—}s_{—}at | [symbol] | 206028_{—}s_{—}at |
| 205513_at | [symbol] | 206067_{—}s_{—}at |
| 205557_at | 209087_x_at | [symbol] |
| 205592_at | 209101_at | 206181_{—}at |
| 205593_s_at | [symbol] | [symbol] |
| 205614_x_at | 209604_s_at | 206298_{—}at |
| [symbol] | 209728_at | 206310_{—}at |
| [symbol] | 209897_s_at | [symbol] |
| 205857_at | [symbol] | [symbol] |
| 205858_at | 209959_at | 206633_{—}at |
| 205863_at | [symbol] | 206756_at |
| [symbol] | [symbol] | 206836_{—}at |
| 205950_{—}s_{—}at | [symbol] | 207173_{—}x_{—}at |
| [symbol] | 211340_s_at | 207651_{—}at |
| 206172_at | [symbol] | 207978_{—}s_{—}at |
| 206207_at | 211735_x_at | [symbol] |
| [symbol] | [symbol] | 208553_at |
| 206310_{—}at | 212077_at | [symbol] |
| [symbol] | [symbol] | 208937_{—}s_{—}at |
| 206461_x_at | [symbol] | 209101_{—}at |
| [symbol] | [symbol] | [symbol] |
| 206633_{—}at | 212158_at | 209301_{—}at |
| 206634_at | 212592_at | 209604_{—}s_{—}at |
| 206749_at | [symbol] | 209875_s_at |
| 206836_{—}at | [symbol] | 209892_at |
| 206932_at | 213273_at | 209897_{—}s_{—}at |
| [symbol] | [symbol] | [symbol] |
| 207651_{—}at | [symbol] | [symbol] |
| 207978_{—}s_{—}at | [symbol] | 210150_s_at |
| 208148_at | 213714_at | 210640_{—}s_{—}at |
| 208173_at | 213737_x_at | [symbol] |
| [symbol] | [symbol] | [symbol] |
| [symbol] | 214043_at | 210869_s_at |
| 208581_x_at | 214453_s_at | 211340_{—}s_{—}at |
| 208937_{—}s_{—}at | 214497_s_at | 211341_at |
| 209289_at | [symbol] | 211506_{—}s_{—}at |
| 209290_s_at | 215028_at | 211560_{—}s_{—}at |
| [symbol] | [symbol] | 211597_{—}s_{—}at |
| 209301_{—}at | 215426_at | [symbol] |
| 209369_at | 215666_at | [symbol] |
| 209757_s_at | 216834_at | 212077_{—}at |
| [symbol] | 217083_at | [symbol] |
| [symbol] | [symbol] | [symbol] |
| 210254_at | 217963_s_at | [symbol] |
| 210640_{—}s_{—}at | 218086_at | 212158_{—}at |
| [symbol] | 218468_s_at | 212192_at |
| [symbol] | 218469_at | 212592_{—}at |
| 210746_s_at | 218625_at | [symbol] |
| 211338_at | 218804_at | [symbol] |
| 211456_x_at | 218847_at | 213258_{—}at |
| 211506_{—}s_{—}at | 219463_at | [symbol] |
| 211560_{—}s_{—}at | 219489_s_at | 213362_at |
| 211597_{—}s_{—}at | 219837_s_at | [symbol] |
| 211634_x_at | 220059_at | [symbol] |
| 211639_x_at | 220075_s_at | 213714_{—}at |
| 211655_at | 220377_at | 213802_at |
| [symbol] | [symbol] | 213808_{—}at |
| 211820_x_at | 220638_s_at | [symbol] |
| [symbol] | 220759_at | 213880_at |
| [symbol] | 221066_at | 214146_s_at |
| 212104_s_at | 221254_s_at | 214349_{—}at |
| [symbol] | [symbol] | 214534_at |
| [symbol] | 222934_s_at | 214537_at |
| 212185_x_at | 223121_s_at | [symbol] |
| 212501_at | [symbol] | 214774_{—}x_{—}at |
| 212859_x_at | 223449_at | [symbol] |
| [symbol] | 223502_s_at | 215182_x_at |
| [symbol] | 223720_at | 215379_{—}x_{—}at |
| 213194_at | 223885_at | 215692_{—}s_{—}at |
| 213258_{—}at | [symbol] | 216623_{—}x_{—}at |
| [symbol] | 225369_at | 217083_{—}at |
| [symbol] | 225436_at | [symbol] |
| 213418_at | 225483_at | 217110_{—}s_{—}at |
| [symbol] | [symbol] | 217276_x_at |
| 213488_at | 225660_at | 217281_x_at |
| 213791_at | [symbol] | 217284_x_at |
| 213808_{—}at | 226282_at | 217963_{—}s_{—}at |
| [symbol] | [symbol] | 218086_{—}at |
| 213993_at | [symbol] | 218330_s_at |
| 214349_{—}at | [symbol] | 218468_{—}s_{—}at |
| [symbol] | [symbol] | 218469_{—}at |
| 214774_{—}x_{—}at | [symbol] | 218847_{—}at |
| 215108_x_at | 227440_at | 219463_{—}at |
| [symbol] | 227441_s_at | 219470_x_at |
| 215214_at | 227711_at | 219489_{—}s_{—}at |
| 215379_{—}x_{—}at | [symbol] | 219837_{—}s_{—}at |
| 215692_{—}s_{—}at | 228017_s_at | 220010_{—}at |
| 215784_at | [symbol] | 220059_{—}at |
| 216320_x_at | [symbol] | 220377_{—}at |
| 216336_x_at | [symbol] | [symbol] |
| 216401_x_at | 228599_at | 221254_{—}s_{—}at |
| 216491_x_at | [symbol] | [symbol] |
| 216560_x_at | [symbol] | 222921_s_at |
| 216623_{—}x_{—}at | 228918_at | 222934_{—}s_{—}at |
| 216853_x_at | 229029_at | 223121_{—}s_{—}at |
| 216874_at | 229149_at | 223786_{—}at |
| 216984_x_at | 229233_at | [symbol] |
| [symbol] | 229461_x_at | 224520_s_at |
| 217110_{—}s_{—}at | [symbol] | 225436_{—}at |
| 217143_s_at | [symbol] | 225483_{—}at |
| 217148_x_at | 229967_at | [symbol] |
| 217165_x_at | 229975_at | 225597_at |
| 217179_x_at | [symbol] | [symbol] |
| 217235_x_at | 230030_at | 226084_{—}at |
| 217258_x_at | 230110_at | 226282_{—}at |
| 217388_s_at | 230306_at | [symbol] |
| 217623_at | 230468_s_at | 226676_{—}at |
| 218145_at | 230472_at | 226733_at |
| 219093_at | [symbol] | [symbol] |
| 219360_s_at | 230668_at | 227006_at |
| 219666_at | 230698_at | [symbol] |
| 219714_s_at | 230803_s_at | [symbol] |
| 220010_{—}at | 230817_at | [symbol] |
| [symbol] | 231040_at | 227440_{—}at |
| 221215_s_at | [symbol] | 227441_{—}s_{—}at |
| 221766_s_at | [symbol] | [symbol] |
| [symbol] | 231455_at | 228017_{—}s_{—}at |
| 222288_at | 231706_s_at | [symbol] |
| [symbol] | [symbol] | 228262_{—}at |
| 223678_s_at | 231899_at | 228297_{—}at |
| 223786_{—}at | [symbol] | [symbol] |
| 223939_at | 232530_at | [symbol] |
| [symbol] | [symbol] | [symbol] |
| [symbol] | 233847_x_at | [symbol] |
| [symbol] | 234261_at | 229233_{—}at |
| 226034_at | 234803_at | 229461_{—}x_{—}at |
| 226084_{—}at | 234849_at | [symbol] |
| 226189_at | 234985_at | [symbol] |
| 226325_at | 235284_s_at | 229975_{—}at |
| [symbol] | 235666_at | [symbol] |
| 226492_at | 235721_at | 230110_{—}at |
| 226621_at | 235911_at | 230128_{—}at |
| 226676_{—}at | [symbol] | 230130_at |
| 226677_at | 236430_at | 230472_{—}at |
| 226757_at | [symbol] | [symbol] |
| 226818_at | 236633_at | 230698_{—}at |
| [symbol] | 236773_at | 230803_{—}s_{—}at |
| [symbol] | 236967_at | 230817_{—}at |
| 227195_at | 237069_s_at | 231040_{—}at |
| [symbol] | 237238_at | 231166_at |
| [symbol] | 237717_x_at | [symbol] |
| 227697_at | 237828_at | [symbol] |
| [symbol] | 237978_at | 231455_{—}at |
| [symbol] | [symbol] | 231513_at |
| 228262_{—}at | 238689_at | [symbol] |
| 228297_{—}at | 238900_at | 231899_{—}at |
| [symbol] | 239361_at | [symbol] |
| [symbol] | [symbol] | 232523_{—}at |
| [symbol] | [symbol] | 232636_{—}at |
| [symbol] | [symbol] | 232914_s_at |
| [symbol] | 240794_at | [symbol] |
| [symbol] | 241527_at | 234261_{—}at |
| [symbol] | 241535_at | 235521_at |
| 230128_{—}at | 242172_at | 235666_{—}at |
| 230255_at | 242385_at | 235911_{—}at |
| 230291_s_at | [symbol] | [symbol] |
| [symbol] | [symbol] | 236430_{—}at |
| 230788_at | 242747_at | [symbol] |
| 230791_at | [symbol] | 236773_{—}at |
| 231202_at | 244002_at | [symbol] |
| [symbol] | 244155_x_at | 238689_{—}at |
| [symbol] | [symbol] | 239657_x_at |
| [symbol] | 244750_at | [symbol] |
| [symbol] | 244782_at | [symbol] |
| 232523_{—}at | [symbol] | [symbol] |
| 232629_at | 1552767_a_at | 241535_{—}at |
| 232636_{—}at | 1553629_a_at | 241960_{—}at |
| [symbol] | 1553963_at | 242172_{—}at |
| 234830_at | 1554343_a_at | 242385_{—}at |
| 235249_at | 1554912_at | [symbol] |
| 235371_at | 1555220_a_at | [symbol] |
| [symbol] | [symbol] | [symbol] |
| [symbol] | 1555745_a_at | [symbol] |
| 237471_at | [symbol] | 244750_{—}at |
| 237613_at | 1557876_at | [symbol] |
| 237625_s_at | 1559394_a_at | 1552511_a_at |
| [symbol] | 1559459_at | 1552767_{—}a_{—}at |
| 238423_at | [symbol] | 1553629_{—}a_{—}at |
| 240104_at | 1559842_at | 1554343_{—}a_{—}at |
| [symbol] | 1559865_at | 1554633_{—}a_{—}at |
| [symbol] | 1560315_at | [symbol] |
| [symbol] | 1560642_at | 1555745_{—}a_{—}at |
| 241960_{—}at | 1561025_at | 1555756_a_at |
| [symbol] | 1563868_a_at | [symbol] |
| [symbol] | 1566825_at | 1559394_{—}a_{—}at |
| 242541_at | 1568603_at | 1559459_{—}at |
| [symbol] | 1569591_at | [symbol] |
| 244463_at | 1569663_at | 1561025_{—}at |
| [symbol] | 1570058_at | 1566825_{—}at |

CCG 1961 Probe_sets (167)

| | | |
|---|---|---|
| 117_at | [symbol] | [symbol] |
| [symbol] | [symbol] | [symbol] |
| 1554140_at | 1555216_a_at | 1555578_{—}at |
| 1554655_a_at | 1555578_{—}at | [symbol] |
| [symbol] | [symbol] | 1559394_{—}a_{—}at |
| [symbol] | [symbol] | [symbol] |
| [symbol] | [symbol] | 1560109_{—}s_{—}at |
| [symbol] | 1559394_{—}a_{—}at | [symbol] |
| [symbol] | [symbol] | 1560483_at |
| 1559696_at | 1560109_{—}s_{—}at | 1560581_at |
| 1559910_at | [symbol] | 1565558_{—}at |
| [symbol] | [symbol] | 200800_{—}s_{—}at |
| [symbol] | 1565558_{—}at | 201579_{—}at |
| 1567912_s_at | 200800_{—}s_{—}at | [symbol] |
| 201131_s_at | 201579_{—}at | 202178_{—}at |
| 201215_at | [symbol] | 202289_{—}s_{—}at |
| 201243_s_at | 202178_{—}at | 202581_{—}at |
| [symbol] | 202289_{—}s_{—}at | 202890_{—}at |
| 201843_s_at | 202478_at | 203038_{—}at |
| 202007_at | 202581_{—}at | [symbol] |
| 202609_at | 202890_{—}at | 203373_at |
| 203131_at | 203038_{—}at | 203434_s_at |
| 203216_s_at | [symbol] | 203476_{—}at |
| [symbol] | 203476_{—}at | 203695_{—}s_{—}at |
| 203304_at | 203695_{—}s_{—}at | 203835_{—}at |
| 203632_s_at | 203835_{—}at | 203865_{—}s_{—}at |
| [symbol] | 203865_{—}s_{—}at | [symbol] |
| 204015_{—}s_{—}at | [symbol] | 204015_{—}s_{—}at |
| 204066_s_at | [symbol] | [symbol] |
| [symbol] | 204114_{—}at | 204114_{—}at |
| 204337_at | 204304_s_at | 204439_{—}at |
| [symbol] | 204416_x_at | [symbol] |
| [symbol] | 204439_{—}at | 204913_s_at |
| [symbol] | [symbol] | 204914_{—}s_{—}at |
| [symbol] | 204914_{—}s_{—}at | 204915_{—}s_{—}at |
| 205493_s_at | 204915_{—}s_{—}at | 204944_{—}at |
| 205573_s_at | 204944_{—}at | 205109_{—}s_{—}at |
| [symbol] | 205109_{—}s_{—}at | [symbol] |
| [symbol] | [symbol] | [symbol] |
| [symbol] | [symbol] | 205489_{—}at |
| 205942_s_at | [symbol] | 205544_{—}s_{—}at |
| 205951_at | 205477_s_at | 205592_at |
| 205980_s_at | 205489_{—}at | [symbol] |
| 205987_at | 205544_{—}s_{—}at | 205870_{—}at |
| 206070_s_at | [symbol] | [symbol] |
| 206084_at | [symbol] | 205936_{—}s_{—}at |
| [symbol] | 205870_{—}at | 205946_{—}at |
| 206204_at | [symbol] | 206111_{—}at |
| [symbol] | 205936_{—}s_{—}at | 206181_{—}at |
| 206298_at | 205946_{—}at | [symbol] |
| [symbol] | 206111_{—}at | 206413_{—}s_{—}at |
| 206432_at | [symbol] | [symbol] |
| 206741_at | 206181_{—}at | 208285_{—}at |
| [symbol] | [symbol] | [symbol] |
| 206785_s_at | [symbol] | 209392_{—}at |
| 206851_at | 206413_{—}s_{—}at | 209570_{—}s_{—}at |
| 207638_at | 206710_s_at | 209602_{—}s_{—}at |
| 207768_at | [symbol] | 209822_{—}s_{—}at |
| 207802_at | 206881_s_at | [symbol] |
| 208029_s_at | 208285_{—}at | 210016_{—}at |
| 208090_s_at | 208470_s_at | 210665_{—}at |
| 208148_at | [symbol] | [symbol] |
| 208605_s_at | 209392_{—}at | 211306_{—}s_{—}at |
| 209289_at | 209570_{—}s_{—}at | 211382_s_at |
| [symbol] | 209602_{—}s_{—}at | 211560_{—}s_{—}at |
| 209436_at | 209822_{—}s_{—}at | 211743_{—}s_{—}at |
| 209687_at | [symbol] | [symbol] |
| 209774_x_at | 210016_{—}at | 212151_{—}at |
| [symbol] | 210432_s_at | 212592_{—}at |
| 210095_s_at | [symbol] | 212942_{—}s_{—}at |
| 210135_s_at | 211306_{—}s_{—}at | 213005_{—}s_{—}at |
| 210402_at | [symbol] | 213050_at |
| 210546_x_at | 211560_{—}s_{—}at | [symbol] |
| 210664_s_at | 212094_at | [symbol] |
| 210665_{—}at | [symbol] | 213423_{—}x_{—}at |
| [symbol] | 212151_{—}at | 213906_at |
| 211276_at | 212592_{—}at | 214020_{—}x_{—}at |
| [symbol] | 213005_{—}s_{—}at | 214446_{—}at |
| 211674_x_at | [symbol] | [symbol] |
| 211719_x_at | [symbol] | 214978_{—}s_{—}at |
| 211743_{—}s_{—}at | [symbol] | 215177_{—}s_{—}at |
| [symbol] | 213423_{—}x_{—}at | [symbol] |
| 212554_at | [symbol] | [symbol] |
| 212942_{—}s_{—}at | 213566_at | [symbol] |
| 213032_at | 214020_{—}x_{—}at | 217963_{—}s_{—}at |
| [symbol] | 214043_at | 218922_{—}s_{—}at |
| [symbol] | 214446_{—}at | 219355_{—}at |
| [symbol] | [symbol] | 219463_{—}at |
| 213380_x_at | 214978_{—}s_{—}at | 219489_{—}s_{—}at |
| 213418_at | 215177_{—}s_{—}at | 219840_{—}s_{—}at |
| 213436_at | [symbol] | 219855_{—}at |
| 213479_at | [symbol] | 220276_{—}at |
| [symbol] | [symbol] | 220377_{—}at |
| 213791_at | [symbol] | 220922_s_at |
| 213993_at | 217963_{—}s_{—}at | 222162_{—}s_{—}at |
| 213994_s_at | 218922_{—}s_{—}at | [symbol] |
| 214433_s_at | [symbol] | [symbol] |
| [symbol] | 219355_{—}at | 223075_s_at |
| 214769_at | 219463_{—}at | 223754_at |
| 214774_x_at | 219489_{—}s_{—}at | [symbol] |
| 215108_x_at | 219840_{—}s_{—}at | [symbol] |
| 215121_x_at | 219855_{—}at | 224762_{—}at |
| [symbol] | 220276_{—}at | 225369_at |
| 215733_x_at | 220377_{—}at | 225782_at |
| 216320_x_at | 220528_at | 225977_{—}at |
| [symbol] | 222162_{—}s_{—}at | [symbol] |
| [symbol] | 222258_s_at | 226096_{—}at |
| [symbol] | [symbol] | 226282_{—}at |
| 217138_x_at | 222347_at | 226636_{—}at |
| 218507_at | [symbol] | 226913_{—}s_{—}at |
| 219093_at | 223319_at | 227006_at |
| [symbol] | 223422_s_at | [symbol] |
| 219525_at | [symbol] | [symbol] |
| 220225_at | [symbol] | 227377_{—}at |
| 221731_x_at | 224762_{—}at | 227441_{—}s_{—}at |
| 221870_at | 225977_{—}at | 227949_{—}at |
| 221901_at | [symbol] | 228018_{—}at |
| [symbol] | 226096_{—}at | 228057_{—}at |
| 222315_at | 226282_{—}at | 228116_{—}at |
| [symbol] | 226636_{—}at | 228262_{—}at |
| 222885_at | 226913_{—}s_{—}at | [symbol] |
| 223235_s_at | [symbol] | [symbol] |
| 223611_s_at | [symbol] | 228994_{—}at |
| 223612_s_at | 227377_{—}at | 229108_at |
| [symbol] | 227441_{—}s_{—}at | 229247_at |
| [symbol] | 227949_{—}at | [symbol] |
| 225575_at | 228018_{—}at | 229975_{—}at |
| 225842_at | 228057_{—}at | 230030_at |
| [symbol] | 228116_{—}at | 230668_at |
| 226676_at | 228262_{—}at | 250680_{—}at |
| 226677_at | [symbol] | [symbol] |
| 227174_at | [symbol] | [symbol] |
| [symbol] | 228994_{—}at | 231257_{—}at |
| [symbol] | [symbol] | 231316_at |
| 227481_at | 229661_at | 231455_at |
| 227758_at | [symbol] | 231600_{—}at |
| [symbol] | 229975_{—}at | 231859_at |
| 228766_at | 230472_at | [symbol] |
| 228780_at | 250680_{—}at | 232010_{—}at |
| [symbol] | [symbol] | 232231_{—}at |
| 229147_at | [symbol] | 232636_{—}at |
| [symbol] | 231257_{—}at | 232903_at |
| 229934_at | 231503_at | 234985_at |
| [symbol] | 231600_{—}at | 235343_at |
| 230110_at | [symbol] | [symbol] |
| 230372_at | 232010_{—}at | 235988_{—}at |
| 230495_at | 232231_{—}at | 236430_at |
| [symbol] | 232636_{—}at | 236489_{—}at |
| [symbol] | [symbol] | 237207_at |
| [symbol] | 235911_at | 237421_{—}at |
| 232523_at | 235988_{—}at | 237466_{—}s_{—}at |
| 233038_at | 236489_{—}at | 238617_{—}at |
| 233463_at | 237421_{—}at | 238778_at |
| 233969_at | 237466_{—}s_{—}at | 239657_{—}x_{—}at |
| 235004_at | 237974_at | 239964_{—}at |
| [symbol] | 238617_{—}at | 240032_{—}at |
| 235700_at | 239610_at | 240179_at |
| 235771_at | 239657_{—}x_{—}at | 240245_{—}at |
| 236301_at | 239964_{—}at | 240336_at |
| 237802_at | 240032_{—}at | 240347_{—}at |
| 238091_at | 240245_{—}at | 240466_{—}at |
| 238175_at | 240347_{—}at | 240496_{—}at |
| 240758_at | 240466_{—}at | 241506_at |
| [symbol] | 240496_{—}at | 241960_at |
| 243533_x_at | [symbol] | [symbol] |
| [symbol] | 242747_at | 242468_at |
| 243932_at | [symbol] | [symbol] |

TABLE S7B′
Overlap of Probe Sets Used in Either P9906 or CCG1961

| | |
|---|---|
| COPA | ROSE |

P9906 (254 total)

| | | |
|---|---|---|
| HC | 96 (37.8%) | 135 (53.1%) |
| COPA | — | 169 (66.5%) |
| HC & COPA | — | 94 (37.0%) |

CCG1961 (167 total)

| | | |
|---|---|---|
| HC | 55 (32.9%) | 46 (27.5%) |
| COPA | — | 130 (77.8%) |
| HC & COPA | — | 42 (25.1%) |

TABLE S7C′
Common P9906 and CCG1961 Probe Sets by Method

| | HC (1961) | COPA (1961) | ROSE (1961) |
|---|---|---|---|
| HC (9906) | 55 (32.9%) | 56 (33.5%) | 59 (35.3%) |
| COPA (9906) | 36 (21.6%) | 66 (39.5%) | 68 (40.7%) |
| ROSE (9906) | 45 (26.9%) | 75 (44.9%) | 77 (46.1%) |

### 5. Overlap of P9906 Clusters Defined by Each Method

Each of the three clustering methods in P9906 identified predominantly the same samples even though they shared only 37% of the probe sets (Table S7B). As in shown in Table S8, the overall identity of samples across all three methods is 86.5%. The primary factor responsible for this being lower than ˜90% is that HC and ROSE identified a cluster 4, while COPA did not. All 23 of the patients with TCF3-PBX1 translocations were grouped into cluster 1 by all three methods, as were 19 of the 21 patients with MLL translocations. Even though the remaining clusters lacked known underlying translocations they were also very highly conserved.

TABLE S8′
Identity of Membership in P9906 Clusters

| | Cluster | | | | | | | | |
|---|---|---|---|---|---|---|---|---|---|
| | 1 | 2 | 3 | 4 | 5 | 6 | 7 | 8 | Overall |
| HC v COPA | 19 | 23 | 8 | 0 | 9 | 19 | 88 | 19 | 89.4% |
| HC v ROSE | 20 | 23 | 8 | 10 | 9 | 19 | 82 | 22 | 93.2% |
| COPA v ROSE | 20 | 23 | 10 | 0 | 10 | 21 | 82 | 20 | 89.9% |
| HC v COPA v ROSE | 19 | 23 | 8 | 0 | 9 | 19 | 82 | 19 | 86.5% |

6. Probesets Associated with Rose Clusters (by Median Rank Order)
The top 100 median rank order probe sets for each ROSE cluster are given. Percentile denotes the ranking of the median cluster rank order relative to the maximum possible. Bold font indicates that these probe sets were also among the 254 outliers selected for clustering. Probe sets marked with an asterisk (including several PCDH17, GAB1, GPR110, CENTG2 and CD99) indicate those for which Affymetrix does not specify a gene, however the probe sets were mapped using the UCSC Genome Browser (http://genome.ucsc.edu/) between exons of the indicated genes. Those with a question mark were also lacking Affymetrix gene data, but were mapped within 10 kb of the indicated gene using the UCSC Genome Browser.

TABLE S9′
Top 100 Rank Order Genes Defining ROSE Cluster 1 (R1)

| | Per- | | | |
|---|---|---|---|---|
| Probeset | centile | Symbol | EntrezID | Cytoband |
| 219463_{—}at | 100 | C20orf103 | 24141 | 20p12 |
| 205899_{—}at | 100 | CCNA1 | 8900 | 13q12.3-q13 |
| 235479_at | 100 | CPEB2 | 132864 | 4p15.33 |
| 226939_at | 100 | CPEB2 | 132864 | 4p15.33 |
| 241706_at | 100 | CPNE8 | 144402 | 12q12 |
| 236921_at | 100 | EMB* | — | 5q11.1 |
| 222603_at | 100 | ERMP1 | 79956 | 9p24 |
| 213147_at | 100 | HOXA10 | 3206 | 7p15-p14 |
| 213150_{—}at | 100 | HOXA10 | 3206 | 7p15-p14 |
| 235521_{—}at | 100 | HOXA3 | 3200 | 7p15-p14 |
| 214651_{—}s_{—}at | 100 | HOXA9 | 3205 | 7p15-p14 |
| 209905_{—}at | 100 | HOXA9 | 3205 | 7p15-p14 |
| 215163_at | 100 | IGF2BP2* | — | 3q27.2 |
| 226789_at | 100 | LOC647121 | 647121 | 1p11.2 |
| 202890_{—}at | 100 | MAP7 | 9053 | 6q23.3 |
| 238498_at | 100 | MAP7? | — | 6q23.3 |
| 204069_{—}at | 100 | MEIS1 | 4211 | 2p14-p13 |
| 242172_{—}at | 100 | MEIS1 | 4211 | 2p14-p13 |
| 1559477_{—}s_{—}at | 100 | MEIS1 | 4211 | 2p14-p13 |
| 219033_at | 100 | PARP8 | 79668 | 5q11.1 |
| 204304_{—}s_{—}at | 100 | PROM1 | 8842 | 4p15.32 |
| 242414_at | 100 | QPRT | 23475 | 16p11.2 |
| 204044_at | 100 | QPRT | 23475 | 16p11.2 |
| 1568589_at | 100 | REEP3* | — | 10q21.3 |
| 231899_{—}at | 100 | ZC3H12C | 85463 | 11q22.3 |
| 220416_{—}at | 99.5 | ATP8B4 | 79895 | 15q21.2 |
| 225841_at | 99.5 | C1orf59 | 113802 | 1p13.3 |
| 227877_at | 99.5 | C5orf39 | 389289 | 5p12 |
| 212063_at | 99.5 | CD44 | 960 | 11p13 |
| 213844_{—}at | 99.5 | HOXA5 | 3202 | 7p15-p14 |
| 218847_{—}at | 99.5 | IGF2BP2 | 10644 | 3q27.2 |
| 201163_s_at | 99.5 | IGFBP7 | 3490 | 4q12 |
| 201105_{—}at | 99.5 | LGALS1 | 3956 | 22q13.1 |
| 228412_at | 99.5 | LOC643072 | 643072 | 2q24.2 |
| 240180_at | 99.5 | MAP7? | — | 6q23.3 |
| 201153_s_at | 99.5 | MBNL1 | 4154 | 3q25 |
| 1558111_at | 99.5 | MBNL1 | 4154 | 3q25 |
| 1556658_a_at | 99.5 | MBNL1* | — | 3q25.2 |
| 238558_at | 99.5 | MBNL1* | — | 3q25.2 |
| 244008_at | 99.5 | PARP8? | — | 5q11.1 |
| 204082_at | 99.5 | PBX3 | 5090 | 9q33-q34 |
| 230480_at | 99.5 | PIWIL4 | 143689 | 11q21 |
| 232231_{—}at | 99.5 | RUNX2 | 860 | 6p21 |
| 211769_x_at | 99.5 | SERINC3 | 10955 | 20q13.1-q13.3 |
| 226415_{—}at | 99.5 | VAT1L | 57687 | 16q23.1 |
| 203827_at | 99.5 | WIPI1 | 55062 | 17q24.2 |
| 242023_at | 99 | ABHD4 | 63874 | 14q11.2 |
| 202603_at | 99 | ADAM10* | — | 15q22.1 |
| 215925_s_at | 99 | CD72 | 971 | 9p13.3 |
| 228365_at | 99 | CPNE8 | 144402 | 12q12 |
| 214297_at | 99 | CSPG4 | 1464 | 15q24.2 |
| 200046_at | 99 | DAD1 | 1603 | 14q11-q12 |
| 227002_at | 99 | FAM78A | 286336 | 9q34 |
| 235291_s_at | 99 | FLJ32255 | 643977 | 5p12 |
| 238712_at | 99 | FOXP1* | — | 3p14.1 |
| 204417_at | 99 | GALC | 2581 | 14q31 |
| 235173_at | 99 | hCG_1806964 | 401093 | 3q25.1 |
| 201162_at | 99 | IGFBP7 | 3490 | 4q12 |
| 232544_at | 99 | IGFBP7* | — | 4q12 |
| 241391_at | 99 | JMJD1C* | — | 10q21.2 |
| 1557534_{—}at | 99 | LOC339862 | 339862 | 3p24.3 |
| 1556657_at | 99 | MBNL1* | — | 3q25.2 |
| 219988_s_at | 99 | RNF220 | 55182 | 1p34.1 |
| 221473_x_at | 99 | SERINC3 | 10955 | 20q13.1-q13.3 |
| 206506_s_at | 99 | SUPT3H | 8464 | 6p21.1-p21.3 |
| 213836_s_at | 99 | WIPI1 | 55062 | 17q24.2 |
| 218581_at | 98.5 | ABHD4 | 63874 | 14q11.2 |
| 214895_s_at | 98.5 | ADAM10 | 102 | 15q2\|15q22 |
| 212174_at | 98.5 | AK2 | 204 | 1p34 |
| 203562_at | 98.5 | FEZ1 | 9638 | 11q24.2 |
| 235753_at | 98.5 | HOXA7 | 3204 | 7p15-p14 |
| 213910_at | 98.5 | IGFBP7 | 3490 | 4q12 |
| 1569041_at | 98.5 | JMJD1C* | — | 10q21.2 |
| 203836_s_at | 98.5 | MAP3K5 | 4217 | 6q22.33 |
| 203837_at | 98.5 | MAP3K5 | 4217 | 6q22.33 |
| 201152_s_at | 98.5 | MBNL1 | 4154 | 3q25 |
| 235879_at | 98.5 | MBNL1 | 4154 | 3q25 |
| 225202_at | 98.5 | RHOBTB3 | 22836 | 5q15 |
| 227719_at | 98.5 | SMAD9 | 4093 | 13q12-q14 |
| 225959_s_at | 98.5 | ZNRF1 | 84937 | 16q23.1 |
| 223382_s_at | 98.5 | ZNRF1 | 84937 | 16q23.1 |
| 210783_x_at | 98 | CLEC11A | 6320 | 19q13.3 |
| 232645_at | 98 | LOC153684 | 153684 | 5p12 |
| 241681_at | 98 | MBNL1* | — | 3q25.2 |
| 202976_{—}s_{—}at | 98 | RHOBTB3 | 22836 | 5q15 |
| 227611_at | 98 | TARSL2 | 123283 | 15q26.3 |
| 209825_s_at | 98 | UCK2 | 7371 | 1q23 |
| 223383_at | 98 | ZNRF1 | 84937 | 16q23.1 |
| 36553_at | 97.5 | ASMTL | 8623 | Xp22.3; Yp11.3 |
| 224848_at | 97.5 | CDK6 | 1021 | 7q21-q22 |
| 213379_at | 97.5 | COQ2 | 27235 | 4q21.23 |
| 209101_{—}at | 97.5 | CTGF | 1490 | 6q23.1 |
| 218147_s_at | 97.5 | GLT8D1 | 55830 | 3p21.1 |
| 218468_{—}s_{—}at | 97.5 | GREM1 | 26585 | 15q13-q15 |
| 227235_at | 97.5 | GUCY1A3 | 2982 | 4q31.3- |
| | | | | q33\|4q31.1-q31.2 |
| 206289_at | 97.5 | HOXA4 | 3201 | 7p15-p14 |
| 227384_s_at | 97.5 | LOC727820 | 727820 | 1q21.1 |
| 203537_at | 97.5 | PRPSAP2 | 5636 | 17p11.2-p12 |
| 226168_at | 97.5 | ZFAND2B | 130617 | 2q35 |
| 225962_at | 97.5 | ZNRF1 | 84937 | 16q23.1 |

TABLE S10′
Top 100 Rank Order Genes Defining ROSE Cluster 2 (R2)

| Probeset | Percentile | Symbol | EntrezID | Cytoband |
|---|---|---|---|---|
| 227440_{—}at | 100 | ANKS1B | 56899 | 12q23.1 |
| 227441_{—}s_{—}at | 100 | ANKS1B | 56899 | 12q23.1 |
| 227439_{—}at | 100 | ANKS1B | 56899 | 12q23.1 |
| 234261_{—}at | 100 | ANKS1B* | — | 12q23.1 |
| 243533_{—}x_{—}at | 100 | ANKS1B* | — | 12q23.1 |
| 202206_{—}at | 100 | ARL4C | 10123 | 2q37.1 |
| 229247_at | 100 | FBLN7 | 129804 | 2q13 |
| 239657_{—}x_{—}at | 100 | FOXO6 | 100132074 | 1p34.1 |
| 202106_at | 100 | GOLGA3 | 2802 | 12q24.33 |
| 213005_{—}s_{—}at | 100 | KANK1 | 23189 | 9p24.3 |
| 207110_at | 100 | KCNJ12 | 3768 | 17p11.2 |
| 232289_at | 100 | KCNJ12 | 3768 | 17p11.2 |
| 208567_{—}s_{—}at | 100 | KCNJ12 /// | 100131509 /// | 17p11.2 |
| | | LOC100131509 /// | 100134444 /// | |
| | | LOC100134444 | 3768 | |
| 213909_at | 100 | LRRC15 | 131578 | 3q29 |
| 206028_{—}s_{—}at | 100 | MERTK | 10461 | 2q14.1 |
| 211913_s_at | 100 | MERTK | 10461 | 2q14.1 |
| 238778_at | 100 | MPP7 | 143098 | 10p11.23 |
| 212789_at | 100 | NCAPD3 | 23310 | 11q25 |
| 212148_{—}at | 100 | PBX1 | 5087 | 1q23 |
| 212151_{—}at | 100 | PBX1 | 5087 | 1q23 |
| 205253_{—}at | 100 | PBX1 | 5087 | 1q23 |
| 227949_{—}at | 100 | PHACTR3 | 116154 | 20q13.32 |
| 231095_at | 100 | PITPNC1* | — | 17q24.2 |
| 202178_{—}at | 100 | PRKCZ | 5590 | 1p36.33-p36.2 |
| 223693_s_at | 100 | RADIL | 55698 | 7p22.1 |
| 222513_s_at | 100 | SORBS1 | 10580 | 10q23.3-q24.1 |
| 225235_at | 100 | TSPAN17 | 26262 | 5q35.3 |
| 225483_{—}at | 100 | VPS26B | 112936 | 11q25 |
| 224022_x_at | 100 | WNT16 | 51384 | 7q31 |
| 202207_{—}at | 99.5 | ARL4C | 10123 | 2q37.1 |
| 202208_s_at | 99.5 | ARL4C | 10123 | 2q37.1 |
| 206255_at | 99.5 | BLK | 640 | 8p23-p22 |
| 223786_{—}at | 99.5 | CHST6 | 4166 | 16q22 |
| 205489_{—}at | 99.5 | CRYM | 1428 | 16p13.11-p12.3 |
| 205159_at | 99.5 | CSF2RB | 1439 | 22q13.1 |
| 212538_at | 99.5 | DOCK9 | 23348 | 13q32.3 |
| 229655_at | 99.5 | FAM19A5 | 25817 | 22q13.32 |
| 206404_at | 99.5 | FGF9 | 2254 | 13q11-q12 |
| 209558_s_at | 99.5 | HIP1R | 9026 | 12q24 |
| 38340_at | 99.5 | HIP1R | 9026 | 12q24 |
| 235911_{—}at | 99.5 | K03200* | — | 3q29 |
| 204114_{—}at | 99.5 | NID2 | 22795 | 14q21-q22 |
| 1562235_s_at | 99.5 | PBX1* | — | 1q23.3 |
| 229414_at | 99.5 | PITPNC1 | 26207 | 17q24.2 |
| 231040_{—}at | 99.5 | RORB? | — | 9q21.13 |
| 46665_{—}at | 99.5 | SEMA4C | 54910 | 2q11.2 |
| 206181_{—}at | 99.5 | SLAMF1 | 6504 | 1q22-q23 |
| 239427_at | 99.5 | SLAMF1? | — | 1q23.3 |
| 203940_s_at | 99.5 | VASH1 | 22846 | 14q24.3 |
| 230306_at | 99.5 | VPS26B | 112936 | 11q25 |
| 221113_s_at | 99.5 | WNT16 | 51384 | 7q31 |
| 226233_at | 99 | B3GALNT2 | 148789 | 1q42.3 |
| 201615_x_at | 99 | CALD1 | 800 | 7q33 |
| 209570_s_at | 99 | D4S234E | 27065 | 4p16.3 |
| 229892_at | 99 | EP400NL | 347918 | 12q24.33 |
| 206070_{—}s_{—}at | 99 | EPHA3 | 2042 | 3p11.2 |
| 237094_at | 99 | FAM19A5 | 25817 | 22q13.32 |
| 227676_at | 99 | FAM3D | 131177 | 3p14.2 |
| 201579_{—}at | 99 | FAT1 | 2195 | 4q35 |
| 204225_at | 99 | HDAC4 | 9759 | 2q37.3 |
| 1566030_at | 99 | PHACTR3* | — | 20q13.32 |
| 242385_{—}at | 99 | RORB | 6096 | 9q22 |
| 221669_s_at | 98.5 | ACAD8 | 27034 | 11q25 |
| 205083_at | 98.5 | AOX1 | 316 | 2q33 |
| 225313_at | 98.5 | C20orf177 | 63939 | 20q13.2-q13.33 |
| 201616_s_at | 98.5 | CALD1 | 800 | 7q33 |
| 209569_x_at | 98.5 | D4S234E | 27065 | 4p16.3 |
| 212371_at | 98.5 | FAM152A | 51029 | 1q44 |
| 229770_at | 98.5 | GLT1D1 | 144423 | 12q24.32 |
| 226949_at | 98.5 | GOLGA3 | 2802 | 12q24.33 |
| 204202_at | 98.5 | IQCE | 23288 | 7p22.2 |
| 213358_at | 98.5 | KIAA0802 | 23255 | 18p11.22 |
| 210150_{—}s_{—}at | 98.5 | LAMA5 | 3911 | 20q13.2-q13.3 |
| 238451_at | 98.5 | MPP7 | 143098 | 10p11.23 |
| 219155_at | 98.5 | PITPNC1 | 26207 | 17q24.2 |
| 215807_s_at | 98.5 | PLXNB1 | 5364 | 3p21.31 |
| 225728_at | 98.5 | SORBS2 | 8470 | 4q35.1 |
| 217650_x_at | 98.5 | ST3GAL2 | 6483 | 16q22.1 |
| 1554340_a_at | 98 | C1orf187 | 374946 | 1p36.22 |
| 212077_{—}at | 98 | CALD1 | 800 | 7q33 |
| 220373_at | 98 | DCHS2 | 54798 | 4q32.1 |
| 232204_at | 98 | EBF1 | 1879 | 5q34 |
| 201718_s_at | 98 | EPB41L2 | 2037 | 6q23 |
| 201719_s_at | 98 | EPB41L2 | 2037 | 6q23 |
| 231455_{—}at | 98 | FLJ42418 | 400941 | 2p25.2 |
| 219271_at | 98 | GALNT14 | 79623 | 2p23.1 |
| 214265_at | 98 | ITGA8 | 8516 | 10p13 |
| 235666_{—}at | 98 | ITGA8? | — | 10p13 |
| 209760_at | 98 | KIAA0922 | 23240 | 4q31.3 |
| 226796_at | 98 | LOC116236 | 116236 | 17q11.2 |
| 228262_{—}at | 98 | MAP7D2 | 256714 | Xp22.12 |
| 212845_at | 98 | SAMD4A | 23034 | 14q22.2 |
| 202796_at | 98 | SYNPO | 11346 | 5q33.1 |
| 222752_s_at | 98 | TMEM206 | 55248 | 1q32.3 |
| 227733_at | 98 | TMEM63C | 57156 | 14q24.3 |
| 242957_at | 98 | VWCE | 220001 | 11q12.2 |
| 224516_s_at | 97.4 | CXXC5 | 51523 | 5q31.3 |
| 220911_s_at | 97.4 | KIAA1305 | 57523 | 14q12 |
| 213136_at | 97.4 | PTPN2 | 5771 | 18p11.3-p11.2 |
| 202478_at | 97.4 | TRIB2 | 28951 | 2p25.1-p24.3 |

TABLE S11′
Top 100 Rank Order Genes Defining ROSE Cluster 3 (R3)

| Probeset | Percentile | Symbol | EntrezID | Cytoband |
|---|---|---|---|---|
| 244463_at | 100 | ADAM23 | 8745 | 2q33 |
| 240143_at | 100 | ADAM23* | — | 2q33.3 |
| 213808_{—}at | 100 | ADAM23* | — | 2q33.3 |
| 204129_at | 100 | BCL9 | 607 | 1q21 |
| 213050_at | 100 | COBL | 23242 | 7p12.1 |
| 205659_at | 100 | HDAC9 | 9734 | 7p21.1 |
| 230968_at | 100 | HDAC9? | — | 7p21.1 |
| 217869_at | 100 | HSD17B12 | 51144 | 11p11.2 |
| 1557252_at | 100 | HSD17B12* | — | 11p11.2 |
| 216028_at | 100 | HSD17B12? | — | 11p11.2 |
| 242616_at | 100 | HSD17B12? | — | 11p11.2 |
| 230128_{—}at | 100 | IGL@ | 3535 | 22q11.1-q11.2 |
| 204686_at | 100 | IRS1 | 3667 | 2q36 |
| 206765_at | 100 | KCNJ2 | 3759 | 17q23.1-q24.2 |
| 203726_{—}s_{—}at | 100 | LAMA3 | 3909 | 18q11.2 |
| 224823_at | 100 | MYLK | 4638 | 3q21 |
| 202555_s_at | 100 | MYLK | 4638 | 3q21 |
| 216012_at | 100 | PDE4D* | — | 5q12.1 |
| 205632_s_at | 100 | PIP5K1B | 8395 | 9q13 |
| 204469_at | 100 | PTPRZ1 | 5803 | 7q31.3 |
| 212104_s_at | 100 | RBM9 | 23543 | 22q13.1 |
| 213243_at | 100 | VPS13B | 157680 | 8q22.2 |
| 226325_at | 99.5 | ADSSL1 | 122622 | 14q32.33 |
| 1552496_a_at | 99.5 | COBL | 23242 | 7p12.1 |
| 219518_s_at | 99.5 | ELL3 | 80237 | 15q15.3 |
| 231513_{—}at | 99.5 | KCNJ2* | — | 17q24.3 |
| 221584_s_at | 99.5 | KCNMA1 | 3778 | 10q22.3 |
| 213568_at | 99.5 | OSR2 | 116039 | 8q22.2 |
| 202780_at | 99.5 | OXCT1 | 5019 | 5p13.1 |
| 239832_at | 99.5 | PIP5K1B* | — | 9q21.11 |
| 213309_at | 99.5 | PLCL2 | 23228 | 3p24.3 |
| 216218_s_at | 99.5 | PLCL2 | 23228 | 3p24.3 |
| 203020_at | 99.5 | RABGAP1L | 9910 | 1q24 |
| 203097_s_at | 99.5 | RAPGEF2 | 9693 | 4q32.1 |
| 218137_s_at | 99.5 | SMAP1 | 60682 | 6q13 |
| 223246_s_at | 99.5 | STRBP | 55342 | 9q33.3 |
| 225496_{—}s_{—}at | 99.5 | SYTL2 | 54843 | 11q14 |
| 1554803_s_at | 99.5 | TRIM72 | 493829 | 16p11.2 |
| 206046_at | 99 | ADAM23 | 8745 | 2q33 |
| 203865_{—}s_{—}at | 99 | ADARB1 | 104 | 21q22.3 |
| 206167_s_at | 99 | ARHGAP6 | 395 | Xp22.3 |
| 219517_at | 99 | ELL3 | 80237 | 15q15.3 |
| 45572_s_at | 99 | GGA1 | 26088 | 22q13.31 |
| 204891_s_at | 99 | LCK | 3932 | 1p34.3 |
| 204890_s_at | 99 | LCK | 3932 | 1p34.3 |
| 222322_at | 99 | PDE4D* | — | 5q12.1 |
| 203038_at | 99 | PTPRK | 5796 | 6q22.2-q22.3 |
| 213982_s_at | 99 | RABGAP1L | 9910 | 1q24 |
| 238894_at | 99 | RABGAP1L* | — | 1q25.1 |
| 203096_s_at | 99 | RAPGEF2 | 9693 | 4q32.1 |
| 215992_s_at | 99 | RAPGEF2 | 9693 | 4q32.1 |
| 232739_at | 99 | SPIB | 6689 | 19q13.3-q13.4 |
| 220613_s_at | 99 | SYTL2 | 54843 | 11q14 |
| 212350_at | 99 | TBC1D1 | 23216 | 4p14 |
| 203588_s_at | 99 | TFDP2 | 7029 | 3q23 |
| 219520_s_at | 99 | WWC3 | 55841 | Xp22.32 |
| 227173_s_at | 98.5 | BACH2 | 60468 | 6q15 |
| 241871_at | 98.5 | CAMK4 | 814 | 5q21.3 |
| 206806_at | 98.5 | DGKI | 9162 | 7q32.3-q33 |
| 205425_at | 98.5 | HIP1 | 3092 | 7q11.23 |
| 215946_x_at | 98.5 | IGLL3 | 91353 | 22q11.2\|22q11.23 |
| 225963_at | 98.5 | KLHDC5 | 57542 | 12p11.22 |
| 234608_at | 98.5 | LAMA3 | 3909 | 18q11.2 |
| 217140_s_at | 98.5 | LOC100133724 /// | 100133724 /// | 5q31 |
| | | VDAC1 | 7416 | |
| 213502_x_at | 98.5 | LOC91316 | 91316 | 22q11.23 |
| 205826_at | 98.5 | MYOM2 | 9172 | 8p23.3 |
| 244387_at | 98.5 | PDE4D* | — | 5q12.1 |
| 1565762_at | 98.5 | RABGAP1L* | — | 1q25.1 |
| 205590_at | 98.5 | RASGRP1 | 10125 | 15q14 |
| 232914_{—}s_{—}at | 98.5 | SYTL2 | 54843 | 11q14 |
| 244043_at | 98.5 | TFDP2? | — | 3q23 |
| 223750_s_at | 98.5 | TLR10 | 81793 | 4p14 |
| 212038_s_at | 98.5 | VDAC1 | 7416 | 5q31 |
| 243734_x_at | 98.5 | VWC2? | — | 7p12.2 |
| 243526_at | 98.5 | WDR86 | 349136 | 7q36.1 |
| 234033_at | 98 | — | — | 4q32.1 |
| 203263_s_at | 98 | ARHGEF9 | 23229 | Xq11.1 |
| 213238_at | 98 | ATP10D | 57205 | 4p12 |
| 221234_s_at | 98 | BACH2 | 60468 | 6q15 |
| 218285_s_at | 98 | BDH2 | 56898 | 4q24 |
| 235952_at | 98 | DGKH-1* | — | 13q14.11 |
| 234912_at | 98 | DKFZP547L112 | 81787 | 15q11.2 |
| 213186_at | 98 | DZIP3 | 9666 | 3q13.13 |
| 50277_at | 98 | GGA1 | 26088 | 22q13.31 |
| 242952_at | 98 | HDAC9* | — | 7p21.1 |
| 214836_x_at | 98 | IGKC | 3514 | 2p12 |
| 237625_s_at | 98 | IGKC* | — | 2p12 |
| 225961_at | 98 | KLHDC5 | 57542 | 12p11.22 |
| 230551_at | 98 | KSR2 | 283455 | 12q24.22-q24.23 |
| 205386_s_at | 98 | MDM2 | 4193 | 12q14.3-q15 |
| 222350_at | 97.5 | BTBD3 | 22903 | 20p12.2 |
| 229715_at | 97.5 | BTBD6 | 90135 | 14q32 |
| 202946_s_at | 97.5 | IGKC | 3514 | 2p12 |
| 225389_at | 97.5 | KCNJ11? | — | 11p15.1 |
| 214669_x_at | 97.5 | LOC729082 | 729082 | 15q15.1 |
| 225332_at | 97.5 | NBPF1* | — | 1q21.1 |
| 213273_at | 97.5 | ODZ4 | 26011 | 11q14.1 |
| 235802_at | 97.5 | PLD4 | 122618 | 14q32.33 |
| 218526_s_at | 97.5 | RANGRF | 29098 | 17p13 |
| 230597_at | 97.5 | SLC7A3 | 84889 | Xq13.1 |

TABLE S12′
Top 100 Rank Order Genes Defining ROSE Cluster 4 (R4)

| Probeset | Rank | Symbol | EntrezID | Cytoband |
|---|---|---|---|---|
| 210356_x_at | 100.0% | MS4A1 | 931 | 11q12 |
| 217418_x_at | 100.0% | MS4A1 | 931 | 11q12 |
| 205401_at | 99.5% | AGPS | 8540 | 2q31.2 |
| 228592_at | 99.5% | MS4A1 | 931 | 11q12 |
| 241774_at | 99.5% | — | — | — |
| 218941_at | 99.5% | FBXW2 | 26190 | 9q34 |
| 225114_at | 99.0% | AGPS | 8540 | 2q31.2 |
| 202123_s_at | 99.0% | ABL1 | 25 | 9q34.1 |
| 203476_at | 99.0% | TPBG | 7162 | 6q14-q15 |
| 214783_s_at | 98.5% | ANXA11 | 311 | 10q23 |
| 202947_s_at | 98.5% | GYPC | 2995 | 2q14-q21 |
| 225833_at | 98.5% | DAGLB | 221955 | 7p22.1 |
| 225073_at | 98.5% | PPHLN1 | 51535 | 12q12 |
| 212730_at | 98.5% | SYNM | 23336 | 15q26.3 |
| 227846_at | 98.5% | GPR176 | 11245 | 15q14-q15.1 |
| 223991_s_at | 98.5% | GALNT2 /// | 100132910 /// | 18q12.2 /// |
| | | LOC100132910 | 2590 | 1q41-q42 |
| 208195_at | 98.0% | TTN | 7273 | 2q31 |
| 233713_at | 98.0% | — | — | — |
| 217788_s_at | 98.0% | GALNT2 | 2590 | 1q41-q42 |
| 224830_at | 98.0% | NUDT21 | 11051 | 16q13 |
| 226832_at | 98.0% | — | — | — |
| 202273_at | 98.0% | PDGFRB | 5159 | 5q31-q32 |
| 225376_at | 98.0% | C20orf11 | 54994 | 20q13.33 |
| 225281_at | 98.0% | C3orf17 | 25871 | 3q13.2 |
| 201096_s_at | 98.0% | ARF4 | 378 | 3p21.2- |
| | | | | p21.1 |
| 203948_s_at | 97.5% | MPO | 4353 | 17q23.1 |
| 1558017_s_at | 97.5% | — | — | — |
| 203949_at | 97.5% | MPO | 4353 | 17q23.1 |
| 1555392_at | 97.5% | LOC100128868 | 100128868 | 7q31.2 |
| 227541_at | 97.5% | WDR20 | 91833 | 14q32.31 |
| 1567458_s_at | 97.5% | RAC1 | 5879 | 7p22 |
| 213920_at | 97.5% | CUX2 | 23316 | 12q24.11-q24.12 |
| 224734_at | 97.5% | HMGB1 | 3146 | 13q12 |
| 206673_at | 97.5% | GPR176 | 11245 | 15q14-q15.1 |
| 224636_at | 97.5% | ZFP91 | 80829 | 11q12 |
| 235232_at | 97.5% | GMEB1 | 10691 | 1p35.3 |
| 208762_at | 97.5% | SUMO1 | 7341 | 2q33 |
| 36612_at | 97.0% | FAM168A | 23201 | 11q13.4 |
| 225240_s_at | 97.0% | MSI2 | 124540 | 17q22 |
| 336_at | 97.0% | TBXA2R | 6915 | 19p13.3 |
| 223101_s_at | 97.0% | ARPC5L | 81873 | 9q33.3 |
| 209049_s_at | 97.0% | ZMYND8 | 23613 | 20q13.12 |
| 217940_s_at | 97.0% | CARKD | 55739 | 13q34 |
| 216508_x_at | 97.0% | CTCFL /// HMGB1 /// | 100130561 /// | 13q12 /// 20q13.31 /// |
| | | HMGB1L1 /// | 100132863 /// 10357 /// | 20q13.32 /// |
| | | HMGB1L10 /// | 140690 /// 3146 | 22q12.1 /// 9q33.2 |
| | | LOC100132863 | | |
| 201266_at | 97.0% | TXNRD1 | 7296 | 12q23-q24.1 |
| 212286_at | 97.0% | ANKRD12 | 23253 | 18p11.22 |
| 200618_at | 97.0% | LASP1 | 3927 | 17q11-q21.3 |
| 227577_at | 97.0% | EXOC8 | 149371 | 1q42.2 |
| 203068_at | 97.0% | KLHL21 | 9903 | 1p36.31 |
| 217787_s_at | 97.0% | GALNT2 | 2590 | 1q41-q42 |
| 239930_at | 97.0% | GALNT2 | 2590 | 1q41-q42 |
| 227700_x_at | 97.0% | ATAD3A | 55210 | 1p36.33 |
| 225694_at | 97.0% | CRKRS | 51755 | 17q12 |
| 202514_at | 97.0% | DLG1 | 1739 | 3q29 |
| 226115_at | 97.0% | AHCTF1 | 25909 | 1q44 |
| 1562948_at | 97.0% | — | — | — |
| 225456_at | 97.0% | MED1 | 5469 | 17q12-q21.1 |
| 208821_at | 97.0% | SNRPB | 6628 | 20p13 |
| 212204_at | 97.0% | TMEM87A | 25963 | 15q15.1 |
| 231124_x_at | 97.0% | LY9 | 4063 | 1q21.3-q22 |
| 218118_s_at | 97.0% | TIMM23 | 10431 | 10q11.21-q11.23 |
| 212272_at | 96.5% | LPIN1 | 23175 | 2p25.1 |
| 220684_at | 96.5% | TBX21 | 30009 | 17q21.32 |
| 216836_s_at | 96.5% | ERBB2 | 2064 | 17q11.2-q12\|17q21.1 |
| 232521_at | 96.5% | PCSK7 | 9159 | 11q23-q24 |
| 205839_s_at | 96.5% | BZRAP1 | 9256 | 17q22-q23 |
| 218031_s_at | 96.5% | FOXN3 | 1112 | 14q31.3 |
| 226640_at | 96.5% | DAGLB | 221955 | 7p22.1 |
| 213514_s_at | 96.5% | DIAPH1 | 1729 | 5q31 |
| 225494_at | 96.5% | DYNLL2 | 140735 | 17q22 |
| 213222_at | 96.5% | PLCB1 | 23236 | 20p12 |
| 212594_at | 96.5% | PDCD4 | 27250 | 10q24 |
| 201133_s_at | 96.5% | PJA2 | 9867 | 5q21.3 |
| 235463_s_at | 96.5% | LASS6 | 253782 | 2q24.3 |
| 200047_s_at | 96.5% | YY1 | 7528 | 14q |
| 201407_s_at | 96.5% | PPP1CB | 5500 | 2p23 |
| 1552931_a_at | 96.5% | PDE8A | 5151 | 15q25.3 |
| 242467_at | 96.5% | — | — | — |
| 213860_x_at | 96.5% | CSNK1A1 | 1452 | 5q32 |
| 212927_at | 96.5% | SMC5 | 23137 | 9q21.11 |
| 227237_x_at | 96.5% | ATAD3B /// | 732419 /// 83858 | 1p36.33 |
| | | LOC732419 | | |
| 200775_s_at | 96.5% | HNKNPK | 3190 | 9q21.32-q21.33 |
| 210203_at | 96.5% | CNOT4 | 4850 | 7q22-qter |
| 214352_s_at | 96.5% | KRAS | 3845 | 12p12.1 |
| 1555772_a_at | 96.5% | CDC25A | 993 | 3p21 |
| 212696_s_at | 96.5% | RNF4 | 6047 | 4p16.3 |
| 235233_s_at | 96.5% | GMEB1 | 10691 | 1p35.3 |
| 225535_s_at | 96.5% | TIMM23 | 10431 | 10q11.21-q11.23 |
| 1555762_s_at | 96.5% | RBM15 | 64783 | 1p13 |
| 204735_at | 96.5% | PDE4A | 5141 | 19p13.2 |
| 228599_at | 96.0% | MS4A1 | 931 | 11q12 |
| 212511_at | 96.0% | PICALM | 8301 | 11q14 |
| 207681_at | 96.0% | CXCR3 | 2833 | Xq13 |
| 224912_at | 96.0% | TTC7A | 57217 | 2p21 |
| 218447_at | 96.0% | C16orf61 | 56942 | 16q23.2 |
| 204206_at | 96.0% | MNT | 4335 | 17p13.3 |
| 227433_at | 96.0% | KIAA2018 | 205717 | 3q13.2 |
| 224617_at | 96.0% | ROD1 | 9991 | 9q32 |
| 1560339_s_at | 96.0% | NAP1L4 | 4676 | 11p15.5 |
| 201015_s_at | 96.0% | JUP | 3728 | 17q21 |

TABLE S13′
Top 100 Rank Order Genes Defining ROSE Cluster 5 (R5)

| | Per- | | | |
|---|---|---|---|---|
| Probeset | centile | Symbol | EntrezID | Cytoband |
| 202804_at | 100 | ABCC1 | 4363 | 16p13.1 |
| 204638_at | 100 | ACP5 | 54 | 19p13.3-p13.2 |
| 205423_at | 100 | AP1B1 | 162 | 22q12\|22q12.2 |
| 212062_at | 100 | ATP9A | 10079 | 20q13.2 |
| 216129_at | 100 | ATP9A | 10079 | 20q13.2 |
| 236226_at | 100 | BTLA | 151888 | 3q13.2 |
| 209498_at | 100 | CEACAM1 | 634 | 19q13.2 |
| 222786_at | 100 | CHST12 | 55501 | 7p22 |
| 218927_s_at | 100 | CHST12 | 55501 | 7p22 |
| 219500_at | 100 | CLCF1 | 23529 | 11q13.3 |
| 1556385_at | 100 | CLCF1* | — | 11q13.1 |
| 201445_at | 100 | CNN3 | 1266 | 1p22-p21 |
| 228297_at | 100 | CNN3* | — | 1p21.3 |
| 228585_at | 100 | ENTPD1 | 953 | 10q24 |
| 1554903_at | 100 | FRMD8 | 83786 | 11q13 |
| 1554905_x_at | 100 | FRMD8 | 83786 | 11q13 |
| 227964_at | 100 | FRMD8 | 83786 | 11q13 |
| 230788_at | 100 | GCNT2 | 2651 | 6p24.2 |
| 202032_s_at | 100 | MAN2A2 | 4122 | 15q26.1 |
| 209703_x_at | 100 | METTL7A | 25840 | 12q13.13 |
| 226531_at | 100 | ORAI1 | 84876 | 12q24.31 |
| 60471_at | 100 | RIN3 | 79890 | 14q32.12 |
| 207735_at | 100 | RNF125 | 54941 | 18q12.1 |
| 229661_at | 100 | SALL4 | 57167 | 20q13.13-q13.2 |
| 222088_s_at | 100 | SLC2A14 /// | 144195 /// | 12p13.3 /// |
| | | SLC2A3 | 6515 | 12p13.31 |
| 202498_s_at | 100 | SLC2A3 | 6515 | 12p13.3 |
| 202499_s_at | 100 | SLC2A3 | 6515 | 12p13.3 |
| 213083_at | 100 | SLC35D2 | 11046 | 9q22.32 |
| 215447_at | 100 | TFPI | 7035 | 2q32 |
| 231775_at | 100 | TNFRSF10A | 8797 | 8p21 |
| 227595_at | 100 | ZMYM6 | 9204 | 1p34.2 |
| 243121_x_at | 99.5 | — | — | 19q13.41 |
| 223646_s_at | 99.5 | CYorf15B | 84663 | Yq11.222 |
| 203139_at | 99.5 | DAPK1 | 1612 | 9q34.1 |
| 211214_s_at | 99.5 | DAPK1 | 1612 | 9q34.1 |
| 223306_at | 99.5 | EBPL | 84650 | 13q12-q13 |
| 209474_s_at | 99.5 | ENTPD1 | 953 | 10q24 |
| 209473_at | 99.5 | ENTPD1 | 953 | 10q24 |
| 229280_s_at | 99.5 | FLJ22536 | 401237 | 6p22.3 |
| 228188_at | 99.5 | FOSL2 | 2355 | 2p23.3 |
| AFFX- | 99.5 | GAPDH | 2597 | 12p13 |
| HUMGAPDH/ | | | | |
| M33197_5_at | | | | |
| 204689_at | 99.5 | HHEX | 3087 | 10q23.33 |
| 1552623_at | 99.5 | HSH2D | 84941 | 19p13.11 |
| 207761_s_at | 99.5 | METTL7A | 25840 | 12q13.13 |
| 207132_x_at | 99.5 | PFDN5 | 5204 | 12q12 |
| 1557948_at | 99.5 | PHLDB3 | 653583 | 19q13.31 |
| 213362_at | 99.5 | PTPRD | 5789 | 9p23-p24.3 |
| 227983_at | 99.5 | RILPL2 | 196383 | 12q24.31 |
| 219457_s_at | 99.5 | RIN3 | 79890 | 14q32.12 |
| 211474_s_at | 99.5 | SERPINB6 | 5269 | 6p25 |
| 223196_s_at | 99.5 | SESN2 | 83667 | 1p35.3 |
| 216236_s_at | 99.5 | SLC2A14 /// | 144195 /// | 12p13.3 /// |
| | | SLC2A3 | 6515 | 12p13.31 |
| 202497_x_at | 99.5 | SLC2A3 | 6515 | 12p13.3 |
| 227594_at | 99.5 | ZMYM6 | 9204 | 1p34.2 |
| 202805_s_at | 99 | ABCC1 | 4363 | 16p13.1 |
| 213346_at | 99 | C13orf27 | 93081 | 13q33.1 |
| 223527_s_at | 99 | CDADC1 | 81602 | 13q14.2 |
| 213060_s_at | 99 | CHI3L2 | 1117 | 1p13.3 |
| 203277_at | 99 | DFFA | 1676 | 1p36.3-p36.2 |
| 208887_at | 99 | EIF3G | 8666 | 19p13.2 |
| 219016_at | 99 | FASTKD5 | 60493 | 20p13 |
| 218034_at | 99 | FIS1 | 51024 | 7q22.1 |
| 225163_at | 99 | FRMD4A | 55691 | 10p13 |
| 239606_at | 99 | GCNT2A* | — | 6p24.2 |
| 230348_at | 99 | LATS2 | 26524 | 13q11-q12 |
| 209332_s_at | 99 | MAX | 4149 | 14q23 |
| 227379_at | 99 | MBOAT1 | 154141 | 6p22.3 |
| 217980_s_at | 99 | MRPL16 | 54948 | 11q12-q13.1 |
| 238082_at | 99 | PLEKHA2* | — | 8p11.23 |
| 232473_at | 99 | PRPF18 | 8559 | 10p13 |
| 220330_s_at | 99 | SAMSN1 | 64092 | 21q11 |
| 223917_s_at | 99 | SLC39A3 | 29985 | 19p13.3 |
| 219257_s_at | 99 | SPHK1 | 8877 | 17q25.2 |
| 203544_s_at | 99 | STAM | 8027 | 10p14-p13 |
| 213258_at | 99 | TFPI | 7035 | 2q32 |
| 210664_s_at | 99 | TFPI | 7035 | 2q32 |
| 210665_at | 99 | TFPI | 7035 | 2q32 |
| 201379_s_at | 99 | TPD52L2 | 7165 | 20q13.2-q13.3 |
| 212481_s_at | 99 | TPM4 | 7171 | 19p13.1 |
| 235094_at | 99 | TPM4* | — | 19p13.2 |
| 212923_s_at | 98.5 | C6orf145 | 221749 | 6p25.2 |
| 206120_at | 98.5 | CD33 | 945 | 19q13.3 |
| 1559916_a_at | 98.5 | CHST12* | — | 7p22.2 |
| 1554464_a_at | 98.5 | CRTAP | 10491 | 3p22.3 |
| 209774_x_at | 98.5 | CXCL2 | 2920 | 4q21 |
| 225168_at | 98.5 | FRMD4A | 55691 | 10p13 |
| 213453_x_at | 98.5 | GAPDH | 2597 | 12p13 |
| 209604_s_at | 98.5 | GATA3 | 2625 | 10p15 |
| 209602_s_at | 98.5 | GATA3 | 2625 | 10p15 |
| 204000_at | 98.5 | GNB5 | 10681 | 15q21.2 |
| 233877_at | 98.5 | GOLIM4* | — | 3q26.2 |
| 203395_s_at | 98.5 | HES1 | 3280 | 3q28-q29 |
| 214950_at | 98.5 | IL9R /// | 3581 /// | 16p13.3 /// Xq28 |
| | | LOC729486 | 729486 | and Yq12 |
| 213923_at | 98.5 | RAP2B | 5912 | 3q25.2 |
| 238091_at | 98.5 | RPH3AL* | — | 17p13.3 |
| 236501_at | 98.5 | SALL4 | 57167 | 20q13.13-q13.2 |
| 223195_s_at | 98.5 | SESN2 | 83667 | 1p35.3 |
| 227518_at | 98.5 | SLC35E1 | 79939 | 19p13.11 |
| 243981_at | 98.5 | STK4 | 6789 | 20q11.2-q13.2 |
| 212369_at | 98.5 | ZNF384 | 171017 | 12p12 |

TABLE S14′
Top 100 Rank Order Genes Defining ROSE Cluster 6 (R6)

| | Per- | | | |
|---|---|---|---|---|
| Probeset | centile | Symbol | EntrezID | Cytoband |
| 242457_at | 100 | — | — | 5q21.1 |
| 204066_s_at | 100 | AGAP1 | 116987 | 2q37 |
| 233038_at | 100 | AGAP1* | — | 2q37.2 |
| 233225_at | 100 | AGAP1* | — | 2q37.2 |
| 235968_at | 100 | AGAP1* | — | 2q37.2 |
| 240758_at | 100 | AGAP1* | — | 2q37.2 |
| 228240_at | 100 | AGAP1? | — | 2q37.2 |
| 206756_at | 100 | CHST7 | 56548 | Xp11.23 |
| 200614_at | 100 | CLTC | 1213 | 17q11-qter |
| 231166_at | 100 | GPR155 | 151556 | 2q31.1 |
| 228863_at | 100 | PCDH17 | 27253 | 13q21.1 |
| 227289_at | 100 | PCDH17 | 27253 | 13q21.1 |
| 205656_at | 100 | PCDH17 | 27253 | 13q21.1 |
| 230537_at | 100 | PCDH17? | — | 13q21.1 |
| 203335_at | 100 | PHYH | 5264 | 10p13 |
| 1555579_s_at | 100 | PTPRM | 5797 | 18p11.2 |
| 203329_at | 100 | PTPRM | 5797 | 18p11.2 |
| 1554343_a_at | 100 | STAP1 | 26228 | 4q13.2 |
| 220059_at | 100 | STAP1 | 26228 | 4q13.2 |
| 211890_x_at | 99.5 | CAPN3 | 825 | 15q15.1- |
| | | | | q21.1 |
| 219470_x_at | 99.5 | CCNJ | 54619 | 10pter- |
| | | | | q26.12 |
| 229091_s_at | 99.5 | CCNJ | 54619 | 10pter- |
| | | | | q26.12 |
| 239956_at | 99.5 | CHST2? | — | 3q23 |
| 1552398_a_at | 99.5 | CLEC12A /// | 160364 /// | 12p13.2 |
| | | CLEC12B | 387837 | |
| 219821_s_at | 99.5 | GFOD1 | 54438 | 6pter- |
| | | | | p22.1 |
| 239533_at | 99.5 | GPR155 | 151556 | 2q31.1 |
| 202409_at | 99.5 | IGF2 /// | 3481 /// | 11p15.5 |
| | | INS-IGF2 | 723961 | |
| 230179_at | 99.5 | LOC285812 | 285812 | 6p23 |
| 202819_s_at | 99.5 | TCEB3 | 6924 | 1p36.1 |
| 232081_at | 99 | ABCG1? | — | 21q22.3 |
| 1561786_at | 99 | AGAP1* | — | 2q37.2 |
| 1559280_a_at | 99 | AK092578* | — | 4q32.3 |
| 1554486_a_at | 99 | C6orf114 | 85411 | 6p23 |
| 1558621_at | 99 | CABLES1 | 91768 | 18q11.2 |
| 203921_at | 99 | CHST2 | 9435 | 3q24 |
| 209087_x_at | 99 | MCAM | 4162 | 11q23.3 |
| 211340_s_at | 99 | MCAM | 4162 | 11q23.3 |
| 223130_s_at | 99 | MYLIP | 29116 | 6p23-p22.3 |
| 228098_s_at | 99 | MYLIP | 29116 | 6p23-p22.3 |
| 226814_at | 98.5 | ADAMTS9 | 56999 | 3p14.3- |
| | | | | p14.2 |
| 238987_at | 98.5 | B4GALT1 | 2683 | 9p13 |
| 225499_at | 98.5 | c20orf74? | — | 20p11.23 |
| 1556593_s_at | 98.5 | CHST2? | — | 3q23 |
| 231600_at | 98.5 | CLEC12B | 387837 | 12p13.2 |
| 214683_s_at | 98.5 | CLK1 | 1195 | 2q33 |
| 201656_at | 98.5 | ITGA6 | 3655 | 2q31.1 |
| 202746_at | 98.5 | ITM2A | 9452 | Xq13.3- |
| | | | | Xq21.2 |
| 210869_s_at | 98.5 | MCAM | 4162 | 11q23.3 |
| 1569484_s_at | 98.5 | MDN1 | 23195 | 6q15 |
| 228097_at | 98.5 | MYLIP | 29116 | 6p23-p22.3 |
| 229407_at | 98.5 | SDK1 | 221935 | 7p22.2 |
| 209593_s_at | 98.5 | TOR1B | 27348 | 9q34 |
| 222281_s_at | 98 | c1orf186* | — | 1q32.1 |
| 239826_at | 98 | CABLES1* | — | 18q11.2 |
| 214475_x_at | 98 | CAPN3 | 825 | 15q15.1- |
| | | | | q21.1 |
| 210944_s_at | 98 | CAPN3 | 825 | 15q15.1- |
| | | | | q21.1 |
| 1556592_at | 98 | CHST2? | — | 3q23 |
| 211623_s_at | 98 | FBL | 2091 | 19q13.1 |
| 234339_s_at | 98 | GLTSCR2 | 29997 | 19q13.3 |
| 225330_at | 98 | IGF1R | 3480 | 15q26.3 |
| 212978_at | 98 | LRRC8B | 23507 | 1p22.2 |
| 215692_s_at | 98 | MPPED2 | 744 | 11p13 |
| 205413_at | 98 | MPPED2 | 744 | 11p13 |
| 223129_x_at | 98 | MYLIP | 29116 | 6p23-p22.3 |
| 232280_at | 98 | SLC25A29 | 123096 | 14q32.2 |
| 202818_s_at | 98 | TCEB3 | 6924 | 1p36.1 |
| 225127_at | 98 | TMEM181 | 57583 | 6q25.3 |
| 241535_at | 97.5 | — | — | 2p25.3 |
| 233867_at | 97.5 | AKAP13* | — | 15q25.3 |
| 212702_s_at | 97.5 | BICD2 | 23299 | 9q22.31 |
| 224435_at | 97.5 | C10orf57 /// | 80195 /// | 10q22.3 /// |
| | | C10orf58 | 84293 | 10q23.1 |
| 242406_at | 97.5 | c1orf186* | — | 1q32.1 |
| 230954_at | 97.5 | C20orf112 | 140688 | 20q11.1- |
| | | | | q11.23 |
| 220331_at | 97.5 | CYP46A1 | 10858 | 14q32.1 |
| 204836_at | 97.5 | GLDC | 2731 | 9p22 |
| 215177_s_at | 97.5 | ITGA6 | 3655 | 2q31.1 |
| 230591_at | 97.5 | LOC729887 | 729887 | 16q24.1 |
| 227805_at | 97.5 | MAP1D? | — | 2q31.1 |
| 209086_x_at | 97.5 | MCAM | 4162 | 11q23.3 |
| 223627_at | 97.5 | MEX3B | 84206 | 15q25.2 |
| 220319_s_at | 97.5 | MYLIP | 29116 | 6p23-p22.3 |
| 223096_at | 97.5 | NOP5/NOP58 | 51602 | 2q33.1 |
| 243612_at | 97.5 | NSD1 | 64324 | 5q35.2- |
| | | | | q35.3 |
| 214620_x_at | 97.5 | PAM | 5066 | 5q14-q21 |
| 202336_s_at | 97.5 | PAM | 5066 | 5q14-q21 |
| 242664_at | 97.5 | PTPRM* | — | 18p11.23 |
| 226342_at | 97.5 | SPTBN1 | 6711 | 2p21 |
| 229594_at | 97.5 | SPTY2D1 | 144108 | 11p15.1 |
| 239361_at | 97 | CABLES1* | — | 18q11.2 |
| 220450_at | 97 | — | — | 4q31.22 |
| 204567_s_at | 97 | ABCG1 | 9619 | 21q22.3 |
| 229720_at | 97 | BAG1 | 573 | 9p12 |
| 243409_at | 97 | FOXL1 | 2300 | 16q24 |
| 202747_s_at | 97 | ITM2A | 9452 | Xq13.3- |
| | | | | Xq21.2 |
| 212658_at | 97 | LHFPL2 | 10184 | 5q14.1 |
| 225611_at | 97 | LOC100128443 | 100128443 | 5q12.3 |
| | | /// MAST4 | /// 375449 | |
| 212239_at | 97 | PIK3R1 | 5295 | 5q13.1 |
| 226143_at | 97 | RAI1 | 10743 | 17p11.2 |
| 1552329_at | 97 | RBBP6 | 5930 | 16p12.2 |
| 225305_at | 97 | SLC25A29 | 123096 | 14q32.2 |

TABLE S15′
Top 100 Rank Order Genes Defining ROSE Cluster 8 (R8)

| Probeset | Rank | Symbol | EntrezID | Cytoband |
|---|---|---|---|---|
| 238689_at | 100.0 | GPR110 | 266977 | 6p12.3 |
| 235988_at | 100.0 | GPR110 | 266977 | 6p12.3 |
| 236489_at | 100.0 | GPR110? | — | 6p12.3 |
| 217109_at | 100.0 | MUC4 | 4585 | 3q29 |
| 217110_s_at | 99.5 | MUC4 | 4585 | 3q29 |
| 205795_at | 99.5 | NRXN3 | 9369 | 14q31 |
| 216565_x_at | 99.0 | — | — | 1p36.11 |
| 214022_s_at | 99.0 | IFITM1 | 8519 | 11p15.5 |
| 201601_x_at | 99.0 | IFITM1 | 8519 | 11p15.5 |
| 204895_x_at | 99.0 | MUC4 | 4585 | 3q29 |
| 206873_at | 98.5 | CA6 | 765 | 1p36.2 |
| 201028_s_at | 98.5 | CD99 | 4267 | Xp22.32; |
| | | | | Yp11.3 |
| 242051_at | 98.5 | CD99? | — | Xp22.32; |
| | | | | Yp11.3 |
| 240586_at | 98.5 | ENAM | 10117 | 4q13.3 |
| 212592_at | 98.5 | IGJ | 3512 | 4q21 |
| 223304_at | 98.5 | SLC37A3 | 84255 | 7q34 |
| 1569666_s_at | 98.5 | SLC37A3* | — | 7q34 |
| 238063_at | 98.5 | TMEM154 | 201799 | 4q31.3 |
| 207900_at | 98.0 | CCL17 | 6361 | 16q13 |
| 201029_s_at | 98.0 | CD99 | 4267 | Xp22.32; |
| | | | | Yp11.3 |
| 214907_at | 98.0 | CEACAM21 | 90273 | 19q13.2 |
| 201315_x_at | 98.0 | IFITM2 | 10581 | 11p15.5 |
| 222154_s_at | 98.0 | LOC26010 | 26010 | 2q33.1 |
| 211675_s_at | 98.0 | MDFIC | 29969 | 7q31.1-q31.2 |
| 239272_at | 98.0 | MMP28 | 79148 | 17q11-q21.1 |
| 212183_at | 98.0 | NUDT4 /// | 11163 /// | 12q21 /// |
| | | NUDT4P1 | 440672 | 1q21.1 |
| 212181_s_at | 98.0 | NUDT4 /// | 11163 /// | 12q21 /// |
| | | NUDT4P1 | 440672 | 1q21.1 |
| 220024_s_at | 98.0 | PRX | 57716 | 19q13.13- |
| | | | | q13.2 |
| 207426_s_at | 98.0 | TNFSF4 | 7292 | 1q25 |
| 208303_s_at | 97.4 | CRLF2 | 64109 | Xp22.3; |
| | | | | Yp11.3 |
| 205983_at | 97.4 | DPEP1 | 1800 | 16q24.3 |
| 207651_at | 97.4 | GPR171 | 29909 | 3q25.1 |
| 213371_at | 97.4 | LDB3 | 11155 | 10q22.3- |
| | | | | q23.2 |
| 1559315_s_at | 97.4 | LOC144481 | 144481 | 12q22 |
| 226382_at | 97.4 | LOC283070 | 283070 | 10p14 |
| 229334_at | 97.4 | RUFY3 | 22902 | 4q13.3 |
| 225244_at | 97.4 | SNAP47 | 116841 | 1q42.13 |
| 203372_s_at | 97.4 | SOCS2 | 8835 | 12q |
| 244721_at | 97.4 | TP53INP1 | 94241 | 8q22 |
| 218862_at | 96.9 | ASB13 | 79754 | 10p15.1 |
| 206150_at | 96.9 | CD27 | 939 | 12p13 |
| 218013_x_at | 96.9 | DCTN4 | 51164 | 5q31-q32 |
| 219777_at | 96.9 | GIMAP6 | 474344 | — |
| 233884_at | 96.9 | HIVEP3 | 59269 | 1p34 |
| 203435_s_at | 96.9 | MME | 4311 | 3q25.1- |
| | | | | q25.2 |
| 239273_s_at | 96.9 | MMP28 | 79148 | 17q11-q21.1 |
| 202149_at | 96.9 | NEDD9 | 4739 | 6p25-p24 |
| 205259_at | 96.9 | NR3C2 | 4306 | 4q31.1 |
| 215021_s_at | 96.9 | NRXN3 | 9369 | 14q31 |
| 236750_at | 96.9 | NRXN3* | — | 14q31.1 |
| 228696_at | 96.9 | SLC45A3 | 85414 | 1q32.1 |
| 223741_s_at | 96.9 | TTYH2 | 94015 | 17q25.1 |
| 219141_s_at | 96.4 | AMBRA1 | 55626 | 11p11.2 |
| 230161_at | 96.4 | CD99* | — | Xp22.32; |
| | | | | Yp11.3 |
| 223377_x_at | 96.4 | CISH | 1154 | 3p21.3 |
| 229114_at | 96.4 | GAB1 | 2549 | 4q31.21 |
| 1552316_a_at | 96.4 | GIMAP1 | 170575 | 7q36.1 |
| 229649_at | 96.4 | NRXN3 | 9369 | 14q31 |
| 226433_at | 96.4 | RNF157 | 114804 | 17q25.1 |
| 220454_s_at | 96.4 | SEMA6A | 57556 | 5q23.1 |
| 225660_at | 96.4 | SEMA6A | 57556 | 5q23.1 |
| 230747_s_at | 96.4 | TTC39C | 125488 | 18q11.2 |
| 1555194_at | 96.4 | TTC39C* | — | 18q11.2 |
| 203756_at | 95.9 | ARHGEF17 | 9828 | 11q13.4 |
| 242579_at | 95.9 | BMPR1B | 658 | 4q22-q24 |
| 212974_at | 95.9 | DENND3 | 22898 | 8q24.3 |
| 217967_s_at | 95.9 | FAM129A | 116496 | 1q25 |
| 226002_at | 95.9 | GAB1 | 2549 | 4q31.21 |
| 207375_s_at | 95.9 | IL15RA | 3601 | 10p15-p14 |
| 208071_s_at | 95.9 | LAIR1 | 3903 | 19q13.4 |
| 210644_s_at | 95.9 | LAIR1 | 3903 | 19q13.4 |
| 215020_at | 95.9 | NRXN3 | 9369 | 14q31 |
| 238297_at | 95.9 | PHACTR1* | — | 6p24.1 |
| 210830_s_at | 95.9 | PON2 | 5445 | 7q21.3 |
| 203373_at | 95.9 | SOCS2 | 8835 | 12q |
| 225912_at | 95.9 | TP53INP1 | 94241 | 8q22 |
| 225108_at | 95.4 | AGPS | 8540 | 2q31.2 |
| 229975_at | 95.4 | BMPR1B | 658 | 4q22- |
| | | | | q24 |
| 202910_s_at | 95.4 | CD97 | 976 | 19p13 |
| 216605_s_at | 95.4 | CEACAM21 | 90273 | 19q13.2 |
| 229604_at | 95.4 | CMAH | 8418 | 6p21.32 |
| 1556037_s_at | 95.4 | HHIP | 64399 | 4q28-q32 |
| 244764_at | 95.4 | HIVEP3* | — | 1p34.2 |
| 222762_x_at | 95.4 | LIMD1 | 8994 | 3p21.3 |
| 236632_at | 95.4 | LOC646576 | 646576 | 4q31.22 |
| 240457_at | 95.4 | NEURL1B* | — | 5q35.1 |
| 1553995_a_at | 95.4 | NT5E | 4907 | 6q14-q21 |
| 219812_at | 95.4 | PVRIG | 79037 | 7q22.1 |
| 52731_at | 94.9 | AMBRA1 | 55626 | 11p11.2 |
| 236766_at | 94.9 | C8orf38* | — | 8q22.1 |
| 221223_x_at | 94.9 | CISH | 1154 | 3p21.3 |
| 209210_s_at | 94.9 | FERMT2 | 10979 | 14q22.2 |
| 238880_at | 94.9 | GTF3A | 2971 | 13q12.3- |
| | | | | q13.1 |
| 212203_x_at | 94.9 | IFITM3 | 10410 | 11p15.5 |
| 209695_at | 94.9 | LOC100131062 | 100131062 /// | 8q24.3 |
| | | /// PTP4A3 | 11156 | |
| 51146_at | 94.9 | PIGV | 55650 | 1p36.11 |
| 219238_at | 94.9 | PIGV | 55650 | 1p36.11 |
| 48106_at | 94.9 | SLC48A1 | 55652 | 12q13.11 |
| 226838_at | 94.9 | TTC32 | 130502 | 2p24.1 |
| 230643_at | 94.9 | WNT9A | 7483 | 1q42 |

TABLE S16′
Top 100 Rank Order Genes Associated with Unclustered ROSE Samples (R7)

| Probeset | Percentile | Symbol | EntrezID | Cytoband |
|---|---|---|---|---|
| 220230_s_at | 96.2 | CYB5R2 | 51700 | 11p15.4 |
| 212188_at | 93.7 | KCTD12 | 115207 | 13q22.3 |
| 242593_at | 93.1 | — | — | ? |
| 1564878_at | 93.1 | — | — | 12q24.23-q24.31 |
| 227435_at | 93.1 | KIAA2018 | 205717 | 3q13.2 |
| 226869_at | 93.1 | MEGF6 | 1953 | 1p36.3 |
| 200866_s_at | 93.1 | PSAP | 5660 | 10q21-q22 |
| 212956_at | 93.1 | TBC1D9 | 23158 | 4q31.21 |
| 205987_at | 91.8 | CD1C | 911 | 1q22-q23 |
| 229288_at | 91.8 | EPHA7 | 2045 | 6q16.1 |
| 229716_at | 91.2 | — | — | 1p36.12 |
| 1556682_s_at | 91.2 | AUTS2* | — | 7q11.22 |
| 226640_at | 91.2 | DAGLB | 221955 | 7p22.1 |
| 238533_at | 91.2 | EPHA7 | 2045 | 6q16.1 |
| 204396_s_at | 91.2 | GRK5 | 2869 | 10q24-qter |
| 240413_at | 91.2 | PYHIN1 | 149628 | 1q23.1 |
| 213164_at | 91.2 | SLC5A3 | 6526 | 21q22.12 |
| 242644_at | 91.2 | TMC8 | 147138 | 17q25.3 |
| 237946_at | 90.6 | — | — | 11p15.4 |
| 229967_at | 90.6 | CMTM2 | 146225 | 16q21 |
| 221773_at | 90.6 | ELK3 | 2004 | 12q23 |
| 205718_at | 90.6 | ITGB7 | 3695 | 12q13.13 |
| 212192_at | 90.6 | KCTD12 | 115207 | 13q22.3 |
| 1559263_s_at | 90.6 | PPIL4 /// | 340152 /// | 6q24-q25 /// |
| | | ZC3H12D | 85313 | 6q25.1 |
| 218613_at | 90.6 | PSD3 | 23362 | 8pter- |
| | | | | p23.3 |
| 203355_s_at | 90.6 | PSD3 | 23362 | 8pter- |
| | | | | p23.3 |
| 221808_at | 90.6 | RAB9A | 9367 | Xp22.2 |
| 227210_at | 90.6 | SFMBT2? | — | 10p14 |
| 202912_at | 89.9 | ADM | 133 | 11p15.4 |
| 205290_s_at | 89.9 | BMP2 | 650 | 20p12 |
| 219837_s_at | 89.9 | CYTL1 | 54360 | 4p16-p15 |
| 213316_at | 89.9 | KIAA1462 | 57608 | 10p11.23 |
| 210629_x_at | 89.9 | LST1 | 7940 | 6p21.3 |
| 220122_at | 89.9 | MCTP1 | 79772 | 5q15 |
| 214735_at | 89.9 | PIP3-E | 26034 | 6q25.2 |
| 209568_s_at | 89.9 | RGL1 | 23179 | 1q25.3 |
| 226207_at | 89.9 | RILPL1 | 353116 | 12q24.31 |
| 212944_at | 89.9 | SLC5A3 | 6526 | 21q22.12 |
| 207777_s_at | 89.9 | SP140 | 11262 | 2q37.1 |
| 226080_at | 89.9 | SSH2 | 85464 | 17q11.2 |
| 230590_at | 89.9 | SSH2* | — | 17q11.2 |
| 223375_at | 89.9 | TBC1D22B | 55633 | 6p21.2 |
| 224967_at | 89.9 | UGCG | 7357 | 9q31 |
| 213618_at | 89.3 | ARAP2 | 116984 | 4p14 |
| 203923_s_at | 89.3 | CYBB | 1536 | Xp21.1 |
| 225833_at | 89.3 | DAGLB | 221955 | 7p22.1 |
| 214574_x_at | 89.3 | LST1 | 7940 | 6p21.3 |
| 207339_s_at | 89.3 | LIB | 4050 | 6p21.3 |
| 217418_x_at | 89.3 | MS4A1 | 931 | 11q12 |
| 200871_s_at | 89.3 | PSAP | 5660 | 10q21-q22 |
| 216748_at | 89.3 | PYHIN1 | 149628 | 1q23.1 |
| 204688_at | 89.3 | SGCE | 8910 | 7q21-q22 |
| 204328_at | 89.3 | TMC6 | 11322 | 17q25.3 |
| 227353_at | 89.3 | TMC8 | 147138 | 17q25.3 |
| 233596_at | 89.3 | UIMC1* | — | 5q35.2 |
| 229040_at | 88.7 | BC40064* | — | 21q22.3 |
| 203922_s_at | 88.7 | CYBB | 1536 | Xp21.1 |
| 204057_at | 88.7 | IRF8 | 3394 | 16q24.1 |
| 218656_s_at | 88.7 | LHFP | 10186 | 13q12 |
| 211101_x_at | 88.7 | LILRA2 | 11027 | 19q13.4 |
| 239062_at | 88.7 | LOC100131096 | 100131096 | 17q25.3 |
| 206940_s_at | 88.7 | LOC100131317 /// | 100131317 /// | 13q31.1 |
| | | POU4F1 | 5457 | |
| 211581_x_at | 88.7 | LST1 | 7940 | 6p21.3 |
| 244230_at | 88.7 | MEF2C* | — | 5q14.3 |
| 1569136_at | 88.7 | MGAT4A | 11320 | 2q12 |
| 1569931_at | 88.7 | NCOR2* | — | 12q24.31 |
| 241387_at | 88.7 | PTK2* | — | 8q24.3 |
| 41220_at | 88.7 | SEPT9* | 10801 | 17q25.2-q25.3 |
| 208657_s_at | 88.7 | SEPT9* | 10801 | 17q25.2-q25.3 |
| 231837_at | 88.7 | USP28 | 57646 | 11q23 |
| 1552678_a_at | 88.7 | USP28 | 57646 | 11q23 |
| 236635_at | 88.7 | ZNF667 | 63934 | 19q13.43 |
| 231418_at | 88.1 | — | — | 11q12.2 |
| 229041_s_at | 88.1 | BC40064* | — | 21q22.3 |
| 205289_at | 88.1 | BMP2 | 650 | 20p12 |
| 37170_at | 88.1 | BMP2K | 55589 | 4q21.21 |
| 225828_at | 88.1 | DAGLB | 221955 | 7p22.1 |
| 214966_at | 88.1 | GRIK5 | 2901 | 19q13.2 |
| 1555349_a_at | 88.1 | ITGB2 | 3689 | 21q22.3 |
| 227433_at | 88.1 | KIAA2018 | 205717 | 3q13.2 |
| 232935_at | 88.1 | LHFP* | — | 13q13.3 |
| 215633_x_at | 88.1 | LST1 | 7940 | 6p21.3 |
| 214181_x_at | 88.1 | LST1 | 7940 | 6p21.3 |
| 242191_at | 88.1 | NBPF10 /// RP11-94I2.2 | 100132406 /// 200030 | 1q21.1 |
| 209949_at | 88.1 | NCF2 | 4688 | 1q25 |
| 206370_at | 88.1 | PIK3CG | 5294 | 7q22.3 |
| 203038_at | 88.1 | PTPRK | 5796 | 6q22.2-q22.3 |
| 204319_s_at | 88.1 | RGS10 | 6001 | 10q25 |
| 220922_s_at | 88.1 | SPANXA1 /// SPANXA2 /// | 100133171 /// 171490 /// | Xq27.1 |
| | | SPANXB1 /// SPANXB2 /// | 30014 /// 64663 /// | |
| | | SPANXC /// SPANXF1 | 728695 /// 728712 | |
| 230970_at | 88.1 | SSH2* | — | 17q11.2 |
| 222942_s_at | 88.1 | TIAM2 | 26230 | 6q25.2 |
| 214958_s_at | 88.1 | TMC6 | 11322 | 17q25.3 |
| 204881_s_at | 88.1 | UGCG | 7357 | 9q31 |
| 221765_at | 88.1 | UGCG | 7357 | 9q31 |
| 220586_at | 87.4 | CHD9 | 80205 | 16q12.2 |
| 229268_at | 87.4 | FAM105B | 90268 | 5p15.2 |
| 225140_at | 87.4 | KLF3 | 51274 | 4p14 |
| 244741_s_at | 87.4 | MGC9913 | 386759 | 19q13.43 |
| 231199_at | 87.4 | NAT13* | — | 3q13.2 |
| 235652_at | 87.4 | SCML1* | — | Xp22.2 |

TABLE S17′
Top 100 Ross¹ BCR-ABL Probe Sets Compared
to ROSE Clustering and Top Rank Order

| | | | ROSE | |
|---|---|---|---|---|
| | | | Clus- | Rank Order |
| Probe Set ID | Gene Symbol | Cytoband | tering | Group |
| 224811_at | — | — | | |
| 226345_at | — | — | | |
| 240173_at | — | — | | |
| 240499_at | — | — | | |
| 202123_s_at | ABL1 | 9q34.1 | | R4 |
| 209321_s_at | ADCY3 | 2p23.3 | | |
| 223075_s_at | AIF1L | 9q34.13-q34.3 | | |
| 214255_at | ATP10A | 15q11.2 | | |
| 219218_at | BAHCC1 | 17q25.3 | | |
| 229975_at | BMPR1B | 4q22-q24 | Yes | R8 |
| 242579_at | BMPR1B | 4q22-q24 | Yes | R8 |
| 201310_s_at | C5orfl3 | 5q22.1 | | |
| 200655_s_at | CALM1 | 14q24-q31 | | |
| 205467_at | CASP10 | 2q33-q34 | | |
| 200951_s_at | CCND2 | 12p13 | | |
| 200953_s_at | CCND2 | 12p13 | | |
| 206150_at | CD27 | 12p13 | | R8 |
| 201028_s_at | CD99 | Xp22.32; | | R8 |
| | | Yp11.3 | | |
| 201029_s_at | CD99 | Xp22.32; | | R8 |
| | | Yp11.3 | | |
| 242051_at | CD99* | — | | R8 |
| 202717_s_at | CDC16 | 13q34 | | |
| 212862_at | CDS2 | 20p13 | | |
| 213385_at | CHN2 | 7p15.3 | | |
| 204576_s_at | CLUAP1 | 16p13.3 | | |
| 201445_at | CNN3 | 1p22-p21 | Yes | R5 |
| 228297_at | CNN3* | — | Yes | R5 |
| 201906_s_at | CTDSPL | 3p21.3 | | |
| 218013_x_at | DCTN4 | 5q31-q32 | | R8 |
| 222488_s_at | DCTN4 | 5q31-q32 | | R8 |
| 209365_s_at | ECM1 | 1q21 | | |
| 217967_s_at | FAM129A | 1q25 | | R8 |
| 202771_at | FAM38A | 16q24.3 | | |
| 222729_at | FBXW7 | 4q31.3 | | |
| 219871_at | FLJ13197 | 4p14 | | |
| 218084_x_at | FXYD5 | 19q12-q13.1 | | |
| 216033_s_at | FYN | 6q21 | | |
| 64064_at | GIMAP5 | 7q36.1 | | |
| 229367_s_at | GIMAP6 | — | | |
| 235988_at | GPR110 | 6p12.3 | Yes | R8 |
| 238689_at | GPR110 | 6p12.3 | Yes | R8 |
| 236489_at | GPR110* | — | Yes | R8 |
| 202947_s_at | GYPC | 2q14-q21 | | R4 |
| 203089_s_at | HTRA2 | 2p12 | | |
| 208881_x_at | IDI1 | 10p15.3 | | |
| 212203_x_at | IFITM3 | 11p15.5 | | R8 |
| 212592_at | IGJ | 4q21 | Yes | R8 |
| 222868_s_at | IL18BP | 11q13 | | |
| 202794_at | INPP1 | 2q32 | | |
| 205376_at | INPP4B | 4q31.21 | | |
| 201656_at | ITGA6 | 2q31.1 | Yes | R6 |
| 205055_at | ITGAE | 17p13 | | |
| 229139_at | JPH1 | 8q21 | | |
| 208071_s_at | LAIR1 | 19q13.4 | | R8 |
| 205269_at | LCP2 | 5q33.1-qter | | |
| 205270_s_at | LCP2 | 5q33.1-qter | | |
| 222762_x_at | LIMD1 | 3p21.3 | | R8 |
| 215617_at | LOC26010 | 2q33.1 | | R8 |
| 222154_s_at | LOC26010 | 2q33.1 | | R8 |
| 241812_at | LOC26010 | 2q33.1 | | R8 |
| 225799_at | LOC541471 /// | 2p11.2 /// | | |
| | NCRNA00152 | 2q13 | | |
| 238488_at | LRRC70 | 5q12.1 | | |
| 203005_at | LTBR | 12p13 | | |
| 239273_s_at | MMP28 | 17q11-q21.1 | | R8 |
| 217110_s_at | MUC4 | 3q29 | Yes | R8 |
| 218966_at | MYO5C | 15q21 | | |
| 205259_at | NR3C2 | 4q31.1 | | R8 |
| 212298_at | NRP1 | 10p12 | | |
| 239519_at | NRP1* | — | | |
| 204004_at | PAWR | 12q21 | | |
| 201876_at | PON2 | 7q21.3 | | R8 |
| 210830_s_at | PON2 | 7q21.3 | | R8 |
| 213093_at | PRKCA | 17q22-q23.2 | | |
| 218764_at | PRKCH | 14q22-q23 | | |
| 220024_s_at | PRX | 19q13.13-q13.2 | | R8 |
| 219938_s_at | PSTPIP2 | 18q12 | | |
| 200863_s_at | RAB11A | 15q21.3-q22.31 | | |
| 200864_s_at | RAB11A | 15q21.3-q22.31 | | |
| 209229_s_at | SAPS1 | 19q13.42 | | |
| 215028_at | SEMA6A | 5q23.1 | | R8 |
| 223449_at | SEMA6A | 5q23.1 | | R8 |
| 225660_at | SEMA6A | 5q23.1 | | R8 |
| 225913_at | SGK269 | 15q24.3 | | |
| 204429_s_at | SLC2A5 | 1p36.2 | | |
| 204430_s_at | SLC2A5 | 1p36.2 | | |
| 48106_at | SLC48A1 | 12q13.11 | | R8 |
| 225244_at | SNAP47 | 1q42.13 | | R8 |
| 200665_s_at | SPARC | 5q31.3-q32 | | |
| 212458_at | SPRED2 | 2p14 | | |
| 203217_s_at | ST3GAL5 | 2p11.2 | | |
| 216985_s_at | STX3 | 11q12.1 | | |
| 220684_at | TBX21 | 17q21.32 | | R4 |
| 219315_s_at | TMEM204 | 16p13.3 | | |
| 203508_at | TNFRSF1B | 1p36.3-p36.2 | | |
| 207196_s_at | TNIP1 | 5q32-q33.1 | | |
| 200742_s_at | TPP1 | 11p15 | | |
| 202369_s_at | TRAM2 | 6p21.1-p12 | | |
| 202242_at | TSPAN7 | Xp11.4 | | |
| 212242_at | TUBA4A | 2q35 | | |
| 218348_s_at | ZC3H7A | 16p13-p12 | | |
| 228046_at | ZNF827 | 4q31.22 | | |

TABLE S18′
Genes/Probe Sets Common to Rank Order
and BCR-ABL1-like Signature²

| | |
|---|---|
| Gene | Cluster |

BCR-ABL up-regulated

| | |
|---|---|
| 216565_x_at | R8 |
| ABL1 | R4 |
| AGPS | R4/R8 |
| CA6 | R8 |
| CD97 | R8 |
| CD99 | R8 |
| CNN3 | R5 |
| DCTN4 | R8 |
| GIMAP6 | R8 |
| GYPC | R4 |
| HIVEP2 | R6 |
| IFITM1 | R8 |
| IFITM3 | R8 |
| IGJ | R8 |
| IL2RA | R6 |
| LIMD1 | R8 |
| MMP28 | R8 |
| MUC4 | R8 |
| PON2 | R8 |
| PRX | R8 |
| SEMA6A | R8 |
| SLC5A3 | R7 |
| TBXA2R | R4 |

BCR-ABL down-regulated

| | |
|---|---|
| BACH2 | R2 |
| CSF2RB | R3 |
| CYP46A1 | R6 |
| IRS1 | R2 |
| KIAA0922 | R3 |
| LY9 | R4 |
| PHYH | R6 |
| WWC3 | R2 |

7. Genome-Wide Copy Number Variation Association with Rose Cluster Groups

TABLE S19′
Copy Number Analysis (CNA) Variations Associated with
ROSE Clusters

| | | | | | | | | FET |
|---|---|---|---|---|---|---|---|---|
| | 1 | 2 | 3 | 5 | 6 | 8 | no cluster | p-value |
| Lesion | 20 | 22 | 11 | 11 | 21 | 24 | 89 | |
| 1q gain | 0 | 14 | 0 | 1 | 0 | 0 | 2 | <0.0001 |
| EBF1 | 0 | 0 | 0 | 0 | 0 | 9 | 4 | <0.0001 |
| IKZF1 | 1 | 0 | 0 | 2 | 6 | 20 | 26 | <0.0001 |
| CDKN2A-B | 4 | 9 | 10 | 2 | 5 | 15 | 51 | <0.0001 |
| TCF3 | 0 | 14 | 0 | 2 | 2 | 0 | 2 | <0.0001 |
| ERG | 0 | 0 | 0 | 0 | 8 | 0 | 1 | <0.0001 |
| VPREB1 | 0 | 0 | 0 | 1 | 8 | 14 | 28 | <0.0001 |
| B cell pathway** | 5 | 17 | 5 | 4 | 12 | 23 | 66 | <0.0001 |
| B cell pathway | 5 | 17 | 5 | 5 | 14 | 24 | 68 | <0.0001 |
| including VPREB1** | | | | | | | | |
| TBL1XR1 | 0 | 0 | 3 | 1 | 1 | 0 | 0 | 0.0002 |
| PAX5 can | 1 | 9 | 4 | 0 | 3 | 7 | 39 | 0.0005 |
| RAG1-2 | 1 | 0 | 1 | 0 | 0 | 5 | 0 | 0.0005 |
| NUP160-PTPRJ | 0 | 0 | 0 | 0 | 0 | 4 | 0 | 0.0014 |
| ETV6 | 1 | 0 | 3 | 4 | 1 | 0 | 15 | 0.0031 |
| DMD | 0 | 5 | 1 | 2 | 3 | 0 | 3 | 0.0059 |
| IL3RA-CSF2RA | 0 | 0 | 1 | 1 | 0 | 7 | 6 | 0.0061 |
| C20orf94 | 0 | 0 | 0 | 1 | 0 | 7 | 8 | 0.0073 |
| ADD3 | 0 | 1 | 0 | 0 | 0 | 7 | 9 | 0.0144 |
| NF1 | 1 | 1 | 0 | 2 | 0 | 1 | 0 | 0.0188 |
| ARMC2-SESN1 | 0 | 2 | 0 | 2 | 0 | 5 | 4 | 0.0291 |
| ADARB2 | 0 | 0 | 0 | 0 | 2 | 2 | 0 | 0.0410 |
| BTG1 | 0 | 0 | 0 | 2 | 2 | 6 | 10 | 0.0442 |
| BTLA-CD200 | 0 | 0 | 0 | 0 | 0 | 5 | 6 | 0.0633 |
| GRIK2 | 0 | 2 | 0 | 2 | 0 | 4 | 4 | 0.0699 |
| ELF1 | 0 | 5 | 0 | 1 | 0 | 1 | 6 | 0.0788 |
| IL1RAP | 0 | 0 | 2 | 0 | 0 | 0 | 1 | 0.0845 |
| FLNB | 0 | 0 | 0 | 0 | 2 | 2 | 1 | 0.1532 |
| DLEU2-7- | 0 | 4 | 1 | 1 | 1 | 0 | 10 | 0.2047 |
| mir15--16a | | | | | | | | |
| C13orf21- | 0 | 4 | 0 | 1 | 0 | 2 | 11 | 0.2097 |
| TSC22D1 | | | | | | | | |
| KRAS | 1 | 2 | 0 | 2 | 0 | 0 | 8 | 0.2869 |
| PDE4B | 0 | 0 | 0 | 0 | 0 | 3 | 3 | 0.3136 |
| LOC440742* | 0 | 0 | 0 | 0 | 0 | 3 | 3 | 0.3136 |
| TOX | 0 | 0 | 0 | 0 | 0 | 3 | 4 | 0.3430 |
| FBXW7 | 0 | 0 | 0 | 0 | 0 | 2 | 1 | 0.3779 |
| RB1 | 0 | 4 | 0 | 1 | 1 | 2 | 12 | 0.3886 |
| FHIT | 0 | 0 | 0 | 0 | 0 | 1 | 0 | 0.5505 |
| MSRA | 0 | 0 | 0 | 1 | 0 | 0 | 3 | 0.6230 |
| ARID1B | 0 | 1 | 0 | 1 | 1 | 2 | 3 | 0.6751 |
| ARPP-21 | 0 | 0 | 0 | 0 | 0 | 2 | 5 | 0.6777 |
| Histone cluster | 0 | 0 | 0 | 0 | 0 | 2 | 6 | 0.6782 |
| MBNL1 | 0 | 0 | 1 | 0 | 0 | 1 | 3 | 0.6815 |
| ATP10A | 0 | 0 | 0 | 1 | 0 | 1 | 3 | 0.6815 |
| iAmp21 | 0 | 0 | 0 | 0 | 0 | 1 | 7 | 0.6879 |
| NRAS | 0 | 0 | 0 | 0 | 1 | 0 | 2 | 0.7695 |
| ADAR | 0 | 0 | 0 | 0 | 0 | 1 | 1 | 0.7992 |
| COPEB-KLF6 | 0 | 0 | 0 | 0 | 0 | 1 | 1 | 0.7992 |
| CCDC26 | 2 | 1 | 0 | 1 | 3 | 3 | 8 | 0.8732 |
| ABL1 | 0 | 0 | 0 | 0 | 0 | 1 | 2 | 0.9109 |
| NR3C2 | 0 | 0 | 0 | 0 | 0 | 1 | 4 | 0.9751 |
| ARHGAP24 | 0 | 0 | 0 | 0 | 0 | 1 | 3 | 1.0000 |
| ZMYM5 | 0 | 0 | 0 | 0 | 0 | 0 | 3 | 1.0000 |
| SPRED1 (5′) | 0 | 0 | 0 | 0 | 0 | 0 | 0 | 1.0000 |
| LTK | 0 | 0 | 0 | 0 | 0 | 0 | 0 | 1.0000 |

The CNA variations are shown along with their membership in each ROSE cluster.
FET indicates the p-value for this results as determined by Fisher's Exact Test.
CNA variations are sorted in ascending order by their p-values.

### REFERENCES

### First Set

1. Pui C H, Evans W E. Drug therapy—Treatment of acute lymphoblastic leukemia. N Engl J Med. 2006; 354(2):166-178.
2. Pui C H, Robison L L, Look AT. Acute lymphoblastic leukaemia. Lancet. 2008; 371(9617):1030-1043.
3. Pui C H, Pei D Q, Sandlund J T, et al. Risk of adverse events after completion of therapy for childhood acute lymphoblastic leukemia. JClin Oncol. 2005; 23(31):7936-7941.
4. Schultz K R, Pullen D J, Sather H N, et al. Risk- and response-based classification of childhood Bprecursor acute lymphoblastic leukemia: a combined analysis of prognostic markers from the Pediatric Oncology Group (POG) and Children's Cancer Group (CCG). Blood. 2007; 109(3):926-935.
5. Smith M, Arthur D, Camitta B, et al. Uniform approach to risk classification and treatment assignment for children with acute lymphoblastic leukemia. J Clin Oncol. 1996; 14(1):18-24.
6. Borowitz M J, Devidas M, Hunger S P, et al. Clinical significance of minimal residual disease in childhood acute lymphoblastic leukemia and its relationship to other prognostic factors: a Children's Oncology Group study. Blood. 2008; 111(12):5477-5485.
7. Pui C H, Jeha S. New therapeutic strategies for the treatment of acute lymphoblastic leukaemia. Nat Rev Drug Discov. 2007; 6(2):149-165.
8. Yeoh E J, Ross M E, Shurtleff S A, et al. Classification, subtype discovery, and prediction of outcome in pediatric acute lymphoblastic leukemia by gene expression profiling. Cancer Cell. 2002; 1(2):133-143.
9. Cheok M H, Yang W L, Pui C H, et al. Treatment-specific changes in gene expression discriminate in vivo drug response in human leukemia cells. Nat Genet. 2003; 34(1):85-90.
10. Holleman A, Cheok M H, den Boer M L, et al. Gene-expression patterns in drug-resistant acute lymphoblastic leukemia cells and response to treatment. N Engl J Med. 2004; 351(6):533-542.
11. Lugthart S, Cheok M H, den Boer M L, et al. Identification of genes associated with chemotherapy crossresistance and treatment response in childhood acute lymphoblastic leukemia. Cancer Cell. 2005; 7(4):375-386.
12. Mullighan C G, Goorha S, Radtke I, et al. Genome-wide analysis of genetic alterations in acute lymphoblastic leukaemia. Nature. 2007; 446(7137):758-764.
13. Flotho C, Coustan-Smith E, Pei D Q, et al. A set of genes that regulate cell proliferation predictstreatment outcome in childhood acute lymphoblastic leukemia. Blood. 2007; 110(4):1271-1277.
14. Bhojwani D, Kang H, Menezes R X, et al. Gene expression signatures predictive of early response and outcome in high-risk childhood acute lymphoblastic leukemia: a Children's Oncology Group Study on behalf of the Dutch Childhood Oncology Group and the German Cooperative Study Group for Childhood Acute Lymphoblastic Leukemia. J Clin Oncol. 2008; 26(27):4376-4384.
15. Sorich M J, Pottier N, Pei D, et al. In vivo response to methotrexate forecasts outcome of acute lymphoblastic leukemia and has a distinct gene expression profile. PLoS Med. 2008; 5(4):646-656.
16. Mullighan C G, Su X, Zhang J, et al. Deletion of IKZF1 and prognosis in acute lymphoblastic leukemia. N Engl J Med. 2009;360(5):470-480.
17. Mullighan C G, Zhang J, Harvey R C, et al. JAK mutations in high-risk childhood acute lymphoblastic leukemia. Proc Natl Acad Sci USA. 2009; 106(23):9414-9418.
18. Den Boer M L, van Slegtenhorst M, De Menezes R X, et al. A subtype of childhood acute lymphoblastic leukaemia with poor treatment outcome: a genome-wide classification study. Lancet Oncol. 2009; 10(2):125-134.
19. Nachman J B, Sather H N, Sensel M G, et al. Augmented post-induction therapy for children with highrisk acute lymphoblastic leukemia and a slow response to initial therapy. N Engl J Med. 1998; 338(23):1663-1671.
20. Shuster J J, Camitta B M, Pullen J, et al. Identification of newly diagnosed children with acute lymphocytic leukemia at high risk for relapse. Cancer Research Therapy and Control. 1999; 9(1-2):101-107.
21. Bair E, Hastie T, Paul D, Tibshirani R. Prediction by supervised principal components. J Am Stat Assoc. 2006; 101(473):119-137.
22. Asgharzadeh S, Pique-Regi R, Sposto R, et al. Prognostic significance of gene expression profiles of metastatic neuroblastomas lacking MYCN gene amplification. J Natl Cancer Inst. 2006; 98(17):1193-1203.
23. Simon R. Development and evaluation of therapeutically relevant predictive classifiers using gene expression profiling. J Natl Cancer Inst. 2006; 98(17):1169-1171.
24. Tusher V G, Tibshirani R, Chu G. Significance analysis of microarrays applied to the ionizing radiation response. Proc Natl Acad Sci USA. 2001; 98(9):5116-5121.
25. Ross M E, Zhou X, Song G, et al. Classification of pediatric acute lymphoblastic leukemia by gene expression profiling. Blood. 2003; 102(8):2951-2959.
26. Martin S B, Mosquera-Caro M P, Potter J W, et al. Gene expression overlap affects karyotype prediction in pediatric acute lymphoblastic leukemia. Leukemia. 2007; 21(6):1341-1344.
27. Mullican S E, Zhang S, Konopleva M, et al. Abrogation of nuclear receptors Nr4a3 and Nr4a1 leads to development of acute myeloid leukemia. Nat Med. 2007; 13(6):730-735.
28. Schwable J, Choudhary C, Thiede C, et al. RGS2 is an important target gene of Flt3-ITD mutations in AML and functions in myeloid differentiation and leukemic transformation. Blood. 2005; 105(5):2107-2114.
29. Gottardo N G, Hoffmann K, Beesley A H, et al. Identification of novel molecular prognostic markersfor paediatric T-cell acute lymphoblastic leukaemia. Br J Haematol. 2007; 137(4):319-328.
30. Agenes F, Bosco N, Mascarell L, Fritah S, Ceredig R. Differential expression of regulator of Gprotein signalling transcripts and in vivo migration of CD4+ naive and regulatory T cells. Immunology. 2005; 115(2):179-188.
31. Horke S, Witte I, Wilgenbus P, Kruger M, Strand D, Forstermann U. Paraoxonase-2 reduces oxidative stress in vascular cells and decreases endoplasmic reticulum stress-induced caspase activation. Circulation. 2007; 115(15):2055-2064.
32. Gomis R R, Alarcon C, He W, et al. A FoxO-Smad synexpression group in human keratinocytes. Proc Natl Acad Sci USA. 2006; 103(34):12747-12752.
33. Chen P-S, Wang M-Y, Wu S-N, et al. CTGF enhances the motility of breast cancer cells via an integrin-alpha v beta 3-ERK1/2-dependent S100A4-upregulated pathway. J Cell Sci. 2007; 120(12):2053-2065.
34. Wang L, Zhou X, Zhou T, et al. Ecto-5′-nucleotidase promotes invasion, migration and adhesion of human breast cancer cells. J Cancer Res Clin Oncol. 2008; 134(3):365-372.
35. Kodach L L, Bleurning S A, Musler A R, et al. The bone morphogenetic protein pathway is active in human colon adenomas and inactivated in colorectal cancer. Cancer. 2008; 112(2):300-306.
36. Rae F K, Hooper J D, Eyre H J, Sutherland G R, Nicol D L, Clements J A. TTYH2, a human homologue of the Drosophila melanogaster gene tweety, is located on 17q24 and upregulated in renal cell carcinoma. Genomics. 2001; 77(3):200-207.
37. Toiyama Y, Mizoguchi A, Kimura K, et al. TTYH2, a human homologue of the Drosophila melanogaster gene tweety, is up-regulated in colon carcinoma and involved in cell proliferation and cell aggregation. World J Gastroenterol. 2007; 13(19):2717-2721.
38. Dunne J, Cullmann C, Ritter M, et al. siRNA-mediated AML1/MTG8 depletion affects differentiation and proliferation-associated gene expression in t(8;21)-positive cell lines and primary AML blasts. Oncogene. 2006; 25(45):6067-6078.
39. Assou S, Le Carrour T, Tondeur S, et al. A meta-analysis of human embryonic stem cells transcriptome integrated into a web-based expression atlas. Stem Cells. 2007; 25(4):961-973.
40. Mageed A S, Pietryga D W, DeHeer D H, West R A. Isolation of large numbers of mesenchymal stem cells from the washings of bone marrow collection bags: characterization of fresh mesenchymal stem cells. Transplantation. 2007; 83(8):1019-1026.
41. Deaglio S, Dwyer K M, Gao W, et al. Adenosine generation catalyzed by CD39 and CD73 expressed on regulatory T cells mediates immune suppression. J Exp Med. 2007; 204(6):1257-1265.
42. Mikhailov A, Sokolovskaya A, Yegutkin G G, et al. CD73 participates in cellular multiresistance program and protects against TRAIL-induced apoptosis. J Immunol. 2008; 181(1):464-475.
43. Sala-Torra O, Gundacker H M, Stirewalt D L, et al. Connective tissue growth factor (CTGF) expression and outcome in adult patients with acute lymphoblastic leukemia. Blood. 2007; 109(7):3080-3083.
44. Boag J M, Beesley A H, Firth M J, et al. High expression of connective tissue growth factor in pre-B acute lymphoblastic leukaemia. Br J Haematol. 2007; 138(6):740-748.
45. Hoffmann K, Firth M J, Beesley A H, et al. Prediction of relapse in paediatric pre-B acute lymphoblastic leukaemia using a three-gene risk index. Br J Haematol. 2008; 140(6):656-664.
46. Baldus C D, Martus P, Burmeister T, et al. Low ERG and BAALC expression identifies a new subgroup of adult acute T-lymphoblastic leukemia with a highly favorable outcome. J Clin Oncol. 2007; 25(24):3739-3745.
47. Langer C, Radmacher M D, Ruppert A S, et al. High BAALC expression associates with other molecular prognostic markers, poor outcome, and a distinct gene-expression signature in cytogenetically normal patients younger than 60 years with acute myeloid leukemia: a Cancer and Leukemia Group B (CALGB) study. Blood. 2008; 111(11):5371-5379.

### REFERENCES

### Second Set—1^{ST} Supplement

1. Borowitz M J, Devidas M, Hunger S P, et al. Clinical significance of minimal residual disease in childhood acute lymphoblastic leukemia and its relationship to other prognostic factors: a Children's Oncology Group study. Blood. 2008; 111(12):5477-5485.
2. Bair E, Tibshirani R. Semi-supervised methods to predict patient survival from gene expression data. PLoS Biol. 2004; 2(4):511-522.
3. Shuster J J, Camitta B M, Pullen J, et al. Identification of newly diagnosed children with acute lymphocytic leukemia at high risk for relapse. Cancer Research Therapy and Control. 1999; 9(1-2):101-107.
4. Bhojwani D, Kang H, Menezes R X, et al. Gene expression signatures predictive of early response and outcome in high-risk childhood acute lymphoblastic leukemia: a Children's Oncology Group Study on behalf of the Dutch Childhood Oncology Group and the German Cooperative Study Group for Childhood Acute Lymphoblastic Leukemia. J Clin Oncol. 2008; 26(27):4376-4384.
5. Wilson C S, Davidson G S, Martin S B, et al. Gene expression profiling of adult acute myeloid leukemia identifies novel biologic clusters for risk classification and outcome prediction. Blood. 2006;108(2):685-696.
6. O'Shaughnessy J A. Molecular signatures predict outcomes of breast cancer. N Engl J Med. 2006; 355(6):615-617.
7. Fan C, Oh D S, Wessels L, et al. Concordance among gene-expression-based predictors for breast cancer. N Engl J Med. 2006; 355(6):560-569.
8. Twombly R. Breast cancer gene microarrays pass muster. J Natl Cancer Inst. 2006; 98(20):1438-1440.
9. Simon R. Development and evaluation of therapeutically relevant predictive classifiers using gene expression profiling. J Natl Cancer Inst. 2006; 98(17):1169-1171.
10. Asgharzadeh S, Pique-Regi R, Sposto R, et al. Prognostic significance of gene expression profiles of metastatic neuroblastomas lacking MYCN gene amplification. J Natl Cancer Inst. 2006; 98(17):1193-1203.
11. Bair E, Hastie T, Paul D, Tibshirani R. Prediction by supervised principal components. J Am Stat Assoc. 2006; 101(473):119-137.
12. Bair E, Tibshirani R. Supervised principal components, R package.
13. Tusher V G, Tibshirani R, Chu G. Significance analysis of microarrays applied to the ionizing radiation response. Proc Natl Acad Sci USA. 2001; 98(9): 5116-5121.
14. Dudoit S, Fridlyand J, Speed T P. Comparison of discrimination methods for the classification of tumors using gene expression data. J Am Stat Assoc. 2002; 97(457):77-87.
15. Horke S, Witte I, Wilgenbus P, Kruger M, Strand D, Forstermann U. Paraoxonase-2 reduces oxidative stress in vascular cells and decreases endoplasmic reticulum stress-induced caspase activation. Circulation. 2007; 115(15):2055-2064.
16. Gomis R R, Alarcon C, He W, et al. A FoxO-Smad synexpression group in human keratinocytes. Proc Nall Acad Sci USA. 2006; 103(34):12747-12752.
17. Chen P-S, Wang M-Y, Wu S-N, et al. CTGF enhances the motility of breast cancer cells via an integrin-alpha v beta 3-ERK1/2-dependent S100A4-upregulated pathway. J Cell Sci. 2007; 120(12):2053-2065.
18. Wang L, Zhou X, Zhou T, et al. Ecto-5′-nucleotidase promotes invasion, migration and adhesion of human breast cancer cells. J Cancer Res Clin Oncol. 2008; 134(3):365-372.
19. Kodach L L, Bleurning S A, Musler A R, et al. The bone morphogenetic protein pathway is active in human colon adenomas and inactivated in colorectal cancer. Cancer. 2008; 112(2):300-306.
20. Rae F K, Hooper J D, Eyre H J, Sutherland G R, Nicol D L, Clements J A. TTYH2, a human homologue of the Drosophila melanogaster gene tweety, is located on 17q24 and upregulated in renal cell carcinoma. Genomics. 2001; 77(3):200-207.
21. Toiyama Y, Mizoguchi A, Kimura K, et al. TTYH2, a human homologue of the Drosophila melanogaster gene tweety, is up-regulated in colon carcinoma and involved in cell proliferation and cell aggregation. World J. Gastroenterol. 2007; 13(19): 2717-2721.
22. Dunne J, Cullmann C, Ritter M, et al. siRNA-mediated AML1/MTG8 depletion affects differentiation and proliferation-associated gene expression in t(8;21)-positive cell lines and primary AML blasts. Oncogene. 2006; 25(6067-6078.
23. Assou S, Le Carrour T, Tondeur S, et al. A meta-analysis of human embryonic stem cells transcriptome integrated into a web-based expression atlas. Stem Cells. 2007; 25(4):961-973.
24. Mageed A S, Pietryga D W, DeHeer D H, West R A. Isolation of large numbers of mesenchymal stem cells from the washings of bone marrow collection bags: characterization of fresh mesenchymal stem cells. Transplantation. 2007; 83(1019-1026.
25. Boag J M, Beesley A H, Firth M J, et al. High expression of connective tissue growth factor in pre-B acute lymphoblastic leukaemia. Br J. Haematol. 2007; 138(6):740-748.
26. Deaglio S, Dwyer K M, Gao W, et al. Adenosine generation catalyzed by CD39 and CD73 expressed on regulatory T cells mediates immune suppression. J Exp Med. 2007; 204(1257-1265.
27. Mikhailov A, Sokolovskaya A, Yegutkin G G, et al. CD73 participates in cellular multiresistance program and protects against TRAIL-induced apoptosis. J Immunol. 2008; 181(1):464-475.
28. Mullican S E, Zhang S, Konopleva M, et al. Abrogation of nuclear receptors Nr4a3 and Nr4a1 leads to development of acute myeloid leukemia. Nat Med. 2007; 13(6):730-735.
29. Gottardo N G, Hoffmann K, Beesley A H, et al. Identification of novel molecular prognostic markers for paediatric T-cell acute lymphoblastic leukaemia. Br J. Haematol. 2007; 137(319-328.
30. Agenes F, Bosco N, Mascarell L, Fritah S, Ceredig R. Differential expression of regulator of G-protein signalling transcripts and in vivo migration of CD4+naïve and regulatory T cells. J Immunol. 2005; 115(179-188.
31. Schwable J, Choudhary C, Thiede C, et al. RGS2 is an important target gene of Flt3-ITD mutations in AML and functions in myeloid differentiation and leukemic transformation. Blood. 2005; 105(5):2107-2114.
32. Lehar S M, Bevan M J. T cells develop normally in the absence of both Deltex1 and Deltex2. Mol Cell Biol. 2006; 26(7358-7371.
33. Feinberg M W, Wara A K, Cao Z, et al. The Kruppel-like factor KLF4 is a critical regulator of monocyte differentiation. EMBO J. 2007; 26(4138-4148.
34. Cario G, Stanulla M, Fine B M, et al. Distinct gene expression profiles determine molecular treatment response in childhood acute lymphoblastic leukemia. Blood. 2005; 105(821-826.
35. Flotho C, Coustan-Smith E, Pei D, et al. A set of genes that regulate cell proliferation predicts treatment outcome in childhood acute lymphoblastic leukemia. Blood. 2007; 110(4):1271-1277.
36. Flotho C, Coustan-Smith E, Pei D, et al. Genes contributing to minimal residual disease in childhood acute lymphoblastic leukemia: prognostic significance of CASP8AP2. Blood. 2006; 108(3):1050-1057.
37. Yeoh E J, Ross M E, Shurtleff S A, et al. Classification, subtype discovery, and prediction of outcome in pediatric acute lymphoblastic leukemia by gene expression profiling. Cancer Cell. 2002; 1(2):133-143.
38. Langer C, Radmacher M D, Ruppert A S, et al. High BAALC expression associates with other molecular prognostic markers, poor outcome, and a distinct gene-expression signature in cytogenetically normal patients younger than 60 years with acute myeloid leukemia: a Cancer and Leukemia Group B (CALGB) study. Blood. 2008; 111(11):5371-5379.
39. Tibshirani R, Chu G, Hastie T, Narasimhan B. SAM: Significance analysis of microarrays, R package.

### REFERENCES

### Third Set

1. Smith M, Arthur D, Camitta B, et al. Uniform approach to risk classification and treatment assignment for children with acute lymphoblastic leukemia. J Clin Oncol. 1996; 14(1):18-24.
2. Schultz K R, Pullen D J, Sather H N, et al. Risk- and response-based classification of childhood B-precursor acute lymphoblastic leukemia: a combined analysis of prognostic markers from the Pediatric Oncology Group (POG) and Children's Cancer Group (CCG). Blood. 2007; 109(3):926-935.
3. Kadan-Lottick N S, Ness K K, Bhatia S, Gurney J G. Survival variability by race and ethnicity in childhood acute lymphoblastic leukemia. JAMA: The Journal of the American Medical Association. 2003; 290(15):2008-2014.
4. Shuster J J, Camitta B M, Pullen J, et al. Identification of newly diagnosed children with acute lymphocytic leukemia at high risk for relapse. Cancer Research Therapy and Control. 1999; 9(1-2):101-107.
5. Mullighan C G, Su X, Zhang J, et al. Deletion of IKZF1 and prognosis in acute lymphoblastic leukemia. N Engl J Med. 2009; 360(5):470-480.
6. Mullighan C G, Zhang J, Harvey R C, et al. JAK mutations in high-risk childhood acute lymphoblastic leukemia. Proc Natl Acad Sci USA. 2009.
7. Borowitz M J, Devidas M, Hunger S P, et al. Clinical significance of minimal residual disease in childhood acute lymphoblastic leukemia and its relationship to other prognostic factors: a Children's Oncology Group study. Blood. 2008; 111(12):5477-5485.
8. Borowitz M J, Devidas M, Hunger S P, et al. Clinical significance of minimal residual disease in childhood acute lymphoblastic leukemia and its relationship to other prognostic factors: A Children's Oncology Group study. Blood. 2008.
9. Nachman J B, Sather H N, Sensel M G, et al. Augmented post-induction therapy for children with high-risk acute lymphoblastic leukemia and a slow response to initial therapy. N Engl J Med. 1998; 338(23):1663-1671.
10. Seibel N L, Steinherz P G, Sather H N, et al. Early postinduction intensification therapy improves survival for children and adolescents with high-risk acute lymphoblastic leukemia: a report from the Children's Oncology Group. Blood. 2008; 111(5):2548-2555.
11. Borowitz M J, Pullen D J, Shuster J J, et al. Minimal residual disease detection in childhood precursor-B-cell acute lymphoblastic leukemia: relation to other risk factors. A Children's Oncology Group study. Leukemia. 2003; 17(8):1566-1572.
12. Bhojwani D, Kang H, Menezes R X, et al. Gene expression signatures predictive of early response and outcome in high-risk childhood acute lymphoblastic leukemia: a Children's Oncology Group Study on behalf of the Dutch Childhood Oncology Group and the German Cooperative Study Group for Childhood Acute Lymphoblastic Leukemia. J Clin Oncol. 2008; 26(27):4376-4384.
13. Wilson C S, Davidson G S, Martin S B, et al. Gene expression profiling of adult acute myeloid leukemia identifies novel biologic clusters for risk classification and outcome prediction. Blood. 2006; 108(2):685-696.
14. Tomlins S A, Rhodes D R, Perner S, et al. Recurrent fusion of TMPRSS2 and ETS transcription factor genes in prostate cancer. Science. 2005; 310(5748):644-648.
15. Mullighan C G, Goorha S, Radtke I, et al. Genome-wide analysis of genetic alterations in acute lymphoblastic leukaemia. Nature. 2007; 446(7137): 758-764.
16. Mullighan C G, Miller C B, Radtke I, et al. BCR-ABL1 lymphoblastic leukaemia is characterized by the deletion of Ikaros. Nature. 2008; 453(7191):110-114.
17. Bland J M, Altman D G. The logrank test. BMJ. 2004; 328(7447):1073.
18. Armitage P, Berry G. Statistical methods in medical research (ed 3rd). Oxford; Boston: Blackwell Scientific Publications; 1994.
19. Bewick V, Cheek L, Ball J. Statistics review 12: survival analysis. Crit Care. 2004; 8(5):389-394.
20. R_Development_Core_Team. R: A language and environment for statistical computing; 2009.
21. Ross M E, Zhou X D, Song G C, et al. Classification of pediatric acute lymphoblastic leukemia by gene expression profiling. Blood. 2003; 102(8):2951-2959.
22. Wong P, Iwasaki M, Somervaille T C, So C W, Cleary M L. Meisl is an essential and rate-limiting regulator of MLL leukemia stem cell potential. Genes Dev. 2007; 21(21):2762-2774.
23. Sala-Torra O, Gundacker H M, Stirewalt D L, et al. Connective tissue growth factor (CTGF) expression and outcome in adult patients with acute lymphoblastic leukemia. Blood. 2007; 109(7):3080-3083.
24. Julie D, Lacayo N J, Ramsey M C, et al. Differential gene expression patterns and interaction networks in BCR-ABL-positive and -negative adult acute lymphoblastic leukemias. J Clin Oncol. 2007; 25(11):1341-1349.
25. Mullighan C G, Collins-Underwood J R, Phillips L A A, et al. Rearrangement of CRLF2 in B-progenitor and Down syndrome associated acute lymphoblastic leukemia. Nat Genet. 2009; (in press).
26. Russell L J, Capasso M, Vater I, et al. Deregulated expression of cytokine receptor gene, CRLF2, is involved in lymphoid transformation in B-cell precursor acute lymphoblastic leukemia. Blood. 2009; 114(13):2688-2698.
27. Mullighan C G, Miller C B, Su X, et al. ERG deletions define a novel subtype of B-progenitor acute lymphoblastic leukemia. Blood. 2007; 110(11, 1):212A-213A.
28. Yeoh E J, Ross M E, Shurtleff S A, et al. Classification, subtype discovery, and prediction of outcome in pediatric acute lymphoblastic leukemia by gene expression profiling. Cancer Cell. 2002; 1(2):133-143.
29. Bhatia S, Sather H N, Heerema N A, Trigg M E, Gaynon P S, Robison L L. Racial and ethnic differences in survival of children with acute lymphoblastic leukemia. Blood. 2002; 100(6):1957-1964.
30. Pollock B H, DeBaun M R, Camitta B M, et al. Racial differences in the survival of childhood B-precursor acute lymphoblastic leukemia: a Pediatric Oncology Group Study. J Clin Oncol. 2000; 18(4):813-823.
31. Den Boer M L, van Slegtenhorst M, De Menezes R X, et al. A subtype of childhood acute lymphoblastic leukaemia with poor treatment outcome: a genome-wide classification study. Lancet Oncol. 2009; 10(2):125-134.
32. Harvey R C, Davidson G S, Wang X, et al. Expression profiling identifies novel genetic subgroups with distinct clinical features and outcome in high-risk pediatric precursor B acute lymphoblastic leukemia (B-ALL). A Children's Oncology Group Study. Blood. 2007; 110: Abstract 1430.
33. Russell L J, Capasso M, Vater I, et al. IGH@ translocations involving the pseudoautosomal region 1 (PAR1) of both sex chromosomes deregulate the cytokine receptor-like factor 2 (CRLF2) gene in B cell precursor acute lymphoblastic leukemia (BCP-ALL). Blood. 2008; 112: Abstract 787.
34. Russell L J, Capasso M, Vater I, et al. Deregulated expression of cytokine receptor gene, CRLF2, is involved in lymphoid transformation in B cell precursor acute lymphoblastic leukemia. Blood. 2009.
35. Juric D, Lacayo N J, Ramsey M C, et al. Differential gene expression patterns and interaction networks in BCR-ABL-positive and -negative adult acute lymphoblastic leukemias. J Clin Oncol. 2007; 25(11):1341-1349.

### REFERENCES

### Fourth Set—4th Supplement

1. Ross M E, Zhou X D, Song G C, et al. Classification of pediatric acute lymphoblastic leukemia by gene expression profiling. Blood. 2003; 102(8):2951-2959.
2. Mullighan C G, Su X, Zhang J, et al. Deletion of IKZF1 and prognosis in acute lymphoblastic leukemia. N Engl J Med. 2009; 360(5):470-480.
3. Borowitz M J, Devidas M, Hunger S P, et al. Clinical significance of minimal residual disease in childhood acute lymphoblastic leukemia and its relationship to other prognostic factors: a Children's Oncology Group study. Blood. 2008; 111(12):5477-5485.
4. Bhojwani D, Kang H, Menezes R X, et al. Gene expression signatures predictive of early response and outcome in high-risk childhood acute lymphoblastic leukemia: a Children's Oncology Group Study on behalf of the Dutch Childhood Oncology Group and the German Cooperative Study Group for Childhood Acute Lymphoblastic Leukemia. J Clin Oncol. 2008; 26(27):4376-4384.
5. Tomlins S A, Rhodes D R, Perrier S, et al. Recurrent fusion of TMPRSS2 and ETS transcription factor genes in prostate cancer. Science. 2005; 310(5748):644-648.

1. A method for predicting therapeutic outcome in a leukemia patient comprising:
(a) obtaining a biological sample from a patient;
(b) determining in said sample the expression level for at least two gene products selected from the group consisting of the gene products which are set forth in Tables 1P or alternatively 1Q hereof, to yield observed gene expression levels; and
(c) comparing the observed gene expression levels for the gene products to a control gene expression level selected from the group consisting of:
(i) the gene expression level for the gene products observed in a control sample; and
(ii) a predetermined gene expression level for the gene products;
wherein an observed expression levels that is higher or lower than the control gene expression levels is indicative of predicted remission or therapeutic failure.

2. The method of claim 1 wherein said at least two gene products includes at least three gene products from Table 1P.

3. The method of claim 1 wherein said at least two gene products includes at least three gene products from Table 1Q hereof.

4. The method of claim 1 wherein said at least two gene products are selected from the group consisting of BMPR1B; CTGF; IGJ; LDB3; PON2; RGS2; SCHIP1 and SEMA6A.

5. The method of claim 1 wherein said gene product includes at least two gene products selected from the group consisting of BMPR1B; CA6; CRLF2; GPR110; IGJ; LDB3; MUC4; NRXN3; PON2; RGS2 and SEMA6A.

6. The method according to claim 1 wherein said gene products include at least three gene products.

7. The method according to claim 1 wherein said gene products include at least four gene products.

8. (canceled)

9. (canceled)

10. (canceled)

11. (canceled)

12. (canceled)

13. (canceled)

14. (canceled)

15. (canceled)

16. The method according to claim 1 wherein at least one of said gene products is CRLF2.

17. The method according to claim 1 wherein said leukemia patient has been diagnosed with acute lymphoblastic leukemia (ALL).

18. The method according to claim 1 wherein said leukemia patient has been diagnosed with B-precursor acute lymphoblastic leukemia (B-ALL)

19. The method according to claim 18 wherein said leukemia patient is a pediatric leukemia patient.

20. The method according to claim 1 wherein an observed expression level which is greater than a control expression level is indicative of an unfavorable therapeutic outcome.

21. The method according to claim 1 wherein an observed expression level which is greater than a control expression level is indicative of a favorable therapeutic outcome.

22. The method according to claim 1 wherein an observed expression level of at least one gene product selected from the group consisting of BMPR1B; C8orf38; CDC42EP3; CTGF; DKFZP761M1511; ECM1; GRAMD1C; IGJ; LDB3; LOC400581; LRRC62; MDFIC; NT5E; PON2; SCHIP1; SEMA6A; TSPAN7 and TTYH2 which is greater than a control expression level is indicative of an unfavorable therapeutic outcome.

23. The method according to claim 4 wherein an observed expression level of at least one gene product selected from the group consisting of BMPR1B; CTGF; IGJ; LDB3; PON2; SCHIP1 and SEMA6A which is greater than a control expression level is indicative of an unfavorable therapeutic outcome.

24. The method according to claim 1 wherein an observed expression level of at least one gene product selected from the group consisting of BTG3; C14orf32; CD2; CHST2; DDX21; FMNL2; MGC12916; NFKBIB; NR4A3; RGS1; RGS2; UBE2E3 and VPREB1 which is greater than a control expression level is indicative of a favorable therapeutic outcome.

25. The method according to claim 1 wherein an observed expression level of at least one gene product selected from the group consisting of BMPR1B; BTBD11; C21orf87; CA6; CDC42EP3; CKMT2; CRLF2; CTGF; DIP2A; GIMAP6; GPR110; IGFBP6; IGJ; K1F1C; LDB3; LOC391849; LOC650794; MUC4; NRXN3; PON2; RGS3; SCHIP1; SCRN3; SEMA6A and ZBTB16 which is greater than a control expression level is indicative of an unfavorable therapeutic outcome.

26. The method according to claim 5 wherein an observed expression level of at least one gene product selected from the group consisting of BMPR1B; CA6; CRLF2; GPR110; IGJ; LDB3; MUC4; NRXN3; PON2; RGS2 and SEMA6A which is greater than a control expression level is indicative of an unfavorable therapeutic outcome.

27. The method according to claim 4 wherein an observed expression level of RGS2 which is greater than a control expression level is indicative of a favorable therapeutic outcome.

28. The method according to claim 1 wherein said gene products are selected from the group consisting of CA6, IGJ, MUC4, GPR110, LDB3, PON2, RGS2 and CRLF2.

29. The method according to claim 1 wherein said gene products further include AGAP-1 (Arf GAP with GTP-binding protein-like, ANK repeat and PH
domains) and/or PCDH17 (Protocadherin-17).

30. A method for predicting therapeutic outcome in a leukemia patient comprising:
(a) obtaining a biological sample from a patient;
(b) determining in said sample the expression level of gene products for at least five of the genes of Tables 1P or alternatively, 1Q hereof to yield observed gene expression levels; and
(c) comparing the observed gene expression levels for the gene products to a control gene expression level selected from the group consisting of:
(i) the gene expression level for the gene products observed in a control sample; and
(ii) a predetermined gene expression level for the gene products;
wherein an observed expression levels that is higher or lower than the control gene expression levels is indicative of predicted remission or an unfavorable therapeutic outcome.

31. The method according to claim 30 wherein the expression levels of BMPR1B; CA6; CRLF2; GPR110; IGJ; LDB3; MUC4; NRXN3; PON2 and SEMA6A which is above a control expression level is indicative of a unfavorable therapeutic outcome and the expression level of RGS2 which is above a control expression level is indicative of a favorable therapeutic outcome.

32. The method according to claim 30 wherein the expression levels of CA6; CRLF2; GPR110; IGJ; LDB3; MUC4 and PON2 which is above a control expression level is indicative of a unfavorable therapeutic outcome and the expression level of RGS2 which is above a control expression level is indicative of a favorable therapeutic outcome

33. The method according to claim 30 wherein said patient is diagnosed with B-precursor acute lymphoblastic leukemia (B-ALL).

34. The method according to claim 33 wherein said patient is a pediatric patient.

35. The method according to claim 30 wherein said gene products further include AGAP-1 (Arf GAP with GTP-binding protein-like, ANK repeat and PH domains) and/or PCDH17 (Protocadherin-17).

36. A method for screening compounds useful for treating acute lymphoblastic leukemia comprising:
(a) determining the expression level for at least three gene products selected from the group consisting of the gene products of Table 1P or alternatively, Table 1Q in a cell culture to yield observed gene expression levels prior to contact with a candidate compound;
(b) contacting the cell culture with a candidate compound;
(c) determining the expression level for the gene products in the cell culture to yield observed gene expression levels after contact with the candidate compound; and
(d) comparing the observed gene expression levels before and after contact with the candidate compound wherein a change in the gene expression levels after contact with the compound is indicative of therapeutic utility for said compound.

37. The method according to claim 36 wherein said gene products are selected from the group consisting of BMPR1B; CA6; CRLF2; GPR110; IGJ; LDB3; MUC4; NRXN3; PON2; and SEMA6A and an observed expression level of BMPR1B; CA6; CRLF2; GPR110; IGJ; LDB3; MUC4; NRXN3; PON2; and/or SEMA6A which is the same as or higher than a control expression level is indicative of an unfavorable or inactive therapeutic compound.

38. The method according to claim 36 wherein said gene products are selected from the group consisting of BMPR1B; CA6; CRLF2; GPR110; IGJ; LDB3; MUC4; NRXN3; PON2; and SEMA6A and an observed expression level of BMPR1B; CA6; CRLF2; GPR110; IGJ; LDB3; MUC4; NRXN3; PON2; and/or SEMA6A which is less than a control expression level is indicative of a favorable therapeutic outcome.

39. The method of claim 36 wherein said at least three gene products includes CRLF-2.

40. The method of claim 36 comprising determining the expression level for at least five of said gene products.

41. The method according to claim 36 wherein said leukemia is B-precursor acute lymphoblastic leukemia (B-ALL).

42. The method according to claim 41 wherein said leukemia is pediatric B-ALL.

43. The method according to claim 36 wherein said gene products further include AGAP-1 (Arf GAP with GTP-binding protein-like, ANK repeat and PH domains) and/or PCDH17 (Protocadherin-17).

44. A method for screening compounds useful for treating acute lymphoblastic leukemia comprising:
(a) contacting an experimental cell culture with a candidate compound;
(b) determining the expression level for at least three gene products selected from the group consisting of the gene products of Table 1P or alternatively, Table 1Q in the cell culture to yield experimental gene expression levels; and
(c) comparing the experimental gene expression levels of step b) to the expression level of the gene products in a control cell culture, wherein a relative difference in the gene expression levels between the experimental and control cultures is indicative of therapeutic utility.

45. The method according to claim 44 wherein said gene products are selected from the group consisting of BMPR1B; CA6; CRLF2; GPR110; IGJ; LDB3; MUC4; NRXN3; PON2; RGS2; SEMA6A and mixtures thereof.

46. The method according to claim 45 wherein the expression of all eleven gene products is measured and compared to expression of said eleven gene products in said control cell culture.

47. The method according to claim 44 wherein said gene products includes CRLF2.

48. The method according to claim 44 wherein said gene products further include AGAP-1 (Arf GAP with GTP-binding protein-like, ANK repeat and PH domains) and/or PCDH17 (Protocadherin-17).

49. (canceled)

50. (canceled)

51. (canceled)

52. (canceled)

53. (canceled)

54. (canceled)

55. A method for predicting therapeutic outcome in a leukemia patient comprising:
(a) obtaining a biological sample from a patient;
(b) determining in said sample the expression level for at least three gene products selected from the group consisting of BMPR1B; CA6; CRLF2; GPR110; IGJ; LDB3; MUC4; NRXN3; PON2; RGS2 and SEMA6A to yield observed gene expression levels; and
(c) comparing the observed gene expression levels for the gene products to a control gene expression level selected from the group consisting of:
(i) the gene expression level for the gene products observed in a control sample; and
(ii) a predetermined gene expression level for the gene products;
wherein an observed expression levels that is higher or lower than the control gene expression levels is indicative of predicted therapeutic failure.

56. The method according to claim 55 wherein said leukemia is B-precursor acute lymphoblastic leukemia (B-ALL).

57. The method according to claim 55 wherein said leukemia is pediatric B-ALL.

58. The method according to claim 55 wherein said gene products include CRLF2.

59. The method according to claim 55 wherein said gene products further include AGAP-1 (Arf GAP with GTP-binding protein-like, ANK repeat and PH domains) and/or PCDH17 (Protocadherin-17).

60. The method according to claim 55 wherein said gene products wherein a more aggressive traditional therapy or an experimental therapy is recommended for said leukemia patient.

61. (canceled)

62. (canceled)

63. (canceled)

64. (canceled)

65. (canceled)

66. (canceled)

67. (canceled)

68. (canceled)

69. (canceled)

70. A kit comprising a microchip embedded thereon polynucleotide probes specific for at least two prognostic genes selected from the group as set forth in Table 1P or alternatively, Table 1Q.

71. The kit according to claim 70 wherein said prognostic genes are selected from the group consisting of BMPR1B; CA6; CRLF2; GPR110; IGJ; LDB3; MUC4; NRXN3; PON2; RGS2 and SEMA6A.

72. (canceled)

73. A kit comprising at least two antibodies which are each specific at least for two different polypeptides selected from the group consisting of gene products as set forth in Table 1P or alternatively, Table 1Q.

74. (canceled)

75. (canceled)
